(19) United States
(12) Patent Application Publication
(10) Pub. No.: US 2026/0132375 A1
(43) Pub. Date: May 14, 2026

(54) MODIFIED T CELLS AND USES THEREOF

(71) Applicant: Peter MacCallum Cancer Institute, Melbourne (AU)

(72) Inventors: Sinead Marie Reading, Southbank (AU); Paul Beavis, Reservoir (AU); Maria Nogueira De Menezes, Ascot Vale (AU); Isabelle Munoz, Fitzroy (AU); Ian Andrew Parish, North Melbourne (AU)

(21) Appl. No.: 19/121,743

(22) PCT Filed: Oct. 20, 2023
(86) PCT No.: PCT/AU2023/051048

§ 371 Date: Apr. 16, 2025

(30) Foreign Application Priority Data
Oct. 20, 2022 (AU) 2022903088

(51) Int. Cl. C12N 5/0783; C12N 5/10

(52) U.S. Cl. CPC C12N 5/0636; C12N 5/10; C12N 2510/00

(57) ABSTRACT

The present disclosure relates generally to T cells that are modified to enhance the efficiency of adoptive cell therapy (ACT) by enhancing T cell function (e.g., CAR-T cell effector function), without affecting persistence. The present disclosure also relates to a pharmaceutical composition comprising the modified T cell (e.g., a population of modified T cells), methods for the preparation of an adoptive cell therapy and methods of adoptive cell therapy comprising administering the modified T cell (e.g., a population of modified T cells). In particular embodiments, the modified T cells described herein are useful for the treatment of cancer or viral infections.

### RELATED APPLICATIONS

This application claims priority from Australian Provisional Patent Application No. 2022903088 filed on 20 Oct. 2022, the entire content of which is hereby incorporated by reference.

### FIELD

The present disclosure relates generally to T cells that are modified to enhance the efficiency of adoptive cell therapy (ACT) by enhancing T cell function (e.g., CAR-T cell effector function), without affecting persistence. The present disclosure also relates to a pharmaceutical composition comprising the modified T cell (e.g., a population of modified T cells), methods for the preparation of an adoptive cell therapy and methods of adoptive cell therapy comprising administering the modified T cell (e.g., a population of modified T cells). In particular embodiments, the modified T cells described herein are useful for the treatment of cancer or viral infections.

### BACKGROUND

Adoptive cellular therapy (ACT) using chimeric antigen receptor (CAR) T cells has been highly successful in treating hematological malignancies, such as acute lymphoblastic leukemia (ALL) chronic lymphocytic leukemia (CLL) and multiple myeloma (Kalos et al. 2011, Science Translational Medicine, 3(95): 95ra73; Maude et al. 2014, New England Journal of Medicine, 371(16): 1507-17; San-Miguel et al. 2023, New England Journal of Medicine, 389: 335-347), but success in treating solid tumors has been limited due to immunosuppression in the local tumor microenvironment. The hypoxic solid tumor microenvironment leads to metabolic dysfunction of CAR-T cells, resulting in their exhaustion. It has been previously shown that modulation of CAR-T cells to promote a less differentiated phenotype enhances persistence and anti-tumor activity, particularly when combined with PD-1 blockade. The less differentiated phenotype is characterized by high expression of TCF1 and CD62L in mice, while expression of CCR7 is associated with increased persistence of CAR-T cells in human patients. Recent data also indicate that mitochondrial dysfunction is intrinsically linked to terminal differentiation and loss of cells with the less differentiated phenotype (Scharping et al., 2021, Nature Immunology, 22(2): 205-215; Yu et al., 2020, Nature Immunology, 21(12): 1540-1551; Vardhana et al., 2020, Nature Immunology, 21(9): 1022-1033). Significant therapeutic opportunity exists in enriching for and/or maintaining populations of cells with the less differentiated phenotype to avoid the problems associated with terminal exhaustion.

More recently, adoptive cell transfer of virus-specific T cell populations has been used to treat or prevent viral infections in patients with immunodeficiency, e.g., patients undergoing hematopoietic stem cell transplantation. However, T cell dysfunction (e.g., terminal exhaustion, terminal effector differentiation, senescence and activation-induced cell death) in ACT commonly occurs during chronic infections due to the persistence of antigen and inflammation.

Engineering T cells to mitigate or avoid the effects of T cell dysfunction (e.g., terminal exhaustion) and the loss of cells with the less differentiated phenotype has the potential to improve the efficacy of adoptive cell therapy. Consequently, there is a need to generate modified T cells (e.g., CAR-T cells) with enhanced effector function and maintained expansion and/or persistence to improve the efficacy of adoptive cell therapy for patients, particularly those with solid tumors or viral infections.

### SUMMARY

In one aspect, the present disclosure provides a modified T cell characterized by reduced level or activity, or both, of at least one IKAROS Zinc Finger transcription factor relative to an unmodified T cell, wherein the IKAROS Zinc Finger transcription factor is selected from the group consisting of IKAROS Family Zinc Finger I (IKAROS), IKAROS Family Zinc Finger 2 (HELIOS), IKAROS Family Zinc Finger 3 (AIOLOS) and IKAROS Family Zinc Finger 4 (EOS).

In another aspect, there is provided a pharmaceutical composition comprising the modified T cell described herein.

In another aspect, there is provided a method of adoptive cell therapy, the method comprising administering an effective amount of the modified T cell described herein to a subject in need thereof.

In another aspect, there is provided a use of the modified T cell described herein in the manufacture of a medicament for the treatment of cancer.

In another aspect, there is provided a use of the modified T cell described herein in the manufacture of a medicament for the treatment of a viral infection.

In another aspect, there is provided a method of preparing an adoptive cell therapy, the method comprising:
a. obtaining a population of T cells;
b. contacting the population of T cells with an agent for a time an under conditions suitable to reduce the level or activity, or both, of at least one IKAROS Zinc Finger transcription factor relative to a T cell that has not been contacted with the agent, wherein the IKAROS Zinc Finger transcription factor is selected from the group consisting of IKAROS, HELIOS, AIOLOS and EOS; and
c. culturing the population of T cells from step (b) for a time and under conditions suitable to prepare an expanded population of T cells.

### BRIEF DESCRIPTION OF THE DRAWINGS

Embodiments of the disclosure are described herein, by way of non-limiting example only, with reference to the accompanying drawings.

FIG. 1 shows the function and cytotoxicity of IKAROS Zinc Finger transcription factors (Ikzf) CRISPR-edited CAR-T cells targeting Her2 following in vitro co-culture of Ikzf CRISPR-edited CAR-T cells with EO771-Her2 tumor cells. (A) A graphical representation of normalized absolute number of CD8+ T cells at day 3 (y-axis; normalized relative number of CD8) across various Ikzf knockout groups (x-axis). (B) A graphical representation of the normalized percentage of CD8+ Granzyme B+ cells at day 3 (y-axis; normalized % granzyme B+ cells) across various Ikzf knockout groups (x-axis). A graphical representation of (C) TNF secretion; (D) IFNγ secretion; and (E) IL-2 secretion (y-axis; normalized concentration) in supernatants collected on day 1, 2 or 3 of the co-culture assay across various Ikzf knockout groups (x-axis). (F) A graphical representation of tumor killing as determined by Caspase 3/7 expression (y-axis; percentage of cancer cells Caspase 3/7+High) and time (x-axis; hour) measured by IncuCyte, ratio 4T/1C. (C)-(E) Data represents the combined data from at least two independent experiments. Control group was compared to other groups using one-way ANOVA, *p<0.05, **p<0.01, *** p<0.001, ****p<0.0001. (F) Data representative of two separate experiments. Control group was compared to other groups using two-way ANOVA, ****p<0.0001. (B)-(F) Results are shown as mean±SEM.

FIG. 2 shows the phenotype of Ikzf CRISPR-edited CAR-T cells targeting Her2 following in vitro co-culture of Ikzf CRISPR-edited CAR-T cells with EO771-Her2 tumor cells. A graphical representation of the normalized absolute number of (A) naïve (CD44−, CD62L+) T cells; (B) central memory (CD44+, CD62L+) T cells; and (C) effector memory (CD44+, CD62L−) T cells (y-axis; normalized total absolute number) across various IKZF knockout groups. A graphical representation of the normalized absolute number of (D) CD101+ T cells and (E) CX3CR1+ T cells (y-axis; normalized total absolute number) across various Ikzf knockout groups. (F) A series of graphical representations of the expression of CX3CR1 on control cells (top left panel), Ikzf1−/− CRISPR-edited CAR-T cells (top right panel), Ikzf1/2−/− CRISPR-edited CAR-T cells (bottom left panel) and Ikzf1/3−/− CRISPR-edited CAR-T cells (bottom right panel). (G) A series of graphical representations of the expression of PD1 and TIM3 on (from left to right): control cells (first panel), Ikzf1−/− CRISPR-edited CAR-T cells (second panel), Ikzf2−/− CRISPR-edited CAR-T cells (third panel), Ikzf1/2−/− CRISPR-edited CAR-T cells (fourth panel) and Ikzf1/3−/− CRISPR-edited CAR-T cells (fifth panel). (H) A graphical representation of the percentage of CD8+/TIM3+/PD1+ cells (y-axis; %) at day 3 across various Ikzf knockout groups (x-axis). (A)-(H) Data representative of the combined data from three independent experiments. Control group was compared to other groups using one-way ANOVA, *p<0.05, **p<0.01, ***p<0.001, ****p<0.0001.

FIG. 3 shows in vivo anti-tumor activity following adoptive transfer of various Ikzf CRISPR-edited CAR-T cells targeting Her2 to EO771-Her2 tumor-bearing mice. (A) A graphical representation of tumor size (y-axis; mm²) and time (x-axis; days post-treatment) after adoptive transfer of 20×10⁶ Ikzf1−/−, Ikzf2−/− or Ikzf3−/− KO CRISPR-edited CAR-T cells targeting Her2. Control, n=19; Ikzf1−/−, n=21; Ikzf2−/−, n=15; and Ikzf3−/−, n=16. (B) A graphical representation of tumor size (y-axis; mm²) and time (x-axis; days post-treatment) after adoptive transfer of 20×10⁶ Ikzf1/2−/−, Ikzf1/3−/−, Ikzf2/3−/− or Ikzf1/2/3−/− CRISPR-edited CAR-T cells targeting Her2. Control, n=19; Ikzf1/2−/−, n=23; Ikzf1/3−/−, n=23; Ikzf2/3−/−, n=22; and Ikzf1/2/3−/−, n=13. (C) A graphical representation of Kaplan-Meier survival (y-axis; probability of survival) and time (x-axis; days) after adoptive transfer of Ikzf1−/−, Ikzf2−/− or Ikzf3−/− CRISPR-edited CAR-T cells targeting Her2. (D) A graphical representation of Kaplan-Meier survival (y-axis; probability of survival) and time (x-axis; days) after adoptive transfer of Ikzf1/2−/−, Ikzf1/3−/−, Ikzf2/3−/− or Ikzf1/2/3−/− CRISPR-edited CAR-T cells targeting Her2. (A)-(B) Control group was compared to other groups using two-way ANOVA, **p<0.01, ***p<0.001. (C)-(D) Survival endpoint, tumor >150 mm². Control group compared to other groups using Mantel-Cox test.

FIG. 4 shows the phenotype of Ikzf CRISPR-edited CAR-T cells following adoptive transfer to EO771-Her2+ tumor-bearing mice. A graphical representation of the proportion of (A) PD1+/TIM3+ T cells; (B) effector memory T cells; and (C) central memory T cells (y-axis; % within CAR+ T cells) in peripheral blood collected at day 12 post-adoptive transfer of various Ikzf knockout groups (x-axis). (D) A graphical representation of the proportion of intratumoral PD1+/TIM3+ T cells (y-axis; % within CAR+ T cells) from tumors harvested at day 9 post-adoptive transfer of various Ikzf knockout groups (x-axis). A graphical representation of the proportion of intratumoral (E) IFNγ+ T cells; and (F) TNF+ T cells (y-axis; % within CAR+ T cells) from tumors harvested at day 9 post-adoptive transfer of various Ikzf KO CRISPR-edited CAR-T cells (x-axis) following in vitro stimulation with PMA/ionomycin/GolgiStop/GolgiPlug. (G) A graphical representation of the number of CD8+ T cells (y-axis; relative number of CD8) from spleens harvested at day 9 post-adoptive transfer of various Ikzf knockout groups (x-axis). A graphical representation of the proportion of (H) central memory T cells, (I) effector memory T cells; (J) PD1+/TIM3+ T cells; and (K) Ki67+ T cells (y-axis; % within CAR+ T cells) from spleens harvested at day 9 post-adoptive transfer of various Ikzf knockout groups (x-axis). A graphical representation of (L) naïve T cell count; (M) central memory T cell count; (N) effector memory T cell count; (O) PD1+/TIM3+ T cell count; (P) CD101+ T cell count; (Q) CX3CR1+ T cell count; (R) SLAM6− T cell count; and (S) TCF7+ T cell count (y-axis; normalized cells count) from spleens harvested at day 9 post-adoptive transfer of various Ikzf knockout groups (x-axis). (T) A graphical representation of tumor weight (y-axis; mg) from tumors harvested after day 14 post-adoptive transfer of various Ikzf knockout groups (x-axis). Control group was compared to other groups using one-way ANOVA, *p<0.05, *** p<0.0001. (U) A series of graphical representations of the expression of IFNγ expression on (from left to right): control cells (first panel), Ikzf1/2−/− CRISPR-edited CAR-T cells (second panel) and Ikzf1/3−/−CRISPR-edited CAR-T cells (third panel) from intratumoral CAR-T cells harvested from tumor at day 14 post-adoptive transfer and stimulated in vitro for 4 hours with PMA/Ionomycin/GolgiStop/GolgiPlug. (A)-(C) and (L)-(S) Data representative of the combined data from at least two independent experiments. Control group was compared to other groups using one-way ANOVA, **p<0.01, ***p<0.001, ****p<0.0001. (D)-(J) Data obtained from a single experiment. Control group was compared to other groups using one-way ANOVA, *p<0.05, **p<0.01, ***p<0.001, ****p<0.0001.

FIG. 5 shows the phenotype of Ikzf CRISPR-edited human CAR-T cells targeting Lewis Y following in vitro co-culture of Ikzf CRISPR-edited CAR-T cells with MCF7 Lewis Y+ tumor cells. A graphical representation of the proportion of (A) CD39+; (B) IFNγ+; (C) TNF+; and (D) PD1+TIM3+ CAR-T cells at day 3. (A)-(D) Data obtained from a single experiment. Control group was compared to other groups using one-way ANOVA, **p<0.01, ***p<0.001, ****p<0.0001.

FIG. 6 shows the anti-tumor activity and phenotype of dominant negative Ikzf (IKZF DN) CRISPR-edited CAR-T cells targeting Her2 following adoptive transfer to EO771-Her2+ tumor bearing mice. (A) A graphical representation of tumor size (y-axis; mm²) and time (x-axis; days post-treatment) after adoptive transfer of 20×10⁶ CAR-T cells targeting Her2 (control or IKZF DN CAR-T cells). (B) A graphical representation of normalized CD8+ T cell counts (y-axis; number of cells/mg of tumor) at day 9 post-adoptive transfer of control or IKZF DN CAR-T cells (x-axis). (C) A graphical representation of the proportion (y-axis; percentage within CAR+ T cells) of intratumoral naïve, central memory and effector memory T cells (x-axis) from tumors harvested at day 9 post-adoptive transfer of control CAR-T cells (black bars) or IKZF DN CAR-T cells (grey bars). A graphical representation of the proportion of intratumoral (D) TIM3+/PD1+ T cells; (E) CX3CR1+ T cells; (F) TCF− T cells; and (G) SLAMF6− T cells (y-axis; percentage within CAR+ T cells) from tumors harvested at day 9 post-adoptive transfer of control CAR-T cells or IKZF DN CAR-T cells (x-axis). A graphical representation of the proportion of intratumoral (H) IFNγ+ T cells; (I) TNF+ T cells; and (J) Granzyme B+ T cells (y-axis; percentage within CAR+ T cells) from tumors harvested at day 9 post-adoptive transfer of control CAR-T cells or IKZF DN CAR-T cells (x-axis) following in vitro stimulation with PMA/ionomycin/GolgiStop/GolgiPlug. (A) Data obtained from a single experiment. Control group was compared to other groups using two-way ANOVA, ***p<0.001, ****p<0.0001. Data shown as mean±SEM. (B)-(J) Data obtained from a single experiment. Control group was compared to other groups using one-way ANOVA. Data shown as mean±SEM.

FIG. 7 shows the phenotype of IKZF DN CRISPR-edited CAR-T cells targeting Her2 following in vitro co-culture of IKZF DN CRISPR-edited CAR-T cells with EO771-Her2, MC38-Her2 or AT3-Her2 tumor cells. A graphical representation of the proportion of (A) naïve (CD44−, CD62L+) T cells; (B) central memory (CD44+, CD62L+) T cells; (C) effector memory (CD44+, CD62L−) T cells; (D) PD1+/TIM3+ T cells; (E) CX3CR1+ T cells; (F) CD101+ T cells; and (G) SLAMF6+ T cells (y-axis; percentage within CAR+ T cells) across co-cultures with various tumor cell lines (x-axis). (H) A graphical representation of the final normalized absolute number of CD8+ T cells (y-axis; normalized relative number of cells) across co-cultures with various tumor cell lines (x-axis). A graphical representation of (I) IFNγ secretion; and (J) TNF secretion (y-axis; concentration (pg/mL)) in supernatants collected on day 3 following co-culture of control CAR-T cells (black bars) or IKZF DN CAR-T cells (grey bars) with various Her2+ tumor lines (x-axis). (A)-(J) Data representative of combined data from two independent experiments. Control group was compared to other groups by one-way ANOVA, **p<0.01, ****p<0.0001. Data shown as mean±SEM.

FIG. 8 shows that inhibition of the entire Ikzf family of Zinc Finger transcription factors (i.e., Ikzf1, Ikzf2, Ikzf3 and Ikzf4) increases the proportion of less differentiated T cells and causes excessive T cell response in mice infected with chronic lymphocytic choriomeningitis virus (LCMV). (A) A graphical representation of survival (y-axis; percent survival) and time (x-axis; days post-infection) of mice infected with chronic lymphocytic choriomeningitis virus (LCMV) in wild-type mice (black line) and Lck-cre; Ikzf2^{f/f}; Ikzf1^{mut/+} (IKZF KO) mice (grey line). (B) A graphical representation of the number of virus-specific CD8+ T cells (y-axis; cell number ×10⁵) in the spleen of wild-type or IKZF KO mice (x-axis) after infection with LCMV. (C) A graphical representation of the TNF and IL-2 production (x-axis) by IFNγ+ virus-specific T cells at day 7 post-infection in wild-type (left panel) and IKZF KO (right panel) mice as measured by flow cytometry. (D) A graphical representation of TNF production (y-axis; % TNF+) by IFNγ+ virus-specific T cells at day 7 post-infection in wild-type and IKZF KO mice (x-axis). (E) A graphical representation of stem-like TCF1+ subsets (x-axis) of CD8+ virus-specific T cells at day 7 post-infection in wild-type (left panel) and IKZF KO (right panel) mice as measured by flow cytometry. (F) A graphical representation of stem-like TCF1+ subsets (y-axis; % TCF1+) of CD8+ virus-specific T cells at day 7 post-infection in wild-type and IKZF KO mice (x-axis).

FIG. 9 shows the phenotype of LCMV-specific CRISPR-edited P14 T cells using sgRNA targeting Ikzf1, Ikzf2 and/or Ikzf3. (A) A graphical representation of the number of P14 T cells (y-axis; number of P14 T cells ×10³) at day 20 post-infection with LCMV-Clone 13 strain following adoptive transfer of 5000 CRISPR-edited P14 T cells using sgRNA targeting Ikzf1, Ikzf2 and/or Ikzf3 (x-axis). (B) A graphical representation of stem-like TCF1+ T cell number (y-axis; TCF1+P14 cell number ×10³) at day 20 post-infection with LCMV-Clone 13 strain following adoptive transfer of CRISPR-edited P14 T cells using sgRNA targeting Ikzf1, Ikzf2 and/or Ikzf3 (x-axis). (C) A graphical representation of IFNγ production capacity after re-stimulation (y-axis; IFNγ MFI of IFNγ+P14 cells (relative to control)) at day 20 post-infection with LCMV-Clone 13 strain following adoptive transfer of CRISPR-edited P14 T cells using sgRNA targeting Ikzf1, Ikzf2 and/or Ikzf3 (x-axis). (D) A graphical representation of TNF production after restimulation (y-axis; % TNF+ of IFNγ+P14 cells (relative to control)) at day 20 post-infection with LCMV-Clone 13 strain following adoptive transfer of CRISPR-edited P14 T cells using sgRNA targeting Ikzf1, Ikzf2 and/or Ikzf3 (x-axis).

FIG. 10 shows the differential transcription effects of LCMV-specific Ikzf CRISPR-edited P14 T cells following adoptive transfer to B6 mice infected with the chronic LCMV Clone 13. (A) A graphical representation of a principal component analysis of LCMV-specific Ikzf1−/−, Ikzf2−/−, Ikzf3−/−, Ikzf1/2−/−, Ikzf 1/3−/− and Ikzf2/3−/− CRISPR-edited P14 T cells showing differential transcription effects on T cell phenotype described by PC1 (x-axis) and PC2 (y-axis). (B) A heat map of differentially expressed genes of LCMV-specific Ikzf1−/−, Ikzf2−/−, Ikzf3−/−, Ikzf1/2−/−, Ikzf 1/3−/− and Ikzf2/3−/− CRISPR-edited P14 T cells.

FIG. 11 shows that exhausted LCMV-specific Ikzf CRISPR-edited P14 T cells have epigenetically unique differentiation states that do not normally exist in wild-type control cells (identified by circles) as identified by single cell ATACseq.

FIG. 12 shows the therapeutic effect of Ikzf1/3−/− CRISPR-edited CAR-T cells targeting CD19 following adoptive transfer to B-cell acute lymphoblastic leukemia (BALL) tumor bearing mice. (A) A graphical representation of Kaplan-Meier survival (y-axis; probability of survival) and time (x-axis; days post-tumor transplant) after adoptive transfer of Ikzf1/3−/− KO CRISPR-edited CAR-T cells targeting CD19. (B) A graphical representation of CAR T cell expansion/number (y-axis; CAR-T cells/μL) and time (x-axis; days post-tumor transplant) after adoptive transfer of Ikzf1/3−/− CRISPR-edited CAR-T cells targeting CD19. (C) A graphical representation of B cell aplasia (y-axis; total B cell count ×10\hat{ }9/L) and time (x-axis; days post-tumor transplant) after adoptive transfer of Ikzf1/3−/− CRISPR-edited CAR-T cells targeting CD19.

FIG. 13 shows the improved function of human Ikzf CRISPR-edited CAR-T cells targeting the Lewis Y antigen following in vitro co-culture with Lewis Y-MCF7 tumor cells. A graphical representation of (A) TNF secretion at day 1; (B) TNF secretion at day 3; (C) IFNγ secretion at day 1; (D) IFNγ secretion at day 3; and (E) IL-2 secretion (y-axis; normalized concentration) in supernatants collected on day 1 or 3 of the co-culture assay across various Ikzf knockout groups (x-axis). (F) A graphical representation of cell number (y-axis; relative number of cells) in supernatants collected on day 3 of the co-culture assay across various Ikzf knockout groups (x-axis). Control group was compared to other groups using one-way ANOVA, *p<0.05, **p<0.01, *** p<0.001, ****p<0.0001.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

Unless otherwise defined, all technical and scientific terms used herein have the same meaning as commonly understood by one of ordinary skill in the art to which this invention belongs.

Unless otherwise indicated the molecular biology, recombinant protein, cell culture and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1− 4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The articles “a”, “an” and “the” include plural aspects unless the context clearly dictates otherwise. Thus, for example, reference to “a cell” includes a single cell, as well as two or more cells (e.g., a population of cells); reference to “an agent” includes a single agent, as well as two or more agents; and so forth.

In the context of this specification, the term “about” is understood to refer to a range of numbers that a person of skill in the art would consider equivalent to the recited value in the context of achieving the same function or result. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

As used herein, the term “derived from” shall be taken to indicate that a particular integer or group of integers has originated from the species specified, but has not necessarily been obtained directly from the specified source.

The present disclosure is predicated, in part, on the surprising finding that modifying T cells to reduce the level or activity, or both, of at least one IKAROS Zinc Finger transcription factor improves expansion and/or persistence of T cells, while maintaining less differentiated populations. In some embodiments, targeting multiple IKAROS Zinc Finger transcription factors, e.g., IKAROS Family Zinc Finger 1 (IKAROS), IKAROS Family Zinc Finger 2 (HELIOS), IKAROS Family Zinc Finger 3 (AIOLOS) and/or IKAROS Family Zinc Finger 4 (EOS), further improves the results observed with reducing the level or activity, or both, of IKAROS alone. Beneficially, the modified T cells disclosed herein have improved cytotoxicity and anti-tumor or anti-viral efficacy, relative to unmodified T cells.

Accordingly, in an aspect disclosed herein, there is provided a modified T cell characterized by reduced level or activity, or both, of at least one IKAROS Zinc Finger transcription factor relative to an unmodified T cell, wherein the IKAROS Zinc Finger transcription factor is selected from the group consisting of IKAROS, HELIOS, AIOLOS and EOS.

### Modified T Cells

The terms “T cell” or “T lymphocyte” as well known in the art and refer to include thymocytes, naïve T lymphocytes, immature T lymphocytes, mature T lymphocytes, resting T lymphocytes, activated T lymphocytes or tumor infiltrating lymphocytes (TILs). Illustrative populations of T cells suitable for use in particular embodiments include but are not limited to helper T cells (HTL; CD4⁺ T cell), a cytotoxic T cell (CTL; CD8⁺ T cell), CD4⁺CD8⁺ T cell, CD4⁻CD8⁻ T cell, or any other subset of T cells. Other illustrative populations of T cells suitable for use in particular embodiments include but are not limited to T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

In certain embodiments, the population of T cells comprise naïve T cells (e.g., CD25−, CD45RA+, CD45RO− and/or CD127+), effector memory T cells (e.g., CD25−, CD45RA−, CD45RO+ and/or CD127+), central memory T cells (CD25+, CD45RA− and/or CD45RO+), and combinations of the foregoing.

The term “cell” as used herein refers to an individual cell, cell line, cell culture or population of cells that comprise the nucleic acid molecule or vectors described herein, or that is capable of expressing the fusion protein described herein. The term “population of cells” may refer to homogenous cell populations comprising central memory T cells, or heterogeneous cell populations that may comprise naïve T cells, effector memory T cells and/or central memory T cells. It is also contemplated herein the heterogeneous cell populations comprise the progeny of a single parental cell. Due to natural, accidental or deliberate mutation, the progeny cells may not necessarily be identical in morphology or in genome to the original parental cell.

The terms “IKAROS Family Zinc Finger 1” or “IKAROS” as used herein refer to a transcription factor that belongs to the Ikaros family of zinc finger DNA-binding proteins. IKAROS is encoded by the gene Ikzf1 (e.g., SEQ ID NO: 1), which is expressed in the hemo-lymphopoietic system. Several alternatively spliced transcript variants encoding different isoforms have been described for Ikzf1. Most isoforms share a common C-terminal domain, which contains two zinc finger motifs that are required for hetero- or homo-dimerization, and for interactions with other proteins. The isoforms, however, differ in the number of N-terminal zinc finger motifs that bind DNA and in nuclear localization signal presence, resulting in members with and without DNA-binding properties. The term “IKAROS” as used herein is intended to encompass all isoforms of IKAROS.

The terms “IKAROS Family Zinc Finger 2” or “HELIOS” as used herein refer to a transcription factor that belongs to the Ikaros family of zinc finger DNA-binding proteins. HELIOS forms homo- or hetero-dimers with other Ikaros family members, and functions predominantly in early hematopoietic development. HELIOS is encoded by the gene Ikzf2 (e.g., SEQ ID NO: 2), which is expressed in the hemo-lypmhopoietic system. Multiple transcript variants encoding different isoforms have been described for Ikzf2. The term “HELIOS” as used herein is intended to encompass all isoforms of HELIOS.

The terms “IKAROS Family Zinc Finger 3” or “AIOLOS” as used herein refer to a transcription factor that belongs to the Ikaros family of zinc finger DNA-binding proteins. AIOLOS is encoded by the gene Ikzf3 (e.g., SEQ ID NO: 3), which is expressed in the hemo-lypmhopoietic system. AIOLOS is associated with the regulation of B lymphocyte proliferation and differentiation. However, regulation of gene expression in B lymphocytes by AIOLOS is complex and is understood to require the sequential formation of IKAROS homodimers, AIOLOS homodimers and IKAROS/AIOLOS heterodimers. Multiple transcript variants encoding different isoforms have been described for Ikzf3. The term “AIOLOS” as used herein is intended to encompass all isoforms of AIOLOS.

The terms “IKAROS Family Zinc Finger 4” or “EOS” as used herein refer to a transcription factor that belongs to the Ikaros family of zinc finger DNA-binding proteins. EOS is encoded by the gene Ikzf4 (e.g., SEQ ID NO: 4), which is preferentially expressed in T-regulatory (Treg) cells. Multiple transcript variants encoding different isoforms have been described for Ikzf4. The term “EOS” as used herein is intended to encompass all isoforms of EOS.

In an embodiment, the modified T cell is characterized by a reduced level or activity, or both of IKAROS relative to an unmodified T cell.

In another embodiment, the modified T cell is further characterized by a reduced level or activity, or both of HELIOS, AIOLOS or EOS relative to an unmodified T cell.

Accordingly, in an embodiment, the modified T cell is characterized by a reduced level or activity, or both of IKAROS and HELIOS relative to an unmodified T cell.

In another embodiment, the modified T cell is characterized by a reduced level or activity, or both of IKAROS and AIOLOS relative to an unmodified T cell.

In another embodiment, the modified T cell is characterized by a reduced level or activity, or both of IKAROS and EOS relative to an unmodified T cell.

In another embodiment, the modified T cell is characterized by a level or activity, or both of IKAROS, HELIOS and AIOLOS relative to an unmodified T cell.

In another embodiment, the modified T cell is characterized by a level or activity, or both of IKAROS, HELIOS and EOS relative to an unmodified T cell.

In another embodiment, the modified T cell is characterized by a level or activity, or both of IKAROS, HELIOS, AIOLOS and EOS relative to an unmodified T cell.

The term “reduced level” as used herein refers to a level of IKAROS, HELIOS, AIOLOS and/or EOS that is lower that observed in T cells in the absence of any modification (i.e., a modification to reduce the level or activity, or both of an IKAROS Zinc Finger transcription factor). The level of IKAROS, HELIOS, AIOLOS and/or EOS polypeptides or peptides can be measured using any methods known in the art for the detection and/or quantification of protein expression. Such methods would be known to persons skilled in the art, illustrative examples of which include immunological detection methods, such as Western Blotting and enzyme-linked immunosorbent assay (ELISA), flow cytometry and mass spectrometry.

The term “reduced activity” as used herein refers to activity of IKAROS, HELIOS, AIOLOS and/or EOS that is lower that observed in T cells in the absence of any modification. The activity of IKAROS, HELIOS, AIOLOS and/or EOS may be measured using any methods known in the art for the detection and/or quantification of, e.g., regulation of gene expression; regulating the development of effector T cell populations (e.g., CD4+ and CD8+ T cells); or regulation of cytokine signaling pathways, illustrative examples of which include quantification of absolute cell number of immune cell subpopulations (e.g., naïve, central memory and effector memory T cell subpopulations), cytometric bead array (CBA) of secreted immunomodulators, and gene expression analysis.

Accordingly, by “reduced level or activity, or both” it is meant that the modified T cells are characterized by: (i) a reduction in the level of IKAROS, HELIOS, AIOLOS and/or EOS relative to T cells in the absence of any modification; (ii) a reduction in the activity of IKAROS, HELIOS, AIOLOS and/or EOS relative to T cells in the absence of any modification; or (iii) a reduction in the level and activity of IKAROS, HELIOS, AIOLOS and/or EOS relative to T cells in the absence of any modification.

It is to be understood that the term “reduced” as used herein, does not necessarily imply that the level of IKAROS, HELIOS, AIOLOS and/or EOS has been eliminated or is reduced to an undetectable level. In some embodiments, the level or activity, or both of IKAROS, HELIOS, AIOLOS and/or EOS may be reduced by at least about 40% (e.g., at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

In an embodiment, the modified T cell further comprises a chimeric antigen receptor (CAR) or a T cell receptor (TCR).

The modified T cells may be provided with a construct (e.g., a vector) encoding a CAR or a TCR using any suitable method known in the art. Such methods include transfection, transduction, viral transduction, microinjection, lipofection, nucleofection, nanoparticle bombardment, transformation, conjugation and the like. The skilled person would readily understand and adapt any such method taking consideration of whether the construct is provided as a polynucleotide or within a vector. The term “recombinant T cell” as used herein refers to a T cell which comprises the constructs or vectors described herein. The term “recombinant T cell” includes the specific T cell and the progeny of the T cell.

As used herein the terms “polynucleotide”, “nucleic acid” or “nucleic acid molecule” mean a single- or double-stranded polymer of deoxyribonucleotide, ribonucleotide bases or known analogues or natural nucleotides, or mixtures thereof, and can include molecules comprising coding and non-coding sequences of a gene, sense and antisense sequences and complements, exons, introns, genomic DNA, cDNA, pre-mRNA, mRNA, rRNA, siRNA, shRNA, miRNA, tRNA, ribozymes, recombinant polypeptides, isolated and purified naturally occurring DNA or RNA sequences, synthetic RNA and DNA sequences, nucleic acid probes, primers and fragments.

The term “nucleotide” as used herein refers to the nucleotides adenosine, guanosine, cytidine, thymidine and uridine, each of which comprise a nucleotide base attached to a ribose ring. A person skilled in the art will appreciate that the terms “adenine/adenosine”, “uracil/uridine”, “guanine/guanosine”, “cytosine/cytidine” and “thymidine/thymine” may be used interchangeably herein with the single letters A, U, G, C and T, respectively, which refer the nucleotide base comprised by the nucleotides.

The terms “non-naturally occurring”, “engineered” or “recombinant” may be interchangeably used herein to refer to nucleotides or nucleic acid molecules that are distinguished from their naturally occurring counterparts. For example, the nucleic acid molecule of the present disclosure may be recombinant, synthetic, or comprise mixtures of naturally and non-naturally occurring nucleotides. Non-naturally occurring nucleotides or nucleotide analogs may be modified at the ribose, phosphate and/or base moiety.

As used herein, the terms “encode”, “encoding” and the like refer to the capacity of a nucleic acid molecule to provide for another nucleic acid or a polypeptide. For example, a nucleic acid molecule is said to “encode” a polypeptide if it can be transcribed and/or translated to produce the polypeptide or if it can be processed into a form that can be transcribed and/or translated to produce the polypeptide. Such a nucleic acid molecule may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

In an embodiment, the construct is a vector.

The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art. Preferably, however, the vector is suitable for use in biotechnology.

Vectors suitable for use in biotechnology would be known to persons skilled in the art, illustrative examples of which include viral vectors derived from adenovirus, adeno-associated virus (AAV), herpes simplex virus (HSV), retrovirus, lentivirus, self-amplifying single-strand RNA (ssRNA) viruses such as alphavirus (e.g., Semliki Forest virus, Sindbis virus, Venezuelan equine encephalitis, M1), and flavivirus (e.g., Kunjin virus, West Nile virus, Dengue virus), rhabdovirus (e.g., rabies, vesicular stomatitis virus), measles virus, Newcastle Disease virus (NDV) and poxvirus as described by, for example, Lundstrom (2019, Diseases, 6: 42).

In an embodiment, the vector is a plasmid or a viral vector.

The terms “chimeric antigen receptor” or “CAR” as used herein mean a recombinant polypeptide comprising at least an antigen-binding domain that is linked, via hinge and transmembrane domains, to an intracellular signaling domain.

The antigen-binding domain is a functional portion of the CAR that is responsible for transmitting information within the cell to regulate cellular activity via defined signaling pathways. In an embodiment, the antigen-binding domain may comprise an antibody or antibody fragment thereof.

The term “antibody” is used herein in the broadest sense and specifically covers monoclonal antibodies (including full length monoclonal antibodies), polyclonal antibodies, chimeric antibodies, humanized antibodies, human antibodies, multi-specific antibodies (e.g., bispecific antibodies), and single variable domain antibodies so long as they exhibit the desired biological activity. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (CU). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments disclosed herein, the FRs of an antibody (or antigen-binding portion thereof) may be identical to the human germline sequences, or may be naturally or artificially modified. An amino acid consensus sequence may be defined based on a side-by-side analysis of two or more CDRs. Included within the scope of the term “antibody” is an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

An “antigen-binding fragment” may be provided by means of arrangement of one or more CDRs on non-antibody protein scaffolds. “Protein scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. The protein scaffold may be an Ig scaffold, for example an IgG, or IgA scaffold. The IgG scaffold may comprise some or all the domains of an antibody (i.e., CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3−CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (iv) V_{H}-C_{H1}-C_{H}2; (v) V_{H}-C_{H1}-C_{H2}-C_{H3}, (vi) V_{H}-C_{H2}-C_{H3}; (vii) V_{H}-CL; (viii) V_{L}-C_{H1}; (ix) V_{L}-C_{H2}, (X) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). As with full antibody molecules, antigen-binding fragments may be monospecific or multi-specific (e.g., bispecific). A multi-specific antigen-binding fragment of an antibody will typically comprise at least two different variable domains, wherein each variable domain is capable of specifically binding to a separate antigen or to a different epitope on the same antigen.

In an embodiment, the antigen-binding domain comprises an antibody fragment. For example, the antigen-binding domain may comprise a scFv consisting of a VL and VH sequence of a monoclonal antibody (mAb) specific for a tumor cell surface molecule (i.e., tumor antigen).

In an embodiment, the CAR comprises an antigen binding domain that binds specifically to an antigen selected from the group consisting of CD19, CD20, CD22, CD30, ROR1, CD123, CD33, CD133, CD138, GD2, Her2, Her1, mesothelin, MUC1, gp100, MART-1, MAGE-A3, MUC16, NY-ESO-1, L1-CAM, CEA, FAP, VEGFR2, WT1, TAG-72, CD171, α-FR, CAIX, PSMA, Lewis Y, BCMA, GPRC5D and HIV env protein. In another embodiment, the CAR comprises an antigen binding domain that binds to Her2 or Lewis Y.

In an embodiment, the CAR comprises a signaling domain selected from the group consisting of CD3ζ, CD28, 41BB, DAP10, OX40, ICOS, DAP12, KIR2DS2, 4-1BB, CD3s, CD35, CD3C, CD25, CD27, CD79A, DC79B, CARD11, FcRa, Fcftp, FcRy, Fyn, HVEM, Lck, LAG3, LAT, LRP, NKG2D, NOTCH1, NOTCH2, NOTCH3, NOTCH4, ROR2, Ryk, SLAMF1, Slip76, pTa, TCRa, TCRP, TRIM, Zap70, PTCH2 and LIGHT. In another embodiment, the CAR comprises a signaling domain selected from the group consisting of CD28 and CD3ζ. In a preferred embodiment, the CAR comprises the CD28 and CD3ζ signaling domains.

The terms “T-cell receptor” or “TCR” as used herein mean a recombinant or naturally-occurring heterodimeric polypeptide comprising an alpha polypeptide chain (i.e., alpha chain, a chain) and a beta polypeptide chain (i.e., beta chain, β chain), which is capable of binding to a peptide antigen or neoantigen bound to MHC.

In an embodiment, the TCR binds specifically to an antigen selected from the group consisting of 707-AP, AFP, ART-4, BAGE, Bcr-abl, CAMEL, CAP-1, CASP-8, CDC27m, CDK4/m, CEA, CT, Cyp-B, DAM, EGFRvlll, ELF2M, ETV6-AML1, G250, GAGE, GnT-V, Gp100, HAGE, HER-2/neu, HLA-A, HPV, HSP70-2M, HST-2, hTERT, hTRT, iCE, KIAA0205, LAGE (L antigen), LDLR/FUT, MAGE, MART-1/Melan-A, MC1 R, Myosin/m, MUC1, MUM-1, MUM-2, MUM-3, NA88-A, NY-ESO-1, P15, p190 minor, Pml/RARa, PRAME, PSA, PSMA, RAGE, RU1, RU2, SAGE, SART-1, SART-3, SSX1, SSX2, SSX3, SSX4, TEL/AML1, TPI/m, TRP-1, TRP-2, TRP-2/INT2 and WT1.

In an embodiment, the TCR binds to a viral-specific antigen. Suitable viral-specific antigens would be known to persons skilled in the art, illustrative examples of which include antigens specific for adenovirus, bocavirus, coronavirus, HHV6, lymphocytic choriomeningitis virus (LCMV), mumps and measles, metapneumovirus, parainfluenza, parovirus B19, RSV, rotavirus, West Nile virus, BK virus, cytomegalovirus (CMV), Epstein-Barr virus (EBV), human immunodeficiency virus (HIV) and hepatitis C virus (HCV).

In an embodiment, TCR binds to a LCMV-specific antigen.

In an embodiment, the TCR binds specifically to a neoantigen.

The term “neoantigen” as used herein refers to antigens generated by tumor cells as a result of tumor-specific alterations, such as somatic genomic alterations in coding and non-coding regions (e.g., single-nucleotide variants, base insertions and deletions (INDELs), gene fusions, structural variants), aberrant RNA splicing, aberrant post-translational modification and gene rearrangement. Neoantigens can also be produced by viral infection, e.g., by integrated viral open reading frames.

In an embodiment, the TCR binds specifically to a neoantigen selected from the group consisting of KRASG12D, KRASG12C, FLT3D835Y, ERBB2IP, p53R175H, BCR-ABL, SYT-SSX1/SSX2, PAX3-FOXO1, TPM3/TPM4-ALK, and EWS-FLI1.

The modified T cells contemplated herein may be derived from any species, particularly a vertebrate, and even more particularly a mammal. Suitable vertebrates that fall within the scope of the disclosure include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars, etc.), marine mammals (e.g., dolphins, whales), reptiles (snakes, frogs, lizards, etc.), and fish.

In an embodiment, the modified T cell is derived from a mammalian donor. In another embodiment, the modified T cell is derived from a human donor.

According to the present disclosure, T cells may be isolated by any means known in the art. The term “isolated” as used herein refers to material, such as a cell, which is substantially or essentially free from components which normally accompany or interact with it as found in its naturally occurring environment. The isolated material optionally comprises material not found with the material in its natural environment.

In an embodiment, T cells are isolated from whole blood using a Ficoll-Paque separation method. The Ficoll-Paque method is used to isolate mononuclear cells from blood using low viscosity Ficoll and sodium metrizoate or sodium diatrizoate. This method is well known in the art and adaptable to isolate mononuclear cells from peripheral blood, umbilical cord blood and bone marrow.

In an embodiment, T cells are obtained from peripheral blood (e.g., peripheral blood mononuclear cells or “PBMCs”).

In an embodiment, the modified T cell is an autologous modified T cell.

The term “autologous” as used herein refers to any material derived from the same subject to whom it is later to be administered into the subject in accordance with the methods disclosed herein. Accordingly, in certain embodiments, T cells isolated from the subject may be contacted with the genome editing systems described herein and cultured ex vivo for a time and under conditions suitable for the integration of the heterologous nucleotide sequence, before being reinfused back into the subject in accordance with the method of treatment described herein.

In another embodiment, the T cell is an allogenic modified T cell.

The term “allogenic” as used herein refers to any material derived from a different animal of the same species as the subject to whom the material is administered.

### Genetic Modifications

In certain embodiments, the modified T cell comprises a genetic modification leading to reduced expression of a gene encoding at least one IKAROS Zinc Finger transcription factor relative to an unmodified T cell, wherein the gene is selected from the group consisting of Ikzf1, Ikzf2, Ikzf3 and Ikzf4.

The term “genetic modification” as used herein refers to any modification to a gene encoding at least one IKAROS Zinc Finger transcription factor, i.e., Ikzf1, Ikzf2, Ikzf3 and Ikzf4, that leads to a reduction in level or activity, or both, of the at least one IKAROS Zinc Finger transcription factor.

In an embodiment, the modified T cell comprises a genetic modification leading to reduced expression of Ikzf1 relative to an unmodified T cell.

In an embodiment, the modified T cell further comprises a genetic modification genetic modification leading to reduced expression of Ikzf2, Ikzf3 or Ikzf4 relative to an unmodified T cell.

In an embodiment, the modified T cell comprises a genetic modification leading to reduced expression of Ikzf1 and Ikzf2 relative to an unmodified T cell.

In an embodiment, the modified T cell comprises a genetic modification leading to reduced expression of Ikzf1 and Ikzf3 relative to an unmodified T cell.

In an embodiment, the modified T cell comprises a genetic modification leading to reduced expression of Ikzf1 and Ikzf4 relative to an unmodified T cell.

In an embodiment, the modified T cell comprises a genetic modification leading to reduced expression of Ikzf1, Ikzf2 and Ikzf4 relative to an unmodified T cell.

In an embodiment, the modified T cell comprises a genetic modification leading to reduced expression of Ikzf1, Ikzf2, Ikzf3 and Ikzf4 relative to an unmodified T cell.

In an embodiment, the genetic modification is a mutation selected from a deletion mutation, an insertion mutation, a substitution mutation, a splice-site mutation, a premature translation termination mutation, a nonsense mutation and a frameshift mutation.

The term “mutation” as used herein does not include silent nucleotide substitutions which do not affect the gene activity. The term “polymorphism” refers to any change in the nucleotide sequence including such silent nucleotide substitutions. Screening methods may first involve screening for polymorphisms and secondly for mutations within a group of polymorphic variants. As used herein, the term “mutation” includes deletions of all or part of a gene, insertions such as an insertion into an exon of a gene, nucleotide substitutions, splice-site mutations, premature translation termination mutations, frameshift mutations, nonsense mutations and any combination thereof.

The term “reduced expression” as used herein means a level of expression that is lower than observed in T cells in the absence of the genetic modification. It is to be understood that the term “reduced” as used herein, does not necessarily imply that the expression of Ikzf1, Ikzf2, Ikzf3 and/or Ikzf4 has been eliminated or is reduced to an undetectable level. In some embodiments, the level of expression of Ikzf1, Ikzf2, Ikzf3 and/or Ikzf4 may be reduced by at least about 40% (e.g., at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

In an embodiment, the expression of Ikzf1, Ikzf2, Ikzf3 and/or Ikzf4 is reduced to an undetectable level. Persons skilled in the art will appreciate that a reduction is expression to an undetectable level is intended to encompass embodiments whereby the expression of Ikzf1, Ikzf2, Ikzf3 and/or Ikzf4 is effectively abolished, i.e., “knocked out”.

Accordingly, in an embodiment, the modified T cell is an Ikzf1−/−, Ikzf2−/−, Ikzf3−/− or an Ikzf4−/− knockout cell.

In an embodiment, the modified T cell is an Ikzf1/2−/− knockout cell.

In another embodiment, the modified T cell is an Ikzf1/3−/− knockout cell.

In another embodiment, the modified T cell is an Ikzf1/4−/− knockout cell.

In another embodiment, the modified T cell is an Ikzf1/2/3−/− knockout cell.

In another embodiment, the modified T cell is an Ikzf1/2/4−/− knockout cell.

In another embodiment, the modified T cell is an Ikzf1/2/3/4−/− knockout cell.

In an embodiment, the modification that reduces the level or activity, or both, of at least one IKAROS Zinc Finger transcription factor is mediated by a biological agent.

As used herein the term “biological agent” means any agent useful in producing site-specific mutants and includes enzymes that induce double stranded breaks in DNA that stimulate endogenous repair mechanisms. These include endonucleases, zinc finger nucleases, TAL effector proteins, transposases, site-specific recombinases and are CRISPR endonucleases. Zinc finger nucleases (ZFNs), e.g., facilitate site-specific cleavage within a selected gene within a genome allowing endogenous or other end-joining repair mechanisms to introduce deletions or insertions to repair the gap.

In an embodiment, the biological agent is selected from the group consisting of a RNA interference molecule and a CRISPR gene editing system.

The term “RNA interference molecule” as used herein refers to any molecule that is capable of reducing the expression of Ikzf1, Ikzf2, Ikzf3 and/or Ikzf4 by gene silencing.

The term “gene silencing” as used herein refers to the reduction of expression of a target nucleic acid in a T cell, which can be achieved by the introduction of a silencing RNA. In some embodiments, a gene silencing chimeric gene is introduced into a T cell, which encodes a RNA molecule which reduces the expression of one or more endogenous genes, at least Ikzf1, Ikzf2, Ikzf3 and/or Ikzf4. Such reduction may be the result of reduction of transcription, including by methylation of promoter regions via chromatin re-modelling, or post-transcriptional modification of the RNA molecules, including via RNA degradation, or both. Gene silencing should not necessarily be interpreted as an abolishing the expression of the target nucleic acid or gene. It is sufficient that the level expression of the target nucleic acid in the presence of the silencing RNA is lower than that observed in the absence thereof. Accordingly, in some embodiments, the level of expression of the targeted gene may be reduced by at least about 40% (e.g., at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

Methods for gene silencing would be known to persons skilled in the art, illustrative examples of which include the stable introduction and transcription of a gene silencing chimeric gene, sense-suppression techniques, anti-sense techniques and RNA interference (RNAi).

Antisense techniques may be used to reduce gene expression in T cells. The term “antisense RNA” as used herein means an RNA molecule that is complementary to at least a portion of a specific mRNA molecule and capable of reducing expression of the gene encoding the mRNA, preferably Ikzf1, Ikzf2, Ikzf3 and/or Ikzf4. Such reduction typically occurs in a sequence-dependent manner and is thought to occur by interfering with a post-transcriptional event such as mRNA transport from nucleus to cytoplasm, mRNA stability or inhibition of translation.

As used herein, the phrase “artificially-introduced dsRNA molecule” refers to the introduction of double-stranded RNA (dsRNA) molecule, which preferably is synthesized in a T cell by transcription from a chimeric gene encoding such dsRNA molecule. RNA interference (RNAi) is particularly useful for specifically reducing the expression of a gene or inhibiting the production of a particular protein, and is known to be effective in T cells (Freeley and Long, 2013, The Biochemical Journal, 455(2): 133-147). This technology relies on the presence of dsRNA molecules that contain a sequence that is essentially identical to the mRNA of the gene of interest or part thereof, and its complement, thereby forming a dsRNA. Conveniently, the dsRNA can be produced from a single promoter in the host cell, where the sense and anti-sense sequences are transcribed to produce a hairpin RNA in which the sense and anti-sense sequences hybridize to form the dsRNA region with a related (to Ikzf1, Ikzf2, Ikzf3 and/or Ikzf4) or unrelated sequence forming a loop structure, so the hairpin RNA comprises a stem-loop structure. The design and production of suitable dsRNA molecules for the present invention is well within the capacity of a person skilled in the art.

The DNA encoding the dsRNA typically comprises both sense and antisense sequences arranged as an inverted repeat. In a preferred embodiment, the sense and antisense sequences are separated by a spacer region which may (or may not) comprise an intron which, when transcribed into RNA, is spliced out. This arrangement has been shown to result in a higher efficiency of gene silencing. The double-stranded region may comprise one or two RNA molecules, transcribed from either one DNA region or two. The dsRNA may be classified as long hpRNA, having long, sense and antisense regions which can be largely complementary, but need not be entirely complementary (typically larger than about 200 bp, e.g., between 200 and 1000 bp).

The length of the sense and antisense sequences that hybridize should each be at least 19 contiguous nucleotides (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides, and so on), preferably at least 21 contiguous nucleotides, 30 contiguous nucleotides, 50 contiguous nucleotides, and more preferably at least 100, 200, 500 or 1000 contiguous nucleotides. The full-length sequence corresponding to the entire gene transcript may be used. The lengths are most preferably 100-2000 nucleotides. The degree of identity of the sense and antisense sequences to the targeted transcript should be at least 85% (e.g., at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively identical, i.e., 100%), preferably at least 90% and more preferably 95-100%. The longer the sequence, the less stringent the requirement for the overall sequence identity. The RNA molecule may comprise unrelated sequences which may function to stabilize the molecule. The promoter used to express the dsRNA-forming construct may be any type of promoter that is expressed in the cells which express the target gene, preferably a promoter which is preferentially expressed in T cells relative to other cells.

As used herein, “silencing RNAs” are RNA molecules that have 21 to 24 contiguous nucleotides that are complementary to a region of the mRNA transcribed from the target gene, preferably Ikzf1, Ikzf2, Ikzf3 and/or Ikzf4. The sequence of the 21 to 24 nucleotides is preferably fully complementary to a sequence of 21 to 24 contiguous nucleotides of the mRNA i.e., identical to the complement of the 21 to 24 nucleotides of the region of the mRNA. However, miRNA sequences which have up to five mismatches in region of the mRNA may also be used (Palatnik et al., 2003, Nature, 425: 257-263), and base-pairing may involve one or two G-U base-pairs. When not all of the 21 to 24 nucleotides of the silencing RNA are able to base-pair with the mRNA, it is preferred that there are only one or two mismatches between the 21 to 24 nucleotides of the silencing RNA and the region of the mRNA. With respect to the miRNAs, it is preferred that any mismatches, up to the maximum of five, are found towards the 3′ end of the miRNA. In a preferred embodiment, there are not more than one or two mismatches between the sequences of the silencing RNA and its target mRNA.

Silencing RNAs derived from longer RNA molecules, also referred to herein as “precursor RNAs”, are the initial products produced by transcription from the chimeric DNAs in T cells and have partially double-stranded character formed by intra-molecular base-pairing between complementary regions. The precursor RNAs are processed by a specialized class of RNases, commonly called “Dicer(s)”, into the silencing RNAs, typically of 21 to 24 nucleotides long. Silencing RNAs as used herein include short interfering RNAs (siRNAs) and microRNAs (miRNAs), which differ in their biosynthesis. siRNAs derive from fully or partially double-stranded RNAs having at least 21 contiguous base-pairs, including possible G-U base-pairs, without mismatches or non-base-paired nucleotides bulging out from the double-stranded region. These double-stranded RNAs are formed from either a single, self-complementary transcript which forms by folding back on itself and forming a stem-loop structure, referred to herein as a “hairpin RNA”, or from two separate RNAs which are at least partly complementary and that hybridize to form a double-stranded RNA region (e.g., a “short hairpin RNA”, or “shRNA”). miRNAs are produced by processing of longer, single-stranded transcripts that include complementary regions that are not fully complementary and so form an imperfectly base-paired structure, so having mismatched or non-base-paired nucleotides within the partly double-stranded structure. The base-paired structure may also include G-U base-pairs. Processing of the precursor RNAs to form miRNAs leads to the preferential accumulation of one or more distinct, small RNAs each having a specific sequence, the miRNA(s). They are derived from one strand of the precursor RNA, typically the “antisense” strand of the precursor RNA, whereas processing of the long complementary precursor RNA to form siRNAs produces a population of siRNAs which are not uniform in sequence but correspond to many portions and from both strands of the precursor.

miRNA precursor RNAs, also termed herein as “artificial miRNA precursors”, are typically derived from naturally occurring miRNA precursors by altering the nucleotide sequence of the miRNA portion of the naturally-occurring precursor so that it is complementary, preferably fully complementary, to the 21 to 24 nucleotide region of the target mRNA, and altering the nucleotide sequence of the complementary region of the miRNA precursor that base-pairs to the miRNA sequence to maintain base-pairing. The remainder of the miRNA precursor RNA may be unaltered and so have the same sequence as the naturally occurring miRNA precursor, or it may also be altered in sequence by nucleotide substitutions, nucleotide insertions, or preferably deletions, or any combination thereof. The remainder of the miRNA precursor RNA is thought to be involved in recognition of the structure by the Dicer enzyme called Dicer-like 1 (DCL1), and therefore it is preferred that few if any changes are made to the remainder of the structure. For example, base-paired nucleotides may be substituted for other base-paired nucleotides without major change to the overall structure.

In an embodiment, the biological agent is a CRISPR gene editing system.

The term “gene editing” as used herein refers to a type of genetic alteration in which nucleic acid is inserted, replaced, or removed from a target nucleic acid sequence within a nucleic acid molecule, e.g., the genome of a cell, using one or more RNA-guided nucleases.

The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546). Briefly, upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence. Suitable Cas endonucleases would be known to persons skilled in the art, illustrative examples of which include Cas3, Cas9, Cas12 (e.g., Cas12a, Cas12b, Cas12c, Cas12d, Cas12e) and Cas14.

In an embodiment, the CRISPR gene editing system is a CRISPR-Cas9 gene editing system.

The CRISPR gene editing system of the present disclosure comprises a Cas endonuclease and at least one gRNA targeting the IKAROS Zinc Finger transcription factors described herein.

The terms “guide RNA” or “gRNA” refer to a RNA sequence that is complementary to a target nucleic acid sequence and directs a RNA-guided nuclease to the target nucleic acid sequence. gRNA typically comprises CRISPR RNA (crRNA) and a tracr RNA (tracrRNA). “crRNA” is a 17-20 nucleotide sequence that is complementary to the target nucleic acid sequence, while the “tracrRNA” provides a binding scaffold for the RNA-guided nuclease. crRNA and tracrRNA exist in nature a two separate RNA molecules, which has been adapted for molecular biology techniques using, for example, 2-piece gRNAs such as CRISPR tracer RNAs (cr:tracrRNAs) or a single RNA sequence that comprises the crRNA fused to the tracrRNA (single-guide RNA, or “sgRNA”).

Accordingly, the skilled person would understand that the term “gRNA” describes all CRISPR guide formats, including two separate RNA molecules or a single RNA molecule combining the crRNA and tracrRNA elements into a single nucleotide sequence (sgRNA).

In an embodiment, the gRNA is a single-guide RNA (sgRNA). In another embodiment, the sgRNA is selected from any one of SEQ ID NOs: 5-12.

Methods and tools for the design of gRNA would be known to persons skilled in the art, illustrative examples of which include CHOPCHOP, CRISPR Design, sgRNA Designer, Synthego and GT-Scan. Suitable gRNAs would be known to persons skilled in the art or could be designed and produced by persons skilled in the art, illustrative examples of which include the gRNAs in Table 1.

The CRISPR gene editing system may be provided to a T cell using any suitable method known in the art, illustrative examples of which include transduction of one or more vectors comprising a polynucleotide sequence(s) encoding the components of the CRISPR gene editing system and ribonucleoproteins (RNP). The present disclosure also provides non-viral delivery vehicles of the genome editing systems as described herein. Suitable non-viral delivery vehicles will be known to persons skilled in the art, illustrative examples of which include using lipids, lipid-like materials or polymeric materials, as described, for example, by Rui et al. (2019, Trends in Biotechnology, 37(3): 281-293), and nanoparticles/nanocarriers, as described by, for example, Nguyen et al. (2020, Nature Biotechnology, 38: 44-49), Duan et al. (2021, Frontiers in Genetics, 12: 673286), and Rahimi et al. (2020, Nanotoday, 34: 100895).

In an embodiment, the CRISPR gene editing system comprises a Cas endonuclease and at least one gRNA complexed as an RNP.

### Pharmaceutical Compositions

In another aspect disclosed herein, there is provided a pharmaceutical composition comprising the modified T cell described herein.

The term “pharmaceutical composition” as used herein refers to a composition that is in a form that allows the biological activity of the active ingredient (e.g., a modified T cell expressing a CAR or TCR, as described herein) to be effective, and that does not contain additional ingredients that have unacceptable toxicity to the subject to which the composition is to be administered.

In an embodiment, the pharmaceutical composition comprises the modified T cell in in a number sufficient to administer a dosage of 10⁴ to 10⁹ cells/kg body weight per dose. Accordingly, the pharmaceutical composition may comprise a population of the modified T cells in a number sufficient to administer a dosage of 10⁴, 10¹, 10⁶, 10⁷, 10⁸ or 10⁹ cells/kg body weight per dose.

In an embodiment, the pharmaceutical composition comprises a population of the modified T cells in a number sufficient to administer a dosage of 10⁵ to 10⁶ cells/kg body weight per dose, including all integer values within those ranges.

In some embodiments, periodic re-administration of the pharmaceutical composition may be required to achieve a desirable therapeutic effect. The exact amounts and rates of administration of the pharmaceutical composition will depend on a number of factors, examples of which are described elsewhere herein, such as the subject's age, body weight, general health, sex and dietary requirements, as well as any drugs or agents used in combination or coincidental with the administration of the composition. Where multiple divided doses are required, these may be administered hourly, daily, weekly, monthly or at other suitable time intervals or the dose may be proportionally reduced as indicated by the exigencies of the situation. Alternatively, a continuous infusion strategy can be employed.

In an embodiment, the pharmaceutical composition is suitable for parenteral administration. In another embodiment, the composition is suitable for intravenous administration.

The pharmaceutical compositions disclosed herein may be prepared according to conventional methods well known in the pharmaceutical industries, such as those described in Remington's Pharmaceutical Handbook (Mack Publishing Co., NY, USA), comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions, which may contain anti-oxidants, buffers and solutes to render the composition isotonic with the blood of the intended recipient, aqueous and non-aqueous sterile suspensions, which may include suspending agents and thickening agents, dispersion media, anti-fungal and anti-bacterial agents, isotonic and absorption agents, and the like.

In an embodiment, the pharmaceutical composition further comprises one or more immune adjuvants.

The term “immune adjuvant” as used herein refers to a compound or substance that is capable of enhancing a subject's immune response to the immunogen including, for example, the subject's antibody response to the immunogen. An immune adjuvant may therefore assist to enhance the immune response to modified T cell in a subject, compared to the administration of the modified T cell or in the absence of the immune adjuvant.

Suitable immune adjuvants will be familiar to persons skilled in the art, illustrative examples of which include an inhibitor of the PDL-1: PD-1 axis, a 4-1BB agonist, an inhibitor of TIM-3, and an inhibitor of CTLA-4.

In an embodiment, the pharmaceutical composition is for use in the treatment of cancer.

In an embodiment, the pharmaceutical composition is for use in the treatment of a viral infection.

### Methods of Adoptive Cell Therapy and Associated Therapeutic Uses

In an aspect disclosed herein, there is provided a method of adoptive cell therapy, the method comprising administering an effective amount of the modified T cell described herein to a subject in need thereof.

In an embodiments, periodic re-administration of the active agents may be required to achieve a desirable therapeutic effect.

The regimen for adoptive cell therapy can be determined by a person skilled in the art and will typically depend on factors including, but not limited to, the type, size, stage of the disease or disorder in addition to the age, weight and general health of the subject. Another determinative factor may be the risk of developing recurrent disease. For instance, for a subject identified as being at high risk or higher risk or developing recurrent disease, a more aggressive therapeutic regimen may be prescribed as compared to a subject who is deemed at a low or lower risk of developing recurrent disease. For example, for a subject identified as having a more advanced stage of cancer, such as stage III or IV disease, a more aggressive therapeutic regimen may be prescribed as compared to a subject that has a less advanced stage of cancer.

In an embodiment, the subject has cancer.

The term “cancer” as used herein means any condition associated with aberrant cell proliferation. Such conditions will be known to persons skilled in the art. In an embodiment, the cancer is a primary cancer (e.g., a tumor). In another embodiment, the cancer is a metastatic cancer.

Examples of various cancers are described elsewhere herein and include breast cancer, colorectal cancer, prostate cancer, ovarian cancer, cervical cancer, skin cancer, pancreatic cancer, renal cancer, liver cancer, brain cancer, lymphoma, leukemia, lung cancer, sarcoma and the like. The terms “cancer” and “tumor” may be used interchangeably herein, e.g., encompassing both solid and diffuse or circulating tumors.

In an embodiment, the cancer is a solid tumor. Suitable solid tumors would be known to persons skilled in the art, illustrative examples of which include breast cancer, melanoma, carcinoid, cervical cancer, ovarian cancer, pancreatic cancer, colorectal cancer, prostate cancer, endometrial cancer, renal cancer, glioma, skin cancer, head and neck cancer, stomach cancer, liver cancer, testis cancer, lung cancer, thyroid cancer, lymphoma and urothelial cancer.

In an embodiment, the cancer is a hematological cancer. Suitable hematological cancers would be known to persons skilled in the art, illustrative examples of which include chronic lymphocytic leukemia (CLL), mantle cell lymphoma (MCL), multiple myeloma, acute lymphoid leukemia (ALL), Hodgkin lymphoma, B-cell acute lymphoid leukemia (B-ALL), T-cell acute lymphoid leukemia (TALL), small lymphocytic lymphoma (SLL), B-cell prolymphocytic leukemia, blastic plasmacytoid dendritic cell neoplasm, Burkitt's lymphoma, diffuse large B-cell lymphoma (DLBCL), DLBCL associated with chronic inflammation, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia and myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, and unclassifiable lymphoma.

In an embodiment, the hematological cancer is selected from the group consisting of multiple myeloma and B-ALL.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human. In an embodiment, the subject is an adult. In another embodiment, the subject is a child.

In an embodiment, the subject has a viral infection.

In an embodiment, the infection is caused by a virus selected from the group consisting of adenovirus, bocavirus, coronavirus, HHV6/7, lymphocytic choriomeningitis virus (LCMV), mumps and measles, metapneumovirus, parainfluenza, paroviruses, RSV, rotavirus, West Nile virus, BK virus, cytomegalovirus (CMV) and Epstein-Barr virus (EBV), human papillomaviruses, Herpes viruses (e.g., Herpes simplex 1/2), varicella zoster, HTLV, human papovaviruses (e.g., JC virus and BK virus), HIV, HBV and HCV.

In an embodiment, the viral infection is a chronic viral infection.

In an embodiment, the modified T cell is autologous to the subject.

In another embodiment, the modified T cell is allogenic to the subject.

As used herein, the term “effective amount” typically refers to an amount of the modified T cell, or pharmaceutical composition described herein that is sufficient to affect one or more beneficial or desired therapeutic outcomes (e.g., reduction in tumor size, viral clearance). Said beneficial or desired therapeutic outcomes may be measured using clinical techniques known in the art, illustrative examples of which include the measurement of imaging biomarkers, tumor size (e.g., as measured by anatomical imaging modalities, such as CT or MRI), quantification of the presence of inflammatory mediators (e.g., Interleukin-1, TNF, TGF-β, etc.). An “effective amount” can be provided in one or more administrations. The exact amount required may vary depending on factors such as the nature and severity of the cancer to be treated, and the age and general health of the subject.

The terms “treat”, “treating”, “treatment” and the like are used interchangeably herein to mean relieving, reducing, alleviating, ameliorating or otherwise inhibiting the severity and/or progression of: (i) cancer, or a symptom thereof; or (ii) a viral infection, or a symptom thereof, in a subject. It is to be understood that the terms “treat”, “treating”, “treatment” and the like, as used herein, do not imply that a subject is treated until clinical symptoms of cancer of infection have been eliminated or are no longer evident (e.g., elimination of solid tumor mass and associated metastatic lesions, if any; or clearance of virus). Said treatment may also reduce the severity of cancer or infection by preventing progression or alleviating the symptoms associated with cancer or infection.

The terms “prevent”, “preventing”, “prevention” and the like are used interchangeably herein to mean inhibit, hinder, retard, reduce or otherwise delay the development of: (i) cancer and/or progression of cancer, or a symptom thereof; or (ii) a viral infection and/or progression of viral infection, or a symptom thereof in a subject. In the context of the present disclosure, the term “prevent” and variations thereof does not necessarily imply the complete prevention of the specified event. Rather, the prevention may be to an extent, and/or for a time, sufficient to produce the desired effect. Prevention may be inhibition, retardation, reduction or otherwise hindrance of the event, activity or function. Such preventative effects may be in magnitude and/or be temporal in nature.

In another aspect disclosed herein, there is provided a method of preparing an adoptive cell therapy, the method comprising:
a. obtaining a population of T cells;
b. contacting the population of T cells with an agent for a time an under conditions suitable to reduce the level or activity, or both, of at least one IKAROS Zinc Finger transcription factor relative to a T cell that has not been contacted with the agent, wherein the IKAROS Zinc Finger transcription factor is selected from the group consisting of IKAROS, HELIOS, AIOLOS and EOS; and
c. culturing the population of T cells from step (b) for a time and under conditions suitable to prepare an expanded population of T cells.

In an embodiment, the method further comprises the step of:
d. transducing the expanded population of T cells with a construct comprising a nucleic acid molecule encoding a CAR or a TCR.

In an embodiment, step (b) comprises contacting the population of T cells with an agent for a time and under conditions suitable to reduce the level or activity, or both, of IKAROS relative to a population of T cells that have not been contacted with the agent

In an embodiment, step (b) further comprises contacting the population of T cells with an agent for a time and under conditions suitable to reduce the level or activity, or both, of HELIOS, AIOLOS or EOS relative to a population of T cells that have not been contacted with the agent.

In an embodiment, step (b) comprises contacting the population of T cells with an agent for a time and under conditions suitable to reduce the level or activity, or both, of IKAROS and HELIOS relative to a population of T cells that have not been contacted with the agent.

In an embodiment, step (b) comprises contacting the population of T cells with an agent for a time and under conditions suitable to reduce the level or activity, or both, of IKAROS and AIOLOS relative to a population of T cells that have not been contacted with the agent.

In an embodiment, step (b) comprises contacting the population of T cells with an agent for a time and under conditions suitable to reduce the level or activity, or both, of IKAROS and EOS relative to a population of T cells that have not been contacted with the agent.

In an embodiment, the agent is a biological agent.

In an embodiment, the biological agent is selected from the group consisting of a RNA interference molecule and a CRISPR gene editing system. In another embodiment, the biological agent is a CRISPR gene editing system.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### General Methods

### CRISPR-Mediated Gene Editing

Naïve human T cells were purified from normal Buffy coats using Ficoll centrifugation followed by naïve T cell purification using the EasySep Human Naïve Pan T cell Isolation Kit (StemCell). Murine T cells were isolated from lymphoid organs (spleen and lymph nodes) using the EasySep mouse CD8+ T cell Isolation Kit (StemCell). 5×10⁶ naïve T cells were resuspended in 100 μL OptiMEM, combined with ribonucleoprotein (RNP) complexes comprising 10⁸ pmoles recombinant Cas9 (IDT) and 1350 pmoles sgRNA (Synthego) as shown in Table 1, or similar mouse gene targeting sgRNAs, and electroporated using a 4D-Nucleofector (Lonza) using pulse code EO115 (human T cells) or DN100 (mouse T cells). For CAR T cell generation, CRISPR-edited naïve T cells were cultured in RPMI supplemented with 10% FCS, sodium pyruvate, glutamax, NEAA, HEPES and penicillin/streptomycin and activated for 48 hours with human Dynabeads (Gibco, #1131D), IL-2 (600 U/mL) and IL-7 (150 U/mL) as previously described by Giuffrida et al. (2021, Nature Communications, 12: 3236). For mouse viral experiments, CRISPR-edited mouse T cells were injected into recipient mice without further culture.

### Generation of CAR-T Cells

Retrovirus encoding a second-generation scFv-anti-Lewis-Y CAR, linked to a human CD8 hinge region and cytoplasmic domains of human CD28 and CD3z, was obtained from the supernatant of the PG13 packaging line, as previously described by Williford et al. (2019, Scientific Advances, 5(12): p.eaay1357).

Retronectin-coated (15 g/mL) 6-well plates (Takara Bio) were centrifuged at 1,200 g for 1 h with 4 mL of retroviral supernatant added to each well. 5×10⁶ CRISPR-edited T cells were resuspended in 1 mL of retrovirus containing IL-2/IL-7 and added to the retronectin-coated plates, which contain retroviral supernatant. Cells were centrifuged for 1 h (1200 g) followed by overnight incubation and a second round of transduction the next day. Cells were cultured at a density of 1×10⁶/mL following the transduction process.

The syngeneic tumor line MCF7-Lewis Y (a breast cancer cell line) naturally express the Lewis Y antigen.

Primary murine splenocytes were transduced with a CAR containing the CD28 and TCR-ζ signaling domains recognizing the human Her2 antigen (scFv-CD28-ζ) as described in (Mardiana et al. 2017, Cancer Research, 77: 1296-1309; John et al. 2013, Clinical Cancer Research, 19: 5636-5646; Beavis et al. 2017, The Journal of Clinical Investigation, 127(3): 929-941).

The syngeneic tumor lines AT3-Her2 (a mammary carcinoma cell line), EO771-Her2 (a triple negative breast cancer line) and MC38-Her2 (a colon carcinoma cell line) were engineered to express the human Her2 receptor. These lines are of C57/BL6 origin, allowing them to be transplanted into C57/BL6 hHer-2 transgenic mice. Using this model, the human Her2 receptor (minus intracellular signaling domains) is expressed under the control of the whey acidic protein promoter, resulting in expression in the cerebellum and breast tissues.

After 3 days of culture, CRISPR-edited human or murine CAR-T cells were co-cultured with MCF7 or EO771-Her2 for 3 days (ratio 3T/2C).

### In Vivo Analysis

Her2+ mice were inoculated with 2×10⁵ EO771-Her2 tumor cells (mammary fat pad injection) and allowed to establish to approximately 20 mm² followed by intravenous injection of 20×10⁶ CRISPR-edited murine CAR-T cells targeting Her2. Tumor growth and survival were assessed.

For viral infections, mice were infected with 2×10⁶ p.f.u. of chronic LCMV-Clone 13 strain i.v. In P14 experiments, C57BL/6 mice were injected with 5000 CRISPR-edited CD45.1+P14 cells 3 days prior to infection. Splenic T cell phenotypes were assessed by flow cytometry at the indicated time-points.

### Flow Cytometry (FACS) Analysis

Absolute CD8+, CD8+ Granzyme B+, naïve T cell (CD44−, CD62L+), central memory T cell (CD44+, CD62L+), effector memory T cell (CD44+, CD62L−), CD101+, CXCR1+, CD8+/TIM3+/PD1+, cell number were quantified at day 3 of the co-culture flow cytometry. Intracellular cytokine staining was conducted by staining for TNF and IFNγ in cells that were stimulated in vitro in the presence of Brefeldin A.

For CAR-T cell phenotype analysis, supernatant was collected every day of the co-culture assay and TNF, IFNγ and IL-2 secretion were quantified by cytometric bead array (CBA).

Measurement of tumor cell death was measured using the IncuCyte Immune Cell Killing Assay (Sartorius) in accordance with the manufacturer's instructions.

### Statistical Analysis

Statistical analyses were performed in Prism v9 software (GraphPad). A p-value ≤0.05 was considered statically significant. Data presented as mean±standard error of the mean (SEM).

### Example 1

### Function and Cytotoxicity of Murine CAR-T Cells Modified to Reduce the Level or Activity, or Both, of Ikaros Zinc Finger Transcription Factors

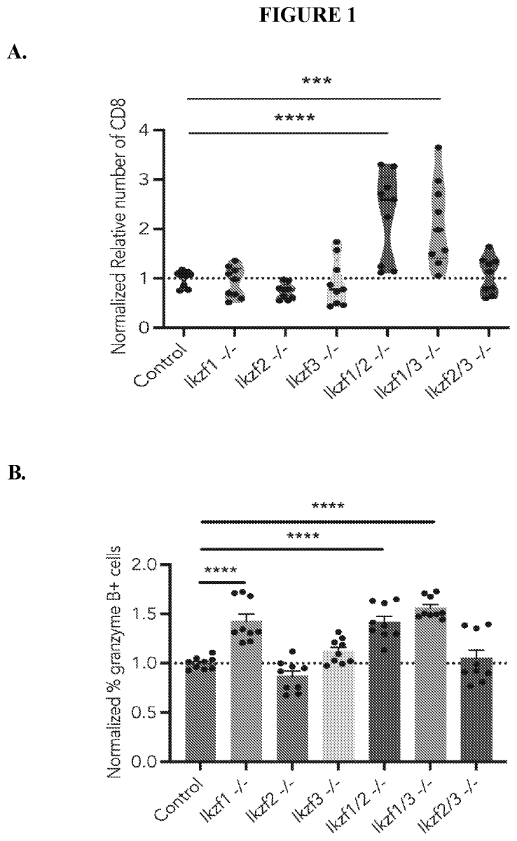

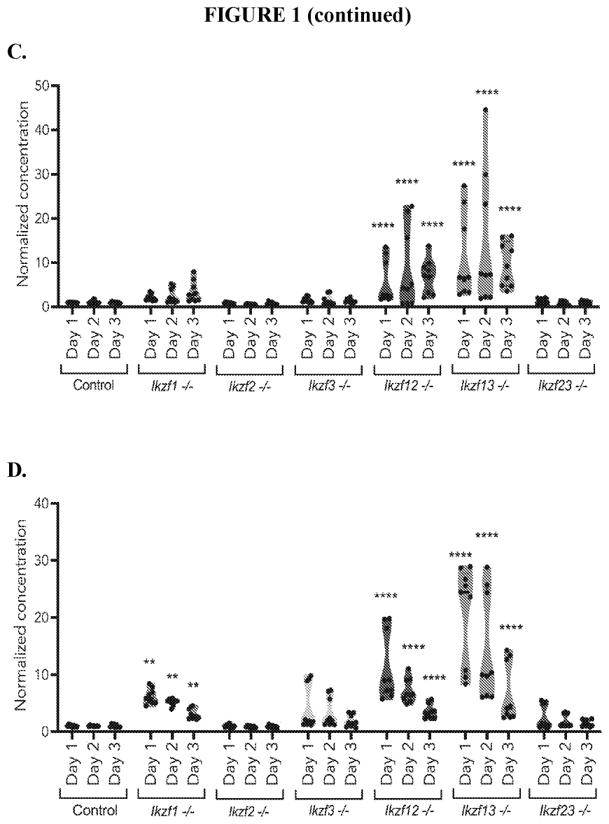

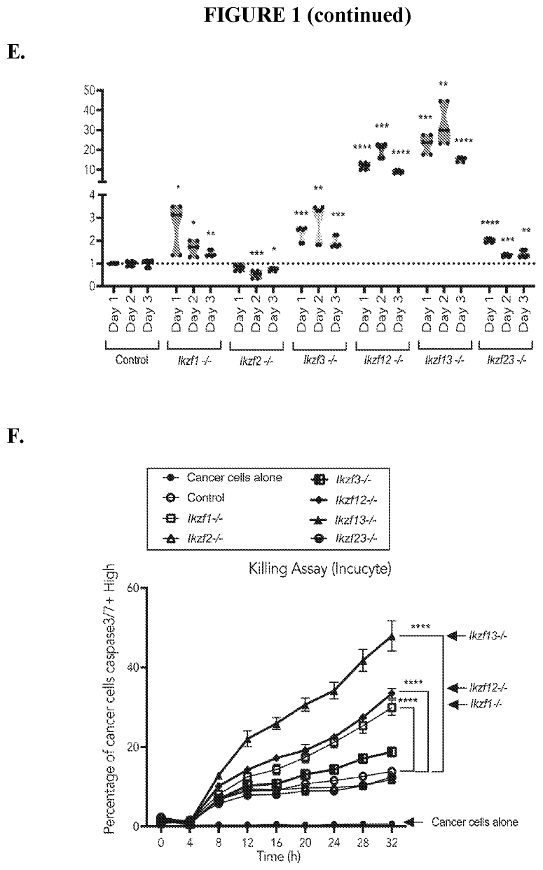

Murine anti-Her2 CAR-T cells were CRISPR-edited to reduce or eliminate the expression of at least one of Ikzf1, Ikzf2 and/or Ikzf3. The CRISPR-edited CAR-T cells were co-cultured with EO771-Her2 tumor cells for 3 days in vitro, at a ratio of 3 CAR-T cells/2 tumor cells (ratio 3T/2C) to determine the effect of IKAROS Zinc Finger transcription factors on CAR-T cell function and cytotoxicity. As shown in FIG. 1A, targeting of Ikzf1 and Ikzf2 (i.e., Ikzf1/2−/−) and Ikzf1 and Ikzf3 (i.e., Ikzf1/3−/−) resulted in a significant increase in CD8+ cell number relative to control, unmodified CAR-T cells. Significant upregulation of effector function (Granzyme B+) was also observed for Ikzf1−/−, Ikzf1/2−/− and Ikzf1/3−/− CAR-T cells.

To investigate the functional consequences of reduced or eliminated expression of Ikzf1, Ikzf2 and/or Ikzf3, the cytokine production of CRISPR-edited CAR-T cells was determined following co-culture with EO771-Her2. In these experiments, it was observed that the Ikzf1/2−/− and Ikzf1/3−/− CAR-T cells had higher levels of TNF (FIG. 1C), IFNγ (FIG. 1D) and IL-2 (FIG. 1E) secretion following antigen-specific stimulation with EO771-Her2 tumor cells relative to control CAR T cells. In addition, the Ikzf1/2−/−, Ikzf1/3−/− and Ikzf1−/− CAR-T cells demonstrated significantly increased cytotoxicity, reflected in the killing of Her2+ target cells in Incucyte assays (FIG. 1F).

Taken together, these data demonstrate that CAR-T cells modified to reduce the level or activity, or both, of at least one of Ikzf1, Ikzf2 and/or Ikzf3 enhances the in vitro activity of CAR-T cells, thus supporting the targeting of IKAROS Zinc Finger transcription factors to generate CAT-T cells with enhanced function.

### Example 2

### Phenotype of Murine CAR-T Cells Modified to Reduce the Level or Activity, or Both, of Ikaros Zinc Finger Transcription Factors

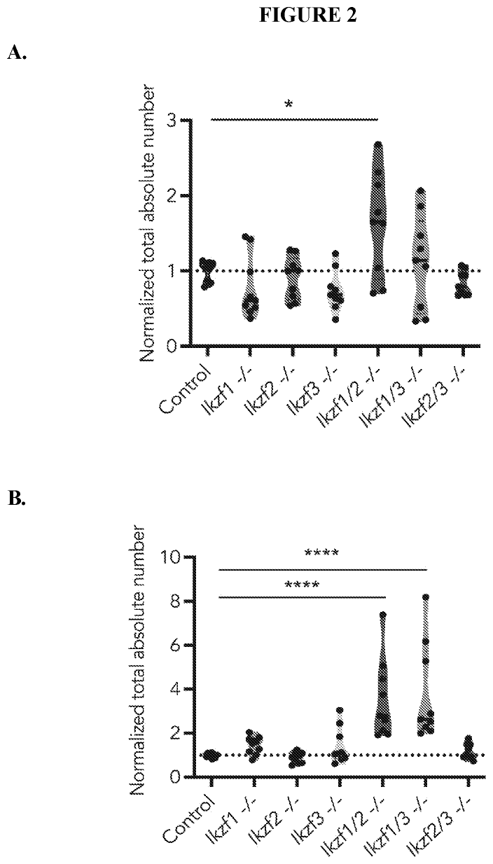

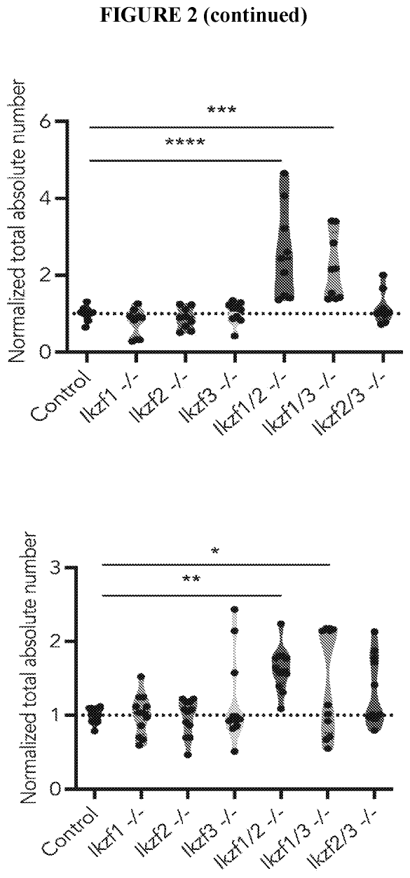

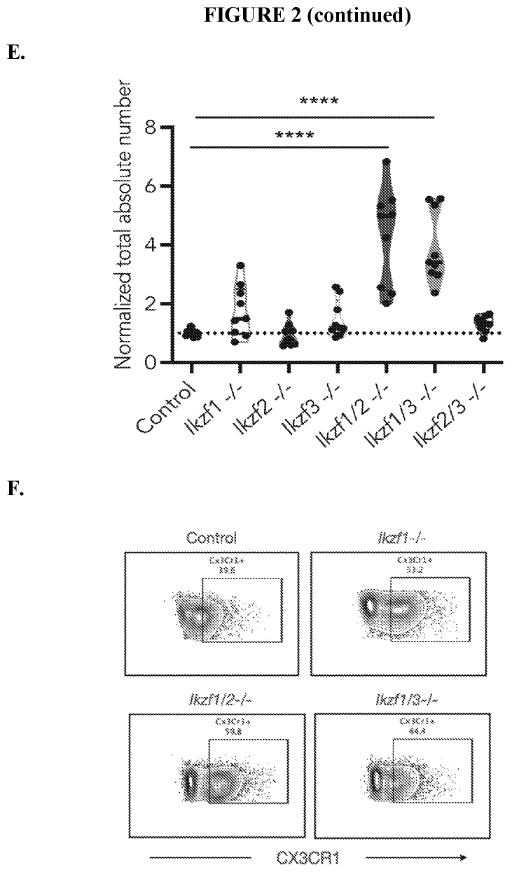

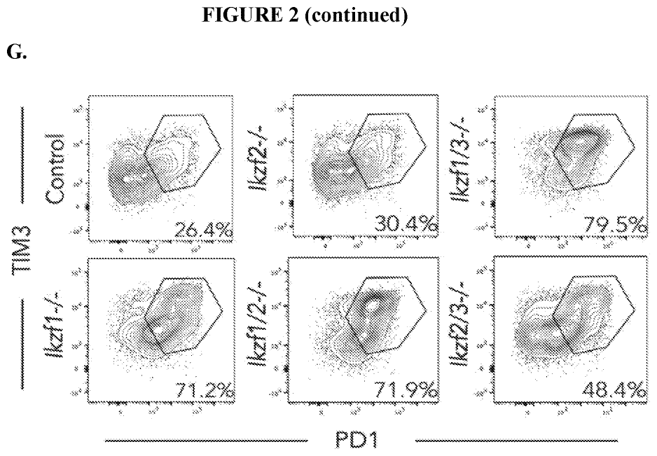

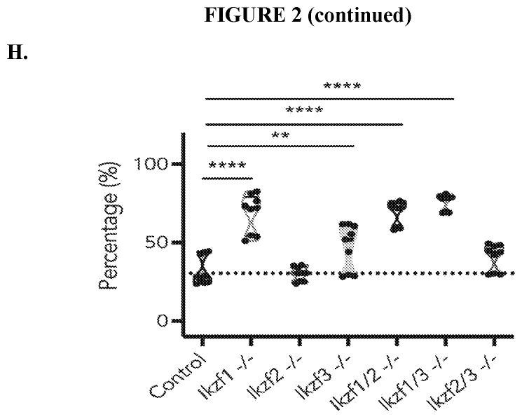

To investigate the functional consequences of reduced or eliminated expression of Ikzf1, Ikzf2 and/or Ikzf3, the phenotype of CRISPR-edited CAR-T cells was determined following co-culture with EO771-Her2. In these experiments, it was observed that the Ikzf1/2−/− CAR-T cells had significantly increased number of naive (CD44−/CD62L+), while both Ikzf1/2−/− and Ikzf1/3−/− had significantly increased central memory (CD44+/CD62L+) and effector memory (CD44+/CD62L−) counts (FIGS. 2A-C). These data indicate that the expansion of cells does not give rise to more effector memory and less naïve/central memory CAR-T cells, which may be anticipated from an increase in proliferation. Moreover, CAR-T cells that retain a central memory phenotype are expected to elicit greater persistence in vivo.

Similarly, Ikzf1/2−/− and Ikzf1/3−/− CAR-T cells significantly increased expression of activation markers, CD101+, CXCR1+ and TIM3+/PD1+(FIGS. 2D, 2E and 2H). These data are also summarized in Table 2. Expression of CXC3CR1 on Ikzf1−/−, Ikzf1/2−/− and Ikzf1/3−/− CAR-T cells was also increased relative to control CAR-T cells (FIG. 2F), as was the expression of TIM3 and PD1 (FIG. 2H).

Taken together, these data demonstrate that CAR-T cells modified to reduce the level or activity, or both, of at least one of Ikzf1, Ikzf2 and/or Ikzf3 promotes or maintains CAR-T cell populations with a less differentiated (i.e., stem-like) phenotype, together with increased expression of activation markers that are indicative of increased CAR-T cell activity in vitro.

### Example 3

### Anti-Tumor Activity of Murine CAR-T Cells Modified to Reduce the Level or Activity, or Both, of Ikaros Zinc Finger Transcription Factors

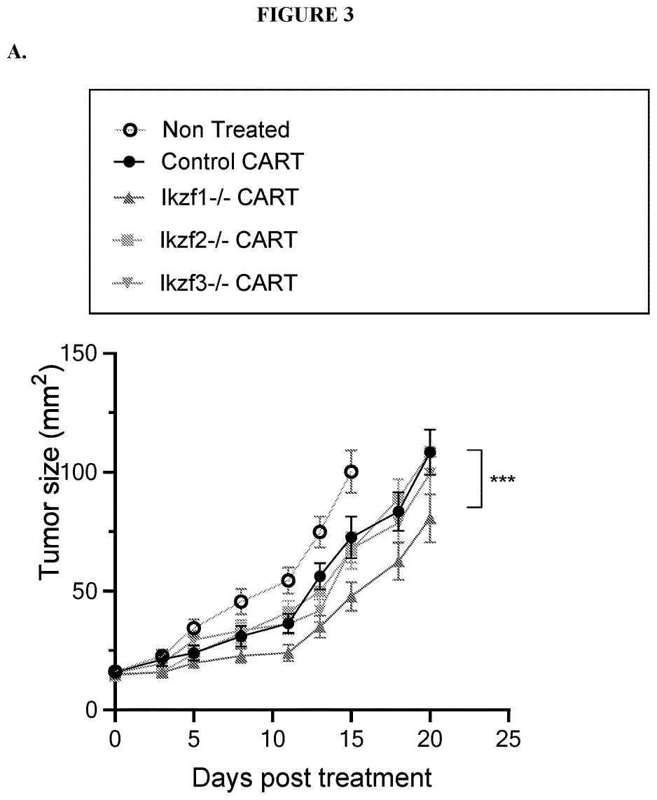

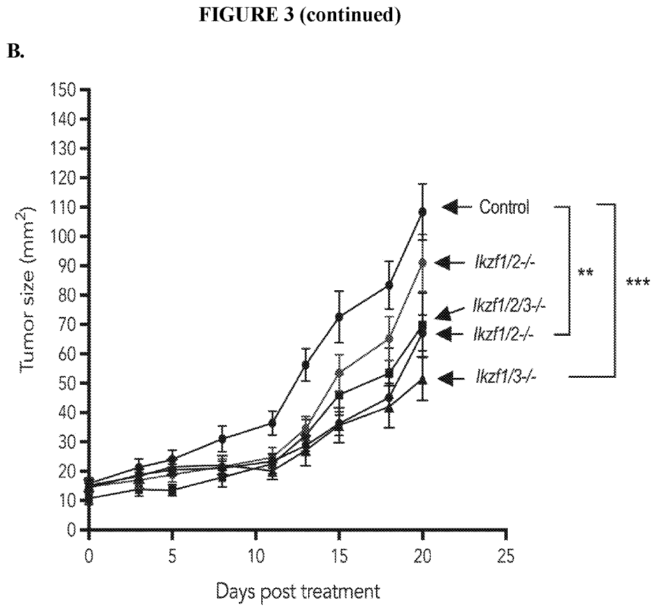

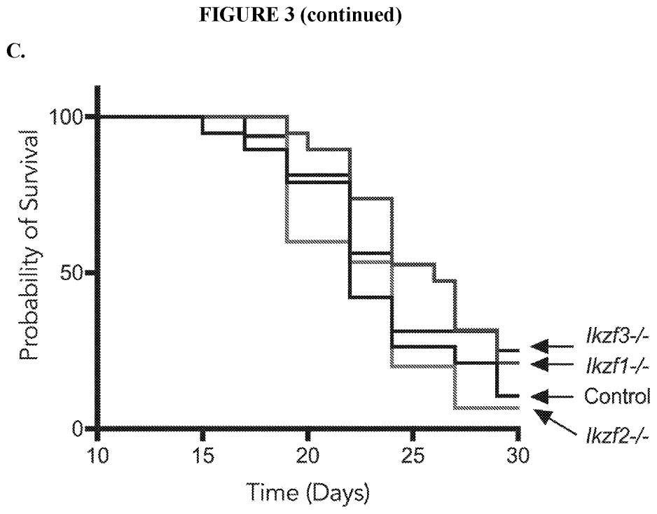

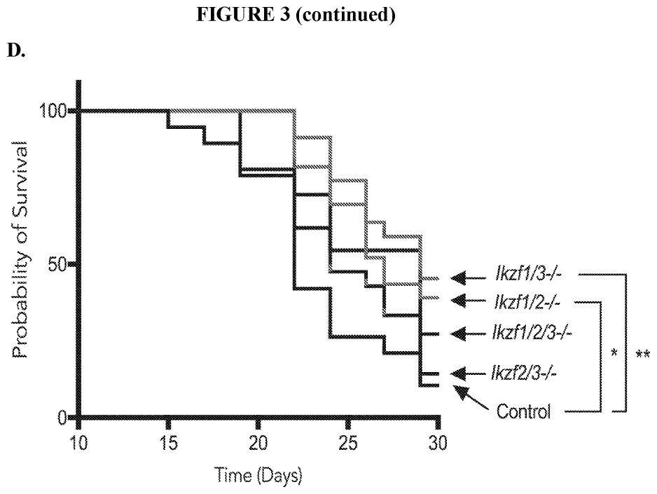

Given that the in vitro data demonstrated that CAR-T cell function was enhanced by reducing the level or activity, or both, of at least one of Ikzf1, Ikzf2 and/or Ikzf3, the in vivo performance of the CRISPR-edited CAR-T cells was assessed in vivo. CAR-T cell function was assessed using the EO771-Her2 lines. Using non-treated (i.e., wild-type) T cells, control CAR-T cells and Ikzf1−/−, Ikzf2−/−, Ikzf3−/−, Ikzf1/2−/−, Ikzf1/2/3−/−, Ikzf2/3−/− and Ikzf1/3−/− CAR-T cells were adoptively transferred into mice with established EO771-Her2 tumors injected orthotopically in the mammary fat pad of Her2 recipient mice. As shown in FIG. 3A, the adoptive transfer of Ikzf1−/− CAR-T cells had significantly greater activity against the established tumors in vivo. Significantly greater activity was also observed following the adoptive transfer of Ikzf1/2−/− and Ikzf1/3−/− CAR-T cells (FIG. 3B). Mice treated with Ikzf1/2−/− and Ikzf1/3−/− CAR-T cells also had a significant survival advantage (FIGS. 3C and 3D).

Taken together, these data demonstrate that CAR-T cells modified to reduce the level or activity, or both, of at least one of Ikzf1, Ikzf2 and/or Ikzf3 enhances the in vivo anti-tumor activity of CAR-T cells.

### Example 4

### Phenotype of Murine CAR-T Cells Modified to Reduce the Level or Activity, or Both, of Ikaros Zinc Finger Transcription Factors

To further investigate the effect of reducing the level or activity, or both, of at least one of Ikzf1, Ikzf2 and/or Ikzf3 on CAR-T cell responses in vivo, the frequency and phenotype of blood, tumor and splenic CAR-T cells in mice treated with control CAR-T cells, or Ikzf1−/−, Ikzf2−/−, Ikzf3−/−, Ikzf1/2−/−, Ikzf/2/3−/−, Ikzf2/3−/− and Ikzf1/3−/− CAR-T cells was assessed.

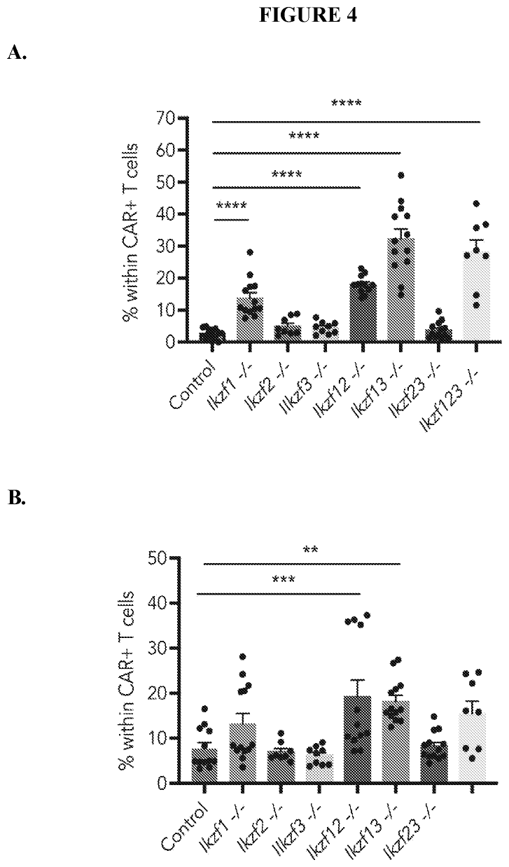

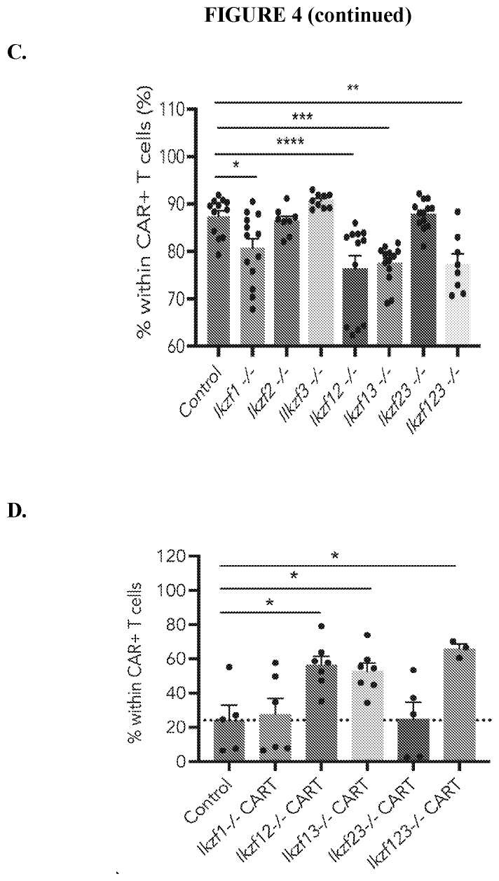

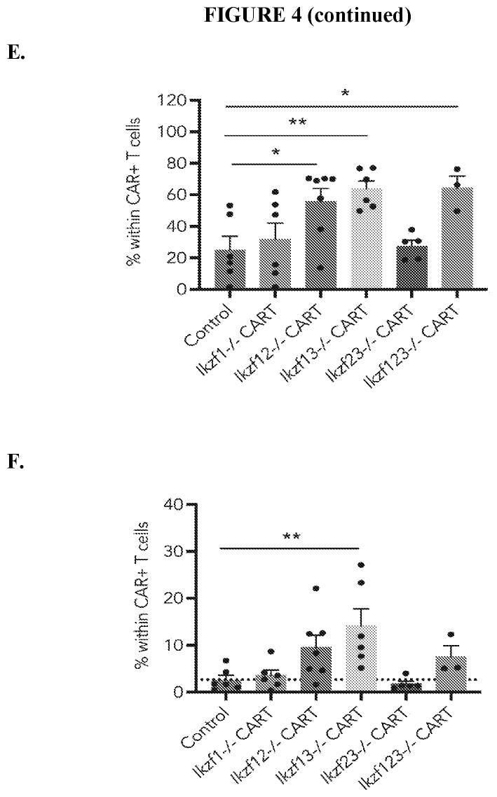

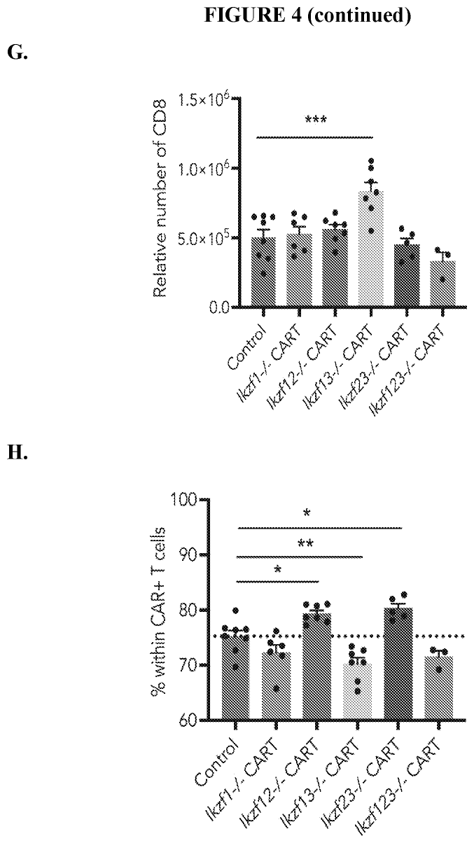

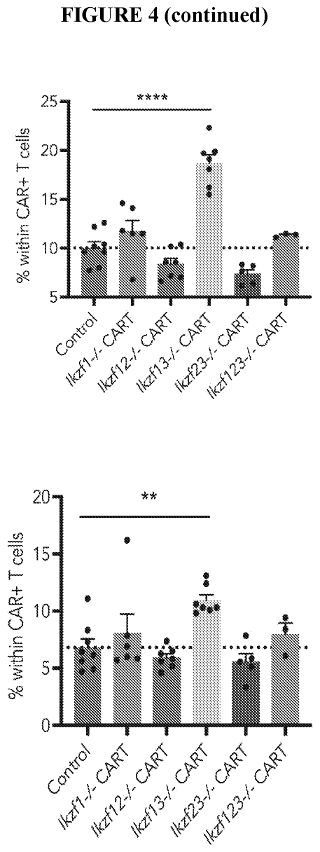

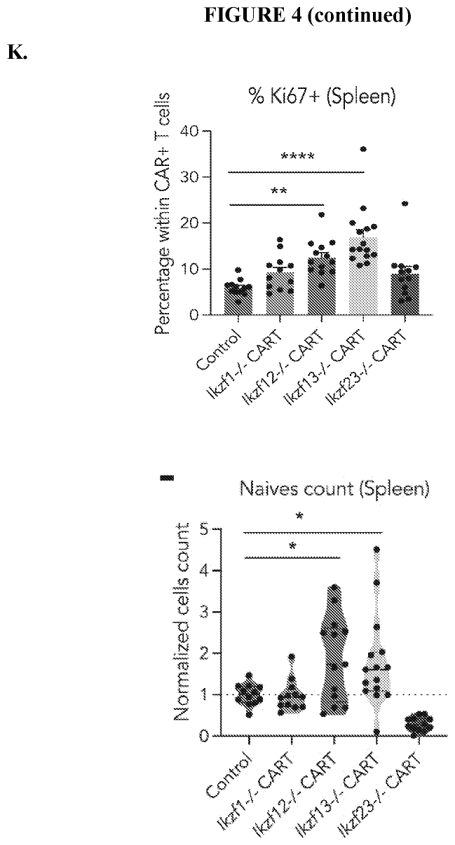

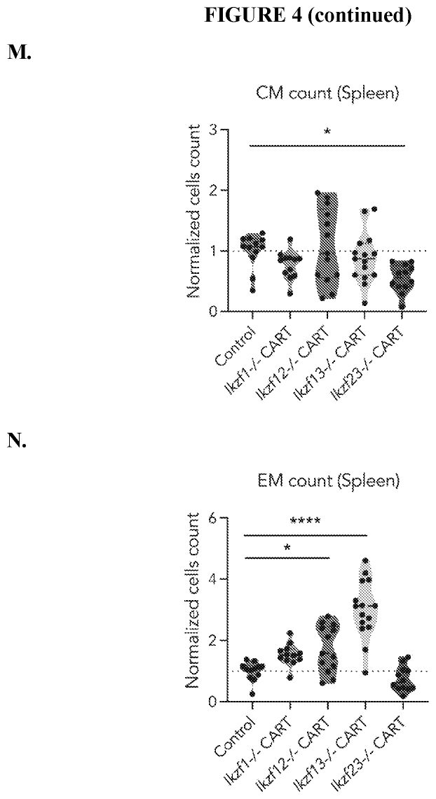

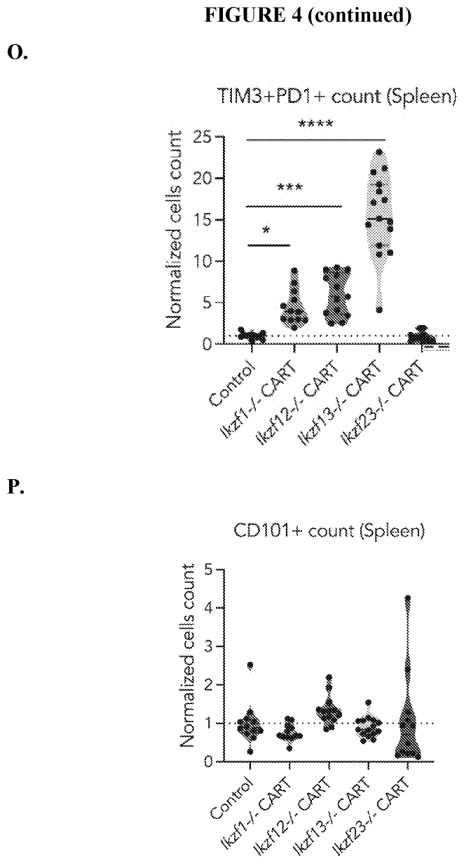

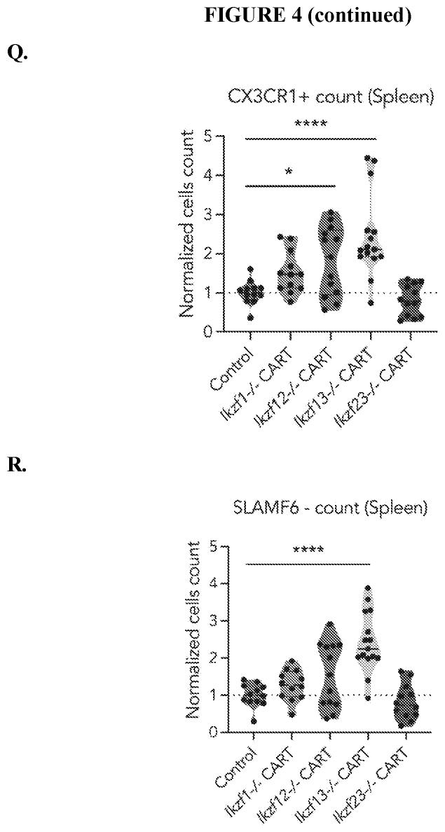

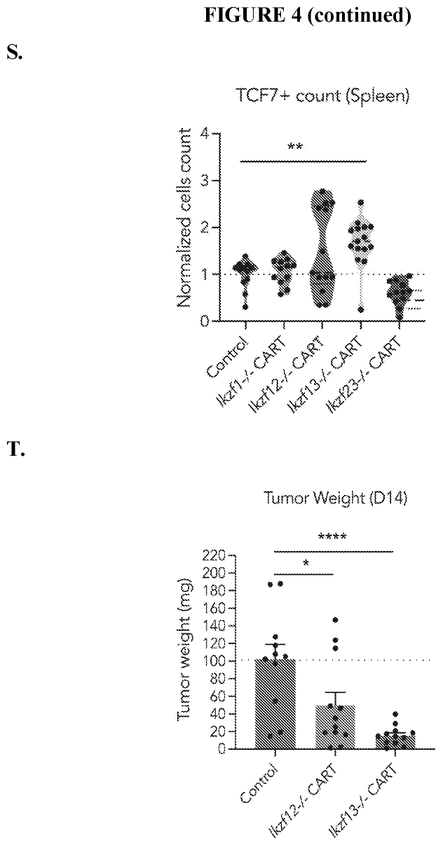

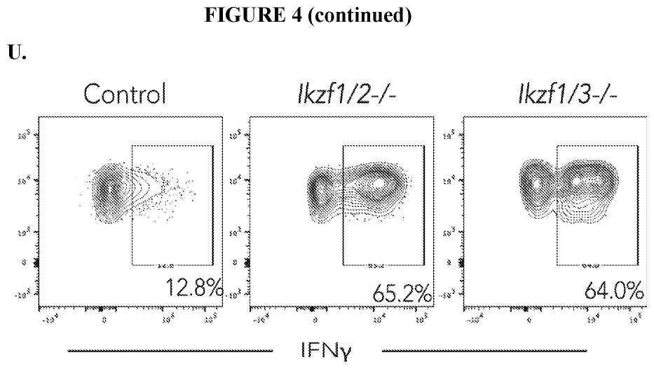

In peripheral blood collected at 12 days post-adoptive transfer, it was shown that the proportion of PD1+/TIM3+ cells was significantly increased relative to control CAR-T cells for the Ikzf1−/−, Ikzf1/2−/−, Ikzf1/3−/− and Ikzf1/2/3−/− CAR-T cells (FIG. 4A). The proportion of effector memory T cells was also significantly increased for the Ikzf1/2−/− and Ikzf1/3−/− CAR-T cells relative to control CAR-T cells (FIG. 4B). By contrast, significant reductions in central memory T cells were observed for the Ikzf1−/−, Ikzf1/2−/−, Ikzf1/3−/− and Ikzf1/2/3−/− CAR-T cells relative to control CAR-T cells (FIG. 4C).

For the intra-tumoral CAR-T cells, it was shown that the proportion of PD1+/TIM3+ cells was significantly increased for the Ikzf1/2−/−, Ikzf1/3−/− and Ikzf1/2/3−/− CAR-T cells relative to control CAR-T cells (FIG. 4D). In these experiments, it was also observed that the Ikzf1−/−, Ikzf1/2−/− and Ikzf1/3−/− CAR-T cells had high levels of IFNγ (FIG. 4E) secretion following antigen-specific stimulation with EO771-Her2 tumor cells, with the Ikzf1/3−/− CAR-T cells also having high levels TNF secretion specific stimulation with EO771-Her2 tumor cells (FIG. 4F).

As shown in FIG. 4G, the Ikzf1/3−/− CAR-T cells resulted in a significant increase in CD8+ cell number relative to control, unmodified CAR-T cells in the splenic samples. It was also shown that the proportion of PD1+/TIM3+ cells was significantly increased relative to control CAR-T cells for the Ikzf1/3−/− CAR-T cells (FIG. 4J). The proportion of effector memory T cells was also significantly increased for the Ikzf1/3−/− CAR-T cells (FIG. 4I). The results observed for the proportion of central memory T cells indicated that the increase in CD8+ cell number in the spleen was not associated with an expansion of CAR-T cells with an effector memory phenotype without a reduced number of CAR-T cells with a central memory phenotype.

The phenotype/absolute number of the CAR-T cells is also represented in FIGS. 4L to 4S.

Tumor weight was also assessed for Ikzf1/2−/− and Ikzf1/3−/− CAR-T cells, relative to controls. As shown in FIG. 4T, tumor weight was significantly reduced at day 14 post-administration, with a highly significant reduction in tumor weight in mice treated with Ikzf1/3−/− CAR-T cells. Intratumoral CAR-T cells harvested from tumors at day 14 post-administration also showed increased cytokine production (i.e., secretion of IFNγ, FIG. 4U).

Taken together, these data demonstrate that CAR-T cells modified to reduce the level or activity, or both, of at least one of Ikzf1, Ikzf2 and/or Ikzf3 promote or maintain CAR-T cell populations with a less differentiated (i.e., stem-like) phenotype. Moreover, the CD8+ CAR-T cells elicit increased expression of activation markers that are indicative of increased CAR-T cells activity in vivo, which is further reflected in the significant reductions in tumor size observed in mice treated with Ikzf1/2−/− and Ikzf1/3−/− CAR-T cells.

### Example 5

### Function and Cytotoxicity of Human CAR-T Cells Modified to Reduce the Level or Activity, or Both, of Ikaros Zinc Finger Transcription Factors

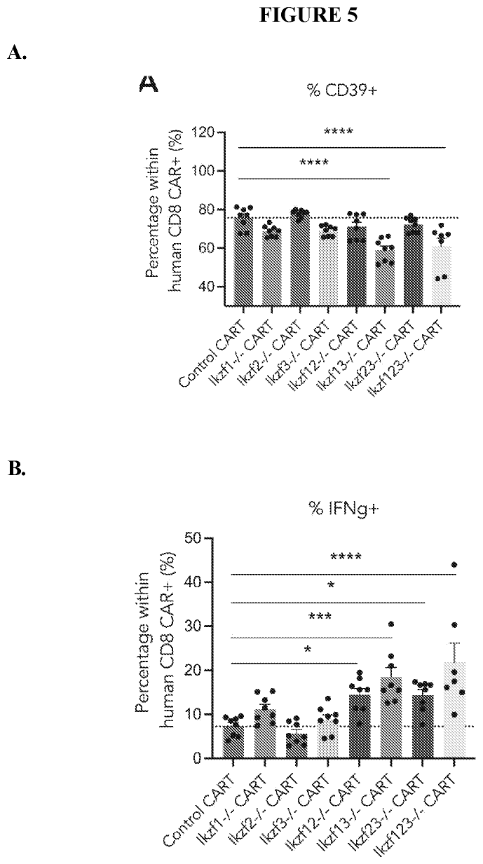

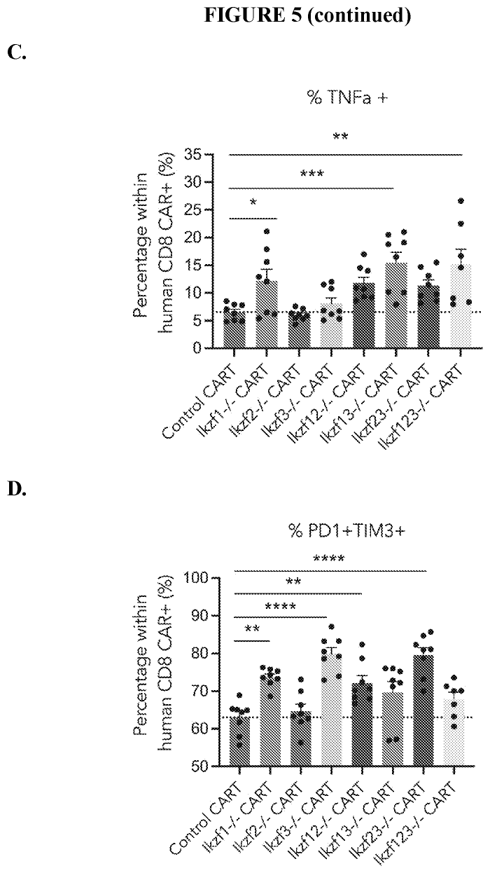

Human anti-Lewis Y CAR-T cells were CRISPR-edited to reduce or eliminate the expression of at least one of Ikzf1, Ikzf2 and/or Ikzf3. The CRISPR-edited CAR-T cells were co-cultured with MCF7-Lewis Y tumor cells for 3 days in vitro, at a ratio of 3 CAR-T cells/2 tumor cells (ratio 3T/2C) to determine the effect of IKAROS Zinc Finger transcription factors on CAR-T cell function and cytotoxicity. As shown in FIG. 5A, the Ikzf1/3−/− and Ikzf1/2/3−/− CAR-T cells resulted in a significant decrease in the proportion of CD39+ cells within the population of CD8+ CAR-T cells relative to control, unmodified CAR-T cells.

To investigate the functional consequences of reduced or eliminated expression of Ikzf1, Ikzf2 and/or Ikzf3, the cytokine production of CRISPR-edited CAR-T cells was determined following co-culture with MCF7-Lewis Y tumor cells. In these experiments, it was observed that the Ikzf1−/−, Ikzf1/2−/−, Ikzf1/2/3−/−, Ikzf2/3−/− and/or Ikzf1/3−/− CAR-T cells had high levels of IFNγ (FIG. 5B), TNF (FIG. 5C) secretion, and PD1+TIM3+ expression (FIG. 5D), following antigen-specific stimulation with MCF7-Lewis Y tumor cells.

Taken together, these data demonstrate that human CAR-T cells modified to reduce the level or activity, or both, of at least one of Ikzf1, Ikzf2 and/or Ikzf3 enhances the in vitro activity of CAR-T cells, thereby reproducing the data presented for the equivalent murine CAR-T cells described elsewhere herein.

### Example 6

### Anti-Tumor Activity and Phenotype of Ikaros Dominant Negative CAR-T Cells

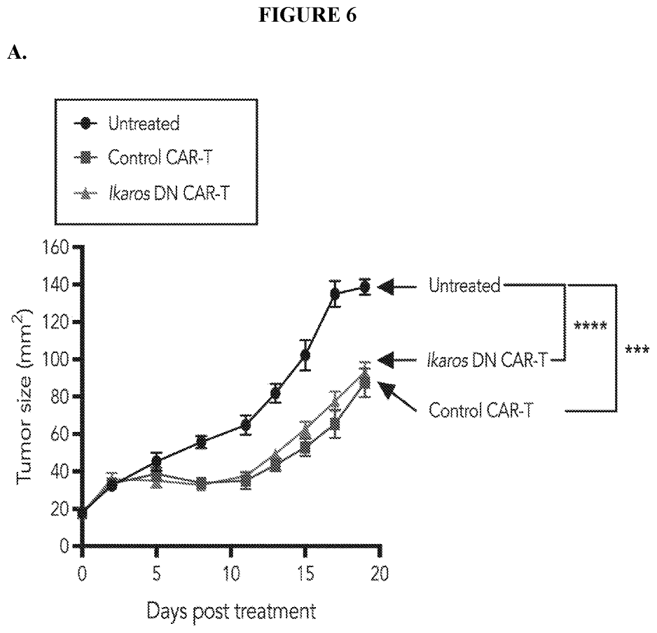

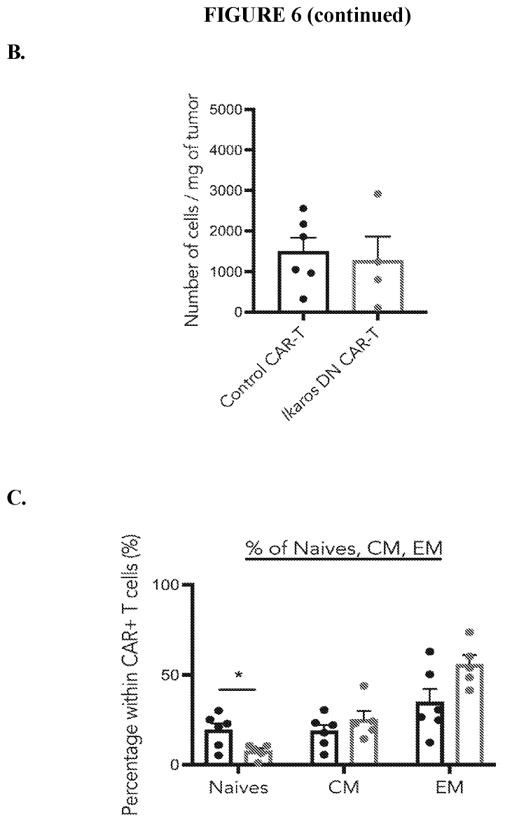

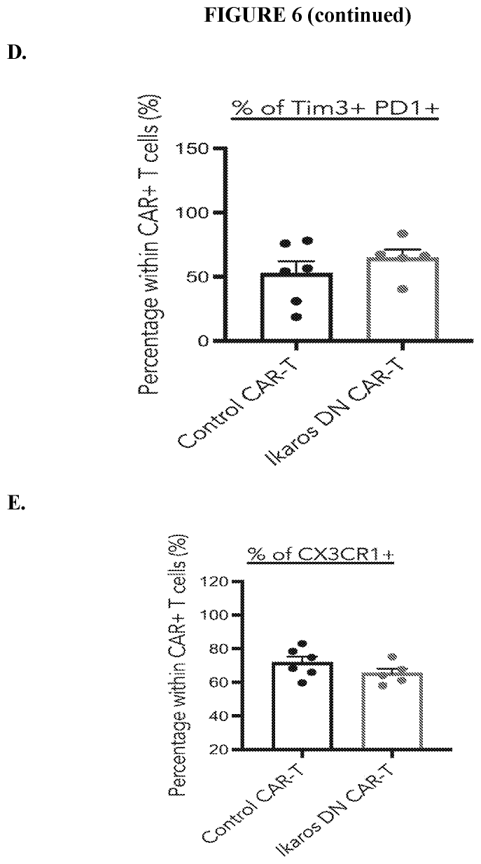

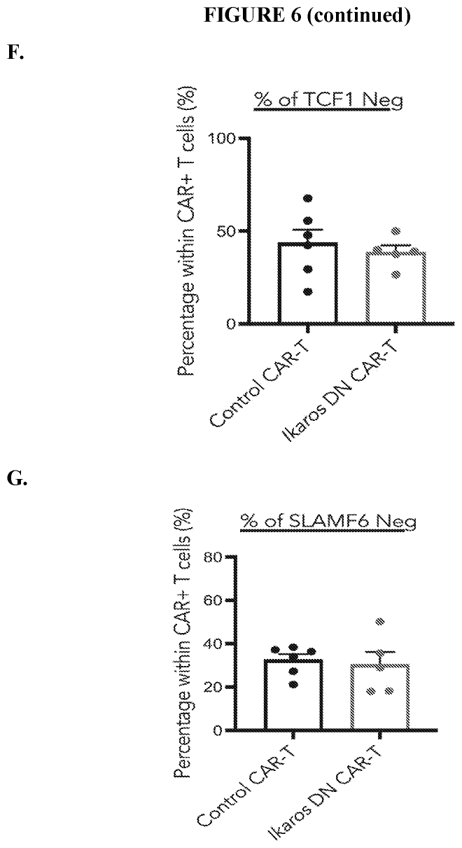

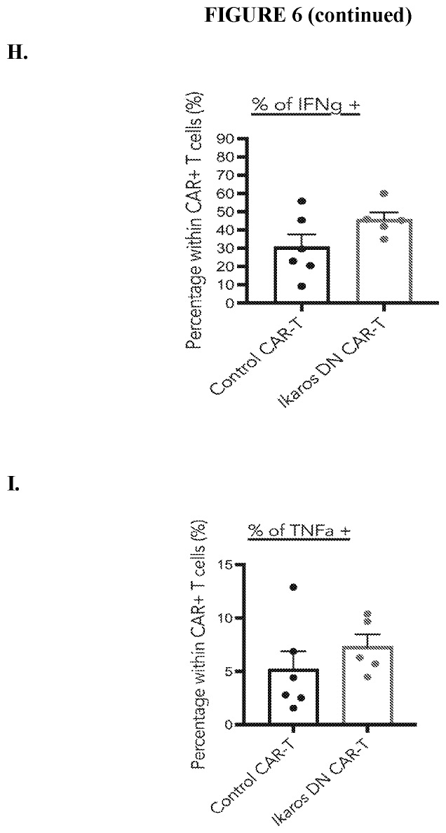

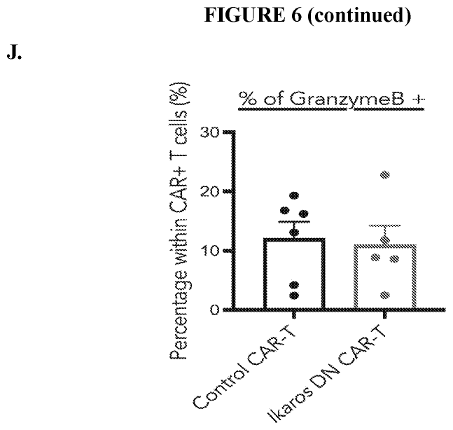

The in vivo performance of the Ikaros DN CAR-T cells generated according to the method described by Papthanasiou et al. (2003, Immunity, 19(1): 131-144) was assessed using the EO771-Her2 lines. Untreated T cells (i.e., wild-type), control CAR-T cells and Ikaros DN CAR-T cells were adoptively transferred into mice with established EO771-Her2 tumors injected orthotopically in the mammary fat pad of Her2 recipient mice. As shown in FIG. 6A, the adoptive transfer of Ikaros DN CAR-T cells had substantially the same activity relative to the control CAR-T cells. Similarly, the number of CD8+ cells within the tumor of mice treated with the Ikaros DN CAR-T cells was substantially the same as those treated with the control CAR-T cells (FIG. 6B).

To further investigate the effect of the Ikaros DN mutation on CAR-T cell responses in vivo, the frequency and phenotype of tumor CAR-T cells in mice treated with the Ikaros DN CAR-T cells or control CAR-T cells was assessed. In these experiments, it was observed that there was no difference in the frequency or phenotype of Ikaros DN CAR-T cells relative to control CAR-T cells (FIGS. 6C-6G). Similarly, no significant difference in IFNγ (FIG. 6H) or TNF (FIG. 6I) secretion following antigen-specific stimulation with EO771-Her2 tumor cells, nor was any significant difference in effector function (Granzyme B+) observed (FIG. 6J).

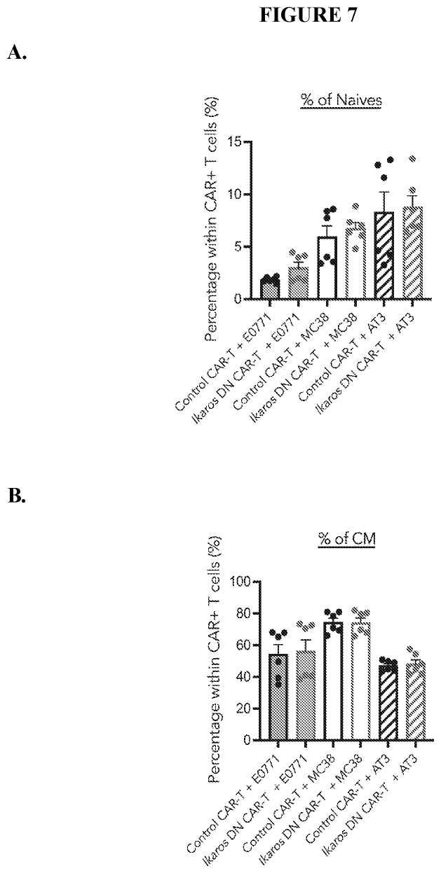

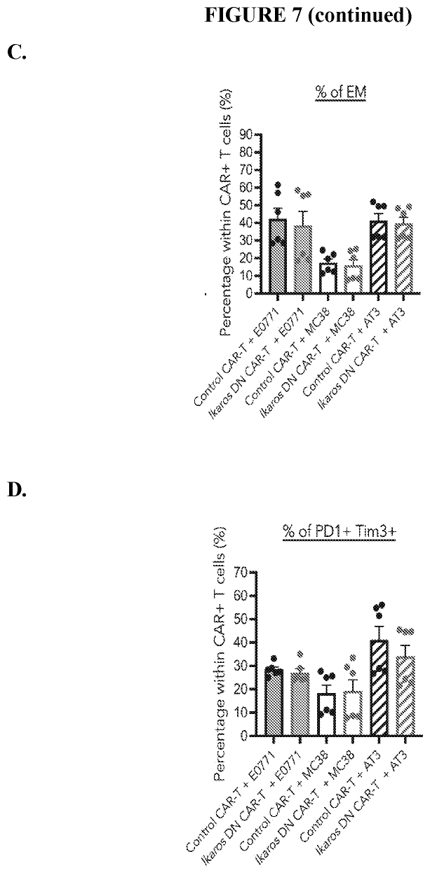

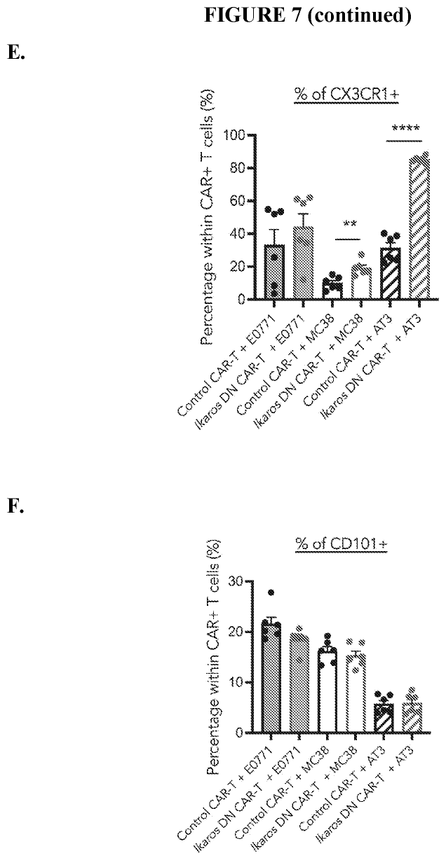

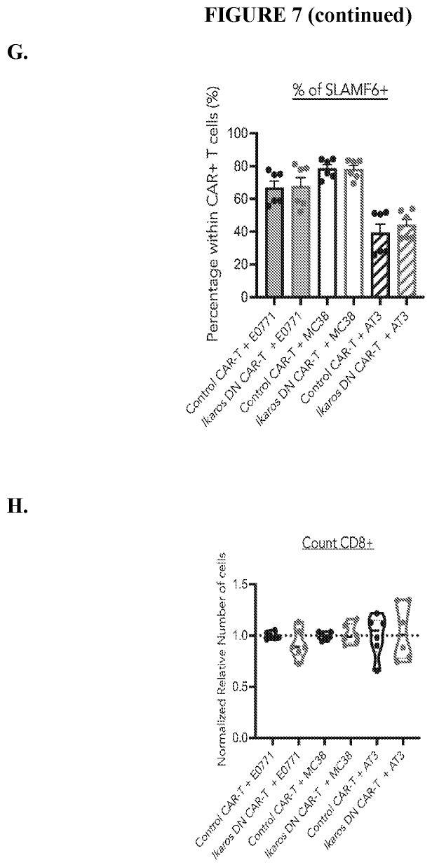

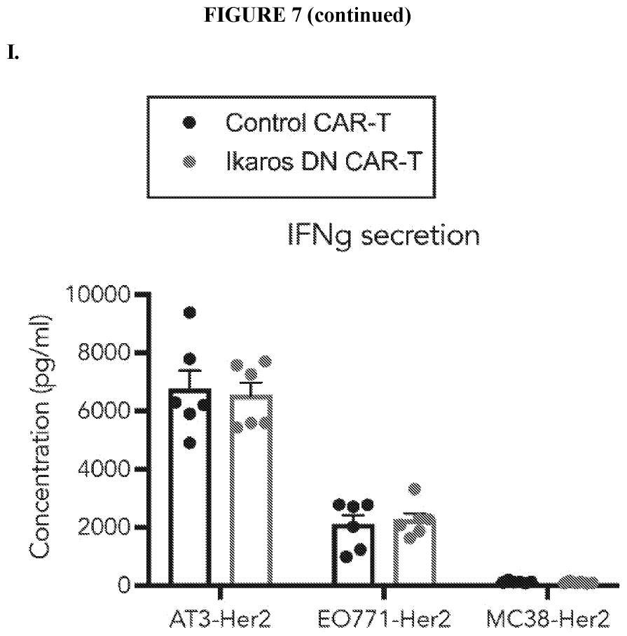

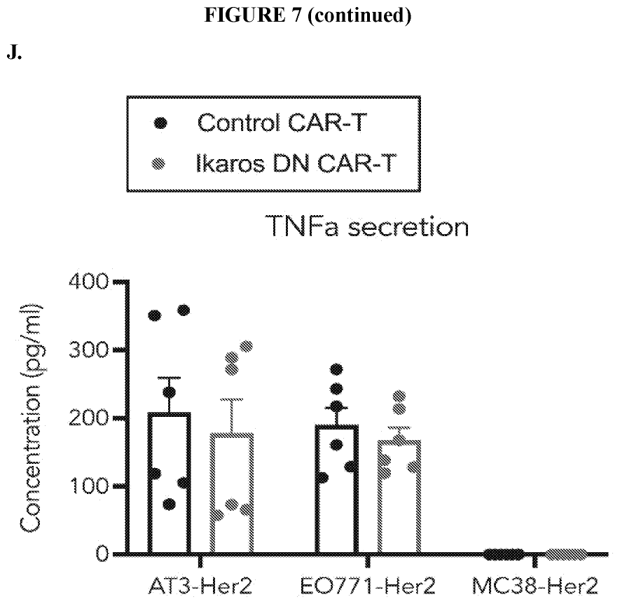

The Ikaros DN CAR-T cells were co-cultured with EO771-Her2, MC38-Her2 or AT3-Her2 tumor cells for 3 days in vitro, at a ratio of 3 CAR-T cells/2 tumor cells (ratio 3T/2C) to determine the effect of the Ikaros DN on CAR-T cell function and cytotoxicity. As shown in FIGS. 7A-7F, there was no significant difference in CAR-T cell phenotype associated with the Ikaros DN CAR-T cells relative to control, unmodified CAR-T cells. Similarly, no significant differences were observed in cytokine production following antigen-specific stimulation with EO771-Her2, MC38-Her2 or AT3-Her2 tumor cells (FIGS. 7I and 7J).

### Example 7

### Phenotype and Function of Lck-Cre; Ikzf2f/f; Ikzf1Mut/+ IKZF Knockout Mice

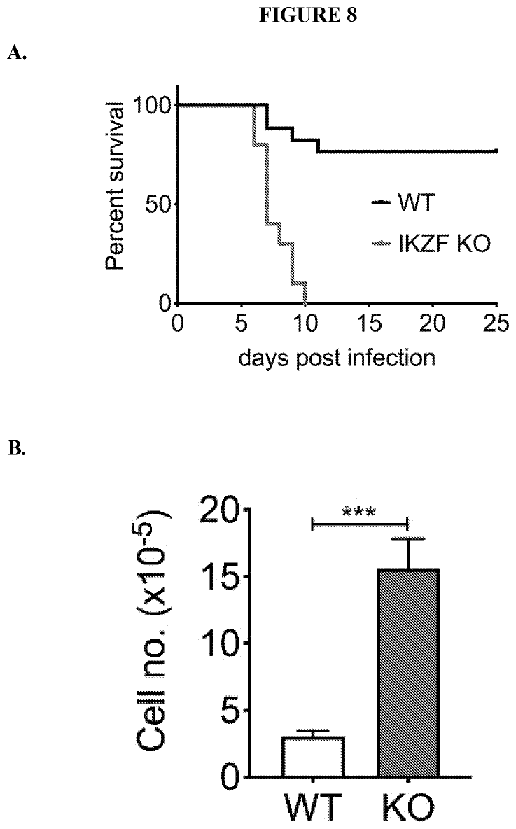

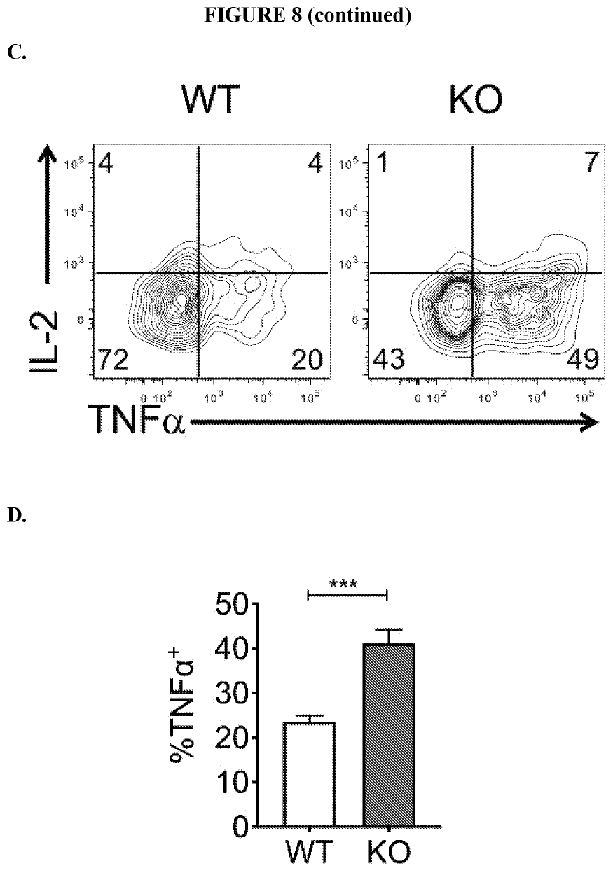

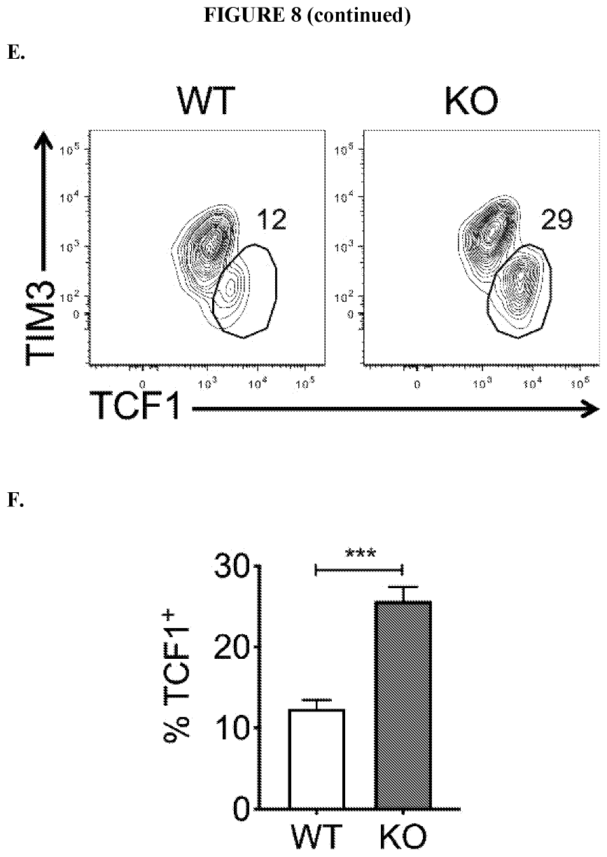

The data illustrate that unlike conditional loss of Ikzf2 in T cells alone, which has no phenotype (data not shown), combining Ikzf2 loss with pan Ikzf inhibition via a dominant-negative mutant Ikzf1 allele (IKZF KO) leads to mouse mortality after chronic LCMV infection (FIG. 8A). In the chronic LCMV model, mortality is typically indicative of immunopathology and disrupted T cell exhaustion. Consistent with this, IKZF KO mice also had elevated expansion of virus-specific T cells with rescued TNF production (FIGS. 8C and 8D). This occurred without a rescue of IL-2 production, suggesting phenotypes are independent of IL-2. Unexpectedly, increased T cell expansion in IKZF KO mice did not deplete TCF1+ stem-like T cells (FIGS. 8E and 8F). Collectively, these data show that IKAROS Zinc Finger transcription factors cooperatively enforce T cell exhaustion in the context of chronic viral infection.

### Example 8

### Phenotype and Anti-Viral Activity of CRISPR-Edited LCMV-Specific T Cells

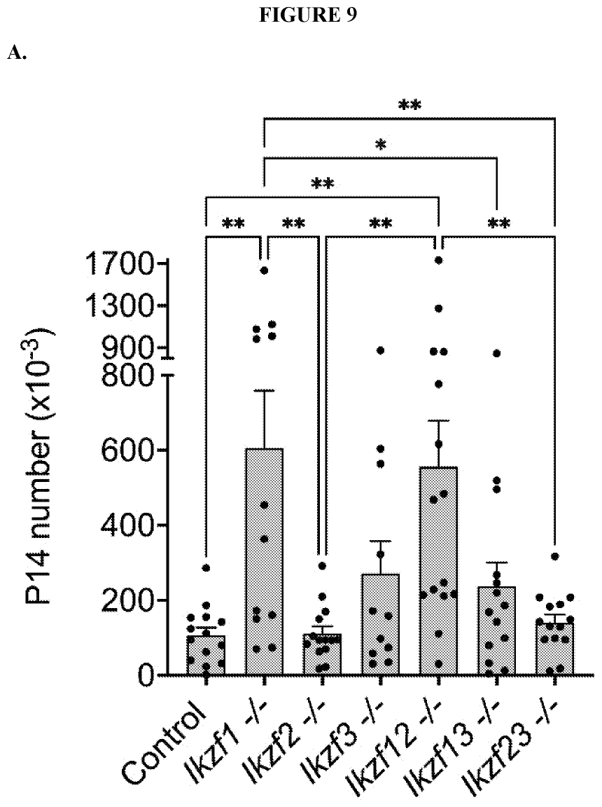

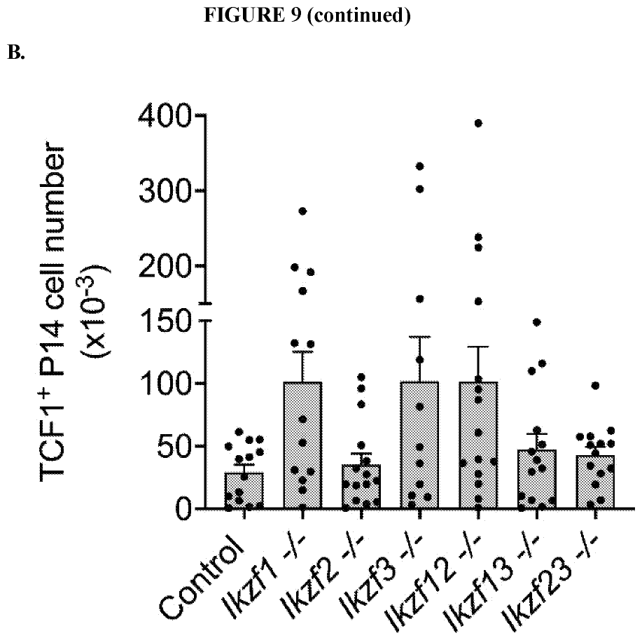

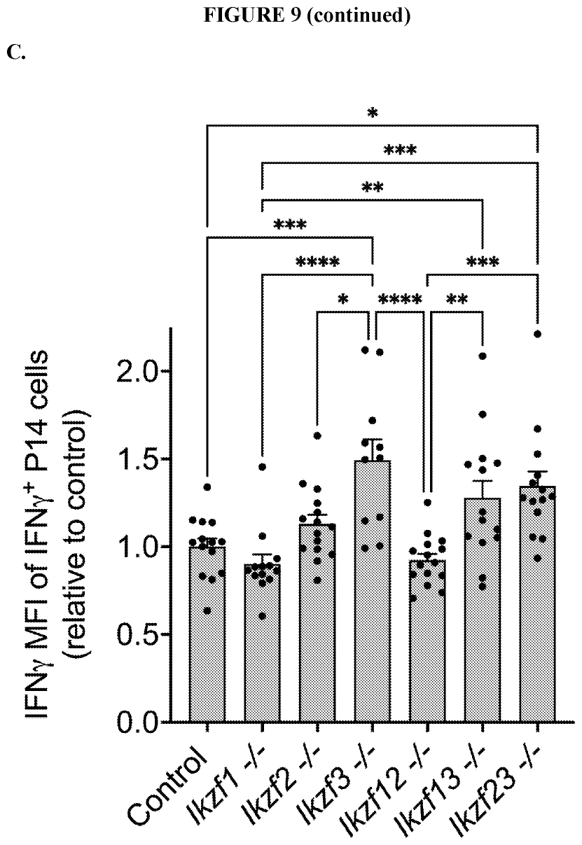

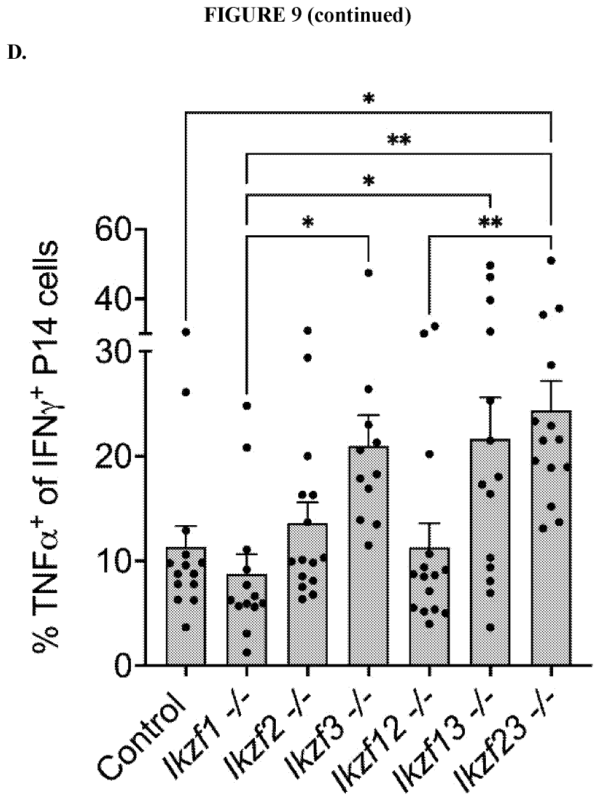

CRISPR editing to identify the specific IKAROS Zinc Finger transcription factors that control exhaustion revealed that T cell expansion is restrained specifically by Ikzf1 with a lesser effect by Ikzf3 (FIG. 9A). Similar to the results presented in Example 7, this did not occur at the expense of stem-like TCF1+ T cells (FIG. 9B). In contrast, cytokine function (IFNγ and TNF production) was solely restrained by Ikzf3 (FIGS. 9C and 9D). Combined loss of all three Ikzf factors was detrimental to cell survival (data not shown). These data demonstrated that Ikzf factors do not simply operate in a redundant manner, but instead have specialized functions in restraining either exhausted T cell proliferation or cytokine production.

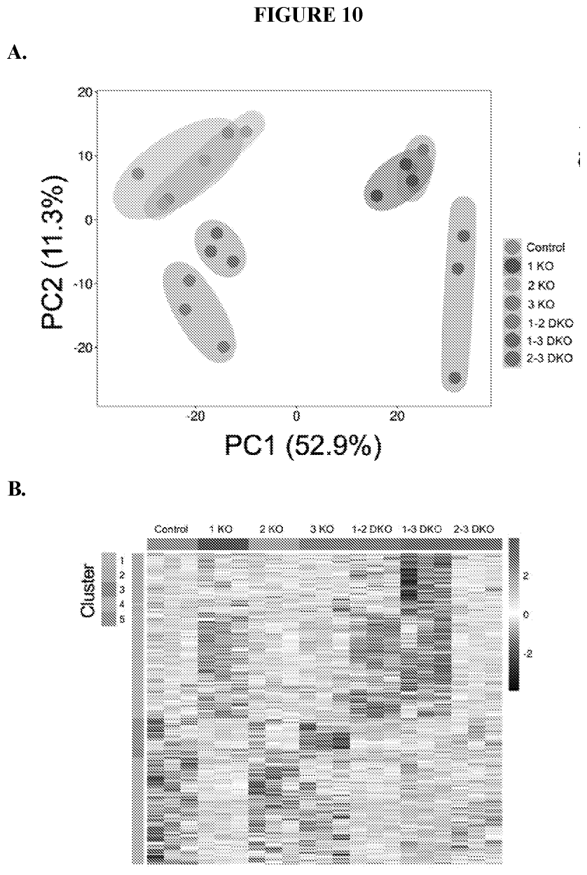

This lack of redundancy between the Ikzf factors is further reflected in the results of the bulk RNAseq analysis presented in FIG. 10. In particular, these data show that loss of Ikzf1 and Ikzf3 have differential transcriptional effects on T cell phenotype. Not only does loss of Ikzf1 and Ikzf3 change the differentiation status of these cells, the unique differentiation states observed are not presently known to exist in nature. These differentiation states may be at least partially attributed to the epigenetic characteristics of Ikzf1−/−, particularly when combined with the loss of Ikzf3 (i.e., Ikzf1/3−/−), whereby unique epigenetic characteristics were observed in both stem-like TCF1+ and differentiated TCF1− cell populations (FIG. 11).

### Example 9

### Anti-Tumor Function of Murine CAR-T Cells Modified to Reduce the Level or Activity, or Both, of Ikaros Zinc Finger Transcription Factors in an In Vivo Model of B-Cell Acute Lymphoblastic Leukemia (B-ALL)

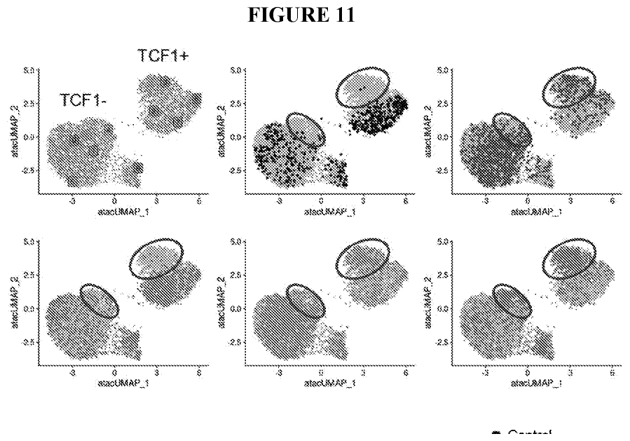

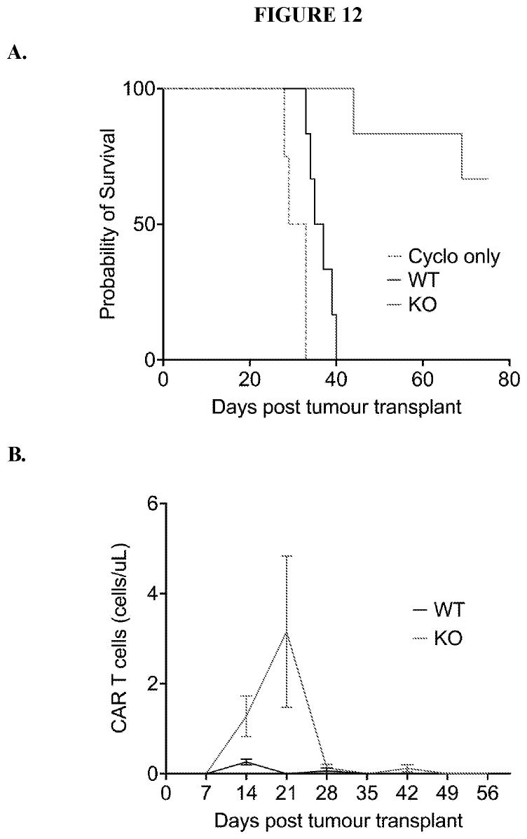

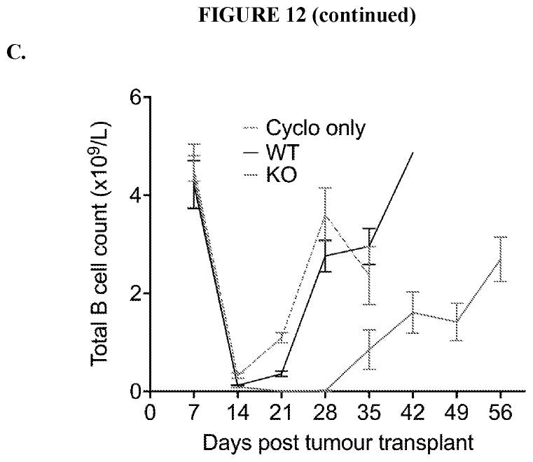

To further investigate the anti-tumor function of Ikzf CAR-T cells, the therapeutic effect of Ikzf1/3−/− CRISPR-edited CAR-T cells in an in vivo model of B-ALL was assessed. In these experiments, B6 background B-ALL cells having a BCR-ABL1 oncogenic transgene were transferred intravenously into congenically marked (CD45.1) recipient B6 mice. 6 days post-cell injection, mice were lymphodepleted with cyclophosphamide followed by treatment with a low (2.5×10⁵) dose of mouse CD19 CAR T cells (Ikzf1/3−/− CRISPR-edited CAR-T cells or wild-type CAR-T cells) the next day. As shown in FIG. 12A, mice given wild-type (WT) CAR T cells fully relapse at 40 days. Mice treated with Ikzf1/3−/− CRISPR-edited CAR-T cells demonstrated improved survival, with some animals surviving to end-point (80 days). In this instance, relapse was not associated with loss of CD19 antigen on the B-ALL cells, rather poor CAR T cell persistence (FIG. 12B). The improved activity/expansion kinetics of the Ikzf1/3−/− CRISPR-edited CAR-T cells in this model was also reflected by B-cell aplasia (FIG. 12C).

### Example 10

### Phenotype of Human CAR-T Cells Modified to Reduce the Level or Activity, or Both, of Ikaros Zinc Finger Transcription Factors

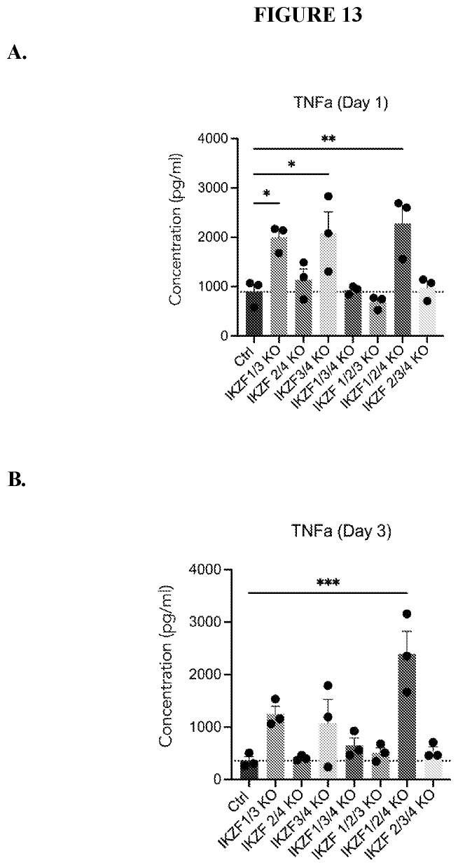

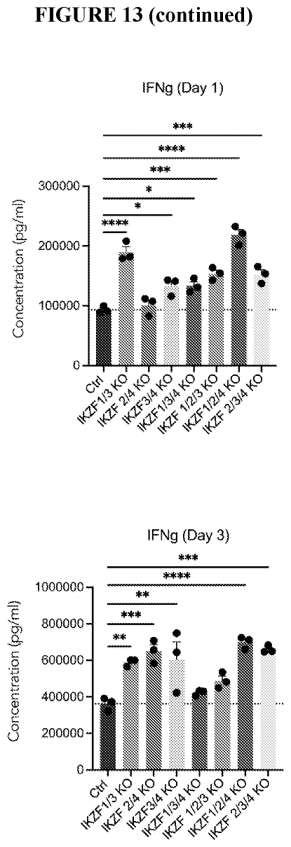

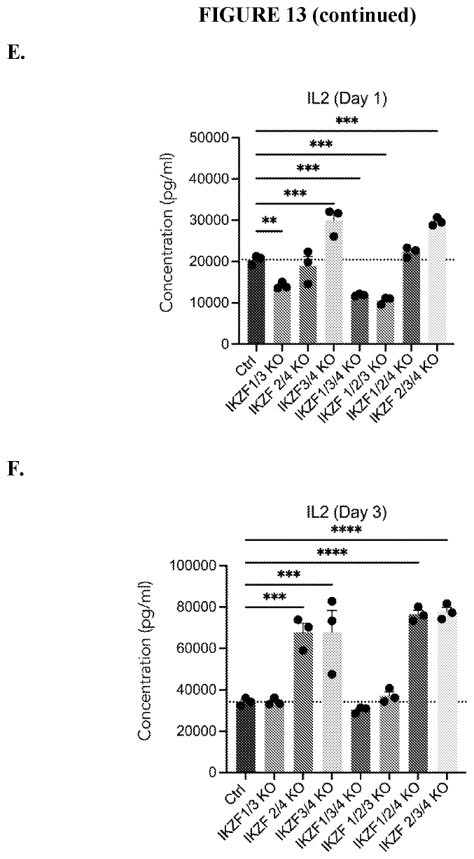

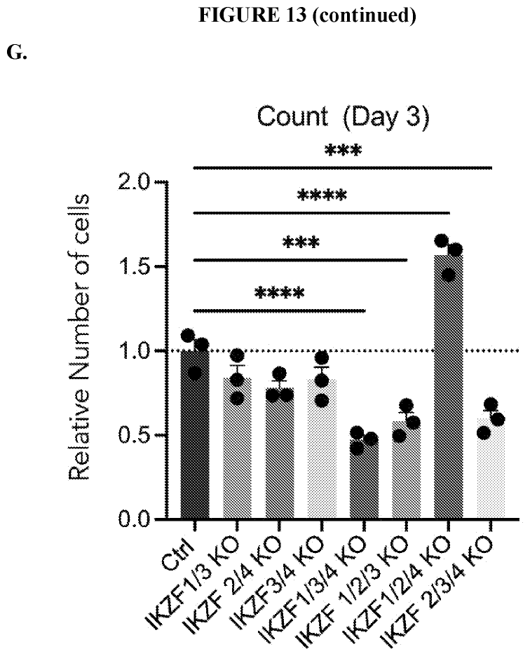

To investigate the functional consequences of reduced or eliminated expression of Ikzf1, Ikzf2, Ikzf3 and/or Ikzf4, the cytokine production of CRISPR-edited CAR-T cells was determined following co-culture with EO771-Her2. In these experiments, it was observed that Ikzf1/2/4−/− CAR-T cells had higher levels of TNF secretion following antigen-specific stimulation with EO771-Her2 tumor cells relative to control CAR T cells on day 3 (FIGS. 13A and B). The effects observed for IFNγ (FIGS. 13C and D) and IL-2 (FIGS. 13E and F) secretion following antigen-specific stimulation with EO771-Her2 tumor cells relative to control CAR T cells were observed more broadly across the various Ikzf knockout groups, noting that Ikzf1/2/4−/− CAR-T cells had highly significant increases in the secretion of IFNγ and IL-2. In addition, targeting of Ikzf1, Ikzf2 and Ikzf4 (i.e., Ikzf1/2/4−/−) resulted in a significant increase in cell number relative to control, unmodified CAR-T cells (FIG. 13G).

Taken together, these data demonstrate that CAR-T cells modified to reduce the level or activity, or both, of at least one of Ikzf1, Ikzf2, Ikzf3 and/or Ikzf4 enhances the in vitro activity of CAR-T cells, thus supporting the targeting of IKAROS Zinc Finger transcription factors to generate CAT-T cells with enhanced function.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
sgRNA sequences

| sgRNA | Sequence (5′-3′) | SEQ ID NO: |
|---|---|---|
| Human Ikaros G1 | UGUAAGCGAUACUCCAGAUG | 5 |
| Human Ikaros G2 | CAACGCCAUCAACUACCUGG | 6 |
| Human Helios G1 | GAUACUACCAAGGCUCCUAA | 7 |
| Human Helios G2 | AAACUGAAAUGUGACGUCUG | 8 |
| Human Aiolos G1 | ACAUCACAUAGUCCAGGAAG | 9 |
| Human Aiolos G2 | GUUUUAAAGUCAGAACCCAU | 10 |
| Human Eos G1 | GGUGGAGAUGUACAGCGAUG | 11 |
| Human Eos G2 | CCCUUCCAUUGCAACCAGUG | 12 |

TABLE 2
Summary of murine CAR-T cell phenotype following co-culture with EO771-Her2

| Number of: | Ikzf1—/— | Ikzf2—/— | Ikzf3—/— | Ikzf1/2—/— | Ikzf1/3—/— | Ikzf1/2/3—/— |
|---|---|---|---|---|---|---|
| Naïve | NS | NS | NS | + | NS | NS |
| CM | NS | NS | NS | +++ | +++ | NS |
| EM | NS | NS | NS | ++ | ++ | NS |
| PD1+ | +++ | NS | ++ | +++ | +++ | NS |
| TIM3+ | | | | | | |
| CD101+ | NS | NS | NS | + | + | NS |
| CX3CR1+ | NS | NS | NS | +++ | +++ | NS |
| TCF7+ | NS | NS | NS | NS | NS | NS |

Control group was compared to other groups by one-way ANOVA, + = p < 0.05; ++ = p < 0.01; +++ = p < 0.001; ++++ = p < 0.0001; and NS = not significant.

1. A modified T cell characterized by reduced level or activity, or both, of at least one IKAROS Zinc Finger transcription factor relative to an unmodified T cell, wherein the IKAROS Zinc Finger transcription factor is selected from the group consisting of IKAROS Family Zinc Finger I (IKAROS), IKAROS Family Zinc Finger 2 (HELIOS), IKAROS Family Zinc Finger 3 (AIOLOS) and IKAROS Family Zinc Finger 4 (EOS).

2-43. (canceled)

44. The modified T cell of claim 1, characterized by one or more of
(a) reduced level or activity, or both, of IKAROS relative to an unmodified T cell;
(b) reduced level or activity, or both, of HELIOS, AIOLOS or EOS relative to an unmodified T cell;
(c) reduced level or activity, or both, of IKAROS and AIOLOS relative to an unmodified T cell; or
(d) reduced level or activity, or both, of IKAROS, HELIOS and EOS relative to an unmodified T cell.

45. The modified T cell of claim 1, further comprising a chimeric antigen receptor (CAR) or a T cell receptor (TCR).

46. The modified T cell of claim 45, wherein the CAR comprises an antigen binding domain that binds to an antigen selected from the group consisting of CD19, CD20, CD22, CD30, ROR1, CD123, CD33, CD133, CD138, GD2, Her2, Her1, mesothelin, MUC1, gp100, MART-1, MAGE-A3, MUC16, NY-ESO-1, L1-CAM, CEA, FAP, VEGFR2, WT1, TAG-72, CD171, α-FR, CAIX, PSMA, Lewis Y, BCMA, GPRC5D and HIV env protein.

47. The modified T cell of claim 45, wherein the CAR comprises a signaling domain selected from the group consisting of CD3ζ, CD28, 41BB, DAP10, OX40, ICOS, DAP12, KIR2DS2, 4-1BB, CD3s, CD35, CD3C, CD25, CD27, CD79A, DC79B, CARD11, FcRa, Fcftp, FcRy, Fyn, HVEM, Lck, LAG3, LAT, LRP, NKG2D, NOTCH1, NOTCH2, NOTCH3, NOTCH4, ROR2, Ryk, SLAMF1, Slip76, pTa, TCRa, TCRP, TRIM, Zap70, PTCH2, and LIGHT.

48. The modified T cell of claim 45, wherein the TCR binds specifically to an antigen selected from the group consisting of 707-AP, AFP, ART-4, BAGE, Bcr-abl, CAMEL, CAP-1, CASP-8, CDC27m, CDK4/m, CEA, CT, Cyp-B, DAM, EGFRvlll, ELF2M, ETV6-AML1, G250, GAGE, GnT-V, Gp100, HAGE, HER-2/neu, HLA-A, HPV, HSP70-2M, HST-2, hTERT, hTRT, iCE, KIAA0205, LAGE (L antigen), LDLR/FUT, MAGE, MART-1/Melan-A, MC1 R, Myosin/m, MUC1, MUM-1, MUM-2, MUM-3, NA88-A, NY-ESO-1, P15, p190 minor, Pml/RARa, PRAME, PSA, PSMA, RAGE, RU1, RU2, SAGE, SART-1, SART-3, SSX1, SSX2, SSX3, SSX4, TEL/AML1, TPI/m, TRP-1, TRP-2, TRP-2/INT2 and WT1.

49. The modified T cell of claim 45, wherein:
(a) the TCR binds to a viral-specific antigen (e.g., a lymphocytic choriomeningitis virus (LCMV)-specific antigen); or
(b) the TCR binds specifically to a neoantigen.

50. The modified T cell of claim 1, wherein the modified T cell is derived from a human donor.

51. The modified T cell of claim 1, which comprises a genetic modification leading to reduced expression of a gene encoding at least one IKAROS Zinc Finger transcription factor relative to an unmodified T cell, wherein the gene is selected from the group consisting of Ikzf1, Ikzf2, Ikzf3 and Ikzf4.

52. The modified T cell of claim 51, which comprises one or more of:
(a) a genetic modification leading to reduced expression of Ikzf1 relative to an unmodified T cell;
(b) a genetic modification leading to reduced expression of Ikzf2, Ikzf3 or Ikzf4 relative to an unmodified T cell;
(c) a genetic modification leading to reduced expression of Ikzf1 and Ikzf3 relative to an unmodified T cell; or
(d) a genetic modification leading to reduced expression of Ikzf1, Ikzf2 and Ikzf4 relative to an unmodified T cell.

53. A pharmaceutical composition comprising the modified T cell of claim 1.

54. A method of adoptive cell therapy, the method comprising administering an effective amount of the modified T cell of claim 1 to a subject in need thereof.

55. The method of claim 54, wherein the subject has cancer.

56. The method of claim 55, wherein the cancer is:
(a) a solid tumor; or
(b) a hematological cancer.

57. The method of claim 54, wherein the subject has a viral infection.

58. The method of claim 54, wherein:
(a) the modified T cell is autologous to the subject; or
(b) the modified T cell is allogenic to the subject.

59. A method of preparing an adoptive cell therapy, the method comprising:
(a) obtaining a population of T cells;
(b) contacting the population of T cells with an agent for a time an under conditions suitable to reduce the level or activity, or both, of at least one IKAROS Zinc Finger transcription factor relative to a T cell that has not been contacted with the agent, wherein the IKAROS Zinc Finger transcription factor is selected from the group consisting of IKAROS, HELIOS, AIOLOS and EOS; and
(c) culturing the population of T cells from step (b) for a time and under conditions suitable to prepare an expanded population of T cells.

60. The method of claim 59, further comprising:
(d) transducing the expanded population of T cells with a construct comprising a nucleic acid molecule encoding a CAR or a TCR.

61. The method of claim 59, wherein step (b) comprises one or more of:
(i) contacting the population of T cells with an agent for a time and under conditions suitable to reduce the level or activity, or both, of IKAROS relative to a population of T cells that have not been contacted with the agent;
(ii) contacting the population of T cells with an agent for a time and under conditions suitable to reduce the level or activity, or both, of HELIOS, AIOLOS or EOS relative to a population of T cells that have not been contacted with the agent;
(iii) contacting the population of T cells with an agent for a time and under conditions suitable to reduce the level or activity, or both of IKAROS and AIOLOS relative to a population of T cells that have not been contacted with the agent; or
(iv) contacting the population of T cells with an agent for a time and under conditions suitable to reduce the level or activity, or both of IKAROS and AIOLOS relative to a population of T cells that have not been contacted with the agent.

62. The method of claim 59, wherein one or both of the following apply:
(i) the agent is a biological agent; and
(ii) the biological agent is selected from the group consisting of a RNA interference molecule and a CRISPR gene editing system.


SEQUENCE LISTING (12 sequences)

SEQ ID NO: 1 - DNA, 102234 bp, Homo sapiens
  source 1..102234: mol_type = genomic DNA
tgcacccaggccaacatggaaggcgccatcctaacttctgccgtgagcaggtgggagggaagagagacgagaggtattccattggttgtctgggaaaatgaattgcaccttcccctcccttgcggaggatcaacttttcccaccccctcgggtgggcactcgcatcctggggccggagcctgaacccgggagccaaggggccccagttccagggacgtgaagctgagcgtacagcgggcgctcccagacactggggaaagtgctttacgatgtcccgagtccctccagtctcgccagcggggcgagcgtgagggtgccccgaccgaccagcggccccgggtgcagggtggcgggcccggcggcgcgcgtccccctccccctcctggcggcccgcacgtgtcgcccgcgccgcgcccccacgggttacgcgcgggtcccgcagcgccgcggccgagccgggctgcccggcccgcggacacagcgccggccgccgcatcccgtgcggggccgcggcgcgatgctgcgctggaatgaggaagcgcggcggcgaggggagggcccgggcgcggtgcgcgcgggggtggcggcggcgcgccgagcgggcccggcgcgggcgagcgggctgcagccggcggcggcgccagcaggtacggcccgcacccgccgccgccccggcggcctttgggggctgagccggagcccggcgcgattgcaaagttttcgtgcgcggcccctctggcccggagttgcggctgagacgcgcgccgcgcgagccgggggactcggcgacggggcggggacgggacgacgcaccctctccgtgtcccgctctgcgcccttctgcgcgccccgctccctgtaccggagcagcgatccgggaggcggccgagaggtgcgcgcggggccgagccggctgcggggcaggtcgagcagggaccgccagcgtgcgtcaccccaaagtttgcggggtggcagggcgcgcgctctggccacccgccgctctgggcggcagctggtggcaacgcaagggcgcggcgggggcggccggcgcggagggggccaggtacggggcccgcgggcggcgctgtgcgcgcggggcagccggtcggccgggagcgcgaaagcctggtctgagccggctgggggcggggagtgtggcggagaaatggggaacaatgcgagtgagcaacttcaggaagtcattgtgaaagaaagctgggaagagctccgcggccaagttagcaggacactctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacgtgagtgttttcaaattgaatttcaataggaaaacttggggtaactggtgaatttaaaaaaaaaaaaacacagtaaagaaaagcggtaaggttggtagaccctggtgtcgctcaggtccgcctctcttttctgaggacagtgagagagttcacttctgtcaagcgtctgttgctctgcactgtgccagcaggtgcaggaccaggccgacatgggacacttctgagcagccccgctgtcaccaggagaggagttctagctcccaaccatatttaaatttatgtagacctacatatacccacggaagtcagcctttataaagtcgtgtgtaaagagttttccttatatttgagccgggagctttctttttatactataaatatgatgagatcgagtctgaacttaatttctgcaagagaggaattatcccggctttgaaaagttagtccttttgctgaccgcaggtttgacgctcaagtcaccaaaccttctcaggaaaacccttagtaatattaaggcatcaggttacttgcggttatatttgaaatgtattttaaatatttgtcaagcatcgctgctgatgcctaaggaacctcgtgagggcttgtttttccttctaatttggaggcatctaatgaccgaaaaccgtagcgattccatagggtctgaccaggcacagctttcaaatgcagcttccctctctctagggactgcagcccacccagactgaatttcaatgcggtgcgctttgcttaggttacccactcacaatttcccactgcgccgcaggcagtatatttcagctttgagataccttgttttaaaattccagacaaaatggtgttgaggaaatgtctccttactagtcccatcaacttctgttaaaagaggaaaatttatggaatttgaaaatactgcgtatgatatttaaactttcatagacattcaaatgcttttaaggccaggttcaatttggttatgagtcgaggggtgggggggacccacatagaaatgtcctgggtcctcttgagtttatttctttgtttgaagatgtttgttcaatgagttttattgtactcatcttttatatggaattttaaaaagtaacaatttcagtattatttatattagaatgtgtcagaattatttccgtgacaaatcagatcatttgggctatggcttaaaatgtacacgaggcaaatattcatgacaagaagattcaccttcttacgctggcatcttgtaaaatgcagaacaagttaaagaaataatgtgtacacatacaaataatgatgtcacattaaaaatactacactattcttgcttgatggaatgtatctgatttccaatttcaccatgaacatatttcatacattttttacatgaaaaaaaacgtgactcttaagtctcacagtcaatcagagctggtgaccagaacattttattgaactaaatggtcatgttttcttccccttttgtttcacggtgagagttgaaggaaggagtttagaaactctccagtacttgtttaattcatcagtgttctaattagagtggtacctcttggaaaactacacacccccctaatgcagaaacatcatagcaataatcacccaccctcagggtctccaggagaccacaagggctgcagataaaagtctggatgtgttaggtttgaccctttcgaagagttttacacaggctcctaaagagaagatcagctgtggccgtttgtagccatttcctttgtcgaaaaactaagatcgcagtgaatgtattagccaagaggtctaaagccctgttgtactgcaggccactgtcttccttgtttgactagagacttggagtttgagaacagtggttctttggtttggatacattttttgttcttgatttggatgtgtgtgtttcatgcgtggttaatatagcatattttcaatataaatgtcaaaaattttgaaataggaaagaactctctatatattaatgtacttatacacacacttcaagattatgcatttattaacagatacatgaaataaattccatgtgcatatgcacatatgcacacagagcgtgcacacacacagcatgcacacagcgtggagtgagaggcatggggcagtgtggaagagttttaacatcaaacagacctgaaatgagtattaaaggccccctttatttttaaacttttactaaaacaagatggatttccctatgttatataatggtgaattttaggcataaataacgttttttgagtgttgcataattgtacgtattaatgtaatgtaactgtggttaacgaagaattcatcaaggatatcactgttttgtggcattttttttttcctcctctaatctttggacttgtgaaataatttcactatgaaataaatgttggttcttgtcatattctaagggagattgatgtaagtggctccactccagcttacagaaggtaaaccacgacctttttgcgttctctgaaaacgcttgtcttccgatgcctctgtttctaagactgacaagcactctgggggcactgtgacgcctgcttctagcggcagagttgctgcagctcctgtcctggctgtgaacattgttctctctctggtgtctctatgttcataactacagagacttcagctctattccatttcatatttgtgctgaataatcattccattttatgggagaaaacacaagatgtaaaagcaacaagtgacccatcctttgaagcttacaagaagagaaacattaatctatttcacgtcttgaaaacagatcagttttattttgctcaaaaagggcacatgtacatttttgatctaggtcttagaaacgtagagtttcagaggatcagcattatacacactgtcacacacacacacacttaaaattcagatgaggaacaagataggaatgaggttttgttagggacgcagagcacctaaaaccaaaggatatcgacagtaacaaagctgtttttactgtagtgctgactgaacactcatgctggtgtcttcatgtggaccatggctttcttgtatttctttgcagtttaataaatgacttcatatctcaggttacctttccacatctcctggaatatatgtttatgtccttaaagtttcagtgtcgtcactttagtagctttagtttgagtttttaaatgtttggtaatattccaacaaatattttttaagacattatgaaaccttatgaagtgccatatattacaagtgagataaaacagcaagcaaaagaaggtttgcagaaggtttttaagtggcgaagtgcgggcctgcccattttggtgtctccttggtggttactcctgagaagggcctggaggaagagcaactgaggcctaatctacaggcaactgccaaattgtttcagttgacgtttttccctctcatgtttgactataataaataggtagttgccagtggagccttcagccaaccacctggtaataaactgttaaaaatggtgcaaaccctaggtcacaggtgtgggggccatttgtcttgcctgttaacaggcctggccttaattcttttctcccatggccatttctgcctttggggaactcacaattcctgttgactaaaagagcacccttttccaccacaagcctgacaaatcagacgtccacataatttctgaactcgttttggttaggacaggaagcacaggctcccttcctgtctgtgttttcctaagagaaaacggtcttccctccttttttgcatatttggcaagtggttccacctttctctgcaccctggtggagtgtgaaggcagcagaggaaccttttggaggaggaagaggacacagaggccctgtagccaggcaccaagatccctcccaggtggctgggtctgaggggaactccgagcagccctaggtcctcaaagtctggatttgtgtggaaaaggcagctctcacttggccttggcgaggcctcggttggttggtgagtgccacacggtttctttgtgtgcttgcatggattggaatagccattgtgttcttccgtcttccctgctggtgtttccacagtgggtggcctgagcccagagcagctccccatatccctgtgcaggccacctgtctcgggtgatggagagcatcattatgctccgtctgaacgctctgctttcggatggccccatgctccacctcctgatagctcgtggcgcggggccacggcttaacaaatggctgaaaatgggtcctaattagtggaaaagtgctttcttcatattttctcactcgagtgtgcagtgattcatttttcttctgcaatcagctcactgctaaagtaaatctgactctcttcccgccattgcacaccaaaagttaactctaatgggtaggaggttaggtttgttgagagagcaatgcagtaaaaagaggggatccaatgtggtcttgtctgtctggtcttcctttcttcgttttttcctcccttgtcttctctgtcattcccttccctccatttgccttgcctttcctgtccttcccttcccttccttcccctctttctttctataattggtggggggtttgcacagactgccaaaacactaagaactgtgtaaagtgtttttgaatggccttacacatattgaagtagatttttatgctccatttttgagatcacacactaaaatctatacctttaaagcattttctgttagtttgaaactatttgaaaatgaacaatgtggtttagattagagtcctgttctgaagctaggagttccactatgaatattgatttatcagtttttgacaaatttttgttgttataccagattttcactggcaaacctagagcaaataaaattccacataagatacttccctagacctaatgggaaaaatgtttaatttagagtctttaggagaaatgagaatgaggaattgaccttttgtaagcttacttctgaggcactctgaagtgtgttccagtgcttttaatggaaactagagagagccagcaaccccctagtgtgagccccacttttaaccggaaaaagtgaccttttcctcctcctttgtgctgagttttgcgtagggcagaaaattaagctgatattcaaagagattcactgcaaaaacatattgataaatcgtatattctatttcattaaattaaaaccatactgctaattatctcaggttgttaaacataaggcaattaattatcattttaaaagttggtaggaagttgtgagtacttttgcagtatgagtgttttcccgctttagtatgaggttgtgtatgtttgcttgaatttacagaattttcactttaagagcagacaatgttttgttaaagaaatgaaatttgctaaaaaggagcatgtaaagtgaaacattaaaaataaataatttcaacttacttaagagctgcagaaaaatctgattgctgtgtttaaaatgaattttcccacatttcgctctcttatggacaggagcattttctgtcaggttataaataaagacatgcccattttttgtacccccacaaatgaggaagttgtaagctctctgaggttttactgatgagccccctccccctgggtttgcatgaagagatcataggccacaaataaaggactacaaaatggggtctaaactatcctggtggggcctgatacccacgtttcgcatggaccttacgatgtgatgaatggttttggcatgagtgtcttaagaatgcttccagattcgggttacaggacagccagcgctgagctccctattgcagaacaaagtaggaatctagaactttcttgctaacaggatccagctaaaacaccaagttagattcttaaatgatgttcttttctgtcattatttgattgttgtcagtagcagtaattgttaccaagccattgatgcttctattcttccctttgcccttctgagacacagctcattttgacttcagtggaacccctcgaaggtggggtgatgagcaaggtgaattttcaaagtaaagctactaagagaccaaactacaatttaaggaacctgatttttgaatcaaattccatatactgtgggtatagttcaacatagattaatttcttatagttattatgaaaaaaatctcatcttgatgatagctgataattttgtgggtgtcgtaaacaaaacagaggtcagaattcagtcccttggggaaaatttccaattagtaggaaaccaagtggcctaccttagtttgaagacacccatcaggatgtctgcaccttttcatcctctctggaggaaagactaaatacccattattgtatataggtcaggccaaagcagccttttatattgcaaggaataagaggtaaatagatatatgtgcaacaatgaatcccctaatgtgtttactctagaacacatgttctttctgtatttatatgtagattttgtagatcttgtcttaccacctgctaatggtagatactgtatctaaataagttgaggaaaatttatagtacctaggaatgtgtcctcagtgggccaatcaatcaatcatgacttcaggttatttttaataaatatacacgtatgggttcataaacaatgggatgttcttgtgaagatctaaataattttacttctttgggactaaataaaatatagcttttgccaaataaactcacacaagcacttattttaatagaagtcaaatggctttgcagaaacttcagttttacaggtgcattgtttgaaatgttacgggtatacaagtggatttctctattatgtacagtgttaagtttgagtttcaaaatgtccacctgaaatgatttacttgtacgttaagataatttaactgctaagaaggcaagataaagcattctttgtgacaccatatggccttgctgagggaaaaacttactgttataagtttgtgtttatctctctttttaaaaaaaaatgaagaaaaaaacgtttaaaataatgggaacacagcagttcctggggtcctctgtctctttatcttattatagtaaattaccaaaaaaataatgacctggggcatgtctgtgtggacccttcttttagaggcagtttctgtgttttgtaaagctgtaggttctattttcattgcacttcatattgctgcacagctcctgaccatgcatgaaggtcctctgaaatcggtaagagggcagaagaaaatgattctaaacttagatttttttaacttaagtgatgaagtgtgaaacgccatttatatttgaggaagctacctaggaagtggctcatgtcgatggcccaaatcagaagagggcctgtaaaagcttctatcaattttgactgtgtatgcttctaccatggcggctcaataaacagcagtattagtttaagagtggatggtacagtagtatagacgggaagcctctcctctccgtgtgaaccgtgcacccctatgagagggtagagacaatacaatatgcctgtaacgtcaggacagacagtcatggccagcttgaactccagccctgggcttcttgcagcaacaaacgtgaacacagaggactgtctccaactccactttctctatttttaaaacaactttttgaatacagtatctgccatcttttcttatacctcactttgaaacaggtggctccactgtggcatttaaaatgttctgtttcttttccctctgtatcaaatacctctttaccaagaaaacattcaaacagcatagtttttaactgtattttgaaaggtttccttagttccctttgacccttcctcttttgcatatcagttcctggccataaaaataaaaaatgctaggacagaattgcacatctgagctgatttgccctcaaaaagtttcacagtggaacaaaccgcaggaggagttttctgtggctcagttaaatgtcgggggagggtggtgtgaaagccaaattggattcctgctttcctgtttaaatcttgtttttcattgttatttgcaccagcaatactctgtggaataatcatgaaaatgtgtagattggcagctaatttttgaaaaatgaaaagaatcagaaatgaaataagagtgctcggaagtttttatgttctctcaacctgttttgtcaaattgttacgaaaacctataaggtctctttgactagatacaaagactttgcacattgccttagctttctcttgaagcatttccttttttaaaatacagtgtaattcacagtgatatgatagatttgcaaaagtaaaatctaccagtctgaagatgaaaggacttgtctcttagcaggaataatgggttttattaaagaggtctgtgacctaaggcattttaaataaattacaggcttggtccctgtctcccccatgtatctactcccttcaatataagcatcattgagtatttaaggaaataaccccaaatgtaactctagtgtagcttcacttgtcagggaggaaaaagtaaatagcatacatttggccaaataaccagaactttactgtagaagttttatgatgaaatttgcctttagtgcagagtattacaaagatcatgtttagtttctagcagtatataagtagcatccatccttatctgtcatgcatttggagtgtgcgacccctgcactgggctgcaacattctgatgggcaagagtgctagggagaaagaggcatcaccatcagactgcacgggttcaagtgtcagctctgtggttgattagctgtgtgacctggggaaagctatttctcttagccttggttctctcatctataaaatggagataatgatgcagatgccttgggtttaattgggagagttaaagacacatttacatatttagcaagtaggtgttgaattctagctctacattggacactatgccaggtgctcaaataaacaagtggacaagacagacaacacccatggtcttatgaggcttaaccatttgcctcttcaatgccagaaacttagtaggttgattagataaagccagtgagtaccagtatccttttctttgcagccttttcctggcacactaaaaatactcagtacatatgaaatatcactggacaaagaatcccccttagagtaccagtggagaaggaaggcatttgcttaaaagcaaaccaacagaaagacattgtaaggcagttgtttaagtctcagagacctataatttttttcttttttcttttttttcatctcgctctgtcgcccaggctggagtgcagtggcacaatctcagctcactgcaagctccaccttccgggttcatgccattcttctgcctcagcctcccaagtagcagagactacaggcgcccgccaccacacctggctaattttttgtatttttagtggagacggggtttcgccgtgttagccaggatggtcttgatctcctgacctcatgatccgcctgcctcggcctcccaaagtgctgggattactggcatgagccaccacgcccggcaactacaattgttcttaaagcttgtagaattactgtgtgctaccaacagacaggctaattttgagtgaccctcagtactttgtacagttaatttggcacgctgtgtacttagtggctttttaacagctataaatttgggctgctagaaaagtagtaaagttgtgattcttgacaggcatctatctgcattttcatttttacttcatttgtctagactcagcttgtcagaattatggaagagactccttgtgtcagggcaagcactgtgaagagaggtattcactgtcagaaaagagaggggagctggaggcagctcagaggcctgagacccgcctccacaggagccccagcaggttcggtggagctctggccacactctcctttgggatgctgaagtcagaatgagttcacttcccagccagtcttgccaaggctcctcacctggaagcagcaactgcccagggctgttggatgtttctccccaggggacagccaggtcccagtcccgcctcggtgtggaaggaggaaaggcagggtccaggaagctgtttcaggacaggcccaaggtcccccagggatgcctttcagggtcagcggaggctgtaaatcagcagggcccacacggcctggaagaggcccctgtgctgtcggcttgcccggcttgcccggctcctagtccggcttctgctcctcctttgtaaagttatggatatgctaatagtttccaactgagactaggaaagtaagtcctacttgacactgtttggtcagaaagagggagagaaaggagaaggacagagagagactgagagagagacagtctcagacaaagggagacggagggagggagggagagacagagaaagagatgggaggtaggtgtgggaggagggagagatgcagaaggcagaggaaagacagacagagatttagacctcccaagtcagtgagcagtccagagttggagtggagggtgcctggtggcttgtgactgcagactccactccccgctcctagaggcacagccatggacagcttctgtcacgttggccctgcacttatctctgcatctatttccccttgtgcaagattcagaactgcatgctccaaaaaaacaataaaagcattcatgttcataagaattgcacaggtaaaaggtagtttgctgatattgttgtattttttactatcgcttcttttaggtcttgcctgaaattgtttgggtttcccaggcaaagtagaaaactgcggtacgtttctgtgaaataattattccttctggcatctccctttacagacctactgatcttgatttttcatttaggtgaaagtttgtgaaaacatgccattagcttgctttgtgattaactccttttactgaatgtgagctccttttaaattgaggccatatcaagcttaaattccatattttacccggcactctgcatttcttccatgtgggagaggaggggctcagtaagtgctttgtaaaatacacagccgaagtgatgcacgtgctaacaaaggagtgtgacaggacttaagtgcccttctagacacttcaggctcccctttgtaagctgtcttggaagaggccacatttcctttccctcaaacagtttctcattgtttgattattcttttagcctttctctggaagcaaagccacttttacgagaaagtcactgctttttcatctcaagagatgcaagtttggagtttggggaagttttcaggtgcccgtcaagtcatcctttatgatgtcagacgagtcaggccacagaattcacagggctcagtgcagaccgaaaacttgaggcctcttgttcagaaattattaaaaattttggtgaacatcaccccaagcaaagagatcccctaagcaccagcccccaagcaactgcactcataagcccatgaagccccctgctgtcagaaacaatgtggttgaaattgtgtatgcacttggaagtgagatggattgcaaaacacaggtctccatgctggggcaggagtggtgatagggcatggagtggaaatgtccagcaggcccacgtgcgaaaatgcagagctctctggctcttgcagacttggctgctgacaatagacgcgctccaggaaggtgctcgctgtggtgtgatctgctgcccacccctagctccctccaggagactggtgcggggactgtttgcaaatgactgcaaaagtaagaaggttcccacagagcagagcttgatttggggaccagccgagggcagtttgtcaggattccggcttgaaactgttctcacatctcaccgcctgaaaggacgagtgtgtccagaggacttagcattgatcacctctgtctccatgcagcaaactcagaggctcagcccgcattccactggaagggcgtttgccagtggtgttggttggaagagccttgactttgccttaggaaacatctttttttaagaattgaaaataacttgagtatgcaacagtagggcatttgttatataaattagttgactagtgtgtagccagtaaaatgatgatggtggtgtgtatttgttaaataaaaagatatgtgtggtattaaattaaaaaatattttaaaacaacatatttgtaatctgtttagtgtcctctttttgtaaaaagtacagaaataaatatacagaaaaaatagtagtcctaagtggtagaaattatgagcattttcttgcctttaaaaaaagttgtaaaagattgtatcatttatgtagcaaaaagttttaagtcagcattctaaaaatttcgtgttgttatagttgctgtgacaagatttaacttctgtatgcttcaccaatcaatacagaggtatttaagacccggtgtgtgataggccgcgctaaaatactatacacatcttcagaaaactagagaactaacttctaacttcctatattagtgtggcacggctgttacaaagatttttctcatttgagtctatcttgcttctttatcattgttttgacagtttcagaagaatcgtggcttttccccttttttacagtaaaggtacctgagactcttgacgtattgctttttggaaatgcttgtgctggtcacatgcttgcatctgggctagtgtgtctggcttccgtgtgctggtggatgcttactctgttttctgaaatactttttctgtacagtggccactagctgtactcctaagccacacacctaccttgaaaattcatgtcacttttagaaatagataaaagcccctcccatccagaaaaagtgactatcatgtatatcctcatcatgactaatactgatattcctgaaattgaaaatacatattccatatgtaccataaaaggtattaaagatatatggagtgatagatatattatatataacacttctaccctcacagttttcagcctaattgagagggtaagatccctgaatcatccatcagtttttcaggtctctgctgaaagcaggccacagctcagatccacacatctgaaccagagacagaggtggccaaaaataaaaagggggacagggggacaacctggtttagagtcaacaaatagactgcattttctggttagtgaaggagctctcctgaaagtcatataccagagcataaatgagcagatttccttgaggtcaccttctgctggccatagctttcttatctgtggagctgccagctgtcatccactttggggcacctgagactgccgagcggcaggccaggacccaagtgcgaaaacacagaacacctttttgtttctactccactgatgctggggttctctccctggtgtttgtggctcgtagtacactctgtggaacattcactatggtcatcgaagggcagcatcttcccagttgtttctttcttttcttttttttttttaatttaaaccgatctgagaagccagccatctgtcagcaaaacaggaaggctcgggctgtctcctgggctcgttttgctgccgtagtgagcgtcacttctccccgtgtaagagtgctggtgaaggctgaggcaagggcccagaaagattgagggacaaagacaggagcgcccgcattgcccatctgccaggctggaggtgtattcattattgatggaggtagtgcagttgctgctcagatatgcagccctgcctgggtaaatgagacattcttcagcaaattgcttcgttttttgattgctgattgtacgcgtgtcaccaagctgactcaaggttcatcgatgcatgctcagtaaattagaaagaacataactatggatcagccaagagaatgaattctgtgcctacaatgacccagggccatttaattttctgcttaattttgttgcagtcagtttgcattttgggttattatgcagtaggaaattaacaataaataacaaatttggtcctcctgtgcttgtaatgatatttttataaatctttgtaatgctgtttttaaaaggatcaaggtctgtgccagtctgatactccagcaagtatgtgaggaggaaaatgcattattcttgctagataaccttgttgttaaatagcataggggttctttatctctctctctttctcatatcttattagtatttttgctttaaactaaaatcccttcctctctttctcagataacctgaggaccatggatgctgatgagggtcaagacatgtcccaagtttcaggtgagaccttatgagatagctgtgtgggaagttcatgagaaaagcttccctggggccggaagtcacagtgcttggtatgctcatgggggaggaataggggctattctgcaaaagaaaagaccatgatggaatttgcctgagtgtttccttcacctgttacaaattatctcactttgagctgaacagaaagcctccaagatgaaattagttttactgttaaacttcaggaaaaaaaaacgggaagagttaaatacatttttgtactgttggaaggaaaaatggctgattggtttaaaacccaaacacatgccaatgatggtacttaaagagagagagagagagaagcttgaaaaacataattgttgggcacagtcatgactgtttgttcattaagcatggacacaacattgctcccctttgccatatatcttttcaagccgtattggatatagctcttctcatccaggagacccaggaagtggagaagtctgtagtaggaaaagcctaagggtaggtcacagactgtgaccatttggcagcactgagggtggacggcgagccagtccaacaaaaccgcacagttccccagtgcatggacataggaagacagctttctatctggccctgtatccagaggcgtcagccccagtagcagctttcatggactttggggttttcggtatttcatatttttgagcctcacagactcacagccagccccagaggctgacttatatttgagaaagttctcagtggcaccttgccttggctgagcgccctcgtgttttgaagtttctatgggattctacaagttggtgctcctgatgaagaccaggacctatgtgtggctgctcccctgcttggtggtttccctggggaaggtgcaggagaggatcttctgagttccatggaactggagatagatctgccaatcacaggcttccttctccaccactcctcagccgctctattcatgtttcagattttggacttaaactctcccaggtgcaaagaacaaacaaaaggctagcttatttttcttttagagtgaggcttcgtatttattacaatataattgccacattctttgtgtaattctcacatttatatcttaaatataattctcatgaatgagaattatataattctctttttgtatatcattgaatattttcacttaatttttaatttttttaatcgtcacaaaataattgtgtacatagacacaaaataattgggtacatagtgatgttgtgatatatacaatgtatagtaatcggatcaggtaaatcagcatattcatcatctcaaacatttatcgtttctttgtattaggaacattcgacatcttccttctagctatttgaaactatatattattgttgactacagtcatcctgcaatggtgtagaacactagaacttattcttcctacctagctgtaattttgtctcctttaacaaatctctccctatcttccactcccccgacctttccagcctctattagcctctgtcctactttctacttataatgatgacagcagcatttgttagtttccacatgtgagtgagaacatgtggctttttaacttttagaatgtggtattcaggcacttcatggtacagttggtaaaagtgaaaatgtgtccaaaagtttgtgattatctatataaacaaaaatggtataaatacaaatatcaattttgcattgaagaacttaccttagaggtatattctcacaagtgcacagagcatttaagcatttgttcactgcagcattgttatcagtattttaaaactatggtacatccatgtacttccacatacagctcttaaaaataaggaggatatgaatgaactagtatgaaaagaagtccaaatacatgtgaaagtgagaatagcatggttctggatggtatgcaaagtatgatctcgttcttttaaaagaaaataaattacatacacatacatattttctatatgcttgcccataacgtttaggaaaattcttgggtgatatttattaacctggacttcctcttggaagactgatggtagaaggaaggggacgagttagggaagaggaggagaaggaaaactttgcttttcatcttctaccttttagcattatttgaatttattttccttaagcgtttactttgtttcgtaaacaaaaaagcacaaaaacaaaaaacgagttaaatgggaaaaaaagcagtttagctctttatagcctctcatttggcttcgccagcctctcactgcagcctcagagagctggtctgggaaacactggtagatgaggactgtaatcctcactcatggaagaggatctcattcactgggtttgctgactgtgactagaagtgattagggtgtcaaaaaacccaagcatgttaaaaatttccagaggccaaaaagatgctttcattgttctgctcttcttttccttgtcgctttcactttgggtagcttctaaattggtattttgcatggtgcatttaaagaaaatgagacccctttggccaatgcaggagtctacactctgatattctagagtcaaagctgaatgctgacacctaggaattcatctctagaatgtttatataaggaatagcccctcagtattccgatctcgtatcttagtaacgaaactaacaaaagcctgattctcctctggtagttttcttgtctttaccataatacaaaataagtaatttgttctgcaccctgactgttcaaaggatagggtagctgggggcggggacaagaatggagaccttattacataagacttcctgaaaaaggaaactctgtttttgtttgaaatgatttggtctgaaatttagtttgtgtacacttaccaaagggattcctatttctaaaacactcatactgcttttgattcctgttaacctttgagcactctacgtaatgatgagagcacttaaagagtcatgtcacttttagtaaagaatcaaaggatactttttctacttcttcgagtttgatctctgcttctccagttaaaaccagtatttgtttttttcatttctaaagttggaagaaatgacagttagttatggcataaggatgtacatttaaccaaataggagttgacattcttggtaagaaatcttaccaagattatgttatagattataagaaatcttaacaagaatatgttcctaaatcatcctcttttcccataaaatattaaagtatcagcaatttcataggattcaacctaatgtatgcgaaatgctagataaacagataaatacttaatatctggctttttttcaaagcactgggttatttgttccttgagatttatcctaaatgtgggctataccctggtttacagtgtctcacagatgtgtagtagtagacactccataagtgtttactgacttgaatccacagggtactgagaaaatgctactgatagacttggaggagagcatatctaaagcaagctaccctttcctttagggcacgtctcactaattctttgggtaaagcgtatttttcttccttttgtgtttttggcagtctttccaaaaatacgtgttatacctatgcattattttttggtttggtttctaaagaaagagtcagccggtgggaaagtgaaggatgtgggaactgagagatctgcatcagcatcccacctctacctcccacgatgggacctgagacagttatttttgcctcctggaccactatagtatcatctgtaacaggagggacttgagccagttgatctctaaggttcctctggcacctgtgaccctaaatagatattggatattggtttaatgctatttgtagtgtgtttttttggggatatggaaaccagaagtttgtttccataaacataaacataaactgtatatatctaaaggatatggaaacctttagatatatataatctgcttacgtaaagaaggtttgtatatattgcagtgtcaatgggaatattttatcaagttaagcatagtaaatcacattgattaaatgctttgtatttaccaaacattacccaaagtgttttctcctttcaacctcacaaggacccacagaagaaaatacagttatcatttccaacctgcagggagctgagacacagagaatttaagcaactgaccggaagtccaacagggagtcagagattgctctggggtgtgatccccacttggaccctagagtggaagcttctccactactttatagagttgagattctatattttgagcttgtatttacccagagaattatatcctcttgggcaattgtgtataataaaacctcatgcatttaggagaggcgggatgacagaactttgttgagtgaattataatctacttgagaaattatttgcttacattttataagctaattataccatatctcatccagttttcccagaacacttctcataggtaatgctttatttgaaacataggccataggtaagttaagtgtaaatgtgtatttttataatttaaccagaagtttatttcatttttctaaataagtgaaattgtattgcatcttctaaattattctatttaaacacttgatgtcttgctgtctccgtctctgtgtgtttgcatgtcattgtacatgttcttaggaaaagtgtgggagcttgacgcaatatataccttatgtttctatgtgcatatagtttaccaaataataccataagtttacttagcatattagaatccatgcacattatttttattttatcttcaccgcaaccctgtgggatagaccaaaatcatgcttttcagcctcctttttccacttgaggaaaggagtcttaaaaaagggaccagtctcatgttcccattcgtcttacaactaattggtcaagccagaaagccagaactatgtcctgggtcactaactcctagtcactgtgtgttagtatttgagatgcctgttggcttgatttagtcatttattttttagtgttttataatccttgcatacttttacattttaaatggttaaccaggcaaattggtttaaaatcagtgcataaaaatactgtgcctatcatgatgggtttcatgaagtgataacttttcatcatggagatcctcagctgtcacagaagatgaggggccctgggtacagaggctcacgtgagggatgaaagtctcagcagcccggacttacactttggggcttttaggcaaatcagacaacctcttaagaactatcactgagttcaggcaaggcgagcttgaattaacacagggcccttggtgggcatgtgaatatatctcacttcactaccatccagttctgactctttactagatgcccctgtacataccaagactgattttttattctcccttctccccatgtggtttcttctgcatagagagttcctattgatcagtctgacccatggtattttagaattgcgatccctactgtttcattattcctttttctcccccatgttgaaaaaaataaatgtcctgagatgcaagatcagggacactggagcactgacatttagttcagtgcaggaactgaaggcagatgtaattcttaagaagcgtacctgttattatgaaccatcctcaacaaattgtagtggatcttgttttctcatagatacagcagttaaattttttaataaaagtaactaagagttatttggatgtattttagcatgcactgagcggaaagtacgacatttcttcattgggtaagtcctgattctttatgatcctcacttggttccagggccccatgcatctaagggtgtctcagagcatcctgcagtgctccagcatgatcgcagggaaaagctataggaggaaaagagtcaataaagtttagtttctcaacctcccacctccaccccataataatgacagctggttaatcatgagacgcgtgcacaccccacacgccctgtacatgtttactcattgggatagcatgtcaggccagaaggctccatggtcatttctatgaaggtactttagcaggtcttcaagaaggcaagtggcctgggtccctgcctccccaaattgcaagctccctgctttatgtaggagacctatgtgtatattacagttctgtgtaagattattttgttattcttacccccacacccaccccccaaccccccgctgccaccaaaaaaaaaaaaaaaaaattcctctgacaaccttcataaagtcctgggagtttgaacaccattgctctaggaagtcatcttatacaaaaataagagttgtgaggtggttcatatacctcctgcgttctcctatttggagtttttccccatttatgaaagaggtgaaaacgctaagatatttagcaattattactttaaacattttctatttataggccgggcgcagtggctcatgcctgtaatcccagcgcttgggaggccaaggcaggcagatcacgaggtcaggagatcgagaccatcttggctaacacggtgaaaccctgtctctactaaaaaaatacaaaaatttagttgggcatggtgggggatacctgtggtcccagctactcgggaggctgagacaggagaatggcttgaacctgggaggccgagcttgcagtgagccaagatcgcgccactgcactccagcctgggtgacagagcaagactctgtctcgaaaaaaaaaaaaaattctatttacagcagtgaaaatagtagtgacttaatgcacattgccaaggctttagcataacatgaacactttcactcaatgtctctctggccttttgtttttccttgggaaattcttataatcctgctccgtctttaactattcattttgtattggctatccaaatatacccaataatgctctttctgaaaatatgccaattgtggtaattacagctaagctggaatattaaattgtgatgtctgttttccagagaatgaagtagtattccccagagcataggcttggtgcctgtgcaggttctattttaaatattccaggaagggttgttttatatactgaggatgattttactggtcttgccagtcgtctgaaatgctggtattactcttgtggaaggtttattcaaacaaacaaggacatttcacacaatacctagtcatgtttttcagacattttaatgtttggttcatcatttgcacacactctcaaaaatctaggtttgtctatgtgttcatatcattttgcctgttgccagctcagtcagcaggcacactctcccaggctgttgctgttttgttagacttcttcaggaccttcatctaaaatggtcttccacacgtagctatactgcataagttcacatcatctgtttcttgcatgtgggttgtgtctcaactcaagtttaagttagatttggaagggcggaaactataggagttgcagcttcagtggagaaaagagcatttcctactagttatggcttcccaaggaaggttagattcctcagagtaggagtgattccccaatgctagaacctttggtcaaatataattctaatccagtcaaaataaatacaggtattctgtaaaacccgatttcattttgtaaatcctactttgtatagtataagcaatttttgtatttgtgtggattatattttattttcctatttcaaagagaagaatttgtattagcagactccctttgcatgcggagaggggatcattttcccagtaggcatggggttcccttccattccttgtccagtcttcttttccccactaagttaagtcaaactaagcagctggtaagatattccctggttcttgcaaagaaagtgagcagatggcagaatgtatagctctaagcagaatacctggtgtggtatcctcaaacacaaattgacaggagggtgtggtgtggcaagctcattgtgggggtaaattggaataagcttacagggggaagagttgacaaaagataggaagaaccttaaaaatatagatgccttttatgcagtgataaaatgtctagatatttatactgtggtgattattaggaatatgtgcaaagattggctattaggatgttcattacagtgttgtttaataattataaaaggacagaaagcaatgtggactcaaaaataggaaaagaatttaaataaatcctagtgtacccgttatacatgaaattatggaaatatgaccctgagcatggaaatatgtacatgagaatgtctaaaagctagttcattttgaaaaacaaaataatgtcacctcatattatttatagtatataaagatgattttaagagtggcagtgtctgggattataggtgattgtatttcttcccttttgcacatctatgttctctcatttgtattgtgtggggagaagtgactttttttataaaaagaaaaaggtatatgcatcccagcagagaagcactggctccacccagtacctgcctcctcatgccaccctctcaagccaaaagccgggggaagcccaggcaccttgaccatgaccgcccgagactcacacttcttctttctcatcagggaaggaaagcccccctgtaagcgatactccagatgagggcgatgagcccatgccgatccccgaggacctctccaccacctcgggaggacagcaaagctccaagagtgacagagtcgtgggtaagtgggtcaccagcggcctctgtgcctgtgaaacctttatctctttgtatttttccaagacagtgatgaagggatgcaagtcattttatccattgtgttccctcaactggcatattaaagaaatatggcacaaagatcagcaggatgggggtcctctggtgtgtgggaggatggacactcacaggccagcatggccgtgagagccacacaccccgcaaaatgtccaagttgaggagcaatcctgcccagggacgcgtctctgtcactgtcctctgtcctcactgcacttgcaggaatatcaaatgttatggattgtagatcgtgaaaattacacacttacgtgttttggcaacagtgcttttcagtgtttggggttagaacaagccacatctggccattttatgttatccctctaatctctagttcttaaatttcagatttaactgaaaatagaaagtttcataaatggtaatttttaaatgttagaaaaaaatcaaagcacacatttacataccttttccataaaaaagtgtgtgtgaaaccatctgcaactccactggttaacttacacatgcgacagtttgtgtttggaaaacttcacccaccctctttatttcaagaaaccaaagacggagagaagttcagtgaattaaccaaggtgaaatgacagagtcagaattgaactgaatttctgactgaaaaactagttctcgctccattataccatgttctaatgagctaatgagtcaacagttccctggaatacctgttttcttctttaaaaagagaaagagctgtgatactgagagctacctattggcataaaagaatatgaaggacatacaggtaaaatgaggtagactggatcaatgtgggtcagctacataagcccccatagccaaggagggaacatgtatgtaaaattctcaggcttatgagaatcacaactgtatgtctataggacatcctaatgcaataatgggaaaatgtgataactgaattttatctacatgtatagaaaacatattttgggccgggcatggtggctcaagcttgtaatcccagcactttgggaggccgaggctggcggatcacaaggtcaggaaatcgagaccatcctggctaacacggtgaaaccctgtctctactaaaaatacaaaaaattagccgggcgtagtggtgggcgcctgtagtcccagctactcgggaggctgaggcaggagaatggcgtgaacccggcaggcggagcttgcagtgagccgagattgcgccactgcactccagcctggatgacagagccagactccatctcaaaaaaaaaaaaaaaaaagaaaaagtaaagaaaacatattttgtatgttttttatgtgtcttagaaaccaagtggactaggtataatggttctttttttttttaactcagcagcacagttaatggcacaaacagataaagatggtggctaaagccatttggtgtagatgaatccagaaaacaagtacatttatgaattattttaccctattggaaaatggctactatatgtggaaagtagaggggaagagaaatgaacttgtgaattatctactctattctaccaaagaaagatacatatatcatttcagggttttccaaagttgggatgcaaaggatcatgtttcattttaatttatgtatttatttttgagattgtcttctatgtattgcaagtgatactggttttaccatgcactgtaccactacgagggttcctttcaaaacacattcatataaattaaaaaatcagtttaaataaaaacatcaagtcaatagtagtacatagatatgataaaagtttgcaaaagtggcacaggaatagtcattgggtggtgatgttccctgatttttttagtccctgaagcacccagcgccaagggtactgttcttcctgcttaacacaggagcaaacagatgctcacaccctgcccaagaacacagaggctcagggactgaaccagaagaaatgatccattaaccatgtaaatgaacagagagcctggatgtcagagtgaggaggccagctcgggtcgctgtggagaaagagatgggctctccctggaatccctgcagctgcccttagaggctgtgagccatcgtggctagggcatctttagagaactgaaagtcaggccgattcctggagagtgaagaatagcacacgtgccttcaaaacagtgtcagtatcctgtcccttacggacgtcatattgttggtgggttttcctgggtgggagatcacccagtatctggttggcattactgatgggttcccagagctgtcttttggttcaaagtcctgccctcggtcccagctgcccccagcacaggtgtcaccttggagtcaggggcgggtttgtcatagcccatgggtcctggccctgccctttatcatgtttgacatgattttcagtctacttttccttcaggatgtaatttagaaaatatgatttcagaattgtgaaatttaaaaaccctagaaaacctctttgctttgcttgtaaatagcagttgatgcagccctgtggacaggcgggaacagccatgtgacgtgggcactgagatgatttgggagattgaatgctcctcagtggggctagaggccacaggagtgataggttactgtaaagaaagcaaaaaagatgcaaaagcctacatgacaaaccaggcacgacacaaaggagaggcttgagaagaggaagtgaaggagggagaggattccacaagaccgaagtgcccaaggagacatggagggaagagagggctcttcacagagctgagtttagagaccaccctccatggttctggaaggaaaccagccagctttcatatgtggtgacaggctgttcattaatttgtagtgtacagcattaaataaatgaagtatgtagagaacattaggaaacaatataaaaaggtaggggaattcgtggataaagggaataacctgggtaccatatttcatcacattattccatgggaataagtcatcagtgcaaaggactgtaaggagtgcacagcaggaaatgaaaagacagaagcagtggggccgagaggaggaaatggagttgctgaaatgcagggtgagcagcaggggcggagagtgccctgtgggtaaggcacacctcagaagggacaggtccaggccctctcctccctggcctggagcttctggcaaggcttttcttctctgagcctcactttcctcttttgaaggtggcaatagtaactgaacctgcatcacagaatggccttgagggttagatggaacatatatagacataatttgttgaagacagagtagcaaagacacaaagtaaaaaggacagaaaaaagaaatgcaacttgtgcaaaatagaactttcaaagatttacaagaagggtaaatgaagctatcgtagcttcacttgggaaaagggagagaacagatgaccagcaccaaggatagaagagggatttcttactttaaaaaaaatggtccaaagaataaacaaactgaaaacagccaaaagagaaaggaagaatttgttagaatagggcaagactaaaggacagagtaagggtgctggccgccacctgactcaagttcaagtcgagctgccactgttgaaatttcaaggagaaatggtgagaagctaaaagatgcaaaaaaaaaaaaaaagtgggaaattatttgctaataatagattgcaagagtgtctgctttagaaagtgacaaggaacacttttggaaagctgtgagctgttatgaccactggcatccttcagttttagacaaatctaatctcaacaaggataacagaccatattaataaacaaattgatgggaatggtggaaatcagttccctatgaggagggtgcaaagttatactgattggtgagtcactcccagaataaacaaataatggtttcacacctcattgcttcattcagtgtggaaggaacattctaggtttgtgtgtaagtttggggagaaggagacagagccagagggcagagcagccctgcagccctcagttggcctccattttcttcccggtctgaagaaaatgaaacaaaggatttaaagtgttgcttgaagaatttaagttagacttaaaggagagatgtggtctttactctccttttggcaaggagagtttagtcatcagaatggtctatttagtgagactgtagcttgttcttctgaagtggtctttttaaaaactagagaactctcacctctctgaagtggttgtgtatgaaactgtttaaagccgaggatgcactcagtgacctcttcaggcaaccccagatttactcagtcagccatggcacacatgctagagaagtaaaaaaatgaattgagacttgttctgaaaactcttgagcaggggctcttaaaagttttttgtttaggaattatgtcagggttggggtctggagagcagataagtgttttgtgtgagaaaagtaaaaacttagttattttcttcccgtattcagtaatccttgcatttttttcctcctcctttatacatacatttttgccagttactgacttctctcttgtacacatttccgtgtcctaaattaaccaagagtcctcagccactaggattctattgacaagcccacaggtctggaggtgtgagtaagggcattcctcccccatgaaatcaggcagcaggggaggcgtttgggtgggtggaacagaggagcaaaagaccagaggacagagaagaaagattgcaggctagaaaacgggatggccacccctgagaaagaggacaagcaaatttagggcttagagtagaattgaagaaaaaacccagggcagaggcacaggaatgctgtaggactgaactgggaccatgattgatgtaagtggagatgggttggttgaatccgtggaaggccaaaggattgaggcccatatgggcctgggacagggcttactaaatgacttgctacctggcttgataatcgggacaacctcaggcagtagttattcaggcgggaggacacatgagtttgaatatagggagataaggtatatttctatatgccctggtacattccagaatgtgcttctctggctctgtgcactggagcagccaactcctgaagttgggaggggatcgccatggtttcatgagtggtttatttcagcttgcatctcacagtgtggtgttcagaggcataggctctaggctccctggcagcactgagagatagaaactgaatcagcacaacttaggtccttcatgttgaatagtttgaacagaatttgatttaccctagcctaattgaaaaaaaaaaaagtagaaaagaaatctaggtattttccctctggactccagaacaaatgattgagtgtgtaaaagcataaatcttttttattcattcaattgaagtaaaaatttctttactcagccatgagttctaagactgcagtgggaattgtaagaggattaccatattttaaattggctattctggtgccaaatctccccttcattttacagtttcaggcagaagaccagcacggtagtcgggggaggggggtgggtagacaaatggtggtaacagtcctctacactctgggagggaaggagcggtgctggggacagctttcaccatggaaagaaagcagccgctctggagattgggcagcaggcttttgagcacggatgcggagcctgcttatctaatcagcagctagaggaagttgtttttatctgcgaaatgcatctcttctgaaccgataaaccccattttagctggggagaaatattatctagcagcatctgagcattagctgcagctgaactcaagcatgttcaaatgtgagagaagtctcaagccgagaactgtttgccgaacttcaagtggctggagtactggttttcaagttttcttttctttcttctttgaggaataactgaaagcatattatttggattcttgttcaccttgcttttttagttcctttcccccttcatagttgaattattgttcagtggtccatggagcaaattttgaaaattctatgttattcatcttgcaatttactaccactatcttggaatgcctcgtgaagacagcaggtattgtcagcatgaacatatccaaacccagatgatcccggttggaaagcctcccctttttcaaatttcccaaacatgaaatatagtattacaaaatgatctgcccacctctaagcttttccatgtttgataagagaggccatcatgtaacaggttcattgcttcgcatttcaaagtaaattttaatttacagtcttccagattcgttttgggtaggatttttatgtctgctaatttgtcaaatagttcaaaatgtttagggatcatgatgtcttccacagaacttcatgacagttttgattttagattaaagtaagtgacttttgtgctatatcagataacatcttgtactaggttttggaatagaccggtgagatcaggcagcagaagtttaataactttccttggaaaactgaaattgatgtgtgtgtgtttgtgtatatgtgcatgtggtacatgtgttgtgtgtgtgcaggtgtctgtatatatgtggggggtatatgtggtgtgtgatgtatgtatatgtatgtgtgtggcatgtgtgtgtttgtatctatgtacatgtggtatgtttatgcatgtagttgtatgttgtgtgtgtcatatgtttgtgtgtgcatgtatgtgtgtggtgtgtgtgtgtatgtgtagtgtatatgtgtatgctgtgtatatgtatatggggtgtgtatgtgtgtggtgtgtgtatatgaatgggatgtgtagtgtatgtgtgtgctgtgtatatgtgtatgggatgtgtggtgtgcatatgtagtgtgtatatgtgtgtgagatgtgtagtgtgtgtgtggtctgtatatgtgtatgggatgtgtggtgtgcagtgtgtatatgtgcatgggatatgcggtgtgtatgtatggtgtgttcatgtgatgtgtatatgtgtgtgggatgcatgatgtgtggtggatatgtgtgtttgggatatgtggtgtgtatgtgtggggtatgtgtgtgtatgggatgtgtaatgtgtggtgtgtgtgtgtgtatgggatgtgtggtgtatggtgtatatatgtgtataggatatatggtgtgtatgtgtggtgtgtgtgcaagtaatatgtatatgtgtgcaggatatgtggagtgtggtgtatatgtgtgtattggatgtgtggtgtgtatgtgtagtgtgtgtggtgtgcatgtgtgcatgggatgtgtggtgtgtatgggatatatggtgtgtggtgtgtatatgtttatgggatgtgtggtatgtatgtgtgatgtgtatgtgtatgggatatatgatgtgtatgggatgtgtggtgtgtggtgtggatatgtatatgggatgtatggtgtgtatgtgtgtgagatgtgtggtgtgtatatgtgttgtgtgtgagatgtgtggtgtgtatatgtgttgtgtatgtgtatgggatgtgtggtatgtggtgtgtatgtgtgtatgggaggtgtgttgtgtatgtgtgtgtgggatgtgtggtgtgtggtgtgtatgtgtgtatgggatgtgtggtgtgtatgtgtggtgtgtatgtgtgtatgggatgtgtggtgtgtgtgtggtgtgtatgtgtgtatgggatgtgtagtgtgtatgtgtggtgtgtatgtgtgtatgggatgtgtggtgtgtggtgcatagtgtgtatgtgtgtatgggatgtgtggtgtgtggtgtgtatgtgtgttgtatgtgtatgggatgtgtagtgtgtatgtgtgttatatgtgtatgggatgtgtggtgtgtatgggatgtatggtgtgtatgtgtgtatgggaagcgtggtatgtggtgtgtatatgtttatgggatgtgtgctgtgtatatgtttatgggatgtgtggtgtgtatgtgtgtatgggatgtgtggtgtgtctgtggtgtatgtgtgcacatgcatgtggcagctgagccactggggtgtttgctgtgcctgggagcaggccatgtgggtaggtaggcctggctctattgattcctagctgttggccctggcaggtggctctgctgttccttcattgtgaggtgagggcgccccctcacccgatcctggctgtcatgtctgtgaagcctgacccagcagggcacagcatagagggctgtccagagcagctgcactgatatcatcatcatctgcattttggcctgctccatgtctgacattcctgttaagcaggtggctttagggagaagcattgacacactgacccctacaaaaagcttacagttcctcagtgataaaacacagggtgcagcccgtggccaggctgggtccccggagaagactcagttggcaaacgccatgtggttgccacagctggaagcactgggggaggaggagccctgcctggccccggcgggggcagggggagggtgcagggggagggtgcagagggaggggatgagtcattttgccacaggcacgcatgtgctggtgaaggggcgtgcgtggggaccatgggtcacatctcggctgtccccttctttaggaaggggtggaggtggcaggagtggggcgggttgcaggctcagaggcacaggaggcccgcactcgctctgaggctgaggaataaagggtgtggtgctggaaagggaaccctcaaacctgctgacctgggaatagcccttcagggggcctgtcagatagctcgcccttgggacaggcggtggcgagaggccaggaggcctttggtgcaggaggggaagaaaggcagggaggggcgtctcagagggcagagaagggcatctcagcagccccgtggtgtttgcccctttgaggcggttccatccgttggctgcattgtccacttcggtgactggggccacagtgattctgctgaatgcaatgggaactgtacccagagtaggggttcgcatgagaccaagggatggtgtctagccacggaacagtctggaaggtagggatgtaagtcatagggcaagttccatctgtgggacactatcagctcaggagggacagccaacaaaacagcaggaccacaggggccctggggagtaaatgcctttaattgtctaagagaggagcaaagaaggagacagagtgagttgtgtgattcgggtaaaatttctagaaagaaacatggcgagaggagttgctggggacaggataatgccctagaaagaggtgtctgggggttggcggtgggtttcccacaagcggctcggtaggattggggcaaaaccaaccatgtgagagggaccgggagcggcacaggagaggaggcagcagctgtgggctgtcaggcatcaggaaccaagggaagaaagaagacaaggagctggttggggtcgggggtcacaggactaggggatggtatttacagggtagtctttttaggagaatgtttaaatgtgattgaagtccagtaagtgggagctagcagaatagaaaggattaaaatctaactagcagccccagggtaggccggcgtgagtcacctatgagctgggcctgggcctggactatcccctaggtttcctcttggcgctcttctcccttccctcagggctctgtgcagtggtctccacacagagaggtcttcctggacacatgccctaagatagcgttctcagcactgttgctccttgccctgccttatagcagcaacgtcaccactcgtagatgaaggactcagctctgatttgctccctgcgtgatccccagtacctgggcatcccccagcctagagtaggtgctgtctgatacattggtattgaatgaatgaatgggaatgtggcactgctttctcaaagatgagggcaaatggagcatgtaggacagtagagagaactttcaaagtgagaggagagaatttgaggctgcatctgatgttcatcatcttattttaggtcactgtttctcagtgggcacgatgatatttgatgtagggggcacttctttgggtgagactgtccctcccattgcaggatatttagccacccacatcacaagtggtaccatggtgattgtgatacccaaaatgtctccacatttctcaggtgaggaccctgctggaaaagccaccagttagcccggacacattcgtcctcacttccttcagtacttcccactcgtaagagctttgaaaattcagaagggcaatctttggcagatttgttaaggttttttaatatagccagtaatgttccagaatctgacatgtgaattttaaaaaaaaactatagagaaattggaccagaaaggttgaaaaatgccaaaccttgcattttcaaccatgcttgcagtgatgactatgtgtcttcaaaggtgcagattaactatggaaagttgcttatttttataaacaaaaaccaatgttaatcttcattttattttgatgccttacactcaagatcacattgaacctaattcgttcaataaatagaaccaacttctagaaaataaagagatccttagattaaaagtgctgtcagagtagatagataacattttctgttgccgggtttttggtcccagttttattttctgccttgttccatctgtgttgaaagaatagttagctctgtgtataaaacaaccaactgacatttcgttaactttccttctacatggggtttcagaaagaaatggggaaaaaaaggctggggggttgagctgtggttgccagttgccacctgtccccctatctggttagggcattttctgatttgggaagtgtaaatataacccagaaaaaacagcttcccctccttttatgcaaaagtagaaaggcttaaacagagtacagccttaacattgccccggaaagtttccacagaacccgttagactaaatgaaagctcttgctctgggggagtgggccatgccaggaagctgccctaccaagccctctcttgctggcaaggcagggctgggcctgggctactgtctcccaccagataagcccagctttcttcaaggccctaacaggaacctacatttcacaaattaccgcagacacgctcacaattttaaactctttgcctgttaggatcagtgagataacagaaacttatagggaactcagaaagcatgctccggtgagcagtgagccacctagaatcagccaactggtaatatgcggaaaatctgccaggttgttggtaaacttttggtagcttgaagtctaccgtggttgggagtatttacaccacagaaatcggtgaacgctgcaaatcaaggctttttctttttgagagctggtttatctgtgcaccactagcttaaatacacttttaatgtcctgacagctgaaatttgacagtgaaatttgaaaggaggaattcttttgggtagggttgtgtgtgtgtgtgtgtgtgtgtgtgtgtgtgtgtgtgtgtgttgtggaggaggagctgggaaagggtgaatctattggggaggactgtggggtacatataatttgtccaacagaagagttggttccaaatgactctttgctgttgaagtcacaaactggaaatgttggagcaactagactctccatattctaaacaaaacacatctttagcatttagtttggttgctgggggagtatgattagaaaaggatttggccaggcatggtggcttacgccgtaatcccagcactttgggaggccgaggcaggcagatcatgaggtcaggagtttgagaccagcctggccaatatggtgaaaccccatctctactaaaatatgaaaaaaaaaattagctgggtgtggtggcgtgtgcctgtagtcccagctactgggaaggctgaggcagaagaatcgcttgaattcgggaggcgggggttgcagtgagccaagattgtggcactgtactccagcctgggtgataactccttctcaaaaaaaaaaaaagaaagaaagaaagaaaaggattctgtgtcttatttatgagctattatgctattgctgttatttggggctgaatgatttttatttggggctgaattatttttaaatgtaaagcaagatgtctaaattacattaatatgttactaaattactatgatttttccagaaaaatattagcccatgcaatattctttaattcctgtgattaagttggtcttctgcaaagaaatttctgttgacttccttagattaaatgcagccctgagataggtgtgcaggtccaggaagtggcctgtgcagttctccctgtgcctgcaggggtggcttcttgaggatactttcgcagtaagggcttagcaatatgcgcatgtctaaggagtctatcagaaaactacctccactgtggaagggctggggattgtatttttgtgctttgaagcacctgacacttgctgtaactccattaactgaagagccgtttggtgccctaatttcaggagtgagttcagctgcagggatctcagagagacaggtctagggtgtgtctccgcaaggcctgctgccaggagagtacttcccccacaggggatgtggctgcaggctgtcgatttccactgaaatcaccaggagatgattctggtgaagccctgaacatccggtgtcactcctcaccaggctgggtgtgatcgatggaaggccttcagcatggccccctgctgcatggcagagcccagtggcagatgtggctgttctctttttcagagtgaaggactgttaccctagaagtgaaatcctgctgaaatgagaaaagtgctgttattatatttctacaggcagctatatgatcatttttattatgattcttggaacctggcagaattttgactgcttttgcaatatctctgtgctctccagttgaacttggtggcaataaaattaagaatctgccagtgccccccaagcatttctactctgtccataatgtttagtgacaattaactattaccaggtgtagccacccttacttaatcttagtgttatggtgcaagtaatccttagtgaaattattgttctgtagagaatgctgtgatctaagtgaagtgactttcatcctgagatgacaatggagttgtttttgtcactgtaaaggctgcatttactcgcagttttaaagcattattgctacttatatatggtctcaacactgatttttatatttgactttgtagctttaagtgtaaaaatttctgttaaggttttatatgtgttagttaatttttcccatttcaataaccatgttttattattgatggagtctcctaaaaatatacccttaaaatctgcttgccctgtgcctgcacacatattttaaatgttgggtatggagtctttttttttttttttttagacagagtttcactctgcagcccaggctggagtgcagtggtgtgatcttggctcactgcagcctccacctcttgggttcaagtgattctcctgtcccagcctcccaagcagctgggattacacgcccgccaccatgcccagctgattttttgcatttttaatagagatggggtttcaccacgttgaccaggctggtctcgaactcctgacttcaggtaatccacctgcctcagcctcccaaagtgctgggattacagatgtgaggcaccgtatccaggctgtatggagtctttctaatcaggacataggcagacagtcctagcccagctttatgccttatgagacgcaacaacgttgaacagtcattgtttgagggacagaggttttacagatggatgataactagcatctgtggaacattatttgtgaaatatagaaatcagaaattccagcgtagcactgtccaaggggaacataatttgacctgcatatttgctggtccatttttagtagtcacattaaaaaagaaaaatgacacaggtgaaattaatttgaatatattttcttaattcagtatgcttaaatattatttaagtatgtactcaatataagcaattgttaatgaaatattttactctttttgaactatgtgtttgaaaccccggatgtattttttttttatcttcaccacacatttcaatttgggttggtcacatttcaagtgctcaggagtcacatgcagctaggggttacctattggacaggcaggcagatcttgagagctccaaagaactgtgtgtcattatattgtggaaaaaaaaaaaactttttagattaacccttgttgaatgttagccaaagactagtcatttatcaactctctaaaagtagtttttaaatcaatttgcttttcaaaacattttctcagtgttgtcacaaatgtatgtgaacatctgctttttaagtctttcattttttgcccacatttgatgtaaagaaaatctaggtccagtccaattttaacatcaattgggtttattttatgtagctgtgttttaagtacaggtcttgtacttgtaggcattttggttaagtacaaagtaacgagaatacaacaacatgtgaacatgtcaaacaaatcagtttttcaaagctaagtagctgaattaaatgtagaaaaacatgacatttaatttttgtcttcatcccttcattgtattcccatgtactctatggtaacttttttaagttgggaagtcctctttaaatttttgaaatgaggtagctgctttatagacatgaggcaggttcccagagagcgcccttgaggacaggctgtaagtaaactaagctgcaaagacagacaccatcacaactatggaattgttttgcaaaaattgtggagagtgatgccccttgatacttcctaaaagatgcttttgcaacctgataaagcaaccgaggagaggtggtgaaggaaaggcttttttttttttttttcaaagagagaaagagaagcagcaagctgcctgcctgcagcctgagaaagcagttccaccttggcacccatgtcctcatagcccaagcggggccaccctggcatgtgaatctgctccgcagtgtgcagcccagaccccagccttcctgtgaaagggacacctcctgctgctaaccgctgctcttcctgcagttcaggagacagcgtgtgctcctgtggcggctgcatttcctgagtacaccatgatccaaaaaggcacagattcgtcatggaagctttgattgggccgttactgtcttttgacctttcttgtttgcttgcccttttgtttctttcctcctttttccttccttcctgtctttcttcttccttccttcctttcttcctttttctttttaattttcctcctttctctttctcctttctttccttccttctttcctctttttttctttcctccctccctccctttctttcttccttcctttctttcttccctccttccctccccaccctccctcccttcccctcgtctcccttcccctcgtctcccttcccctcccctccctccctcccttcctcctttttctttccctttcctccttccttccttcctctcttcttttctccatccctttcttccttttaacatttgatcatgggtaccatttcatcaagattctgttacttaggtccaaggactagtgaccaaattgtttttcccaggagtaaacttttctacaagacatagagaaatgactgccagtgccatcaggcacaccatcctgtgtgataggtaacctaaaataatggtacagcggggagtaactggaagagtcacaactacacaaatctcttgcattttcctgatcattaaatgaattagggatttcctggtttgagatttgtggacaaggagaactacttctttgtacaagaggcttgtgccaggagaagacacgctgtggttacaatgttactgtctctagctatggtcatttcatgtctttaactgagtgtacctagagtgtaatacagtgcagcagtgatgggctgaaggcctaatgcaccagcacacatgcctggaagacctaacgttgagaatccgcatttttttcatcactcacatttattttgcatctccctgttttgattaccttcagagacccaagcacttgcttttgtatatttgagtctgggttttattttccttttagaaatagtccaaaatccttttagagcctagacctttgaataaagtgctaacttaacctgaggaatgctgtttttttgttcacaaataaggcttaattgtgaagtcgtgagaattgctttctgagctgacttataaatttacccacctctttttccccaaacccccaaaaaatcctccagaaggtgagcccagcttgttgtaaaagattttgaagcagaagtcctgggtttaagcttcagttctctgccttaccagaatcgggatgtgtggggcaggcctacctcctctttcttcatctgcaaaatacattgaatgaaatcatattttttcctaagtatatgacatgtaaagtggtatttcaaaaactcattaatctgcatgaccaggtcagaaaagacacaccttttgccttgtgtctagtgtgtgactgtcctcacatgtagatgacccaggccacatccgctgatgttgtcttcctttacttctttgtcccttccaatgactcctttatgaagtcaatcagcagtgtttctaagccaggttccttctcttcacacatccccaacccacagggcaagtcatccacattttgcctctctattttttgtgctttgggaaacacattttacaacttccaaactatttgcctaagttcatctgagcacatctagaattattccactcctctgcatcagtgctaaaagtacaatcatgtataagctgttatactcccgttattttgtacctcatgtgcaggtagattgcaagttccttgcaggcaggtactacatctacttgaattgcttcacgatcacataacacaatgcagtgttaaaagtagaaaagtaattgacatcttgactaacagaaacactaaattcagtgtattgtgatgatacctttttaagagataggctatagaaatagaaaatcatcttagttggaattgaaagtcaagaagcaacattttaagtaagagatgaaaatacttaaaagggaaaggagcagtgcttacacagatgaggggaaaaaataaaatgtatcatctaaaaatttaaatcaaatatattaggttggtgcaaaagtaactgcgatttttgccattaaaagtaatagcaaaaaccacggttacttttgcaccaacctaatagatgggaaaaataagaggaatgaatattttaagctttgctatataattaaaatattcttagaagtctggagtctgtgaaggtcacaccctctggtcttctcccagcccatagggtataaataatctgaattgacggcatccagggatctcagaaattattagtacatcccacagtgaattaccaccttactaaaatattcatgggtatatactatggatttgttttatcctatttagtcttaaaaactataaagaaatctgcaggcttattaacatattactcagaatcatattgtctccaaagcacaaactgaatcagttacaagatattggactagagatcatggcaaatcagaggtacataagacctagttccgttgtggagctaaacaaactgcagagacctaaagggaagccttgcaccacactctaggtttggagctcaggttttgagtggtgtcagcactccagaacacatgggatccccgggaggtggaaattgagccgtctttggagaatcagctaatgagacagatgcatgttaaatgtctgttgtggcccaggcactctgctaggcagaggggtgaaccagaagaatgagattcatggggccaaagaatttgccttctggtgtaagaaaagatggaggcagcttggcagaaagaaaaaaaaggtaaaagatagaaatgaaatacagaataatctatctcctcatcccacaagcaattctgcctggtttgtggctcacgcctgtaatcccagcactttgggaggccgaggcaggaggatcacttgaggtcaggagttcgagactagcctggaccacaaggtgaaacctcatctctactaaaaatgcaaaaaaatttagggccgggtgcagtggcagttctgcctggtttgaacatctggttcagttaaaatacttcatgaaaatatttagcttgatgcactgaaatccatcactgcttttgtgggatgcactgactgcagtctgactgttctcctagtggaaaggggagcaatagcttctgtctgcatgtgcatgaaacagatggaaatttagaagagattctagtgcagacaacttcatcccacccccgctgcccacagagctagccatgtgcacggtgtgtttgaacaatgttatgttgctgctgtgcacagagatccccagtctcataaaacatactggatggaaggctgtcgagagatcatcaggccccagtgcataagttcttcctaagcatccttaacaggtgatcacctcgcctattgagaaccctcaggaggcagcccacttacggaagcactgattgttacaaaagccctgaactaaatctgcttgtctctaaagccagccctgcagatgtctgcagtgtaactactttctcttctctcaagcagttcagcccttcaaatcattgacaacagctctacaaagccctctaaatagcccgttctcctggctaaagtgtgttggtttcctcagcatatgatgaaataaacaccatgatgcctttgccttgctttttcttgctggtctcactcttccctggcattcttcaatgtgcccaagtccttcctagaacattgcacccaaagggccttcctggtctttccagggaatgcagaatgaaaaagaaatcccatcccctgcctcgaaaggagctcttctgtgtgtaagtgtaccttccccctcagggcataatgggaaacagtttgccaaagctaactgtgtaatttcacatgactcactgccgttagtctgagaaagcatggcatattaaactcattttccatgcttcccaactttatttactattccatttctgaggggcagagagctgagcaggaataacaccttccttgctcctcctaatggggcacatgtgcacatataaaattgtgttcgatctttgcagccacgacccatgttaactgtcaactgagaactcatgagtcagtttctcaggaacagccctgaagacattatctcctgcatcttgtatttgtgcctttgatatttggagggaagggtaggatctggcaattatcgttatctcatttggttcagtgtatttcatttcagcctgttaagatcttttggaaatactgtatgtgctagttacagggacagtgataaataagacagcattcctgtccttgagatgctatttccacagctccatgagatgcctagttaaaaacagagccttttttggaaacagcctactaatctgttaaaataataatggaaaaagataaagttagcatacctgagctgcaagggctgcctcctggtctgatctctgataaggatcagatcctagagcctctgagatccctgtcctccctgtctgcacaagcactcgcagaaggagaaacagtttacggtggttcatcatgactttgaaccaagcttaaagtcaaagtcatactctttaaaccattggaaaccaagtttttgcgagttgcctaaagtgggcaaaaatcccacaatgcacctgagcacaagcaggaaaacatgccattgtctccccaggagccctccttgacttctactttactttatccattgacactagtcttataagtagctttgtctgtccaatttttaattgaatttcgtttttattttcgtgagtggaaatatcactttgaaagaaatcagtcctctcctgaaatccagaattcctggagctcagtttacatgtttgacgcgtgtcacatgattccacaagtcactcaagggcaggaggattaccatcgatacggaaagatttcttagaaagcttaagtgaaagggaaaccagggagaaagtgtgcttcgtgagaataggtatgcggacggctctttgtgacgctctgtgcaaccctcgtgtttgcattgaaccaagctgtgctgcagatggacacccatcccatttccccgactcataccagggaggcccactttgcaaggtacacagaggggaccacagagcagggggccatgcaggggaccaagggacattttagtgtaaccaaagtgtgaagtatgccctttatttcaaaagaataaagaaaatcacaggttttcccgctgatatgccagggacatttccaagagaattccttttttgagagaaatctcctttgatatcccatcagtcagccatactgcataattgttagatagtgaaagaaattcattttttaagtttgtcagaaaaataaattctttgaagtcttaaattgttgcctccggacatgacatatctggctcttccagaatccatgttagtcctagctgaggaagaaggagaaggagaggggcgtttgttgattattgattttgtaagatgccccacacgttggctattagcagaattctcacttctaaaaggaaaatgagtgtgagctatgttcaatgagaaacagttatttttgggacattctttgaggtaaaacacctccttaagatgctgcttccttattgctatgggaccaagattagagcaagaacatagtggttttcagaccctggacatcatccacagccgcagcagaggccctgcccacttgaacaatgagacaggccaccattttgtttcgagaatgagcaaagtgaaccaccatgccagatattgttaagtcagcaactcttctgaagatggacatagtaaaaaataaacaaacaaggcagcacttgagatggtcatggcagagcaatctcaacaagcgatttgttattttgcacagtgatgtacccactcattgaataaaatgcaccagaaccatgcactacagatgctaaggagagttgtccttcaacaaaggagataggccccactggccgtggggcagtttatgttatttggtccttgtcatgggcaggcatggccctttctatgcttcacagatgaggaaggtccctggcacaggtccagtctccagcatggcctagaggtggcaggtgctacttagcatcgccagcctcctgcttggtcatggggtcagccagtttataacacgaacggaggttaatgaactgatcttcccatcgcacaactggtatgaacccacatcttctgattataaatcttttgctctttaactcttagtcattaccactgtctagtgtaggcctgtgtgttcatggcctttgttgccaccaaaagatcaactattagctgaataacatactgagacatgttggtgttgtgttctaaatagcactagtaaactgttagggaaatctgactatatagctactatccagtctagtttttcttgcgaagtgtttgttgagtgtgtaatgaggagtaaggaaggtgaataagaaatggcctgagtctttataaaataagcaaggagagaaaacagttttgatgagaagcgcatggaatttttagaagataggacgtgtattatgtacctataagaatgggtaggattttagaagagatggatggggaaaaggaacagggagtgggaacaaaacgtggaccaaggaagagcaggtttagccatggaagcctcaccgccggctttggtttatcgtgggccaaggggacagaccctgtggggagggctggcagcagggaggtcttccagaagtctatcctgcaggcagtaacagccacccagtctataagctgagtgggcatgggggtgatggggaatgggtggggagttattggggtaacttaccccaaaatgatagctagctggaaccatttattctattgcattttatcaataaatcttataggaagtaccatcctagtgaaaaccctgtcacattgagggcattcatgcttatgttttaaaacatgttattgggtctatgaaaaataaggctgaaacctatgagcaccctccatgcaaagtttcagtcaagactttggaaacaagacagtgtcttactcactttataaattcattcagaaagccgtaggtttgaaaattccaaacttagatgtaagaagctctgagaaacacatgaaatcacccccacatcagtagagatgtctcagcagacatgggaagaggggcagcagggtgtagggaggtggggcagccccggggtgggccttgcaggctgggcttgaatcccatggccaccaccgtccgctgggaggcctggagccggctgcccactctctgaccagcacatgttgatgctgtatccttgaagggaccgtggtctgacatcctgtgatgcagacctgaatccagcacccacagggtctgcacattccctctttgaggtggagcccagctccagaggctggtccctgactctgtttctcaagaagcctgtacagatgttcccctcaccactgtttccagtcacctttggctttcacggtgcagatgctaagtttgattttcagagcccatctgggaatttagtgaactgaacaggtagcatttctgaacccacccataacccatgccctccccactgattttgaaagagagtttgctgcaggtgactttgcagctgggtagagaatcttggggcaggattccgaggcaggcagatgagtgaggataaattgggttctgacggcacgttacaccagtggactctaacgacgacctcacctcgtgcacagataattctgccttgtgcttaaccgttagaaatgtgtcactgaagtgtgaacatattatgctgttagatttcccatcatttctgttctttcattccctctcatttgcattggttactcataaatgtagatctttggtatgatttgtacaactgccgggtgtcaatctgtgaaagaaatagcagagcaagctgggctctggtagcgctttatccctgcgtgctggcttgcccgggttgactcagaggcagtctcacattcagctgcgctggggccaaggacccagggagccaagtgtgtttctgttttctgtatttagcaatttaagacctgcgtttaaatactagctatgcattctagcaaaagaggtttatattttaacacagtaactcttaattgtttaattcagttcgtgtgttacctctcggaataaaatagtggaagccaattaactatagacttcattagtttggatttaagatcaccaaaacatttaccacatctcaattgttcattacatgctctctcttttttaatgcagtttttataatatggggagtgggggtgtggatttaaccattatatttttatgatattgggagtagatttaaccattatgtaaattggattttttaattttaaaaagtcacttatcttgatgtaaaatcatgtcttagtaaccttgataaactaagttttgcatgattacaccttaaggttaaaacatatttcatttcatcatttcccagaaggggcactgaattcacccatttcctgtttttcatctcagaatgttctgtgtttcctccatatctactttcccggcagcaggaccctggaagcagtcacaccaacctcatttaccccacctgagatttgtgcgctttgaacatagttgcaatcaaatcaacaatatctttgctcagaaatggatatgtgaagtaaaatgtgctgccctgttcatgtatgaaatcattcagctagctggccagtgagcccttcattgcaacaaagatttttctagagcccctgcactatctggggctatgtcaggcctcacactcctgctcacacgtttggaggctaccttggccagtattaccttaatccagcatttaggggaaggagcatttcaagactaaattttctaaactgctcaagcctacctcattttatttcttgtgtattttaacacttttggatgaggactcttcctagaacctactaacaattcccccccgcccccatgccaagattctttaaagactttcttgaaaacgcttcagtcttttcttcttagctcaaaagtactatcctaaatactagctctggcattacagggagttaatttgtgggcacacacagtaaattataaaccccttaagcagagaagattgtattatcagtttattagttttcttattcattccttcagcacacatttctgttgcctccaatgcagcagagaaattgacttctacagtttccacaacagtccagatttcaactgtgtagtgctctttcagccagaaagtacacttgtgtttccctggccataggtcctgaacctcacttctgaaaagtcattgtgcatagagagctaatagctgtaccctaaatgatcctggctttgaattctcttatctgcttggatagtattatctgtctcttcctctgcattctaatttgctacttctaatctgctgggaattacaataagaaagaaccatttaatcatttttacaactgtgcctaaagagagtgtgtgtaagtgccgagagagtgtatgagggacttgcccatgagtaaatgcatgaattttaggtcaagggttttttgcttctcttttggttgattacctcagagatcagttttactttctttctcattcttgacctatcatcactagctgatatggatgatgtgtacaacttctgagtaagaataatgtcaatgggacgggatgggattggctggtgattctgttgatcttaaagtttatatattttaagtttagtgtttcagaatgagaccaaagcggtgacattttcaacctcttcggtctctcttcagttttttcattttaagtttttgttgtgcttctatcacttaaaggaagcctccaagttgaaatcaaatactaatgacatttttatctaatgtataaatgtgtttttattatttattaggaaatttattttacttggccctcagccatgacatatcatggcataatcactatcctaaatttgtatatcttatccttgcataaggtaagactttctatgaattacacatatttgtattttcctctcttacatattttaagaatttttttatactttgttttctgcaaatgaaatattgctcataagccaggtgatggctgtccacgcctcttttcccctgcttacccttgcttagatcttatggtagaatcttttcatagaagacacagaaagacatgaaagaaagagctggagaagcctgaggggctgcccagtgagtggtctgctaggatgctgtgccacagcccaggcacaggaggcagggagagcaatggggcccttcccttccaccaacattcagcagaatttgcagctccatgtttccaaagcttccagggcacttgcatttagagagagagagcaagcaagctgcctttcctcttcctcagttctgccagccacactcttgccatgatgagcagtttcagccaaaagctccttcccctgccctaacacctcctgcaaggcctgaggtctggaagccacctgcgcctgctggcccctcctttcttgtttctgcaatggatgttgtggccctgtgagggagaagagaaaaagaagttgccctcctccctctatcctcacctcctgccatgctgtcacccttataagagagaagggctaaccatccaggctaatcctccagtgatgcaggagaggacatcctggccggaagagtcagagcttccaggtgagctcaggtgggtcagcccccgaggctgtgaagagcccaggggccagtagatgccacttttgctccaggaagaatcttcaactgtgtcctttttattcaaggggctctcttttcagcgaatctctagatgtactagtcacaaacactcgcatttattaaaatgtatccatgatcccacaatccttttacatatttctctccaggagtatcacatttctgagggccctgtgcctgttttctgcaggaagttgctgttgtcccgggtccccctgcccccagcacctctgttacaagaagcagacccttcatgccacactgggacccagggaggccccaagccaggatgctgggatcttagtaaaggttggaatgatgtcagaacatagaggaggcagaattccccccatagcatcatcctggagggcgctgatttgtgtgctctgccaggttcatctgtgactcaggatttaaaagccccaggtgtggtgtcccttctgtgcctgcaaggtgcgtctttagcagttctccctggtgtggaaggatcagtggttcttgcagcctaggcaccctccacagcaagcccaacacaggtgctgtgagcagctggttcatgaacggtgatcctggggagaagaggaggatgaaaatggaaaaccagtgcaaaggtgtgtcatccagttggttactgctgtgggtgcccgggcttccatcccaccagagcccccactagccagggcagcctggcggaggtgagcatcattctgctttctggctgcacctgtgtggacagagcccgttccaaagcctcccaggcacagggcagacatcaagcaaggggcagagctggggcgagggctctgggtgcccccgttattgaacacaggcctgaagggagcctaagaggtggctggcaggagatactacaaggcagaaagacagggcagcagtctttgtactgtacgtttttgttttttaagagagaacaaaaagtcaaggttgagagatgagaaatactcttgccaaaaagagaacaccaaaatcctggagtcacgggtcttccattaccctctctagcatttctgcttgtcttttccctagtattggtgaagaatttttaaagtaaccaacccataccgttggttacaggccctgtggtcaccaggctgtctccccatgatggggatggagagtggttagtgcagaaacttagacctcccctccagcttgttgaatgccctgaagtttatctagaagggaagatactcaagcgtaggatttcagtgatgcatttggacagcatcagtactatgcttcagcgtcaaaaacgtcactttgggtaggaacaatacataatgcgtggcaatgccttttgtgacacctgttcaggagattcccataggaagcttctgaggcagagtcctcaggtgaggagggggacaggcctgggtcttgggggaaggtggagatgaccagcctcatgcctctctccccagtggcctcagtctccatcaggcaaatcttgagaagcctcctccattctgcaggcaaatgactgagatgtgtgagctctgcttcccaacaggcggaaattcacatggggaagggcacctgtgaatggccttctagaactatcaggaagttctggatttagtaccctgtgagagcagatggtcctgggtgcactcggatgatcctgcctagccagctggattgcagagtggtcatcattatcagtgagctattgaggggttgagagcagtcagtgtcatgtataaggatgggttggggtaccggccaacgcccgccttgaggcctggccgccggatgggaggcgagagcacaagagagtcctggctaggtgtgccccgctctgcagggccacaccatccactggaaaccctgtgcccactgggctgcgtgctaagaggctgtggccaatgctcttgtccaaattttactgaatctgcagtctctcttaattcactccaaagtttaatgtgttagcttgcgaataaataaataaataaataaataaataaataaatggaaagaaacagtcctcagaaagtcccagtcaaattttaattccaacagatattcagcagtttcctctaagaacaatgagagttctggggctagccagtgtttctcttggaaaataaggaagagggaaagcggtgcattcatttaaaaacctgccctagggaggcagcgttccctgtgaactccgaggccacagtgagcagagcaggctggaggcctcccggctgtcctgcccctcctgccacatgcctgtgaaaatgctagataaggactttttctcagcaacttccacgctccttcagtggggatgtctttgactcagagctctgccactggttatctccacgaacagaaaatgccacagatgggttaattcactgtgttgttctcattttccctcagtttcaggcttttctctccttgcctgttttcctcgcttaaaaaatgatgtgggggtccctaaacgcatctaccccgatagatttatgttttcttttccattagtccactttgcgtctcagccctaaaattaagtttttgattataatgtaaggaagttttaccatattttaactctggctttttaaatacaaaagaaaaataacagaatggccttctagaactatcaggaacttctggatctagtaccctgtggggacagatggtctggagccagcgcacttggatgatctctctcagccatctggaccatagagtgaacttgttactgtccctccaaggctgacaactatgagctcatactcttctagcagtttaatttagacccaagaaaggctgtgtgtgtgtgaatttgtgagagtgtgtgagtgtgtctttgtgcctgtgtttctgtgtgtgtctgtgtgtacatgtgtgtctgtggggtgtgtctgtgtgtgcacggctgtgtgtctgtgcgtgtgcatggctttgtctgtgtgtgtgcgcacgcactctgctaagctactcaatgcaattccttactcttacttccctttctgtcacttctccataattctttgtatttcggttgggctggtatctcgcggcggcttcctcttttcctgcattcctatatttcattatttgctcttgttcctcttctagggcttttacaatatagccaggaggatgtggaaacccagttacaagatgacacacaagcacagtgtcacaatcgctgtgcttgggccctacctcctggagaccagggtgggacggtgtctctggatatggagggaagggcggaagatcacggggtggcagaaggcccgactgtccggtcttctggaagctggggtctcgtggtcgctgggctggtggtgctctaaaacctagagtaaccgagagcaggatgactgcacccctgactgccgactcaccgggcttcggagggccatgggtgtgtgattccacttgtgatcctcctacttccccagcccaccctggtggtctcccacttctctctctctgcctgtggagtgttataaccgactccttcttctaggcatctccatcccaaccccagaccagttcccttaaaccacctcccaccacaggacccccacaagcagggacctctcacagttcctgttgcctggggacttcatctcagcttccaaggcctctggaaccaggccctgcatcctgggcatgactgtccccatgattctctgagccagccaccaccccaccacccccccaccgccaagtccactgctgaccctgctgtagccccccacctccacttgttgagattccctcttcctgcttctcattttgaatgtcacccttctgtgacttctcacaacccccctgccacccctccctccagttggcatggcttagtcttcctaagaacatgtgctgcttcccagtacccagggctgctgtgcaccgtggcacgtgagtttcggtgcatcttcctcctgctgtttataaattgcaacatccctggacagatcttggattttccctctataatttctctggagactccctcggggtttgcacacacatctgtggaatgcccaagttggagatggtcctccctgatgtgtagtgtgggctattcagagggcctcatttcataaactcattaacagctaggacgcgaagacttggagaagttagctcaggacacacggagtgggagggaggcaacgagttaagaaatgggctttggagtgcctcgaatttggcctccacttttcttttctactgtcttacctggtcttaggcaggtcgggtgaccatctggagtcggagtttctctcatttgtaaagcgggggtgatatttttctcacagggctgcttcaagggttaacaaagttaatgtctaggaaggacatagcatagcaccttcacctggttgcaagcccctggagtcacatggcaacagctgtggaaccaggtaaatgacttgacttacatgttacacgctcttagttgttcgatttgtaaatgggaagagtgtgtgacccaaattagtcgtttccagatatttcctggtaaatggttgttgaatcataaaactagaaagatgggaaagaaagggaggaaccccccttacttcgaagaagtgttgttgatgtgaaggacaggactttttgagacactctcatcctaagaaacagctgatatctactaggaaaaaacatgacatttgaagtttcttcctaagagatgtgaggttttgacagaagtgccaggaagccagaggtgtgtgagtagtgggccaatctcgcgtgaaggctgtgggacggggcaggagaggggcagccagcatttcctgagcgccagctaatggcggggacctggcacctttgctctgtgactctccagctgtatggtgacatgaggctggcgttttctttggtttcatacgtgaggaagctgaggcttggtgatgtcctgtgcatggctcggactccagagctactcagaggcagaggccgtctgtaaacaccttcaggtggttggaggcagggtgctgcagccaatgctgatcactcacaagccaaggctttattgtaagggctaaataaaataacatatgcctagcatttatacagcacggggctttttaggaaatagtgagacgaccatggagaggtggaggaagagagaagggaaagaagaaaaagaaaaaaaaagccttaaagagtttcttaagagaactatatattacaaagtccttggagttttttttccctggttagacataagtttacatgaaacgttaaacggcttaaagtagccacatctccttccttatgtcttctcacagccacttgtgagtttcttatcaagatacagaattatggccaagtgcggtagctcacacgtgtaatcccagcactttgagagactgaggtggaaggatcatgtgtgctcaggagtttcagaccaacctaggcaatatggtaaaaccctgtctctacaaaaaaatacaaaaattagctgggcatggtggcatattccaatagttccaggcactcgggaggctgagacaggaggattgcttgagcccaagaggtcaagactgcagtgagctgtgatcgagccactgcactacagcctgggccacagagcaagatcctgtctcaaaaaataaaataaaataaaataaattctgcaacaagtcacaattccttgctcagaaatctctacaggtgtgcttctttgttaagaaatggaggaaacatgaatttcatcctgacttctgagtcttgtagagaccagatctgactcctgcagccttctgcccacagagctggcaggcgggggaatgtgcgcagagtgaggaggagctgatctgacattccgaacagtggagactgctgcctatttggccgcaccactgtccttctgcatggaaaagtcacagtaaataatggactctctacctgtgagtcgattttactgagctgcctttttaaatatatgtgtagaaagggcgatctctgtgtgtcatttgcacatgtacatacacatgtacacacgtgcacacgtggtcactcatgtacacatgtgtgtgcaccagtgcaccagaccagatgcacccacccaattccacgctcctcagccccttgctttcctctgcagacagccagttcagctacagttatgccagattgcctgcactctgatctttaccatcaccagactctcagcttgagctcatgcctgagaaatccactttctgggaggaaggcagaagaaactgccaaggcaggactgaagacttgacctccacctgtagtggggtcaggtcaccagaggctggctgactccccaggatttctcagcagagtattacacaaataccccttccttaaagattagcaaccacttaaagacacaaagtgtgcaggactgtacccatggccgagagcggcgggattaagagggcaactgagttgttcttcccatccctcctcctctgtgctgagtcaccttctgcttggaattctgacagggacccgttgggcttctggaagggagggacagcagaggatccaccctctgtgtgtcctgggggagattacttatctctggcctccctgaagcagggcctgggcttctggaatctttgaggctgacaccctgccagccctggggatgagagggaatggccgtgtctgtctgcagagcctgaggaggagctgagcacagcctccagagttctcttcagttgatcgcttaggtggacaaaggccacagaaatggatttaaactcctcagccctttctttgcatgtttgttctcatttgaagtcagaagtgatatgtcctacacctcaagaacgtgtgaaatgcacatacaataaccccatttcaggaagccaagtccagcttaacagtcaaaacatttccttcagtctttagtccttcactttgccgaactcccttttacaccggcagcaacagtttaacctgttgcttctgtaagagtgtgctactgggaaaaccacatctaaaacacgtgtgcagttacatcagctagagcacatgctaaacagttgatcaaaggctcttgccttgtggcccacgctgcagacactctgacgactgccgagctccgcagccccatgtcgtcccttccgcactgcctgctgtgcctctcctctccatgtggcagggaacacagccagtcatcaccatgtggctctgcccggcgctgccccagcatgtcctgacagggcctagatatggaaaggtggctctccatgcacacaccccaagcccctcctgcccgccgtgtgacccacactcttatgggcagcccagttattttgtagcatttcccttccttatcattttggcccagtgatccagcacaaatctcccttattagaataaaatttggaatgacaaaattaaatttcatttttcacttatattgaggacctcacactcttcacccctgccacgatccctgacaagagccttcctatctaatcattgttcctccagccctcttagttttcttcagcctttcttgattgcctgaatgtccctttcccttctccttttaaagcatgaaccaagctttcttaccccgttctcattatcatttttgcattttcttctttgcatatgattctccttaaattataaaacttgggggtaatttctagaggtgccatcatagtgcttctgtctactcagtgtctttagaatcagcaaatatcatttttacaaaaaagtagtatttcttccaaaaaagagtaagcaagaaggttacaacactgggaaaatatccctaagcctgttcttcaacctgttgaatgttttcccctaaattgttatatggagatcctggacccggaagttggctgacatgaaacaggcctagcagggcagctgaggaaatgctccaacctcaggatccaggaagattgcacatggcaccaaaacaatcatttaaaagctaatcctggccaggcacagtggctcacgcctgtaatcccagcactttgggaggccgaggtgggctgatcacaaggtcaagagatcgagaccatcctgaccaacatggtgaaaccccatctctaccaaaagtacaaatattagctgggcatggtggtgcgcgcctgtagtcccagctacccgggaggctgaggcaggagaatcacttgaacccgggaggaggaggttgcagtgagccaagatcatgccactgcactccagcctggtgacagagcaagactccatctcaaaaaagaaaaaaaaagctaatcccataaaagaaccatcattttgaacctgctgtttcctttcttgtactctttagaagtacccatttctccctttctaagccataggtgtattaattggagctttttctatcttaattagttcactgtgaacaattaaaattgtgttaataaaacaaaaacaaaaataggactggtgcctagttgtactacatgaagagagaaagggccagacattggtttttcctaatcttctgtcaagttctccaaatcatctgctctggaaggtagctccaacagctgggatttgaagtaaagcatagtgactttggccatcactgacatgtcccatttgaagcaaccaaaactgtccctcgacctgacactcatccctgaaacaccatgagggtaagtgaggcgcttcggaaggtccactcaaccccattgccagatagagtaagtgtctgccaggggcatttggagctgaagggaagactgtacagactcacggtttgcaggcactgaaggcgttttcctgccttctttttcaccttcagtggacttgcaaagcactagccctattgtttttcccatctgggaaacatggtgaatgttggttggttgtagctaatcctatgggtcttgaggtctttgttgacaagaaggtagatgttatctttatctgcgtgtggctttctactaaaacatgagctacagggctctcttttttgttttaaagcatttttccataaggttcacccttactattgcttatctgaataatattacctgctgagaagtttattcattgctcaccagttgtagggagattttgacacaggactggaggatttttttctcatcgtaacagtgcagacccatggaaagcttggaagcagttgtgaccggataagagcaggttgaggatgataatcttagggcaatgagcaggttgattgagaggggtgcctgaaagcaaggtccccatgatgcaagcaaacaaactcacatgccagacggtggacaggaaacacaggcaggatgtggtgcaggctgggctgccttggctgggcaaaggcaggggcttcatggccatgctgggatgtgacgtggttggaaaacaataagaatggagacctctccaaagacctcagacataatttggccatgagaggcaagggtagaggcgagccccctgtgggttggaatgtatggaatccagatgaagcaccatcaacatgcatgggctacataggagagctgggtctgggggaaaagatgggatttgggtttgggtgtgcatgttccaggcatggagggaagaatgggcagaatgacgaggatcaagccctggtggagcccccagaccagggccctgggcatatttccacttgccttcttctgcaaacttttgtgtgcctgttgggtggaaggtactatgctggggccatccaggatccggagcagtgtaagacatcatggcctgcatggtgtgacccttgacatctgaaaagaacgagctggtggagtgggaaggagacaagtggcagcagagttgacatcatcaggactgactgcaggacaggcggtgaaacgagccactgaggagttacccagtgccctggggagcttagatgtgagacttacaccacccagcactccccagctctgtgtccctgggtcccactgaacttgattgtattttagttgcctcttttctaagaccggagcaacaataccttccttgagtgtgcatgtgcagtccatagatggcagccagtgttctcttcccatgagaagactctgagcctcctcactagagggtggtctaaaaacaaatggatccatccacagaaggtctgaaaggttttcatagaatcttgacttggtagcacttcaggatctaactcgtgctttccagaagtgtggcctgtggtgttctgtctggagtatttggtcctcccatccacctccaccttgctgtcctgtcctgtgactctcactagctcagtcaccgggtcaggaagccctgccatgaaatgctagatttggaggcattgccaaggtaccacaaaccatactcaaacaaggtcataatagactagtgatctcactgtggctaacaggttcatgtgatttaaagaatgacagcattttttctaaattttataaattttataaatttctcctaaatacaaatctgaatgtgtttacctttagacagattttcccagaaaattgtcagtgttctcgaatttgaaagtagatcagatctcctccctctagttttgaaaatctcaagtaggtttagcctctccccagagtgacaacggcacaagaagtttataattttatgcacagctaaatgcaaactgaaaagtgtggtttgtgggtatttcatttttcccataatgttcatcaatttggcaattcaaataggattcagaatccaaggtgttcgcagcattttatatgaaaatgtgcctgagagtttggggaatagaaattcttaccaaataagttaaatttgctcttgataagaatatattttgtaaaatgtagaaaactgagaaaaaaagtttgacacctgttttatctgcatccttttattaaacagtggcctgaatttactaaaggacagagaacagggttgagaggtaatcactgaataatagcttcgctaatcccacagtgactgcttcttatgacaagcaagagggtcaacttagaggcaggactgtcttcaggggcaaaagcaagtcagcacctacaaacctctccaagagagtgagaaaaagcaggttgtatggctcattttgcagcttgccagaagcaatgtgggaatattgtcagactgcacagacctggggccagctggctcctagagtgcgaggagctctcagtgactgctgtgcatactcacctgctctgtgactttgttcagtccctaccctggatgtgtggtgaagggtgccaggcttagctgctggaccagtgaggtccctgagggggcagggctaagaggtgtagcagtcgcacttgaccagtcattgattcactggttgggaatttgttcattgaggggacaccattactccccatgcctcacgtgcaggtgagtgtgtgtatatttgcattaacacacaaacacacttatgtgtgtccctgtatgtgcagctgcagtaccttacacatcttccagtgcctctcaagcatcataaaaacagcttccacaaaacctctactacccacctggcatccacagagctcccagtgattggctcccaaaagatactgaactttggcatggaacagagtgagtttccacatttgctgttgtacataattacctgtccagctagaatagtacacacttcttttactgagcccagaagtttatgtgcctgaacacggatggtacaggaacaagggtgagcgcttatcaattcagcattactctggagacatgaaacaaactaataggtacagctatattttgttttttaaacaattttgtgaaccacatatttgggacaaaaatcaacaagtaaatttgaattacactaggcaaaggactgattcttttgaaacgtgattgacattcaacgtctaactttccaaaagaagacatacatgtggcccacaatcatatgaaataaagttcatcatcattgatcattagagaaatgcaaatcaaaaccacaatgagataccatctcacaccagtcagaatggttattaataaaaagtcaaagaatgacagttgctggtgaggttgtagagaaaaaggaacgcttatacactgttgaggggattgtaaattagttcaaccatgtggaagacagtgtggcaattcctcaaagacctatataccatttgacccagcaatcccattactgcatatatacccaaaggaatatgagtcattctaccgtaagacacatgcacgcatttgtttgttgcagcactattcacgatagcaaaaacatggaatcaacctacatgcccatcaatgatagactggataaagaaaatgtggtacatataccccatggaatactatgcagccataagaaaaagaatgagatcatgccctttgcaggaacatggatggagctggaggccattatccttagcaaactaacccaggaacagaaaaccaagtgcagatgttctcacttttaagtgtgagctgaatgaggagaatacatgggcacatagaggggcctatcagagggtggagggtgggagaaggaagagtatcagaaaaaataacaggtagtagatttaataccggatgacaaaacaatctgtataacaaacccccatgacatgagtttacctatataacaaacccgcacatgtactcctgaacttaaaagttaaattaaaaaaaaattaatgtctaataatatattacagtattcttcatattcaatggcaatgtgtgaagtgggaagtgtcttgacagaattcggtgtttcaaggcttacactttgatgcccaagactgcacaaggctacattttctactggtgagacaaattccagacgcattgcattcagatctaatctcttagctccttaatcttcagggtactggtaaacatgaagacctccccagtgctgtagtcatcatgatatgtacagcaggtggctgagctctggatgtagactgcagggatatattaggaagttaattctcaaggcaagtcatcttcaagcaccatatcagcatgatcagcaatataagtagtatctcagtgctttgttgtttagtcagagttttgtactctatcacccattgtaatgttcctatttgcaaaaggtaatacataccctttaaaacatctttgctttttctcccattatcgagatgctagcagcttcataaagcagaataactaagggcaaacagattatataaagggttggagctcaatgaagacaacaagaacagcaaaggttattgtaaaactggctgcttgcaggccaacaagcacatccatatggaggcaatcagtttatgctacctctgtctgtttgatgggattcataatattgactttatccattagatttggactaccagggaataaaataagcagatggagagtaaggatttgctaggaaataattcagccagtcactttgaaagctgttcaagaaacagctttcaaagtgtctctcaaactatgtttgcccattatcccaataatttatttcccaataatttcatgggaaaagaaggaagttctgtggtcagataaatctggaaaacactggtttaagcaaagttcagtaggtctgcttccctgcaggtcacctcagagtctttactctgctaacctaggaactcatccaacaagtttaatttaacagctacactgtgtacgtcactttaacagtcactgagctgtgactcttgggggaaagattgtgcgtgtgtgtgtgtgtgtgtacacatgtgtgcacatgtgcagaatctaccaaatcttaagagaaaggaacatgctgggaaactgtcctgtgaaagagaatagaaacctgaagatttgaggcagtgatagcatttatgaaagcagcagataaggactaatcaccaaaaggggtagctcttttgttggttggggaaaacaggaatttttcccccacccaatgtgctgcattttctaattttctatgaacacttcctaagaaaaagctgaatgaagaacatttgcgatgcaatcagctcattaagaaacacgcacttttgtggagatacgtgctgtcccaggagatgctctgcgaggagccgagtgtttggactggagctgctgaatggtttctcacagttctagaatgtttggggctgcaccctctaagatgttgaacccatcagtaattgctccaaaccactttatgggatataatgctgtgagttgacacctgaggggattgtggtcctgttcatgagtaattacttttctgttgcctatagaagggccagcaatagcagatgagtagctgaacagtggttttgagtaataaaacgttcttttttaaaaaaaagtaatgctttctgttaaactctgactatactctctcctggtatcacaacccagctttctttttgccttctttattgcagttacatatggggctgatgactttagggatttccatgcaataattcccaaatctttctctcgtaagtatatgccttgcttctggaaaacaaaagcatgccttcatctcctatcatgtaaatatcgtacgtgcatgttccttcatcaacccccgagatacattaaatattcactgttctattcgttagacacctaccatatcatttttgggtatttataccataaagtgcaaaacgaaggtctaggcagttgtgcggtgtcctgggagcatggcagtggagtaacagtaagggttggagtcacagtagcactgatgggattgttacttcgcagatgctgctggacagctttgagattactcctgtaatcctttttattgagtgtgaggataaaatggccatattttctgtcaccaacaaagaactgaacatggtttcaagataatttttacaattttgtgggacaggagaaggagatatgctcttcatctgtggttctggagtattaatttcagaactgagaagagaaaattgcaggcctccaggccgttattctgtggtgcatcgctgctggagccaacgtactgttcctgcttggtgcccgcaggaccttctctgatctgagctgttaaaccaaaaacagatgtgaatttcatttttcttttttagatattgagctgataaatgataaatatatttgggtaacatcctcaaatgagtactttaaagaacagagagtgcttttgaaaatgtatagagagctgaatatttaaagccatacaagttgataatccccttaggacctgtattagtaaatcctctaatactataaagcaacatcagaaaacaggtcgcatttgttaacactgtgacaattataattataggctctaatttgaacagtgccagtaacagttaaagacaggtgtctgacatcctggttatcaaaaactatctggtgtctaaggaatttaacagtatcaaagtacatctttttgctcacaaactagattggctacttttccagcctatagaagttacagaaatctttcttttactgatattcctttgattccactttaaagcaatagcttgatacctactttttgagatgtatatgtatgtacttgtaaatgcttatatatgtgtatttgtttacacgtgagcttatatgaacatataaaacattatggtattggacagagaaatgatattgatttgaagaatcatattgttgaagtattttgagaagtcaaatgcacttgagagtaagctaatgcatcctaaaacatgtttctgtgaagtctgaggaaggtgtcagccagagcatatgttgaagcagacccttcagtgaagccttcagtctgtaagaatcgctgcatgtgaagatgtgttaatgttatgatagttacagtttttataaaagagatgatatacacttggatatgctttctgtctatatttatgcaaatgtgtccataaggtattggtgtctctctttctctcccactctccccagtgttggaattgtgactatcttctcacacaagcggctacttggtcttgatgccttcccccgcaaaacagcaaccaaactgttctgggccaatatcaccaccttgtggtcatgatgaagaattgccccctttgccctcaacacctcttttcttcttgaaaattaaaaacaacccctttcaccccctctactgtccttattccagtttgtgtccgtagttgctggaggaaagaaaatgcctattgctcttttttttattctctttatctgtttactcttctgcctttcttttctctccctgacttcattccatttaaaggccttaaattgcaaataacgaaaaaagtattttctaataatataccctagtgggacgaaaacaaccttctacattttaaatgatattaagttataataaatggttccatgaactttaaaacgcttaaaagttttataaatctcttttagaggcaacacaaaaatatctatatatatatcttctcttcacctcaaattttcatttaattaccgtgattcaagatatatcttaaatatttccatttcatttgatttttacttgctagtcaccagtgttgcaagaatatttctgtaccatttttcaagtaaaatattgagcatttcaagcttacatggcctaaactcacactagagtgactacttcggactatcttttcaatggaaaaatgcgttctgagtaccaaacatttactttttcagtaaatatgaagacacattctaaaaaggaaagggaaatgaagaggaagcttaattcattggtcaggtggggactaacagtgtacattatacctatgcattaaaaaatgtttaattatccatgagcacatacagtgcctggcacagattcaggcctcttggctcctcttggcatctgagttccttggattctccagattcttcaatccaaacttgtctatctaccaaagtccaccttccatacaggggtgtggggtctctcttagtactgttgggtgccaataacatagaagccctccaagataggtcaatgagattttaatttttcccaactttaaatagcatctgggaaggaaagcagcttttgactaggagtttgaaaacatccactcttcatatttattgtcacatttattgagtaataaaattgggctcttgtccattatctctcagtcaatgaaagagcccacggtgggcatgagccagagtgttccggagaaagagaggaccagtctccatttcatgttctatatttaatcagttcaattcagcaccttactgagtttgacatttttctaagtaatggggtggggggttacaaaatatttataactatctttcctatcttcagggagatgtaccatttcactgaagcaataccacagctaagcagttccagctccgagtaacagaggaaggagtgagcgatttaggaactcacagaagggcacggccaggcaactcgggccctggcaaatgttccatagagacagcatccatggggttgaaccaagcacaatgcctgcaaatcgggcaggcgggttcatggaattgattctagggcaggtatgatcagcaccatttaaggagtggcagttgttaagaaactgaagggatttctcatatggcattttcaacagaattcagcaggagccttgcatatcccttgggatgggttccggggtattatggagctgccaggaggtgctcagcttcagtaagtgccagtcattccttcccaacctcctccttcattagtggaagtatcagctggtgtcattgccttcttcaggaggatggatttcaaaatgaagggccaacaaaactcacattctcgcagagcctccgtcacaactgcatgtgttcactgccatcaacaggccaacacccacttgttcttttcttctaggtaaaggaaaaggtctgtgggacaaaggccctgaactcctcactccttgaatgggaggcatattggtggaggctgtcacacaactacaggtggcaggaacctgccatcagggttcccatcagcctccagggcttgcttctctgccctgtgtgtggacaccttccatcactggaaacccctctctcagaagaaccacttcttgccctcatctcccacttgcctctcctcggcaccttctccaccctcaggggccaagctgaggaagtgtgcatcctgtgccaccaaccttgctcgcaggaattagtttcagtccccagtgactgactcttccaaacctgcaccagcctatgctgcccttttgaaaggaggcgtgcgtacagcctggctggtacagaagattccagatttcagtgactacaggacgctgtcaccccttctgtttttcttttttctgtccacatctctgattgagcataccttcagggaaaaccccagaaacctttttcacacttacatgggaagccaagacactctcatcctgggcttgtgtttttaaaatctatattttaatttcacaggtccttacatttatgactaatagatttcagattttgagatacaatctgtagttgcaacttatgaagatagtttcaagccctgatttctgtcatctatgaaaacagtaagcaagttgcttgtattctcctcctagctggataagagacccccgttttcatggacagcctccttgggtggacagaagtctcctgatcttgtctatgtaaccagcacccactttgcatttttccgcaaagaaaagaggagcttaaccccatttacgggaaatccacgcactgcttgaatcttacgcgccccgtggtgacggtgttgcccactctgttcccttcccaccaaatcctgccagcacctggcagtcatccagtccttgtcttagacttcaccaacctttcccgtcataacatggcttagaagttgtccgaatagtgctagaagctttcagccctggaatgctcagcatctgccatctaacttccatttaaaacgttttgagcatttgctgatacaggttcctctgtcctttccccattattttcagttccttggccttgcccatagtgatctgtggatattgtctgcatactcttttttagaaacctagagcatcgcagtgtccttaattttcctcttcaaactctatatattcctgagctgatcatccctactgctgttcctacagaactgtgtgactaatcttctatatatgtccctacatctagccatctaggtggagatctaggtacctatctccctaaaattgtacttggtatccacattgacctcaggacagtatgtgataggctcttgtgccttttcgactaggagtttgaaaacatccactcttcatatttattgtcacatttgttgaataataaaattgggctcttgtccattatgtctcagtcagtgaaagagcccagggtgggcctgagccagagtgtttgtgtccctagttctcttttggttctgttacagctctcagtgacagtgtcattgaggctaacaaggacagagtactgctttcagccaccatttgtccaatgagtggctgatctccaggcctctggctttgagaacatctgtgatatctaaggcagcatccattgtgggctttcccccatgcttctgtttccttcctgtcacatagctttgcctcctcctgcaagcagcctgctgtagcagaaccggtgttcctgaagccagaaacccaaaggtcgtgtccaatgcttccctgctgttctgctccccacctgcaagcgcccacacactgatcaacagcacacgccatagagcatgccaaagaatcccagaaatactcattcttaatgatccagaagaacaagtgatagcttctggcaagttctaataggccatatgtgcccatgggaaagcaagagtcaattctctctagctggttgcctctgaatttatgaagtcaagccgcgtaggggaacatgccaagaggattatcccaatgcctgttctgcgtggtgtcctttttctagcctcggaagcactaggcctacaccttcgctatagccctcgtcttagcgcctgtatttgaaaaacttgccacaccaatcttaatttgctccattgtgttctaatctcattttaaaaaccacaaggtaaatgattaaaaataatcttagattaaggagtacacagatctttgcagcctcattgtgtttccagagcgggctagtgtagacactggtgaacaaggagggcctagagaattagctttctcccagaaagatgcacagcctctacctgagagtaccaattccagagaactcaatggtcattaagcaccattgcctcgacagccagccagcctcacttgcctgcctatctcctttattttccaagtatcttgtccctggcagtggggagaggttagcaggaggctgctcagatgctctcggtctctgatcttcaggatctgaaggggagagcatttgaagaatccccattgctggatttctcaggacaatcctgcataatgccaggatctgatggaggagacaggcagctctgttaatcctctggtgcatcctcacttctgtggtctccaagtccaccatgtcccagttaattcatttcattattcatctgtaagtcatttccctaaagagctaataagaaaacactggcagtacaatcagccctccatatccatgggttcagcctctggaaattcaacccactgtggcttgaaaatacagtattcaagagacatggaacctgtggatatagaaggcagatttttcgtatccataggttctatagggccattttagggacttgcacatctacagaccttcaggagtctcagaactgatgcactgcagataccaagagatgactgtaagtggttaggaatttggatgctggggccaggctgcctgcagttaccttccagctctgccacttgccagctatgtgaccttagcaagttgttcaacctctctgtgccttggcttcttcaactgtaaaataggataatgatagcacttcccttatggagtccttgtgaaggttaaatggcagagtacaattaatgtgctgtgtgcccagcgtgtggtattggggttaggtgaaagactgtactgggatcactgggggtgagcctccatgtgccatgccccaaccactatcctcagctaacttcttgtctgtcagtgttaggctggtgctataataatttccattttcatggatgaggaaatcaaggcacagagaagttacatgacttgcctaagatctcagtgcttttaaatagtgcagctaagattccagaccaggtatttttatttcagtgtctggactgtagatctctaaatcgagaggaactccctgaataaaataagcttggagtgctgttaactaagttggttatttaagacagttgttctcagtcctcagtgtacattagaatcttctggagggtatgttaaaacagactgctgggcccaccgagagttcctgagcctgcaactctgggttgagcctgagagtctgtgcttctagtaagttctcaggtgatgcttatgctgcttctcccaaattttgagaaccattgattgaaaatgttgatcaaaaattatgtggtctaggctgagcacagtggctcacaactgtaatctcaacactttgggaggccaagtcaggtggatcactgagctcaggagttcgagagcagcctggtcaacatgacaagacccatctctacaaaaaacaccaaaaaaaaatggctggatgtggtggtgcacacctgtagtcacaactactggggaggctgaggtgggaggatcacttgagcccgggagacagagcaagaccctgtctcaaaaaagaaacatttagtcttgattgtcatctatctcattgatcattttacttggcaaaatttacctacttactcttattagtctgtaaaaatggttattaaaatggtggctttcagtgtaactacaaattctcttagtcattatagtgttggattcaccaatgtatcatcagtcagtgtttctgaagtgtgtaaatgaaataactcctactttctttaggaagaaaaatattcaaatgacataattaccttgtgatgtgtgacttaaaggtaacagtaatgcagagtcaatagtggtcattgtattcaagacactaaaagttcacctcccaccccccccccccacccaccacccaacaaacacacaagcttctttccctttggaaaaaaaagctcttccagatacctacattcataaactatcccaattaacccttcagcaagtggaagaagtgtaagaaaggatacctcttctttagaacacagggtttgtttttatgttatttaagataaacaggaattcaaatggtcatgtaccaaagcaacacaaagaacttccggaaatctgaaagggaactgtggtcagaactctgagcatttttatgtttactgagttttgtcccaaagtttattaatgtttacatgccacaaggaaaggtagcatcacaataagagacgtttttcaggcttgataaccacttattaggtattttgccaaacaagttcacacatcctagagagctggattgtgtgacccagaacccaccctctagggcaaggtgcccatctgatgggtagggtgtaggagtaggcctcagaccactcctgacgtgaacctgcttaaagtgagggcccaattctaaagtgggaactatgtaaatacctttctagtgcattttcagataatcgccactgggcctatggatggagagggctggcagatctccctgtaaccccaggtgcatcccgaggcctgccaccgaagcccactcaaggctgaatgcacggcgagctcaggctgctctccccttggtatttgctaagaacttctgtttagtagctctccacacctatttgattgtctttttgctgctgtgttgttttgttgagttttttttttgcaatgacactgagtggcctcctgtattgtttctttcagccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggtaaggcctggctcagtttttcctttagtggcctggagaaggtgcatggggtttgaaggaggaaagcatcctgtcttccttgtgttctgagcatgtttctaattgactggtagctcagttgttgcaagcgattggttccaagtggtaccgagtcatagagtccttgttctggtacagccttgtaaaggacttctcaacacgtaccaattccaccctataaataaaacaagggaaaagtgaacagcatcacatgagaggcttggcgagggctgctattatagtaacacatactaagtagtctcagctgagccctcagggtacgtgtgctgagtggtcaccctccacaaaacaaaaaatcctgatacaccaaaacttactcctcaaagtttccactgagaaaccatgagtaaaatgtgtgtttaaattgtatccaaactaacagggtttgatgtttagaaacaataatcaatgatggaatagcagcaaaatcgagttttcagaaagacctcagatgagctttcaaatggcttgccctctaacaggaaagactttgaatcagatgcttctattgccactggttatcagctcaaattcctaaagaacttcatcccaaataccctgtcttgctgaaaggtttactggaagtataagagaatgtcatgttctgtgtccagaaaggaaggaacacgggcaccctagtgtcagcgagttgtgctcaggctcacaggatgccccctcaccagaggcgtgggaacacggcgagccccagctggccgcgctctgccactgtttctaatagccggtcacgttgatggaagtgtcacagagttgtccaacagaactgtccagtcagaaaaccaccactcatggtgctggagtgtcttagaagtaaaatatgaataacacacacttatttaactataggcaggaggtttcttatgaattgaagagaaacttttctttgcgtgggaagctgttctaaagttgggtaaaacacaatagatccaccacctctagcagccactgatagctgaaacgtgaaacatagagaccctaagctatcactgcctctgagctggcattgttaggtcatcataaagctagcgtctcccactgcaaaacccaagaggaaaaaaatagttgaaaatcctattttaaaggcctagcagattctataagataccttgggaaaatgatgacgatgacttgaaatcagaccctcgtatgctgcttccgtgggggcgaacaaaaatatgttcatcaaaaattaagcagaaatgcagaaaattttgtgagccaaaaaagctgtgttatcaggaaatgcagatatttgtggggttgtaattttttatatttgaatcgggcggttttcaaaatgatctattccatttgtagtgtatctgaaaacctataaaaataagttgatatcaatagatatccatcttccataaaaattcaacttctaaaattaagcaaactttgctttttctaatggcccctttatcctcaaattacccactgaaatagacggatcacactcagccctaagtgaagcaagcgtgcatgagagtagtcccagcctcgcctttgtaatgaggtggaaattaacatgaaggtaggctaccctgtgatagacacttaacaggatactcggggacccatggtaatacatccctgataaggaatagacctcacaaatgaactacttgcctgttaattcatttaaagcctgactgtacagtgaaaattctcttaaataaatatttgataagtgaatcaaattctggcttactaaattgccaaaatataatgactgcctgccttgataaaaagaataattacatttaatgaataaacctgccaagtacagatatgccaggtggcacctgctgtttgctgctcacttctcccaacaaatagcagctaggtgccacctgcacaccaataagctgagtttttcacttacggaacaaataactttcagaagtcaattttatagtttctgccttgccctttgttaaaaaaatacacacacttgaagcaatgaactcatgaattgttttcatcatgcatttccacttgcctaaatataaagggtcccaggtagatacagaaatacctggtttggccaaacttggtttgaataactagacatgctagaaaaggttttcattttcctgggatctgagtggaatatgttagaaaaggcatgctttctgaattctctatgcttaaaacatttctagagcagtgcttctcaaacttgaaagagcatgtaaatcacctgagatctcgtgaacatacaactctgcttcagacctggggggaggcaggagagcaagcattcctaacaagctcccagcttgtggagcacagagccgcctcaggcagcagggcagtgcagctgagcagtgacagtgaacagggccattcagagggctctccacctggggcttaggtacgacgggaatccccactggacaggctaggacttgcactgtggccattgttcctcctcctgcccatggctgagtcagcttctcagtcccttccaggatacactgagaggattcaggggcggtctcgcctctgcccatatccccacgttggtgaagtaccactggcgccattttctaatcagctcatcggcaccagcacgtcacttcccctgttgtgcagctcagttttatatttctgactcgggtgtaatagtaacatcttccccaccaatgtctcagggttattgtgagaaccagagaggagagaggtaggaaggaagcaggaaatgcagggtgcaaatacaccacattattattctaaataatgggatttttaataacaaagaccaagaagattgtctgtgcctatctagttcccatctgtaagataaaaaggtgcagtccctgagcctcctaaaaacagcaccctaatggcactgctcctcccaggtatgattgcagtagacctacgtcagttcttgcagttcaaacacattttttcactttcttctgaaatgccctcaaattctatgacatagtatccatgaagacttccctttttacccagtgaagttactagagggggttcaaaccttcttacagagggttcctaaaatgggagatctgtggagtcattgggattaaaaattaaatttcaaattcatgacatcaatgtcctcatcccagttaaataaaagtaaatacaaccttgaagaaatttgacctactcagctgcaatagctgttgacctgcagaaggcaaatgatggaccccggccacgcaagaacacacatggcgtgggctgcgttcttgtgccccagctccatggacgtgtcttcttagctgtggacccctccaaaggaaactgcttgagtggagtgctctgtattgtcactcctgggaatgctctcttaacacccccatgccttgtgctatgtgttcataccagcataggcactcagaagcaaaaaaggtttccagtgaaatttgtttgcaggcaggtattaagacatcagacactgcaggatccaagtgaacccctgcccgcctgcctgcctgccctgctctgctctgcagaagggcatgtgtctgtagcaatctgccctttctgtctgcgatgggcagagaccctggctctgcctcctgttttccctgtagccctccagacctggggagagacatgggactccagcacctgcccagcacggaagggggtctttcccaggtgtaagtgtagggctgcattcactaatttagggtcataccacaaacactgttgtgtcaccaggatgttgtcacatgcttattgtgagcatttttgtgacatgagctttctcctgagaggccaccctgtccaaatgagagctgtagttggaggcacatccgtctcatgctgcctgggtctccctgcaccagctcactctacttttcctttttatccacttggagttaaatgcacacatttatgcacaagtgcacagaaggcccagaactgttcctaatagataattaaatgatctctctccagtggaaactctcctgggctacctggctgctagctgttttagcctcttcatccagtattgatttacatgcatcggtttgtaactcagacagtatagttagttagtttgtttcaccatcaccttccaacctgcttacgtttaaactccttctctctgcttagctaatacagatttctgtaaggatttggagaaataccagatacctcctggttagtatcaggacctctccattggcctataattgaacatcactgtaaattagtttcagcttttatagtaatgcatctaacccgccagcttaaaaaaaaagtgatgataatcatgaaattcaagtgtcaatatagacttttaaatctggtgtggatgttaatagtgccagatatgcttctgtgtaatcatttttgagaagtgctgtcttctttaccataatcacattaatcaatcgggaaatggttaaatagacattttaatacattgttttttcctgagaaaaaaagtgactccaggaaatatgttgttacaatatctcaacaccaggaattgtggtcacatagcattcttcttctaaataaagcatttgttttacaaaagagtttcatgtttacttaaacgtgacaaatgtatcactcttcgaagtgagagttaaatgaaatgttccctgatttagattccaagacctgtgactactcagtaggacaatagcatatttcttttactcagatacctgtttatccctttgtcagctccagaatggagacacaggtgctgccacacagaggagagggtcaggtgttttcttagtcacaaatgcagcagtctcacccctgaaagaattccttcataacaactaaattgcacctcttagaatacctgtacttaaaatataatttgtcttctatggatatagacaaataatagcttaaatgttttaggatttgtaaagaaagaaaaagctagaaatctcaatgcaactctgacttgtatgtcctgccataggaaggtttgggacttttctttggaagatgggaggcttccttcacagtttcttaacttctcattttattcaatctttctccatctctctctctctttctctcatcctcacgctctctgtctgtctctctcctgatttgtctgcttagggacagacaggtaatggagaagaaattgaaagggaaaatacagggaaaatgtaaatgaaaagaagttattataactgttataatgaagtgcctttcatttaaatataagaatgtaacgctgaaatgaacttgtgatcctgaaatgcatttttaatgagtttcctttttattttgctgcttcagtggcatattttaaagaccctttgaaaaaagccacattaaaaaaccccaccaaatgccacaacacgcaaaattggatattggtttattccaaaactgctgatcaggaagagtggctcttcatcacaaaatgttgcagtcactttatttaaatgagtttgaatttgcattgtgatgtgccattctgatttagggaaaaaaaattagattttcagatcaaattgacccagccagtgaagcgttaaggagctggcaggtttagtctgaaagcctcatgttctatcaaacctgcagccggtggaagtcaccaggcccccgtgggaaacaactttctcgtagcatcgtcctcatgtccccacgctgagtttagttctcaccagctctcctctctccgtcccaggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccggtaggtcccctggatgcagtccggggctgtctgggtgtcccgggattcctccactctgcccgcctgggtcccggattgtgtccttgctggctaaccctgagtccctcccagctcgcagtcctgcatcgggtgtgagctgttgcttctttgacattgccccatcccccctccccatattctccttttcccactgcaccagggagattgggcgcaggacccccatgcacatacacacacagaagtctcaccttaggtagcatgtttcagaacaggcctcctcatcccgtttgtcaggctggtatggtttccctaaaggattttgcgggaaatgttttcaccaagtgtactgctaagacccaaaacgttttcaagtacaattctttctgttgttaagtcccgctttggagtgttttacacaggtgtaatggatagcttttttgcagagctggtagagaagggtgatttagggcgcacccacccagactgagccccgtgtggctctcacaccaaaaaccagccaaggccacagttacagaggccagtctggggctgttaccagattttagacagcagcctttctctttgaattagacagttaaagtacaacccacataatctggagtcttgacaagatcatcataggcataaacgctctatcattctcaaaacagtctcagcctgcaaagttcaaatccactaaagtttggttagatcctctgcctcctgagaaatggtcctgggtgtttcattatccagcagtcccataattctacagggcagaggaagagagggctcttggccggcctgtcatggatcatgtttgcctacagtgtggtctatacaacatgacatggcacaggtctccttcataccgtccagttggggatatttgctgtagcatactgcatgagacttcggaggcgaaaggttgatggcttttgtccttcccctcaaggagcttcctgccccagccagccaccagccagggcccttctccaagcagcagcctctctaagccggtgccctgggggatggcaatgcctcagaccagctactcctcacccaccctgggtagcaggataaggaggagcctccctcagggaggcagacgtgtgttctttgtgaaatatctgcagcggcccaggccatctcttcccaaatgtgatgcgtgtatttgatggttgagggttttagaggctgctcattgtgtccatctctttatcacacatttactgagcacccctgggtgcccattctgcacagaagacctcaggtgcacacaaggaaagcaccaatgtgttacaggaggcatgacagagtgtctgaggggacagtgggagcataaggaaggggacagtgggtcctggggtggaaagtcaggagagacttcccatagaagggaggggcttctgaagtacatgctggaaggcgagtgtctgaggtttgctttagagacattctaaactagtgtgtgagtcggcagtaatcccagagaggggtgcggcacccatgttggcaagaactcggtctcactcctggcaggtggtccctgcagtagttcacccagttggctggagacaaaaaggagaggagtcacaggggctggtgcatcacctcctctccatcctgacctccttcctggcggtgcacatggagaaggatccccacatgctcgccatcagaaaattgtcatgatttgggtgatttgacttcctaaaaacttccaagaaaggagctataccaagctgaggagttgcttgcccgagagggcgggcagatcagcaggggggccatggagtgggacacttgccttgttacagagggacagggagaacaggtggttcccctgagataggaagacagtagcatgacggtgagcatctttaaatgttcacatgggtgatgagcatgtatggtggtcccacctggagacaaaggtggtacctgggcctgataagcaaggcccatggaggcctcagctaagagaactgtgcagtctgtctgccatgctaaccagtggtctaggaagaagcttcacatctgttgtttctaaggagccatgtagacatagacacagttggcagcaatattgccttgggttagtgggaagcaccacttcccatgtcagtggaaaacacagacactcgttcttaggttgacaccaaccataggtcatcatcactcctagattactctgattgcaatggaatctctcatgcccatcagtccttaaaagcagagctgctctctttcctttcttgttgtggagttaggaacatgtagtggagtattttatgttattctggctccaggcctatgtatttcatttgaggcatgatggtttcctattctaagtaccactaagtattgagtagttataaaatggtggtagactttggactgtgtatttctattctggatccacggatgagcaagtggagaaaacccatcatggatgcagggctgctggggatctgtctgtccgaaaatgctatagagacacacactgctcttggggaaaaaaacagtgatgatttcttcaaaatgatttcactgaagtgaaataaggactttggccttgctctggcaaattcaaatgtggccacactctactccattgtggaatggtgactcccctgtcccatgaaaaaccaggatcaacatcacagctttctctagcaactggtggtggcctgaggtcttatgaactagcatgttagcagggatagagttagggaatttgtccgatgctgacagcaagagggtgctgggggagctggaggggtggagaagtgatctcccttcctgcctcatggcttcctttggaagttgcaagcttagaatttctttcccaaagaatattggactatgcttcaagagacacttgagttcagttgcttacagtgaacatacttcttatgttccagagttaaactcaatcatattttcagaaaaatagatattcagacatcatgtcatatttacaggaagctatttgatcatggaatttactgaagtgacagctttttcaggaaagggtatcgagtggttatctatatcctattaatttctaaaagcaatgctcaataaaatgtgcaattttaggcaatattctgtgtttcaacattatttccttatgttgggggtacatcactttatatagataaagaaagatccttttcattctctattaaatggtcgagaagtcaaattttggctttcatgttggtcttcttactgtagcttctgttcacctaagaaagagatttaaccaacataagctttggctaaaccattgtaccaaacatttctatttggatgctcttaacttttatacaatactttgaaatgtgttcaataaaacaacatgagaaagaagtagaagattctgctattaggttggaaattgatattccttacataagtgattaaagatactttattcagtttatcccttaaaatggtatctactgaagtccttaaacttaggcatgatattaattcctggctagtctttctttttcctaaaatatcactcaataaaatccaagaaggactaaaactcattatatagaactgcactgcccaatacagcagccactagccacatgcagtatttttaattataataagttaaaattaaacaaaattaaaacttcagccctcagtggcactagccacactttttgtgtccaacagctacatatggctggtggctgccatattggacagcactaatagagagcatttctatcatcataggaagttctgttgaacaaggctgatctggaagctacaccatcctaaagctctttggcaagaaagcagagcttttccataagctcagtttatgaacaatatattttgtattttctatgtatatgtatatgagaatacgtatacatagaaatatatgaaaatacatatacatataaatatacatatacataaaatagaaatacatatacatagaaaatacaaaatattttgtattacatatacatatgcatggaaatacaaaatattttgtattatataatgcaaatgtaagatatatttatattatgtaagaaaatacatttgtgcaataaaacgcttatttcattcaagcatatttgtccagcatggtttcttagagactgaagttacccagtttagcacctggaactgaattcactctggctgtgttagatgtagcaccagcgtcatgacagatccagcccacagcatagactgaggcctactctggaggcaaatctgcagacaatgtggccaacacatgggccttcctcacatctctgtccagtcgctggcatccacgttataacacagcagtggctcgttttctccagttaaaggctaggcttccgtgattgaagcaggagagaatttggtagtcagtaaagtagacaaactaagacaaaacaagagtcgcatgagttcttggtacaattaaagaaacccttggccaccaacgtttttaaattcaggaatttcaccaagtccgtaacagttttagctctcaagggtagaattttttttttaacttttttggtaataattgtattgcatgcattccccttacacagaaggctggcatttaattggggtcttgaactcaattgtgttttctgcagttggtaaacctcacaaatgtggatattgtggccgaagctataaacagcgaagctctttagaggaacataaagagcgctgccacaactacttggaaagcatgggccttccgggcacactgtacccaggtaagcgctgctgctcggaggccagcctggtgggctctccccccagcacggtggggaaggagggcgctctgcatgcagccttaggagcagagccttgggcctgcttcctgccggggctaggagggagggaagtttttggccaatagcatcagtttcaccagaagcacgttgtgcttcccagctttctaggtcctcatctgaccagagagagcttgattttaaaacccttcccacttccaatcgggagaaactcctaggatagcagtgaccttgaaagttttggggttgttttttggatgttggtgattttaaaacaacaacaaaaaaaacacctcaagtagtgatatttctttgtaaacaaaataaaatgtaaaatattgttttgaaacaattttttaacaaagttgatcaaataagaactttcaggctgtgattttaagcatcagttcagactgagggcagggtatccactcctgcatggagtgtgaggttgaagtttgttggaagcccctggtagttgataatattgaggttgttgaaggatgagaggaaaggtcgcttctttcttaggagctgtgatgctccactgtattgcataacgagatagcacttgattcagaccccagagcctgtagaataaatgatgtggagagagtattgaaaaaggctggatttatctgggggaatggcaatgttataaaaggttcttccctaattttttaagagataagcaatttataaagagatcaaataaaactaatctctttcaaaacagtattcatttagcactacccctggaaaaatcatcactagactgtgagtcgaaatttaacgaatggaggaaaatttattaagcctcattcacctttcggactttaacaaatggttcaaaagggaaaattcaccagagtaccctcagattattaatggcagatgagagcaggaaaaaagaatgttgagaatggctaaaaattgattccaattagtcttggcatggagaggcaacatctcacctggccagagccctgcagccagagtgctgtctcctctgccagtcagccagggatctggggcttacaggcgattcctccaggcctcaatcttctcacctgtgaaatgggaagataccattaaattgtcatttggcttctttcaaaatctcaggtctagaatggaaaggcattgaaggtgagtggaagagaagaaactggatgttaaaataataataatgtaacagttattaattcacatggccagaccccagggcagaagactaatggacgagaggatgattatgttcctttgaaataaaatgtcaacattcaaatattgtttttttctgtaaaaaataagtgaatgggtcttggaaggaagtgctcctaatgatgtagtttacatattaatgcttaataaaccattttatttactcaacaaatattcactgaggacttataatcaataataccaagccctattgaatacagtaatctaatatacacatagaattgaaaagacaaaatgaacaaaagaaacagacagtatatcatttctgtttcctccactgttttcattactaaagaaacaagctgcagtacacagccggctgagagctgcttccgggggagcaggagaagcagcactcatttcactgagacctgggtttgagttctctgtttttgaatgtgacccagagctcatcgcttaaatgcgactgataattcgtgcctggccaccctcatagcagcgctctgaaagaaacataaaggagtggatacacgtgtgagcaggagcctagacatgtgtcaggtacttacctaaacatggatgtttaggtcactgacatactgacaccatcaagctaattcttctctattggtctctatgtcagtaaggctacgtgccagtaatgtgtattaaatattaccctgtggtgagtatcaatttcaccctaaaatgttcatttattccctttggtggttaagttacttggactaccacagtgatgcctctatgtccccacactcaggaggtatgaaagcgtctctaggaaggagcagtggtctggtctcagtgtggcagcagacaaagccaggctcgcacttaattacattgaattatttctgtctggtatggcctcaaaaggtgcaaaatgatcataaatatctttcgacagtcacctttggtttttgcccttgccagcacctggcccttgtaggtgctgctggtttaaggaacttggagtattaaggggatccataaaaggactttgacagaagtcttcctgaactgtgcttagggccaggaaggcaggcaggggcttggtcatttcagctgtgctgtacccggatgagtggaaactgtgccttctgatgcatctctcctcctctgttcccctctaagaaccccttgtctgtgctcagatggatgagcaagtagtctcttctcccaccccaaaaacctccttagatggggtggattcagtaaaatcaaagcctcagttaggcaatataggaaaagatgaatttttgtgaatataattttctttagtaaaaggcttttggtcacctcttataatttctcaatgtctttatggaaaaaataagtttctcttgagcctttggtgcacaaacagagtcaccagcccttggaggtgcatgcatctaatgacagagcagcctgtgactcgcgagcatcaggctcacttgttccttgctgtgattgactctggggggtggaggcttttgggaggccctgcgccatggtgcagaggaagacccctgacctctgggagccctgggcaggctgtgggtgtggcttggcagagaaggatggacatgtcaggaagcatattagcttaatagatgaagtgtgtgaccagtgtaattgggttttatggatttatatttatcttgcatataaatcagggtgtggcctatatatgtttgcccatttaataaaaaacacatattccaaatgttaaaatataaaatataccagcaacaatagccaatttgaaatatatgttttcaaagtaacattcataatagcaatgaagacataaatacttagaaatgaagaaaaccagcaagccatagtgaggcaagaaagaaaacatgggagattggagaggtagagaggtctggatgccgtccatgggcctctgcccaaattagtgtatcatttcaacataaaactggcttgtaaattttactggaatctggtaaccatgtatcataaagtatctggatatatacatgagataattggcaactttttagaagaagaataatgtcaagtgatgtatgaaaatgtagctgaaatggatggaactctttgaagaatagacaaacaaatcaataatagagatggataccctactgaagaccccatgataaatcagcatgaaatatccgaggcaggaggaagcataaggcaggaacggtattggaagaattgatcaggcatttaagataatcagtttacattatcatctccttatacactaggataaattgccgggaaatgaaagagttatatttttataactccaaccacaataactgatgtaaatatagatccagctatgcaggatacaaagttgtatcttcaattatggaaagagatgacatagaacaattgagaatgactagaaatggcccacacatcagcatttacctccataatttctaaattttctacagcagtcaggggtgctttgattttcaggagtttgttgtatgctcagggataaggggctctgggtttttcctccacattgcccagatctgctgtaggctcttgggggccacatttttgtatctccgttcactgtgctggtccatgaagtcagatgttagatcacacagttctttgcgtctcagagggctgctgttagggactgactgaggtagcagatttgggagtattatgaaaagttaaaaggacttgtcaaagggacatcaatacttaatcccagtccagcctctggtgatggtggtggtgcctgtgcatttggacttaaagaggatgatgtgtggtgagcagctgttggaggaagaagcatttgcaggtggctggtcattggcattgctccggctgcctctgcctgtctggaagtgttgctgggaagattagaattaatctctaggaagggcctggctcttgtaggcacttaacaaatgtcagatttaacattggacgcgactgaaccctttaaacataagcctttctaaactggcctctctgtctttgactttagtcattaaagaagaaactaatcacagtgaaatggcagaagacctgtgcaagataggatcagagagatctctcgtgctggacagactagcaagtaacgtcgccaaacgtaagagctctatgcctcagaaatttcttggtaagagttaaatgtttgctgtctcttaaaaaaaaactatgtgggtgttttagatgcaagtagaaatgagttgagggtggaagaaagggaaaaaaatcttattttttcaaaaggaaaaattggtaagcttaacattccttaaatatcttagaattttttccaataagtatcttaaaaataacaaacctcccatcagtttttcctagatttgattttgcagcatctggggcctgccctgtgatctgcctgtggacatcgctcttaggggcggctgcaccagcgtgcacagggtggagagtttgggcctggctcgtccgggggacaccacactgcaggacactccaggcctggccggcttctcagagcttcagatcctcatttttcatatgaagctcctaatgctccccttatgggggactctgaagggttaatgggaggaatcatacagtgactgacccctgagaagtgtccagtgaagacagggcttagctaggattgctgttttgcctaatgctctgcgggattaaaaaaaaagaagaagaagaacaagaccattcgtctctctaggagcattgcccagagtaggtattagacacaccaacaccaccatccagccagacgctgcagggacagtgagccagggtccgagtggaaaggcgctaggcttgggaaccagctcagagtcaatacagagccaccgccactcaccaactctgtcagcttagtaaaatggctctgcccctagagccctggttccatcctttagtatctcacagggtgattgtgaatatcccatgactccaagattgagaaaacgtttagaatccctcggtgtgaaggttaactctgtccggaaagaggaccagtaaaagcttcatgaggctgagatgcactttggaagaggaatagagtttcagcacattctaggtgttggaggaatgggggaatctaggcagatgtttaaaatcaatgagaaaccagaatgctgaccatgagggttggagtgggggcctaaggacatgacggaggagcagggtgtgttcccagcttaactcaggtacccatggggaagcaggaaaagtgaaggtgtcctaggcagctctgccacaggatgaatggcttcagatgccaggtgagcgagggacccttcattcagtcagcaggaaagaagcactggcatattttttatgagaacaaaggctaggatagtaaagacagcaagtaccaaaaaatgactggaaaagggagactgtggaggcagtggcagcaggcatggaaagaagggcttgtgaaggggaaggggtggtgtcagaggaacatagggctgggggcagggattaggtgagggaaaccatgagtcacactgattctagagtagtgtgcccttgatgaaaaggataacaccaggttctaggaaaagatggggttctgtttttgacgtgttgattttcaaggacttctggtgtttgtgacacatggggaaattgtggtgggagagaggtggggccagaacaggggctggtgaggccaagggtcccagagggcacctgttgacctgcaggatgacatgaagggggaaggacagaggcaaggccaagtcctgggcaccagcctccctcttgcagcttcaaatagggctccattttgaccttttgattaattagaggtttgtcataggttgggggttgagaggagcaagggagagaaggattcagtgtacaaaaagaatgaaagccactggctgagccagtggggagttgtccacacacacatgagcctttggaccatgagaacgagggaggccttgccttcctgaacggagtaggagtgaggtcctgtgctgagcgtaagcagtgggattcccacagcactgggcacagagcccacgggctgcctcctgagcagccagcatctgcctggggtggacacagtgacagagagatgggtggtgactggggtatgggcagagataaggcagcaagtgtgtgcaaggggagtgaagggttactgaccttaagaagcagggatggcgtcctctgtcaggtgaggagcctggagaatgctttggtgaatgaacgtttgcagccccttttagcttttggagacttgaaaccaaaggagagattcatctgtgaaactctactggagccactccccaacccccacccttgtgagaccacaatgtgggcgttggcttgagatgcttctgtgttagtagaagaaataaacaacacagtgctctgatgaggcaaagcgaagatgaaaaaggagttcccaggggacatagtaggaacagtggacgagggtagcagaagaggagtttggagcaaaagactcacaagcagctgcataatctgttggtgcttggcagttcatttgtaaaaatgatgcctcttcctgccctaaaatacctaccttacccccgcttcaacttgatgagatttccatcagtcactcccaatgtgtcacagcttctgcagccctaaaattaaaaggtgagtgagtctctgaggcccctctccacttctcggatgctgagtttagccttcatgtgaatgtggaaagactaggaatacagctgttatcacacaagctggcccaatagtggttcagttgagagagccccatccttcagagtcagctccagctaggagtgactggtggccttgagcatggtgctgggcttagtgttgccatctgtggaatgggtgtgggtctgttgccctgcctcctcccagagctattctgaggctcagaaggggtgatggatgtgatggtgctcccaacactagaaagcatcttaagaatgtaagattttcatgatgactgttgctcagagtggctattatagttttgctttattgttctataacctatgattaaaatttttaccttaaactttgacgtgagtgtgaataagtatttgttttgccagcaacattcctcaccactggggccattaaagatctccccctctgagaccatcaaatacaggtcaacaggactgattaatctaattagaaaagggcttgtattaaatagcaatgataattgttgtttttagtctgtctggtgtttgacttgggaacgtttttaaaatagagaaaagcacaaagaggaaaacaacaattaccaatattcctgctacccattataattatctaggtatattttcttcttttgtaagaaaaagaaaccctgttatattgttaaaataacacaaagttaatataaagaattttaatgcaaagattaatgttttcaaatcaccacaaaacccaacatccagaaattaccaatattaaaagtagaaaagtatcattctaaatattttctgttgcatatgtatgtgagtggataggctgatgaattaggtggattgatggataggtaaatatgaaataaatactttcataaatattccaacttatcatacatgccttaaattcaagaggtgaaaaaagacccaaacaaaactagagaagcggcttattttaaatatcctctgacataaaggaatattatatttaaaggatcctctaagattaaaaatatgtactatgaaaaacattaagaaatttgaattttttttaatccatttgtttcaatttaagcagcatctactggctcactgctttgaaaaataaggacagtattccagttcacattcagtgttccagtgttcacattatcttattatttttacattgtccagctttgtaatattcacattctattctgtaatcataattcatagtagtttagttatttattactaactctatttaaatagattcaaggatcagaccctgcccttttcttcttatttatgtttattttgattaatctcttaattgattggactttacattcaagcaacttttttaaaaaaaagtttctatagatgttctatttctatcattgtattgtttttgaggatgttggcctgttgcctttgtatttgatgagcattttgacagagtctatggtcttgggccactctttctttttctcccttgagaactttttagattttgctgatggcattgcttgttgaatgttgctgtggaaacatcaagtctagtgtaactgtttcttcttcaaggtgatttgcattttattcctgaatgcctgagggttctttatttaaccttgaagttaaataccctaattaggatgtatcttggtctattcattcggaataaaaaattcctgccattttgtctagagagtcccttttttttctctttatttctgggaaattctcttttatataaatatgttttgttccatctattgtgatctctgttgagggataccagttgtccatatgttagataatttgtcttccatatctgttaacagttcttaaagttttttgtttatttctttgtctatttttacatttactcactgttctcttgtggttttcctctgtcagtaatttaatttttagtagttcctgttctattacttgctatttttaatccatgcattaattttataataatattattttgctccttattttgtctcctgagacccgaaatctcttttttcctcttactctgttgcttttgcattttattttgaatacttttaaaattgattccatgttatgaagcaattatgaggcatttcctctcttgttggaattaacgattttttcccctaggagggactctatggtctgtgttttacttcctttcttcccctgtatttctagaaaatattttcctagtaaccctgacatttcttttcatcttgcttattctagttggtctgatatagcttgattgacatttcagccttcttcccactatatttttttttcctgtgagagctattgggttttctaaatcctgaaagaatgccaaagatggggttggaggagtttggtgaggcaaagtgcagcctttgttaaaatacttttcctttgctctctctccctcatctgaaatttagttaaataccctaagccatcagcactgtacctagttggggaatgctttcatccccacaggagattctctggggctttgggccatcttccccttcagtgtagaccacagaggactttgcttctgtcccagggagcccgcaggggctcacttctccatgttcatctgattcttgtcagccaaggtttcaaatgcttttctgatcagaacagggaaaagatacctatctgaatcatgtctttatagatatgaggctatgagggaaaattctgaggttattcttgactcacacctaaagatttggaaatgagattagcagcaaagctttgccctacatctcatgtcagaattttctgtttctttctagtctttgagtgtatgtgtgttctcacacacgccataatgaaatgcatattatatataattatgtgtatatataatattctatgactatacatgacatgttcctttagctgattgctgttaagagaaatttataggtttttatttttcttgttttgttgggtattaaggaagagaaattctatggtaattttcatgtggcacagtaatctggcatatatgttgatttttttcctacacccatttgttgtgataccaagtttgaaaacaacagatttcagtggttgcttgggaaaccacagaaccatgacttggggagagacaggatgattaggtgggaaagcacccttttggtggggctgtaaagacttttatatttagcaaaattggctacaaagtccattcccctccttttcttgccttgatttggtagagggatagacttggatacaaactagaatggattcattcttctctggagttagtgtaacaagacatttagctgctcaacacaaaaacagaaacaaaaaaattgtgtggtttcagcagtgctatacaattactttttctgacctttaatggagagaaacaccacttctttggtccctaccatcagcttcatagggttttcatcctgttctgtttctgggagggcgtaactggccatgcacaagttttttttctctaatcagagtatgtgccacttctgaccaccagtagatgaaaacgaatggaaaccaggctattatatgatacatatccattacaaaataagacatgaaactcaaaggtactttatggtataatggggcatatattcctggacaattcttaatggtcacagattttataaaaggactattagtaaatgtatgaattacagagtaatttatccttctgttagtaagaaccagctgatgacctcagtgtcaggtgcatcgtggaaggtgttgggaccttcccttgccaccaccctcaccagccatcatcagccataacctgcacattggggaagttttgacttatccctcacttttgcccctcttcaagctgttctttccacagtgaatgagaaggccacttcttccttcaaacctttcagtggtttccattttcctttagacaaagtctctgcctagctggcctctgcctgcccctcctgcctacctctcgagcactgcccccacctagggctctggttccccaaccttcactcggtcctgccacacctcccagccccttctcccttcagaactttccttcttgttgtccccaacactgggacacaaaaccctccttatcaaccctccttatctggctgactcttacaagatcagaaacctgtgtaatgctctcatggcacgctccccttgtcttcgtggatttctcagatgggaaggaattatccatgcaatcacacataaacttctacctaccctcccctagtagctgtctgctgctaaggatggggaccattctcacttactcactgttctgtccctctgcccagtccagatgtgttgaaggatggaaatatacagagtagtggtaaaatataaaccgttcagacattccaaggatgggctcatgtgctttgactcattaatgtaccactgctgaaaacagaacacagccgcagtcttgccagtaagagtgcagttactgtaattaatgaatttgctaattaagccatgatttcatactgaacttatgaccaacatattgagaaggtgtgtcttcaagaaaatttattttttgtattaagatatttactccaaagctaattgaagaagccaaatctaggctctggtttcaccattgccagggaaatgagctcatggactcctatgaactgatgatgttagatcagaagtttctcaaggccagggcccaatcactgctgaggcgtcaacagtagttccttgtacatcaataattctcattacttttaaaaaataacagatgaatagcaactattttccctgtagctcccttgctgtgcctcctaccctccaccacatgtttctggggagccctgcttcgggcctgccaactacagagaattacttttgagtatcccttccactctcatctcaagacagagttcatctacctttgggttatttgtcaaaaatgtgtcattttattacaaaaaatatacaatcatcatgtattttgattaaattttacactagattattaaaattattaaatacaattattaaaattaataatttaacatatcacatattttaaatatattgtatataatgaataataatataattattgtctattttaattcaataaatgtatagtaagttagccagttgtaaattactgagaacactctactgaaaaagcatcatttcaaatacactatttaaaatattaaatgaaatacaataacataattaaactaatctttggttcccctatttatgtattcatttatccaacaaaatctccttaagtgcttataatgggtaggtcctggctcggtgtcccctagacagacgcatgggccttcccccagcccgtcagtatggtgcaggtgtgatgtgtccgcaggtgtgtgtgtatgtgtgcaggtgtggggtccgcaggcgtgctgggcccccaggccgtgttccccttcccctccccggttgtagatttcagctgttgctgccagacctgaccggttccggaggtggccgcgccccactcactgtcgcctgctttccacaggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcatttacccttttctgatctcctggaaacagctgcctgcctgcattgcacttctcttcccgaggagtggggtaaatttaaaagtcaagttatagtttggatgttagtatagaattttgaaattgggaattaaaaatcaggactggggactgggagaccaaaaatttctgatcccatttctgatggatgtgtcacaccttttctgtcaaaataaaatgtcttggaggttatgactccttggtgaaaccttctcacattctcattggaagcatatgggttctcaaggaacgaaatccctttggctgttcccaaaactcatgtcttttcttttatggccttacaactccaacacagggctaggcacaaaagacacactcaagggattgagcaattggtaaaagacctggaggaagtttgacaaccaagaccgaaactccacgttcctccaaagcccaggaaatccttctgaaaacttctggccttttctctgcaccagaacccagaacgggctggcacagtgttgctgtgcggagctgaaggcaaaatgttgctgttgcctgcacagatctgtcttctctgcctttttgcaaatcatccgcccctaacttcctctaaggaggcagaaagggattccccgtggctcttagaatagcaaccctgccctcagcatcctaggctcccactggcttctcctgccattaaacaggcctgagcaaaaccattccctagttttgagttgccatgagactccctcaccgactccagccttgccccggctccagtgcctccaatgatctcccaggactttgtttcccaaatgcagcttgtaatcatctgagtggctgt

SEQ ID NO: 2 - DNA, 153937 bp, Homo sapiens
  source 1..153937: mol_type = genomic DNA
aaaattacctacagagtggtgcttaaggtactgttgatgacttaatttccaatatatctttttttctcagcagaaaaaaaaaacctcaacaactatttctgggattcttcaaaggagtaagagtacggtgcctgttccgactcttctagcattgattttcttcacaatctgtgatatggtttctcaggaacaccgtttggcagtcctgataatttcatgtgttttcaaatttcagccatccagactacacagttcactattttcagctcctttcattttccttttccctttaattatagaagtctctattccctcttttcgcaaatgtggtcaggaaagatactaaatgctactatttgtagaaagaatatgcatttgtttttctcttgacggttcacttaagttctttccaaataaacaagctgtggttgatagtgataggtaacacttcaagggtagcaactttttgtactcatccgttcctttgactttggaagacgtggcacatgccccgggggagaaaaagcaagtcaacactttagtttttaaacttgcactgttaaagcactttcaaaatagtaatagttgagcttcatctcagacatgaatgtctttctcagtctattccccaggacccctcttatcccgcttcaggtcccggtcaatgcggtgcgctttccgaaatggggccgcagagttgccggcggagaccagtaggtgggaggctccacgcacactccccgactcggccgggacgcggctcccggactccgcgcagggcgccaagatcgccgccccgctcccttcccccggcctccccaccaatcccagtttgaaagaaaacttcacgcggccgaaatgaacgtcacctcaccgctccaagcctccagccggcagaaaacgtgagatttattatgacacggggaggggagatgggggtcgccgtggcacactcggaagcgggttacctggcccccgtcgggggcggggtccgcagtccggggaccaacgggctgggggcgacgcggccgggcgcgcgggtgggcgcgagggagtgcgcgcgtcgcggagcctccggggcagcacttgtcctgtaatcgattgccgagcgcgccgagcggcttagcgcgcagacacgcgcacacgcgcccgccagcccgcgcgcgcgcgcacacgcgctctcatacacacacgcacaggcgctcgggccccgcacacacgcacgcgcgcgcacgcacgtccgctcgcccgcgcccggggctccgctgcccgccgccagccgcccgccgccgccgccgccgcagccgccgccagcccgccctccgcccgcgggcgtctgcgcgagcccggccggcgggggagatgtgctcgggctccaccggatcggtttctcggggtttgaccagctgtcccgggctaaccctgctcctcgctgaagatggaggaagtaaaaacaggattacccttagctacagatccactgccttagtttccaccaccaactgcagtgcacaaacacacgttaggcacaggaaagaaagaaagacagaggacacattaacagtaaacacaaacaaaagggtgatgggattattttactgcatgcactgctgaggtaaatctccgcttggcttttgcgtggtgaagaacagctcttgttttgtcttctgattcatagattatatataaatatgtgtatgatttgcaaaggggggagtgttttcaaagttaagtgtaattatatagcatctcgttttgcactgtgatgtgatcaaatgacttgttgctgttaaccagtaataataataataatataggtatttttctctgggagggatggattgtggttctgattagggtaatgatttttgtaaaggacgctcagggaatcgggttgaagatgatgtcctctgccttaatttatcgtcccctccttgtaatctgtttctgggatgttaatcgattaatcagttctcatctctatagctgtcatgaatttggacttgttttgctttgttttaacttgaattccaagaagaaagattgggtaggctggaataaattgaagctgtccagatagaaggtaatatccagatcctgttggaattaaaaaaaaaaaaaaaccaaaaaccctggtatgacacatgtaaacaggcagcagttaaatcactttgacaggttctagtatgggcgaaagagatggaattaagatttatttattttttttcttttttgccaagggccaaaaacagtcctgtgaagttggcctgggttcctagctgggttctgcctgactgtccctcccctctacccgcccccccccccttttctttcagttttcttgtcttagcttttggtcgattcttaaggaaccggtggatcaagttttttctcttgttaatcgcattgctatagcactgactgacctctctctctctcttttttttcctctttcctgaaagtacgtggtgccattcctgagtgacttttcctactagaccatccctaagggacgagggggaggggtggaaggaggaggaggaggaggacgaggaggaggaggaggaggaggggggtgttcttctctctcatttcttggttttgtttatcagccgatctgtttgctggatttgggcttggaatgacccacctgtaaagtgcttttccttcctcctccccttgaactctgcagggggcttggcttggagggggcaagggagggaaagagagaagggggaaacacaaaaaacttctttctttctccctccgtttatcttcagcccgacattgtcacctcctctttgaggggttagaagaagctgagatctcccgacagagctggaaatggtgatgaatcttttttaatcaaaggacaatttcttttgtatgtacatttcgtttctctttctcctttttctttttccttctttccttctctgagggttaataaccatttcattgatggggcagccttctttcgcttctccctgacgtttctttctttttaatatgtgtgtgttaaatgtgtacatttttaaagagataagctcatttgtaaaatatccgaagaaactgctcctcttttaaactttacggatctctttctttaaagccttatgtaatcataatttaaaagaatgtgataacagtacagcaggttgaaatttaagtgctatgtagtgactgagttgggaatttgatctgattattccataaagaaagaaataaaagtttttttttggggggggggtaagggagtgtggtggtgaagaaggggaggggagggagaagggaagtggttttggagacaagaagtgaagctgcctgttttccagtaagtaatggagaaattccaggagccagggctaaatatacttggcgatcaatacagaaatggctcattgtgaataatttagctgctggtgcagtgagagttttgaaactaaaggcaaaaaggaccaaggccaaagggggaaaaaaaagtaaaaatcttctcaggacttgtcaattcttaaacttttttccccacagaatacagttagattatcgatcttttcattatgtgaatcaaagctagggggtgtcatctcatacagtttacttttgtttctgagaattatgttctgttgtgtggcgtttgtgcatgagaataattgcccaaagttttttttttttttaaatgattctctctagcacaaccaaagtttatgaaattttcaaaatcagactgtattaagtaggcttggcatttgtgtccaagcatggtctggcaggaaattaaggttacattgaacaggtgcttacacagaacttggggtaggttaaaatgtcaatacaagtcagggagttggccaactgctgtagaaatcttcaagacaccgttgggcactaagttttttgttttgttttgttttcttatgtttgtttgtcctccccctccctattagaacatgatgaaccaacttgcaacaattgtgaatagagctatctgtatactggggaagtcctcaaactgcagtgtgattgtgggaagtagtgaatggtaaaacacttaaatcccagttaaagctgacacatttgagctataccccctattttttcttaaacccaataaattttgattaagagagtcattccctttttgctctagcccatctcacagcacacttatctttcctatcctatctccagcaggaatcagatcatttgcagaaagagttcagtgacttgcaaatgagcacatacagtacactgtgtatgtttctgggcacaggtggcagcagcaacatggactaactgcttggagcttcaaataagttaagaatttaatggcagatactgaaaggaatcattgtattaagggtataatcttttgttttgcagcattgcactttgactatggaaacagaggctattgatggctatataacgtgtaagtattagttttcattgatttgttattggtaataaagttgcctttgtattacctgtgttttaattcctggattatgaaactttattgagaattctgtactcttttttctctctgttcacaatttcacctttgaatgagtttgagaaaatgacagcactgaagtgaaataagaaggaaatatagaaacatactaagttaagtagtgtacttaaatcaatagtagctagcagttcaacactttcctactttttataatttactttgttgtaagttgagaataggccaggtttcatatatgtctattaatatggtgtgtactaaatatttacttctctgtaacctaatttttcagacatattatgtgaaagagaattagctttcctaacttgacatttctttagctacctgtcttttccatcctctctctgatagccagcattttggaaaatgctcattataaattctgtgtgcttaacaattaaattagatgttaaaagaaaagttatttctttcaactgaaagatactttagaaatcaacaacttatgtattgagataataaagttttgttaattttttttattgtgggttgattatggcttctcatcatttcattcaactgtatatttctaaatgatatttccttatagtttctaagggaaaaatgtattaccttacagtttgaaggctatgcatgccattgcgtgtataaagcaaagttagtaattacatcttatgtgacaatgaatagaaaccaaaagattatattttgtgaaagcaggtgacaatgagctttcacccgaaagggagcactccaatatggcaattgacctcacctcaagcacacccaatggacagcatgcctcaccaagtcacatgacaagcagtaagtcttcattttatgatttttttttgtgtgtgtgtgtctctccagcaacagactttaatttgttaggtctgtggggtcctctcacattgtggtatagctactgttatagattttgctaaagaaagaacaacacagatttattcctgttcattctagtattataaaacatgagcctgcttactagtctgaactatcccaaggtggcatttgggagctggtgcttacctgtatcaggtaagaaaggttagaatttgctggttgttgttttgacttgccaggtttatgactggattttttgttgttgttgggtaaaaaatgtggaaacgtcacttccaaaggactcttcacagttaaaaaatgaaataattttatgttgccttggtagaaagctaaaccgtcagtgataaaattacagtgttacttgcttttaaaaaatgtgaaagtttcttctccaaagcattggaagtgaagtgtaattgaaaatttatagaactcatttatatccatttctgtagcaaagttgaaaggcttttgtccagagaagaactgataaaatttggaaagatgttttgaatttgtaggaaaagacagttttggctactgtttttgataaagggtcatttactgatgtaatatacattgtttttatgtaataagatgcaacttacatgctgtcagtgacagaagacaaaatactttattaagaatatggttgcttttttcttaattataatattttatcagtttagttatcaagtgcaagtgctttattactcttactcaaaagtaatttgaaatcttgttttctttatactttagatttatattttattcttacatgttatagaaaggacattggacttacatgtaaatataattaaatttacaagttttctgttttggcaatatgtgaaggaaaggcattctctttccttccccccccccgaagatttaataactaagaaaagtgaatcatgtacttaattatgagcatatttgtaaatgtattctgactatccagagagagcaactttttctttttacttctaacagctctcgaaactgtaagttattataaaactgcaagcttagtaattagtaaatactactgaaatttaagcaatttatatagaataccattgaatacattttgcaatttattaactttaagtaattttagctattgacttgaaattatgattgagtttgaatttgctttatttatttaaaagttgtaattcttttaagtaatgagcacttcctaagaaaatttaattatagaagcagtccttacaattcccgtatcatttagaaagaaaaaactagtaaagttagcaggtgaatgtatactaaattatcatactaatctcataatgaatcttttgcattcatgttttttgctagttgatttgataaaaaatatactatgactacaatttaggcaatagaatttttacatttcaatatactagttatttggtgtaatgtttttatttctcagataactcacacaatttcctaaagagtaaaaagagataatatgtagtttctattagttagctattggctttagaaacattgcatgtatagggtagggagtaactgttaatcagagacttgactgtcatcccctagacacaaccacaggaaaatgcttgtcaagtggttaattggctgctttatttctgaatttgattctggttttgcaagtacttgcttcagaaaggcagttcactcatctttttaaataaacagtgcagagtgtaggcatgatcaatttgaacaaataagaactgtgcttttgacatttgagcttttctcacccaacaacagtaaacttatgtttggtagagagttttttaacttactagcctgtggcaattccatgttagtacaggttttggctaagatgcacgtgttttcaggttcttttccaaagagttgcaggttaaatattttggctctcacttcagagctgtctcttgcatcctttatgtagcctctatttacagtatctaggttacccacttaggtgttagatggagaagatacccatttttcctctctgttagatctaggagaatatcagcattttagattgccaaagcaaatagtgatagagaggaactctcgtctattaattagaacaagagacctagaattggtcactctgatgtttttctgtgacttggaataattttttgtgtgcatctggcctgtcaaatcacttgtgtctgagatcaccggttttcctaaatccctttacatgcattgttgactttgtttaatgaaaagaacttatttattttctgcattttttaatatcttcaaatatacttcctggagggtgcctgcttccaaaaacagaattgcaattttgattgaaagtgttaaagctaaaagtaaatgtgaaaacctttaagacagtttctagtagcaggaaggagaggatgggaatatatgtgtaaggaggaaaaaagttatttttaaacagttaaaaaaataaaataagtaaattttttttttttttttagtaaaacagctaacattctggccagaaaagtatcaataaggcatgagagctcacttattttggctttaggaaggtggtcagtactactgtaaggtcccagcatttgtttaagctatttggtaactcttctaagatgaagggtttctgtgcaacttgctgccagctattgtataaaaataatgacggcagctatcattatttgacagtgctagaatagagcactggtgacttgggagggagagaactaaaaatgttataatgcctgacagtttttggttttaaaatttagcatctaagataaacttaagaagaagtaagaggtattagtgatcctaacattttataatgactacttcctatggttttgaatgaattttcttagagaactttaaactttttcttctggaatgaggatgtctttgttaagaagatggacattgttgcctgagatcattagtgtacatattgagtctgctgacaatcttcggtgtcagggatgacagtatatgccttgatggctattggaaatacacagtgttaagtgatttttaaaaatagaatctgggacttaagaatctgagacagacatctcagacagaacaatataaaaataatccatattgtatttcggcccatcagatgaaacttactttgtgattccttctgtttcttggttagctcatttccaattcttgcatagttgttcagtattggtttcagactgttctatactggcaggtgactattttgaaaattgtttaaagttcaaaatttttaaattaattttatttttcttatctatttttttataatgtttatgtataaatttactttgcaggtagttacttaaatgggtatgaaaatacatcatgatgcaaaatttgtttctattactttaaagctgttaagtggatcaactgtacagtcaatgctatttatcagtctgatggccaggtggcatgtatggaatcggaagaagtagtaagtctagttttacaaggctctgaccaataatcaggtcagggaatctagtttattcttttcagagtacttcacattaatttttttcttagctagtttccatttttagaacctagcgaagcagaagtatattatgatttctatttttcatatgtggaaattgaggcttgaagaaatcaatttacctcttagagaacttagcagaaagatgaagttatcaggattgggacataaaattgtgatattaccttgtgggttagttagataactgctatctctacaatgtcaccttctcagggtgtctgccgtgtccaatactggcccatatttttgtgacaaatatctcagaaatagaagagttattatgagggtggagtgatatcagattatcttttcaggcgggtgttctaatgcactttttgaaatagagcctatattatgggacttaccagctttctgagttttcttctgtagcttagtttggcagtcacccttgcttggatttctgtaggacacaccctaatataacaccagatgggcttgatagaaaatcttctgttttaagtcgccatacttaggtttgtgtatatacccaatgtactttacatttagttcttgtttattagggaattaaataaaaatagttctgatttaatttctttacaggtatcagagataaatgtcataactatgtaccaaaagagaaaaaaagggaaaaaaaaggaaagtaaaagaggtatagatttgttggtaatcaacagcaaaagtgctgtaactttttctttgcacctcattccgctcccaactccctatctttttagtctgcttagtactgagaattaaagtccgttttcaaaaaacaaaacaacgtatggcagaaagattatccttatcatctctatctttgttcaaaatctgtctttcccactagaagtacttttttaaaaaaaggtaacaccattgattgtgttctattgagagtcttcctgtaagccataatgaattttcatctctgggttccgtgctcttctctcctaacttttgacatgttgatatattttgaatatttacatatttttatcatatttggaaatatcaatagcagagttccagtcctaaaatgaggtagaatatgttgtttttctttacttttttcccttcttatctgtttatgttaaatgtatttgttggcctgttggtattttccttttgcgaattaattgagacccaatatacctctgttttgatataggtaaggaatatttagggatggtttcctttattataaatgttataatcaccactagagcgagagaaggtagaggggcatcctttattgagtgcacttagtatgctagatgctttacttattatttcactttgtctccacatcaattctgtgaggtaggcatgtaattactctattttataacacttaaaactgaggctcagaagcagtagactgtgagtggttaaaaatatgggctttgcaattatgctggcctgggttagaattcctgctttgctgtgtgaccttggccatgtcacttcacatctctgagctccagtttccttatccgtagaatagggataagcaatacctacctcaatgggctgttgtgagatttcaataaggcaatacatgcaaaatgctcagcacaacttctggcacatgctaagactcaataaataatcactgctgctattactcagagcagtcaagttacttttttatggacctatgacttgtaagaaaaagggtttgttttcaaatccaagtctatttgaaatctgggtcttttgctcttttctgtatattgcacaggtccagggaagaaagatagggatttctgataatttagggtaaaaattgaagcaggaaccctccgttatttaaagcatctatgaagattttggttaagaaagaaatagagtttgtctatatgtcagaattggggaggggggtggcgctcaacattcctgagtatagttttgtggttttgatgatatgtaaagcaaatcactgcatattattggtgttccagatataccagtcattacaaataacaaaagaaaaaaattcccttaatagagtaacctattcctacagagaatcaattcttagagagtcctcatctaaaatctaacctcatttgatgcttctggtagtaccataaattctcacttgatgatacctaataaaattgttggtttaattgttgattgtgcacttcgggggtagtagtttagatgttctaaatttgaagtaggcttaaatgcaattattaattataagcatttgacattttgctgttcaattctagttgatcaattcaataaatatttgtgggatacctcatatgtgccaggtattgtaaggtgaatccaaaggtgaataaagagtatagagtccttttcctccaggaactcatactctagtgggacagacagacattaaatagggcattttactgtagtaagcttagtgtcttgattattacgggggatgagaggtggtgctcatttggaaatattttttagagggggagactgcttgagccgagacttattaaatgaataggatttaggagtaactggaaggccttttcaataaataatatatccacaataagaggtgagaaatagcatgcaatattctgggaaatacaagcagattggtatttctagactataaagttggagtctaggaattcgaggccaggaatgactttggagagttaatcaaggcaggataatggaacatcttgtatgagaggaaacttggcttttattttcatggaagtcaatggttttaacgaagtaggtgacttcagtcttttgactgacatggatcgatttgattacaatgtggggaaaaagattgtggatggggaccagtctctaggcaggtgcatatttaacaggctgtttcaatgattgagtgagaagtcatgcaagctagcagtagtgggataaaaaggaaggagaaggatctaggtaatatttagcatcttaaagtagacagtacttgggtagtagaattaaggagacaatagagtcaaggatggcaacctttaaattacgggatagatggtggtggtaggttctgtcagtgaagatagcaaatactggagcaggtttccgggaaaagatttagcttagttcaattctggtttgaagcacccacagggtacaatttgggagagggatagcaattaggcacatggatatagactgaagtatgaattttcctaattagtgaaaactaatattagcccatggtttggttacttttctgcccatattactttaacagtagtttaataccatattttgaaaacattcatgtctaagtaattctcagtatttgatcagtggccttatttcctcaattgccccattaaatcaaaattctatattgagttttatgtgcacaggctttaataaactctttgcttaatggaatcctttatactggtctcagaagttgaattaattgatgtgattgacccactgatttttaaacaaaaagctcttcaacattttgtggtatatctaatatcataacttctgttttttaaaaaaaatcataagattaatgtatgccatatactccagtgcctctattctaatgatctgtctttattctgtaacctgtttccttataaataaatatgctattgaaaatattacatacttattacaggttgttagctaaacattggccagttctgccacattaatagctaaggaatttaatactcctcaccagagcctttttgaagactttgagataaatatgcagatacaatatatattattttaaaatgattttgtatagaattaatattttaaaaatataatatcataccccttgggatgaggttacctagatgggacagtatagcatagataacatcttaatcagcataattattctgaaatatttattacagaattccatcttaaaaatgtaagcgctgactacccaagacaccatagaattccaaatgaatgttaatgctagaaacacttcttctgagatttcctgtatgtggagattcagaactccttagagagatatttttcatctttcgatcattgatccagcaaacatttattaagcacttactgtgtatgatgcattgctggtgctggggctcaaaaaataggtttggggtgttttctagagaaggatggtatcttcaagggtccaaatgaattgatattttgtgtgagatcgtaatactgcttattgcatttagaagtaaatgagtgtgatgtgctaactgtgacttacatccattatagattaatgagctgcttctcaccttttacctccaagagtttttcaaatgtgtgtaaatttgaggatgtacagtgaaacagacaaaagggcaacaagccacttagataggttctcagaggataacatttccccacaaaattatttataatgcaggatttggcactcatccttgcaagttctgaaactagatttgtggcacacttagagtggatctgtatctcaaggttgactgacattaaacaggtttacaaaactgatatttaaaatcagaatgccaatttatgtttagatattgattaaaaataaagcacacacacacacacatgcaaccaacaaacataggagctggatcactccagttggcttgactagatcattttgttcttgtgaactgacctatatgggtaatcagtaaaggtcatttactgagcaaggaatctgaataaagggagttagttatataattcaaatgtttaatggcattagactaagagaacctaatatattcagcatcacaatagagatagcattgctatttggataattatggttgaagtcctttgtccagttccctctccttttaatggacatggatttagattccctagttcaaccaaaatacttctgagattcatttttttttccccttggcttcctaggctaaatcccccagattcagtctgtaacctatattaatacatacaatcaggttacgacgagttctttctggcctgagatccattcattttctttagtgtgatttatatttctgctttaatttgtaaaatgttgattttatagcctaactttatgaagacaatcttatagtgcccataaaggatacttccttcaactactattgccataaattcgaacttccattcacagaaatctcgacagctgatgtggtcagcagtttaagagttcttaatatctattttagatgaagatggaaaatagcactaccacctaggaaagatccttaagttagctggtgatatagaggcacttccagtttataggtcagaaggaaaaaaaacaccaaaccagtctcatgaaattattgaagtgatctgaccgcatcaaatgagattttatagcaaactgttgtatttcatgttagttagtaggaaagctgtcttaattactgagtgtgaattaagtaacttttcaaagcatatagatttatttgtgggccacaccttgattataaagaaattaatcaggttttccagatgtgaatctttggggaaaatggtcatatctcttcatccctagcatatgcattgtatgcattgtaggagccctgtaaatatttgttgaatgaagaaaatacttaagattagcatattacatatgtctgtttaaatagtatgcttctgatctacttgaaaaaatttggaaacttgatttcttagttgttgaaataatccaaaaatcctaatttacttgtgattttctctgtttggggatgtgttatttttataatgtctcactctgaaaaagattttgtaatcttcaaattggtcccaaaagctgatctctaagtttctaaacagttcttatgttttctgattctatacaaccatgttcagtggaatccacatgaatgttaatgtagatgtttccatgacaccaccatcatcatcttcattactactattaatatcttgatatagcacatttaaggttacagatttaattccatatacttgtaattttaaagccagttctccatcacttaaactgtttttagtagcatgtacaagatggaatgcttcaattagcttttcttatgcactccagatattttgatgagtgtataattaatcagtgtgaagtcacgttctaataaattaacattctgggtgtctacacttttgacgtttttgtcattgctctcattatcattgaagctgtagttctccttacccttgtctatgtcagctctacctacttcttactaagacattcaacatgcaggacttgatcttattttgaagtgctagggtactacgttgctgtgttttcagttgaattaacagtgagaatttctggttgagttctaaataggcataatttcttctacaagtatataaaacattttctttcatggccattttcataaatatccttgttttgttgtttaaaaatacacattttaacaagtaatatttacatatggaaatatcttataggtcttctaatcagacttatgccaatatgcttattattcagatggtctgaagtagatgactttattttcaatacatctgttactttttagactgaaaagtggcatttctatagtttttagggctgaggatgtatatttaattcgtgattgtaaaaggtctatttctattagaaatgcttgctcagatcacccaaatatcatctaaagcttgaaaattgagctattcaataatttttaggtactaaaaataggtggtacctaatcatttttgtgtactgctttaaaatgacttacgccttaaaaacactgactcgaatgtagtatttggaaatgtaaaatgacttctattacattattacatttttgaaaactacttgaaagcagcaatagattgtgtctggacataactgagaattataaaaagaaatatgagatttttctgtgagtctgctgtttttaagttctcgaagcatgaatttacacactgccatctgtctgaattatgacacattaatgacctaatgatagttttagatttaatttttttttggaaataaaaacaaaaagaaacacccccgtacctcaacaaaacacaatttaaaataaattattttttacataaataaatcatagtaatgctgtttgcataaagttttggggatgcatttgaatagttttagttggtgttacattttgagtacaatacctgtaacaaatttgaagagatattttttcagccagagaagctatagttaactagacaattgatttcatacaactctatgtatatgataataccatttatgtttttaaaagcatatatgtgcatgtgtatgcgtgtgaatgtgtgaaaacaaataataagaacaagaaggaaacgtcaaaacttgatatagttgactttgggatatcggagggtaaacagggtgttttatattttattctaaatagttctacattctttaagtctcttacaaaaaataaattcatatattatttgtgtaatttaaagaaaagaccaatgaattcagtcaaccatcctctaaaagaaagtatggagccagaaaactgatagtagctctgattacatccatttaactttgaaatttcaataaaattatagactcataaaaatgatcagctgtcatcaatgataaatactgaaatcaggcattttcatgtacaaatgatatcttcagatttattgaatactattttactatagatttagtcaaaatataaggtattttctttttcttttattctttcttttttcttttctttttctttttctttttttttttttttttttttgagacagtgtcccgctctgtcacccaggctggagtgcagtggcgcgatcttggctcactgcaacccttcactcccaggttcaagcaattctcctgcctcagtctcccgagtagctgggattacaggcgcccatcaccacgtccagctaatttttttattttttagtgtagacagggtttcactaagttggtcaggctggtctcaaactcttgacctcgggctatccacctgcctcagcctcccaaagtgctggattacaggcataaagccactgcccccggctctttcttttcttttttctttctttcttttttttttttttgaaacggagtctcgctctgtagcccaggctggagtgccgtggcgcgatctggctcactgcaacctccgcctcctgggttcaagcaattctcctgcctcagcctcctgagtagctgggattacaggcatgccaccatgcccggctaatttttttttgcatttttagtggagacggggtttcaccatgttggtcaggcgctctcgtactcctgagcttgtgagccaccgtgcccggcattgtattcttttcttttcttttcatctcttgtcttgtcttctcttctcttttctttccttttcctttcttctttctctctttcttttcttcctttccttttttcctttttctttccttttcctttcctgagatggggtcttgttctgttgttcaggctggagtacggtgtcacgattatagctcagtgcagcctggaactcctgggctcaagcaatcttctcaagtatctggggctacaggtgcatgccaccatgcccagctaatttttaacaatttttttttagagtcagggtctcactatcttactgtgttgtccaggctggtcttgaactcctggcctcaagtgattctcccgcctcagcctcctgagtagctgagtttacaggtgtgagccaccacacttggctaggttgtttacagtggcatttccaaataatgttatatactctgaattgttttatgagtaatattcattcatagcatgaagtgtcatacactaaataatgtacatataaatattaaaatgacatatcaatattaaggagacattgtggcatctgatggcaaaagaaatctgagtagaatgagttaaaacatagattttcaattctatcttgcatgtaacaaacaagtttacttttaaaaaagagccttagtctctttttcatattcatcaatttaaaattccctcgaatgttgctaattttttacattaaaactattaaagagaaactatttttagcaagacaattctggtatgttgctaatggaggtaggacctgcgaaaataacttaaggcctataaaagaaagagcagagctgtgaagtatagagagcatagaagaaagctggcaagaagaaagaaagaataaaaataacagattcagaagtggaggacaagccacagactccacttgagttagtagatccactggagagagcaatagagcaaatgaaaaatagcgtcttttcctcccctttttctttcttcacaaaaaaataaataaagagaaatcagaatttataagtgaatcaaggaagctgcctgcttaagtgatgaggaatgtggcaataagattagaatggtttctggtagaagctaaatgaataagcatttagattcgggagaaagaagcatacttcttcagatatttgaaagccagggagtaggagatgggggtagattggttaaaagaactgggtcctaagggattgattctagaacaattagccaattaaagcaggataaaagttagtggtgcctagttattaagttatattagttataaaccagtgctattatcagtggccatcttggactaatctattgcttttatcttttgcaaaaatgattgtgtactatactgtgtgtgtaagagatatcctaactggtcgtattcctaaattatcgtggatgcggtaaactttgatcttgcccctcagttgtctttttactttagtttagtgggaaaggagattggaagggggttagggagactgtaaggctgccagaaagaactttctctgctttttttccccctcttgggagatggcaatgaagaaggaacttgatattgagcagatgagatgaggttttgcatccagtttttgtcctctgatcgctggtttttccattttcctctcgaagtgtctaagacacaggcactgagggttccagaagactttcttgagatcatactctgttatttagaaggctcaggttggcttttacccattgagacatttttaaaaaattaatctgctttaatactgtagtaaaaatttatcagttgtgagataaactttacatgtgtatcattgttgaagaaaaggtttactaagcccataaataacaataataaaaagatcagggtatatgtttaacttgcttaaaaaaacaaattttaagtctactaagaataccattttatttgtaaggtacttttttcaaaatgcttttacatctagcatagaatgtgctgatcaaaacttgagcttaatatttatgcaaccagtgtcagagtaatctactctttttgatagtacttagcattactggaaaatagtgaatatttattttatttatttatttatttatttatttattttttcgagacggagtctatctttgtcgcccaggctagagtgcggtggtgcgatctcggctccactgcaaccttcgcctcccgagaagctgagattacaggtacctgccaccatgcctagcaaatttttgtatttttagtagagactgggttttaccatgttggtcaggctagtcttgaacttctgacctcaagtaatctgcctacctcagcctcccaaagtgctgggatcacaggcgtgagccaccgtgcctggccagtaaacatgtttttaaaaattacagaattgagcaagttttcctgtttctctccctctgtggtatataaagaagtgggacctaatatatgtcaaatgtgaggcccctagacagatattttatctcttggaagctcagctttatttgtaaaaaggggatattgctctctgtcagaggagtggtaagcattagacacaatatatgtaaagcttccagaacagtgactggctcatggtaagttagccagaatgatagctcctattttattgttagataaaaagaatatgagaaaaaagttgaattacataataactaagaaacaggtcccacaaacatatatcaaatagtcttctaggaaggacatgcttttagtccgcttttaatcaacaataattttaaaaattagtccaaagcattgcatggaacataacatatctggatcacttactgaatatttctgcttttcatctgtttgagtgctgtcaggtagacagactcagaatttttgtttgaaaagaaatgtgtatgcagatatgagacaattattaattctgttgtattcaaatcaattactgaagaaatgaaagtaaaattacctaattaaattgaatcacacataaacctactctgtttgtgacgggtataattgaaatggtaattaagtggtagtaatgcatttcatttgaggatggcaattcagaagatgtgtttgctggcaggtcttggctgcttggtgtacctccctaggtgaccttgtccagagtgagtttttaggatggacttatttacttgtataactagatgtgttggtgaatgaattcctagtatagctctgaaattttattaatttccatacttatggtgctctatgtgttatattaaattcatgtctctgttgcatatctggaattctcagaaccacatatacataccccagcagtgttctttttttctctcttgtgtagaatcaaggagaaaacagaatattcagtttaaaaggatttcacattagagtacacataaacagacattttcctgagtgaagtttcaccttttttgggtcagagttactattgtcataggcacaggacagtcgatccctaattccaaatgatcttttttttttttttggatgcctttatcacctcctatcctgttttatcattgcttaatttcttgagtacatcttatctcctttgtgtgactggaaactccttgagggcagggaaaatgactttgtaatctctgcagcctgagggtagcagacagaatatgacttttagcacctaaccgactgggatagtatctttcctatgctgtgttttagttacctcatttgtaaaatagggaaagttagtgcttgtctcttggagttgttgtgagggttaataaacttagtaaagcttttatcacagggcctaatacattttaagaaataatttaagaaatattcatttatcttttccacagaagctaacacagtgccttatacatagtagtctctcaattaagaagatttgctgtttattttaaaatatattgtgagttaatatccctatgtgttggttatctgattgactgaatgcattagagacgtctctcagttattccagacattattattcagattaaccccacaggttacttcagttatgctatctgtgttgaggataccagagaccaaatcacccagctagtcttacccagaaggcagttttaaaataaatgctctaacgatggctctagcttagacaacttttaactcttttctcaacactcattcaaacaatactacattggactaaaagcttctcaagtacatgacttcaaaagcttcacatggcttcaaaatatacatcttttcagccagtgtcactctagaatttagagatggattttataaaacactttaagggatacctagagaatcttgaaatgtatatacaaacaggtcagaggtattattggaaaatcttaaagatacaatttaaaagttagtgttactattattattttgagtaatgaatatttctgatctttatgatccaaatctttcaagtcataaatcagtcatttagtagatatacatgaaaatgacatgtaattctaatgttgtttttaataaactttaatccagtagctaaatctcaaagatcatttagaatcctatgaatcaaatggctctcccttaaaactgttaagacagttctgttgattccagattgtgagccagacctccattttcttagaggagttgtgataattatcacatttgtctacaattattgtaacagtacttgggttcatatacacaatctttacaaaagatatttcccccagttttatcccaacatttacattccccctcagttgaattcttactagaaatatttgcttaaatatgtccaagcaatttgttacatgagcaatcattatctagttttacaagcatttgtttgtaatcattcctatagctgcaagaaataaggcagtatatttaatagcttgaagaataccatgtattctctttgtttacattgtaacagatcatcttcttaagaagtaatcttttattgtatctctttcactgttttctttaacgttacaaaaaaattttgtgttatttggaagctcatgaaatataatttgcttggcgtatggttctgtgaattaagcccataatatctaactggcatatataggaaagcaatgtacttaatcctaactgaactgaaaattcaagcttgtatccatatatttttagtagcaatttaatgataatcaagtcatatgcatttatgtgccaaaaagaactgtttgtggtaatcaatctgtaattttctgtttgacatttttttcctttgaattactgtaaaatactttcagctactagtaagattattttaaatcacaattaaaattgatttttccctgcttgggtgggtcttaaggttggtggggaacttcagtgctcccccaaatgcttccttttcaagtgtgaagtttacggatattttggatcttactgtgttctcaggatcatgtctctaggtaggcacagctttgatgactgatgtctattaattgtgtttgtcagtttataacaaagcatttattgctctaaaatgctctcatgctttggtattttgacttctttcctgcctgtgagagaccctagcttagtgattagtttttgtttctgagtttctgtttttaccaagtgtttttattttctagattactggctattgggataaaggaaatgatccagttagaggacatcagaaaactagaagcttttgatagaacaacaagatctagtgtccctatctaaagagaagaaatagagaaggtgtgccttcattagaagtgttacaatcaatagctgccaattacttagtgagcagaaaacatctggtactcaccttcatctatttgcagcctgtatatctttatgtacaggtcctcttctctctttctcagtttatttctttagatactgtagatgctttcatgactttcaactctgcatctagcactgtttctttcctcaaaggaaatcccacatccagtctatcagaaatccttggctctgcttctaaaatacatcctgaatttaaacaattctcaccaggcccgctactgtaactttagtctatcatcatttcctagtaccttaagcctaaaatattgcaacagtttaaccggtcttcctgcttccattcttgcttctctgtagtttgttttccacacaatagacagagcctgactttctaaaacccaaatcagatcataacaattcattgctttaaacacaaaagctttccactgaaactagaatacaactaaaattcctttccaaagtctgtaaggtcctaaaggctgtggccctgcctccatctctgacttcctcttctctattctttccctgttcactgaacttttaatacctcttccctttttcttttccgggacatggcaagttcgttcctgtctaaggacctctgtacttgctgtttctttggtttgggacacgttttcttccttgttttgcatgaaagcctccttcttttcttatttccatttaactgtcgcctccttggcgaggctttgcctgagcatcctagataaataagcccctctccaggcattctatgacattatcctgttttattttctttattatacttaatagtatctgaatttacttaatttgtgtatttatctcttctcccaacacacagttgaggaacatttggcttgttcattcatgtagtagggccaggaatgtaatagacaataaatgtgaactcacagatgaactgactccttgttctttcccttccataaatagaattctctgtgtgtgtctataaagcaaacaaagtgttttacactgtagtgttcattctaaaccctctattttagaaacctgtacattgaggcgaaagtcacatggggagagagtagtacaacaacgattaaaaaaccttatcttggccagatgtggtggctcacgcctgtaatcccagcactttgggaggccgagacgggtggatcacctgaggtcagaagttcgagaccagcctggccaaaatggtgaaactccgtctctactaaaaatacaaaaaaaaaaaaaaaaaaagaaaaaaaaattagtgtggtgtggtggcctgcacctgtaatcccagctactctggaggctgaggcaggagaatagtttgaacttggcgggtggaggttgcagtgagctgagatcatgccactgtactccatcctgggcgacagaaagtgagactctgtctgaaaaacaacaacaacaacaacaaaaccttatcttctgactcttggcttggtgttccttgctttacatgatacactcttatgttttggggtgaggccctgtagaagtagttaagagtactgattttcatattacatagacctggtttggagtcctggctgtgcaccctacttttgggttaagccgtaagggattactgacactccattgtttttcaacctaatagctgtgtgaccttaggaatgttccttagatttcctaaatttagttccttcagctgtaaaatggtataataataataatacttgccttaagttttgaagaaataaatgtaacaattgcatagagcacttagctcattttttggcacgtttgctgttgtgatttatagactgcaaatatttgcatatagctttttaatatttattagataaggcttctagggtttgaaaattaatgaataccaaccccattctaaagttaccacttgcattctaggaaatacatttgttttgccttgttttaagatttagaataccttttttattaaacagacaaacaaaacaagtttgcatcctcacatttcaccatttcacaaatctattgtggtatttcacaaatctattttattttataatggtaactttatttagctttttctatttgcttttatattatacttaatagtattttaattattatggttgatcatcatggtaatttaatagcctcttaggaagggaaatgctttataaaaacaatcaaatctaatttctttagttcctaggatattttggaaatacatgtattcttaagttagttaattgggtggaaaaattgcagttataattctttagaaaagaatgttgacatatctaaccaaaaattctgatgatagataggacttaaagagaaaatgtaaacaagtgtgttattgatttcaaattacttttatttgggttggattagctgtctttatgctaggactatttgtgtgtaatatagatcaaataaggtgttcagaatgggatgaactatctggaaggaagtggtgttatctatactactacagctggaaaatatgacaattacaaacttaattgatcaataccgtatcacatcattattcaatgattaaggactgctaggtgaaactgacataatggggttttctttttggaaaatagtatcttagttgtattggtattccactggatatttagtaaaagtgctatttactgagaacatccttaggatttagaaagagaactatgtagttttatagttacagagattctggtgcagatttgggaagaggtttgaaattagtatgtaaagtcagaacagcatgacctcaactaatgtactgtttgcccttgaccccttctccacaacctgccactaatgaaaggtctttgataagtgtaactgccttatacccattttatttacagttttcatagctaatgaataataagaaaattccaccaggacctgattgtttacctctctaatagcaatttacaaatggtgtatgctgtggctgttacaaaccgtgtcctgtaatagcatttgatgtaagtagctgctcaccattccatattgtggttggagctggagaaatagaaaattggtcatttcccggcagatagcagtaactttcaattcagtcagaatgctgtatgggctgtgataggtttataattattacccaaagattgatggttttttggtgtgtgtaaatttgtgcagcagtaatccattaagaaatcaataatcattccattctgatgtgtagttcctattcactgttataaatgtgtaccgtgagctgaaaccaatgtgttttgctttgttttttgaaattgtatttgacagtattggaaatgaggaaattattataatattttttcttcaatcaaatttctttttctttgtgttcccttgtaccatatgacgtacctattgcaagtctgcatttttctttgaaaaattttatttaaaactatttcaaatgtttctgtctcctttttaccatcatttactttgttgtttaataattacaaattatttataagcttaaagcatctcagtgtttctttctgtaatctggaggtgatttattaccaactctatatggtcaggaaagtaggaaattcttctcttgtaaaatacaattttaaccttaaaacgataatctcataaaaaacaactctaatgtttatttattaccggtacctcctaggatgccattgtgggaagctaggattaaggcagcattgaccctactctcaaaggagcttagattttagagaaagaaaggcagaaatcaacacacataacataagggtgtgaagaaatgggaaaagtggtaggggaaatgtcaaccctgaaaccatccattacgtaagttctgaactggaactttggtgttatggcatgatagtgctagggatgatggggtgtgggtgataaacttcatcccacaggggatgtggccagcattacaaaattctgacatagtgtggggctaaagagcagtctaaatagagattctaggtaaaaatgatataggaacatagtataaagttaaaacggtagagaaagatataacatgaaaagtaaaagctccaccactcaagtaccctagtcctcagtttcctgtgctaccaattccttaattatctttccataattttttggtatatactagtacaaatcatgtgtctacatattttgtttcctttcctattaatagtacaaatagggttgcatggtacatacttgccctatgactcaatttttcatttaggatttttttttaataccaacgttgttcttgggtgcatataatacctttatacactgatgatacatggtgatttaatttgctcctcattcactgttggacatttaggttgtttctatgtttttgttattatgtagacattgtttcagtgatattcttgttcattcatttaccttctttacatccttaggataacttagctgtgtatttgctatgtcaagaagtctgtgcaatgaatattttagtagataaagtgtggccaaattgctctttggaaagtataacaatttacactacaactagtaatatgtgagtgtctgttttctccccttgacaaaaatgcattttatccattttggttcatctgaatgggggaagatggtatctacgtatgtttgacatttcttaattacggttttttaaacataatttcaaatgtttgttggttggaaattctgtatttaatttttgttcctgaagaatatttctgcttcagtgtctgggagaaattataattaaaaactctattaggaactaggagaaagagagaaaaaagattttattcgtgattcatgatttgaggcatatttattaaaggataatggcagggaatggatgatttgatgttaagtagaaattttcaaatgttatacactctagaacagaagatgtgacttttatttctgtatctgctctaatttcactagttatcttggtaaatcacataagaaacccttccaaagtatttagcatagcagttcaatgaagttcaggggttaatgaaatctgaattcttatatcaagaaaccttgtcatccatatagccattctattaggggaaaagattgtgtatctaataagaagtagcaaacaaagaatacagaaagaaaaataaacctttacttaaaaattatacaattaattacatttaagttgctaggtgaggtataagggaatagataccttgtgcaggatgtaaggaataaggacctgatttctttttctttcctatttcaagactggtagagaactctgagtttgtcaattgattagcaagttgaattccttggaaacacataggcaaagcctacccttgagaaaagatgaaacctggtactttgttgttgttgtatacttattcaggaaataagttgtatacttattcaggtttagactcaccctgtactttctacacatgggaaactgaaatactcttgagagcagagaaagttcattgcatgacatatccacttgtacgtgaaaaattatgcatccaagtgtgtcgtgtagttttgtagctcctggtctgtcccgggaacgggatggtttgcagactgttagagagtatgctgaaggttacctaggacctgggtttgttctttgacctaaagataatccctaggcagaaggcttgtacctgtatccctttcccccttctgcacaatggcagtttcatgacagagctgattgatcttctttgctttttgcctgtaaaatcacatttaaaaatatcaggcttccaagttcttttgtgtggctgtgtgtgtgtgtgtgtgtgtgtgtgtgtgtgtgtgtgtgtgtgtgcgcgcgcgcgcgagcgcatgtgtgcgtgcacacaaggacatgcacccatgccattcatctcactgaaaataatccttgaatcagttacttaaaagtgaccccaaaggtcatttagaacaacttccttaacaaagcaagaatatccttgacagcatgcatgacaggtggtctctgggattttgcttgactgcttcaagtgctagaggaaaggaagtttactgttccattgctgatcagctctaattcttagaaagttcttccttatattgagccctcatctgcctgtctgaaatgtcactgcttttttttttcactaagactcaagctaaatatctcacaagttttagatgctttgttatgtattagtgcttgtagagcaggctttcatgtaggtttttagagtgttttgcattggcccttgtatttagaatactatcatcatttcttctgaggaatgatgctctaggtgtagtgcttaacatatatattatgtctttttccacctttgtaccaacagctgcaagtaaagcatgttaagtatggtaaataacaaaaccaaaccaaaccaaaccctgaatttattttcaataacttgtatcttattgttacttttaagcactttactcttttaatttatttatttattttattattatactttaagttctagggtacatgtgcacaacaagcaggtttgttacatatgtttacatgtgccatgttggtgtgctgcacctattaacttgtcatttacattaggtatatccttttaagtcttttattcgacaattcaaataacttatggttaaaataaaattttgtacaaaaactatgcctattttggaaacagaaatccattgcttatgtttccatttacatatacaattttgactttgtgcatagaattttgagcagatactataaaacctgatgctttgaggcttaaaagtaggacataaagtgaagaaggctatagggagatgttacaaaactcacatgaaggaagtatgatcatctttcaggattttactttgcttaggttgtggaaattcatcacggttttccagctgacatttcttaagaaagattcctaggtgaaaaagataatatggagtctactgaaagtggttgtaggcatccacatttcttcctctgaggatattagcaacacttgaattagcttatgacttcttatttctcccttgcctgagtggcagttttactgtgcctttattggatttactgttgtaattggatatggaattgtgccgggaagcatatatttcaatatgtgcaccttagcaatctaaatcgttaggatggtgactcactcacatttaacagggaaattagctggcttcgtctttgtgtgaacatttgaggaggagctgaccctagctgcagaaatatcacgtgttccaggtcacctgtggcaaaatctagccatgtgaaggattggagtttcttttgattatctaggctagattttgtcatccacctctttactttagtttggagaattatttaagtaggagaatcactggctgagaaatgattggatataaatttaagagctatgttagcccatttaattatgtcactgcagatttggtaagagtgtttcagcaaggtcacgtagtacttttctggtctgccatcatggcaagagacattatgaaattcttaggaaagaataatccttaaaaccaatttatttttaaaatagttatgtcatagggtctttttctcaaacttgcttgatcgtaagaatcatctaagatacttgaaaaaaataaacagtcccagacctcaactcaagtatactgaatcaaacaaagtgttcaagggaggaggagcctgcgactctaattctgataatcaggcaagtttaggaaatactgtcagatagtatatgtgcagggacttgggaaagaaaatagtggttgctccaaggagctaagatattacagcattttaccatcaatcactaaaatagtctctgcttttcagaagatggtagaagaacgtgactgaactgcatctttagggttgggtggtaggcagggagagttgtagctcttctcaagaaaaaactaaaaatgtaagcatatatgcataattttattattgtggtttgccctctctctctgtctctctgtctgtcttttcttttcctctacctgcctttgataatttttcctttgcttggctttctaggtctttttgattgagatgttttataaaatcatgtgaatattttcacactgtattttattacactctctattatttatttctgcacttctctctcagttgtattccttcagcctgttgacacaaaatagacacatgttcctgtttcagccagggagtttgtgcatctgttgacacttcagaaataatcagtttagatttcaacagctgattagtgtgctgttctaaccaaactgagaaggaactcttcagaatggaaacagatattattctatttagttttttgtacaaatatgtaataatggccattttaaaccaatgctgtggttataaaaggcctttgctgaagttttgtttttctttattgtacctcaaagaggctatcaaaatgtttagtgaccagtattccacaagttgtaatatgccaggggtaatcacaaacatttcataatggtccttgtatttatagcaactcaatgaatgttagaaagaatattggtagaattccaggaatatcatacatgatctgctaacagtttcagaactccagcttccacattgaattactactgaatgtaagaaatatagctaatggtatcagtgtagcacccaagtactgatttgcctaagataggaagccatatctccatgagctggagccaaaaggaaagaagagctcaacattttaaatgcgcttgtacaaatacttacctggttgaccttatatttctccaacagtctgaagaaatagatcagaaataggatcagggagcataatagaatcagtagaaatattatataatagaaacggaatcagaaagcataaaactagtttctagaaaagatcctataggataactattatgcatcttgatcagtcagcaagtatctatttagtactttatgaaaaatattgtgtataggacagagaagaagaatgcattttttcctttaaagagaatcagatttcacagttacatcatacataaataaaaaaattaaatagcagtttaatattttagatgggagtttgagacattaggcaaacatagtaaaattattttcatataattaaagtacagcagcttgggcaacatggcaaaaccctgtctctacaaaaaatacaaaaaattagctgggcgttgtggcacgtacccgtagtcccagctacttgaggggctgaggtgagaggatcccttgaacccaggaggttgaggctgcagtgagccaagatcttgcaactgcactccagcctgggttactacgagactgtctcaaaaataaataaatatataaagtgcaggataaagtgatttcttaggactaattgtaggtcatctgccttccagttagagttatagaattaatcattgcctttctgtagttttgtcacctacttctattacaaaaatttgtttgccattcttttttctgaatgactccccttacctgaggaatgcagtctaataagctctttagcattttatactacagcctttactaaacagcccaatctatctttttgtcttcatctcctgaaattccttttcacatatactgtgcttcagccacagatacttgccatgtcttgaatataacatattctttcaaattgccttttctttgcatgaatccttctcctttccttggatttctgtactttttctcaaccaggaaaactctcaatccttttcaaaggtccagtgtaactgcttatttgaatttttagttattccccatatttctatagcactgagtatgtacatgtatctattatagtattcaccacattgtattgctattgttttatgtctttgtctcccatattagattttgagctcctctggggcagactatgtcttgttcatctgtgtacttataattttgccactgtacctggcagagaataggtgtaccacttcccattgttttatgaacaaatgactaaaaggagagtgaatgaactttcatgaagtggatgggagtaggaactggccttgaagaaagagagatggagttggctgctttacctggggaatgaaaagagggagagacctgggctcacataacatgtagtctgccttcaacagcaaattcttttctccaaacaaacagatactatctcactaaagtcgaataagtgagactgcttctttctttgccaggtacttactgactactaactaattatttttcttctccttcaagattgctagtaaaggccaattttaatctgatatcacatgcccttatgcacacaggatgtggtagggatccatatatgatggtgacctctgaacccgagtctgtagagaaagccttgcattcagtacccaagtccattcatcttggcttcaggcatattcttataactcagagctgactaagatggatatgtctgtagctgtgcctcatcatagggtagagtattaatacacaaaattactagggacttccttatgtgtgtcatctgtaacagaataacaacagaacagtggtacatcctttcttttttttcctccttctcatgtttcttgtctcccacactttcatctcttgtttatgctggctcgcagcattagattttttatgaaacttcctctgactaaagttagggaaatttgtttcatctcagtcatttgctctttgtcgagctgacctatttcattaaaaaaaatgaggttgtggtaggatgagtacttagaatgcaggaagggggcatgccaccagtgccctagcaaaggttatacagatagggctggctttagagaattacctatgaattgttttggggccccttttactattctttttttctttcttagcacttccatttgcaaacccacttcccttgtggtacttgcttgttgcttcactgtcctgctcagtcctgcctgcctttcagggttgcgtgattctgatacgtgtcttcacaatctttctttattcaaaccaaaacctgtctgtggacactaggaaccctttccccagctttccatcctgtcatagaacaattcaagtaaatgaattcaagctaaggtttttgagactgacatagtttaatagattactgttgttttatagtggcataaatgctttaatagagatgtctctagaaattggttcccaagccatagtagtaaatttctgataatggagatttcaggtaccagattatgctggagggtatggagaagaagtttgggagagtgattatttttcttttgttttctgtcaaattaataccattaaagtaactgtctttgcagcccaaaggtagtaccagacctgagtagagactcttttttcacaagcacaaaatattagcactgatgactgactttgtagttacaaaagtgttagagaatattagaatctgaaagtgactctttggccattcgttgaccctgttccaaaaatttttctgggaaaaaaagttttaggtggcatctgtaaagctaaacaagaactttgattaatattctttcaactgccttttcagtttgttagagttagtgttttttacttttgttttgttttaatataaggaacttctagttatagttttacaaatatagaacacatttacaaatgtctttaaataccacataacatatactcgatagtcttagaacagagccctccacctagctagtctcttctggataagcagttctcagtcatgtgtagtgttgaaaaggtgtgctcatttaattatttccacagttgtcaaacctctgccatggaaaagaatttcaaccattttgataaagcccctataaaaccacagcttctaaatatccttaaaaatggaaagccacaggtatacaaaatatattttgcaaaaatataccaagtgtttctattgtggtgtccaattcacttattctagaatagaaggaaagcctggtgtgttggaagacagagtaaaaagtcattaatgtaacagagattgtcttttaagtccatttataagccgtctctctttttttaatgagaatgcctttttgttctgtatcctgattatggtagtggttataagaatctatgcatgttgtagaactgaatgctaaaaggaaaaaaatgctcatgttttttgtatgacaactttaaaaataaaatgaaggggttaaagaatgtccttttcacttgaaacactgctaaaaatggtggttaaattccattgaaagaaacaacaataaaaaggcttagttaatgtggcaattgtagtagggttaatagtattatacaaactaacactatgaggaaatacattttaaatcacaaatcagaacttgggaaatacactttcccctgttcattgtcaagagtgggaaacggaagtattgtaattagccttctggtgcaagtggcaataaatgcagttgcagtgactgcagtgatggatgtagggactcaggatcagaatttgaagagggtagacctagagtaattgttagagcccctgcccttagcagagcatgtcttgactgttgtgcagttagcagctccttgtgcctagcattttttctggagtaggtgttcaataactactgattaaattctggggcctaggttcaaatcttggctctacttaccagctatgtgatcttggatatgttacttaacctctccatgcctcattcctctcagccctaaagggttgaagaacatctctctcataaggctgttgggagaattaaatgatattaagcttttaaatctcttagcaaagtatggagcatataataagtgttccaataaatgtgagatatgattattcagtaactgtgggttgaatggcagtatctcgtaaggcttgtgttggagaggcagtgttgaatgacagttgctcctttcagtgctggtctgtttattggggtcaggagtttttgtgaaattaatttctttcgctgttcagcacagaatactcagatgcctaatattttaatatttgtttcttctcttccttatgtacctcaaattctgggctttttacctacggaattttaccctaagtgctgttgagtacaaattacgaggtacaacagttagatgaattttttataatatgggttactttttccaaattgtgattattggatgtgaaaattgtgatttttataaattatattttaatgatatatgttttttaataaacatttttgatagaatgaatgcaggttaattaaggagaaggggaacatgcacaagtaaaaggaagtcagggctaaagcagtcataattgtacattcagagccaattaccatacaaaatgataatagcagatttttgtgtgtgagacagggtctcactctgtcacccaggcttgagtgcagtgatgcgatcatggctcagtgtcaacttcgacctcacagcctcaggtgatcatcccacctcagccttccaagtagctgggactacaggcacaggccaccctgcctgtataattttttaaatgttttttgtacagacaagatcccactgtgtttcccaggctggtcttgaactcctggactcaagcaatccacccgccttggcctcccaaaatgctgggattacagatgtgagccactgtgcccccagcctgcagaaattcttaaagagcatttactgtgtattaggcattgttctaagtgctttgcatataattcacttagcaactcaaagtatatctgtttagattattttctatgaccatgtaaacatggaaatggatgggattgtataatagaaattatttatgacataattaattttcctttaacattgtacgtcctcaacactgtttcatgtttctaagcacatacatacattataattattaacagctacagagtaattctttctgtggtaatggactaactggtatctcttttttgtgttcctgtaacgaagaaagagtttaacatttaagatggtaataatatgatttttgtttttctaaaaatagtgttattaactagaaggtcaataaagtataatctgttatccctttacattaaaagaggttcaaacagtgctttgctatatttaagagtttaaccttgtttttgccttgttttccttgttttacccatacacaaaataagaaagctctgatgttacacagctttttttttttctgtcatacacccatacattgtaataatggggtggctaattatttgctaaatgctttaaagtaaaataaaatccaagtatttattacaccatgtgaatattttgacttttttggtttactgacatttggtgtttagcatttccattgtcttttggttttgacgggaaaaacttggtctaataccaattatgactgttccaagatagtaaatggaacatacttgttgagattcacatggtttgagtcatgcattgcctttacctatccttttaatttattgttaaggcaggagttcaaattatggactggtttcagggtgtgaacatcagtctctgattataattcaggagatatgtggatctgaagttgatggacaggctgcctggttagccaggcaagctcccattatctcaagaacctctgctgattagtttgctatcattttgagagacctcactatgaaaacagacattttcccagggtggcagagagataaaggaaatattcgctatgtggttaaattcttccattgttttatgcctatggaattttatttaatagtatagcatatttattcaggaggagactagtgttgtttactctctagtcactgttttccttcctcatacctcaaattaggatcgtttcatatttaagacattgtaagacatgatactattttcttagaccagtgcctgacattgattaaaacttcagtaaacgtatcgaatactgaatgaatggaattaggtttagacctgtgtaggaacatgcagtatgctttattataatgtatgcgattgtatatatttattatccagctttttcacagctgccatcatccagcattacctactataactgtgattccgccttctttgagccagatgtaaaactacactgttgtaacttttaagggttaacgtgcatttgccttcattctagaaataggtttatgcttttgtttctttgattttttttttttttgagatggagtctcactctgtcaccaggctggagtgcagtggcaccatctgggctcactacagttgcctcccgggttcaagtgattttcctgcctcagcctcctgagtagctgggactacaggcgcgcgctaccatgcccagctaatttttgtatttttagtagagatgaggtttcaccatgttggccaggatggtctcgatctcttgacctcgtgatccgcccaggttgccctcccaaagtgctgggattataggagtgagccaccgcacctagcctgtgcttttgtttcttaagaggtttttgcattgatcaaaatcatattaaatcagttactgttcaggttcacattttcacactttttggaagacttttggaaaaatctcttaccctgagtttcacattcattttcatacactttattttgggaaggcatttggttcattcagtgtcatacatcacatggccaattaaataatcactattaattccatttctcaagtccatccaacagtaaataaatggaagaaataggaaaggaagaaaaaacttggtctttttaaatatttgatttaattcaaaataacgttttagatgtttctgtaacaatttacatttaggatctctcaaagtcaaactatcagaatacttgtcagagtaaaaaaaaacccgttgcacctttgcccctataacacacacacatccacacctttttattatagaggaagatttgattttagattactctgaggaagaaaattttttttcaggaaaacctattatccttagcaaatataaacactgaaaatatattgaaaactttcagtcagctattatttgaactcaaaatttggagttgggtgtcaatgttagagattttaggaagacatttaaggcagagaaccaggctcaagagatgtattagacgtaactgattattacaggtatattaggaggagtttgggattgcagttgcagtgggagataggcattctggccattgctagcacttgtcatccttgtgatgagtataaaaatagctatttacctttatttctgtcaaacttgtgtatccttgcccctccttgctcaatagatgccaactttaagaatataatcccaggcacatttagaaacatgctaaaacaaaacaaacaaacacctctctccactccttccctaaaacaattagaaaaaacgtatgtgccaagtatcaataggtacagctgaagaaagagagataaggggaaaaaaaccaagcaacaaccaaacttcttacctccaaagtaaattaactttgaaacatggttctttgttttttagagaaacagacatttgcagagttaggggatttcttgagattgtacctgaaagtaggtagtatatttactagaggttttaataccatttgttttgtacaatggataaatcccagcaagcttatgtaatagtaaaattatgtgtaagaaaaagctattctccatgggctctaaagttaactttgaagaggccccaaaaaaactgagttaaggaagagataagatcattctttcctccaggctgattttaagatgtttaaactcctaaatactgtaaagattaggctactctctgcataccatttcagaatgagttggagttgtaccattatagatctccactaagtctcttagtgttatcaacttctttgatagtcattggagcaagggacatccatataaagtaggaataaaagagacaacttaatgattatttaacttagctaatatttactggttatctcaaggccttgaaagagaagatccagttcagattcactttaaaaaacaatcagatatttagattatattggcatgctcataattattttttttagatgataggaagtctgtatatctaatgatttctccacagtttttgtattttggggatgcaagtttttgctttttttagcaactgtaggatcaagggaaatgttttgtcaaacaaatcaaatataatatttcaaacatatatttatatgtattaattcatttaatctcaataaaaccctatgaggtagatgctagtattgccccgctgttatagatgaaaaactgaggtgcaattaaaagccattaagaggtaaagctgtgattcaaaccttggttgcctagttccagaatccttcgtcttagctaatatgatatttctaacactgtaaattttaatttattatgggttggggaatcggtgtaatgtatagaaaccaacactttagtaaaaatgatatggaatagaaaacgttagaatatggaatattgagctgggcccagtggcttgcacctgtaattttagctatgcagaaggctgaggcaagaggatcacttgagccctggagttcatggctagtgagctgtgatcgtgccactgcacaatctgtcactgggagacagggtgagaccctgtcttaaaaaaaaaagaaagaaggaaagaactatgattatgtgtagtattgtttcattaagccattttcataaacattaatatgtatttagacatacatagacatatgtatatagtgagttgcaataaaaatatattttttattgtctgtctgggtcaaaaaaaagtttgcaagccactgaactacacaatctcaataccttgaaaatagttgatgttcagtaaatctgtactaagttgaattaatgtattgttaaataaaaaataacccataaacagtgttaggataaaatacttctgctgagcatataatttccctatggagtaggactgaaaatgttagtctcacagttcttaaccaatgaaactgtaattatcaaacataatctattagtttatacctgctcctcaacctaaagttctcctctgcaaatctttctggttggtaattttttattgctttaaggcttggcctcttgtcctttaacaatatcaagtaataatagtccaaaaagaagaggcagtttctaaaaaaataaattacaatttgaattccctgaaagtaataatttgttattaacaaaagaaatttatttcagcagtgcaaggatggatcaataataggaaatctaatatttatcatattaaactaaagagaacaatcatatgattttctctatagacagtgaaaaaacataggacaaaaattaacaaccattctctacacccagtaaacagaaataaagggaaacttcttcatagtgatcaaatgtatgtatgtgagtccaaaagccattatgattcataggaaaacacatacgcatgctcagtaatttcagaaactaggtagattttgtttgttctctttactcttttttaatattgtgctggacgaaactgccaatggatttagataataaaaaataagattataaaaatttgataggtagagataaaataatcactttttgcagatggtatgttttgtatccataaactgcagagaatcagctgaaagacaaataattcataaacaataaaataatttagtaatagggcagtttaaattaacatgccaatatttaaatgtgtaatatttgtattaaagaacatgcagaagcgtacttaaaatggggaagatatactgtgaagtattctttcacaggtagatgcaatattgcttatatcttatactgaacttcaggataaactctgaattggaacaaagatatatacatgaaagttaaccatataagtaatagaagaaaatgtgaaagtcttattatggtcttactatgggaagctctttttaataaaattcaatatctaaaagtcaatttcaaaggattaattcatttaaaataataaaaatttctacatggcaaaaaatagcataaataaaaccaaaaggtaaacaacaagctggaggtaaaatttgcaactcatatgatgacttaaacggttaatctctaatacataacagctcctagtaattcctgaagtacagtaagtatctaatagaaatttggttaaattattctaacacacagttcacagaaaaataccattggctcctacacatataaagtttttcttaacttcacccaaaataggaggtatataaaactatactgacatagcatgtctaattcattatattagcaaaaaactgaaaattttgtgtcatgctgtgttggcaaggctgcaaggaaacagggccttttatgtattgttgatgggagtataaatttatataaatgtgaattggaacaaatgcttataaactttgactcagtactcctgtttctacaaatttatcttataataatttgtcaaatgttgagtgcacagatttattcattgcagcattgtttttcatatcaaaagatgggaaacattgtgcaaacaatgcccatcagtagtggattgattaaataaattaggtatatccaataattgaatattatgcaagtatataaaaaataagaatcatgaatatggaaagatttcgaaaatatattgctaagattaaaaaaaaggaaggggcagaagaaaataagttgggtaaaaaaaaccccagaaatgtttactaataattatatttaaaaactcataggataaacaagaaggtaatgaaataattaattgtttgggagtgtatagatagagggatgacagctgagcagatgaggggctgggatggaacaagactcttcactagatatacttttatactttttgatttttgaaccattcaaatatctacctatttgacaacatcaaaacaaaaggaaaaaaacactaataataacaactaaaccacagtaaatattagtttttttgagaaaatatttctatagggtgagactgaaagtgtcagcccttcaactgtttgccagtggaactgtaagaaactggtgtaatccattaatttgcttccccaacacccaatttagatgtagattccttacactcagatcatttgttgatggttttatttattgtgtatatttaataattttaattgcattttatatctttgtttcctacacattaactccatcaagcaattaatattgaacagtgtaattaagtggtagttttaaaacaccagttgtacaaaaatcaagaccagtgttcattttagaatttaccatttttatacaatatgcaactttagaaatgataatattatttaaaatttctatagatattttaccttgcttatgtttttcttcttgaaatatagtattgccaaattgctgtactgtaacatcatgataataatggtagtgttaggaatttattttcattagtggtcatagtacagttaatataaatatatagcacataaatataaatgagaacactaaatatctgtttggaaatgggattcaagagctgtgtcttaagttttggtaggattcatttaatagctgtgtgaattacttaacctttcagaaagttaatttcttaatctgttaaaaaaaagtgatactaatacatgccttacagggttgtggtcaaggttggagtgaatgtctttataatacctagtatagtatcttcaatgcaagcatctattattcctgtggttgtgttacttatgatatgtagccctaaaattgcaagatgaatattaacaagacaatatgcacagtttgccactacctgtaagatttgatttatttgagtaaaataaaggggaggtggattataaaattttcactgctataattttgtgtctgcttgcatgttttacttgctgagaatttggtagggtcaagtgttaaaatcacttctaaaataagtctcctcaaagtgattatttaatacggagaattaattactaatttgaaatattttaaatagtggtcagttatttaaaaaataatatattaattggatagaataaaatggagctgttaaaaaccatgtgctttaagaatgattatggcatagacattttttgaaatacatacattaaaaagattatacaagtttatatagtaggatctcaaatatgtaaagaaaagcgtgccatatataaatagatgtatatttcatagaggaaagatgagatgaatatacaccaaatgtgaatagtggtcatttttgagtgttgggattctttgtgatttttgtttgatttatacttttatagattttacatttttttttacaataagcatatattacttttatattagcaaaaaaagcaatatgttgtattgcttttttatgtttatgtattcttcgagattgaattgtaggatgagtagaacctaaatatatgtaccttcttttaaagaaaagcactttaaagctatatcaactttgttagtggaattttattccagaagagtagaagttggcacctggaagcactcttggaccgttttcctattgaggaggggtttattttggtaaagtaacactaccactgtagcccgatggctactgcagtgggatcagatctcaggtcatatcagttcagtagctcagcttgtagctctgatctctcaaactgtggggcacagtgagaagggttttttagaagctggcccctctgtcttttcatgcacacaaatctgcttattatgaccacagttcattggggatggcagaagtagtacataaagctaatgtaagcataatattttatattatgtaaaatatttacgtcatgtagaagaaattttttaaaatttgtaaaattttgtcagaaaaactttttttcctcaataaaaatcacctactttccaatggataattctacatcttatatagtccttatttcattttttcacaggttatcctagttgacatcaaagcatgcacccattttgaatatttcgggaaatctcattaaacactgaaaaggataatgccttgtgggttgctccatagtaatttcaatcgtttttttcccaagtttacattgaccccattctcctatcaactcagtagggtttcacggccgttgttgcttccttttgcctaatttagcatcatgcctgatggtttacatttcacttaatcatagctttcgagatgtagcatacctctctttgttctagagatatgaaaactaacctacacagttagaacacaggtaaacttcaggtcttttgatttcgaattcagcatgcattgtgttctgtcgtgttccagtattcttgatagttctctaaaatgttgtcaagaagggcctaagatctttgtgatctcgccaaagagtttagaagtatgtatgtgtgttgtttgtttttcctcctgcaagaattcctgagataaattatctaaattttcactgaaaacaagaattcctgagataaattatctaaattttcactgaaaactctaatacatgaaggtagttacacagttccagcaatgactgaacttgaaccatgatttacataccttccctattaagtgccagtcaggttctcgctacagatgagcaaatctgctttttccacagtgagaaaaacatttagagacaattctacaattagaattttgtgatactaatggaaagtgcgtgtatgtacacgtacttaaaatgagctatacaatttgttgggagttcagtgatccctcctttaaaaactacaactgtgaagctataagacctcacagtgatcatctctttgttgacataaatgtgataaaccttgcccaagaaatatataattctttgtttgtcttgctaagccttaaagttctcatgggcaagggatgagaaagggtcttctgaggactattattttttttgtcttgtgttaacagcctgagtgctttgtgatggtctttcactgtgtgtctgagggaaccctaagtgttgtttctgactctgagcaggctaatttgctcatcatagacaatggacctgttgacaaacaggtacattctccttaccgtgtccttctatatgtgatatagttgatttctaggtttatgattttccccattataattaatcagcagcatgatatatctgctgtttatcatataatggctttcttgatgggtggcataaggaatattttaaattaaaattaaaatttttttgacttttgctctgaattggagagatactttttggtacctttgctcaagattacatattaatcacatgttaaatatatgtattacttaatgtgttcatcatagtacaaaatatgaagaatagagttatgagtgagacttgagatgtttcacttttagatttttcaagtggcaagaaacttttagatatttagtctcagaaatggaaaacctacttaaaaaacattttttttcttcttttcttttcttttttcttttttttttttttagacagggtttccctcactctgtcgcccaggttggagtgcagtggcaccatgatcatggcttatgtggcagcctcgaactcctgggctcaagagatcctctcacctcagcctctggactagctggggctagggacatgcgccaccaccatgcctggctaatatctctctagatccgaggtctcactatgtctcacaggctggtctcaaacacttgggctcaagcgattctccagccgcggcctcccaaagtgttgggattatagacgtgaaccattgtgcctagccatttttttttttcgtaagagtagttagaccaactcttcctttaaaaaacaggcgcaaggatctcttaaacagaaaaatagattggtgccgtctttccataattttcttttcaaccacaaaagagatttttttaaaaaaagattgattttaaagatatgtaaatactaattatttggattataattattggtcattatggtaatgccttttttaaaaagagaaatagctaatcatgaagtctgacaatttaaataaagacagtttaggtaaatttatatcttaaagtgaaaaatggaatcacttaattttgttactatattttaggtcataaaaatatactgattgaagaaaacgttttcaaacaaattgtacatttatcttttgcccaagaaatctatttttatctgagctaggtaaaacttaagtcagtgtaaaacaagttgtttggaaactcttaaatctttttgaaaacattacagcgaaataacaaaagttttccagaagagtctggttaagcattgagctagagcacagcattttcacaagagaagttttaagcatgatgttaccctatcaatagtcgtggctgaagaagttctttgatttttgtttgtttgcagaattctgatttgtagcatttgataatcacaaatttaaaaaggatctttgcacttgtgttttgattttggtcacaattttataaattggtatgtttcgatgtttttgttaggaaaaaaatgtaatatactgacataccaccccccccttccccccccacacactacacttctttaaatacgccagataaaatcatttaatcaagctttttgggaatgtttgagtgtgtcagaggtgacgtattgtgtatagacaaaggtaagagaaaaccagtccaggaatgagaacatcattcctgcactgtatgtgcatgtgtgtttttcctttgctctaatggtattcctcccttcccccaaaacctctggtgtattttcactaataatgatgtcaggttaggtacttttgggaacagttggaaaggaaaacagcaagggagaagaggattggatcttcgggctatgattgggagggaaggcagtctagggaactggtttctgtgggtagctcatgagggagagctttgcagtagaagaggagtcaaaccttttgggagggtcaggggaataaattggacatttcagggagtccctggaattgaaagaagcagtcaaagcattaagggaagtctttttgacactactcaaaggaagccagtgagggaaagcaatgagctcctttgagaaaattctgggataggagcatgggggcctgggaggggagggaatctctggagtacctttgttgttggctccacataggtccctggtgtgtgaaaggatctagataggtgtagttataaatgggttggccctgtaaatctgttcatatatgtgaatgtctttctgatttcctcttctttttacattcttatctgagtatgggaatggctgaaggctttattgctatctgtgtgttgcatgctatgtggatcttttaaggcgtgaccttatcaaagttggcatctaaatgcaaaagaaaatgcagggcaagcttgttctagttggggttcctgtcaccagaccatagcaatttggaacaatggatacattacctgaattcactggcagcaagcagagtccctgcttctatgcgtgcccttatcagtgtttaaaatagtgcctcgtactctgcgtactatctagaagagaacacctccttcccattctcttcctttccctattctttattaatattttggggggaatgctttcatttcaatagttagaaaatgtggaaaaacaagattgcaaggttgaggtgaaagtagtacaagattggaagaatgaaactcttttttcttcactcagtaaaatgatacttttggctcagtgcctggggaacataggcgagttaggatggtgcagacaggactagcaacaagtttgttaaggaatgggctgagaaccccttgcctctgccctgtggtttactacagaatttgagttgtaactttttctttttctttttttttttttttgtccgttagaagcagtggtagagagattacacactaagctttaaaaccacagcaaaacaaaacttttaaaacacttgtgcggtaacctcagaagacaggttaacacattctgttttaactgtactattaatgtgttttgcaagtttgcaatacctctagacatggaggtctttttcccccttacattttatgtatactgcaaatcgaatccgaggtgtgtcttgtgagcattaaaataatgaaaacaaaacacagttttatccaggaaatatataattctgaatactaaaaatctttttaattgatcatttatgtaatatatcatcacttttattcaatacatttcaaaatatactactctttttagcaaattcaaagtccttttaacattttcttataaatctgatatcactatatttgtaactaaggaattttatggtgagattatttgagctttatttctgtaacagttgttctaagttataaaaatgaacatctttaacacatctatatagactttaaaatagcttatttgtaatttaaactatcttcaaatattttgctatattcctgtttcccctaattattcaaagtttacaaaacaaacatttattgacgatcagtgatgtcacagttttattgtaagaagacatttcctttcagagaattaacgtagaaataaggaaggttaaagagatttcctatcctttccttaaagaaaaaaaggctatttatcacttgccatcttatactcattcattcattctctcattcactatacattcagtaagtgcccactaaacttttattaagcctttcttttcgttcagtctgttgatttgataaatcagaagattatctttgataagtattcatataatactttttattatccttttgtgattgtttattaagctatctgtccaagctttgaagacagtctgcaatggttcaaatctaagcattgccattagttgtgatatgtgtgtatacatgtctgttggtctctctatcatcatttatcacaatttatcattgtttacccaaaggaataactagctactgactgagtgaccttgtatgaataaattaatatctctgagcctcagttgacttacctgtacaaagtgagaatacttatattttttaagattgttagattagttgtatatttaagccccctctacggtatttagaacatgtgagtaacgtggttacttattattgtttatattgccattatcctgaccactatatggacttgatcctccagttatttccttgggtccatgccatcttacctctcttatttttccattactgcttatctcacaatattcgagagtaaaaaggaacctttttacaatgaaaggctgcccatttctcacatgtgctttcctcttgatgccttcatgcttgctgttttgcttactgttgtagagcagcagaatattactgaggaagcacttctttttaaaaaaattttctggtacagctggaaatatgacaagaagaatagaacaattgattattagttaaatcaacatattattctataaattcaatagaattgagacccccccccttcctttaaatgttatatttggtcttattaaaattcattttgctatatgctgtggatttttatgcattatttttgtttaatttctaatgaagagttattgagattttatgagattgacgtcttatttacttaaaaaatttcctattgcttgtacattgggagcaaaccaagaacaggtgtaaaaacaacctcccaacttgtcttggtgaattgtgaattgctctggtttattcttgtttgtattacatttaatggacaagtcctgtatggtactatacattttaattcagttttgaaaattatcaatttgcaaattttgtctttttccactttctttttctatttcaagagcttattacctttgctccagctggataaaagttgggcaaacagataccctgacaactgccaacagcaccacgccttggttttcccgggtacagaaagtagtcaatatttctgggactatgcatctgtcccatctcaaagatgccgctcacaaattctagtttaaatacagaattattgaaaacatgaaaatcaccacttctttaaaaatatgtattgtatgtgcttctttcctggttttaccaaaacaagtaaaaaagatccatgtagcagatttaggtctacagcctgcaaattgcttcctgtttgcttgcatggtgacaatgttctaagtgccagttttgaatactagtaacctgtcatggatcagttatctgaaatggcaggtttatttgttatgggaggaatgacattatttcctgtcctttagacagagctaaagtgagaataaagtagtaagaaagaatgtttctttgcactctaggtgtgagtgtactatcgttcgtgctactgatttgattgccacagctactatcattagcatcttggaccaaacatggaacaagctgtactatatttcatgtatgatggaatacagagactgattaaaaatactcttaaggtgctggattattcatttgatagctgattgttataagacgttagggtcgtactttcctttgtggacataaacatttttaataattgctttttaaaattaaatttaatttggagcctaggtaaggatttccttcatttgaactaattttttttttgtaatctcagaattatcttaatagtaggtagtgtcagttgccagaaaaacatcaatgtgaaggaaatttgtatatcataattctttcctagtgttttctttaggcagactacagtgaacgtatctattctaattctcattccttttctggataaccataggtaaaggtacttttaatttgacctctgaaagaagaaaaagtttttccaggttggagaaataatgagtaatgctggttagagactgcctctcatcttcttctcaaatcttagtcactacctattctagtgtttaaataactaatgaaaacatttttatggtttctgagattttttttaaagcaggcttgaaagatatgaagtaatttactgataaaccaaattttaacattatagacataagaatgttgtctgtccatctcattttgtttcattgtaatatttttcctcataggtatagggaaaataggtgattttgtttagtcaacaaagggttgtctcttctctctctctctctttctttctctctctctttctaccatggcagcccaagtagggttcaaccgtgtaggtaggatctgtattattaattttcattatgccaaaactgacaaaacattacgttgaaatcataaagcactagaaaagcaaatacctagaaagtattatctgttctgtaagaaaaacaacaagctaaacattttaatatattattgagtttgttggatttataatattgggacttctctttatacaagtacctggcctttaaaaagaaatactacctagagtagccacaggtactgttaatgttgccaagtattcctaagtatattgtagttagattagtgtgaggaacttccttaaaaaatttaagtgtaaacctgtaaggttactcttattggttgtttttcatagtttatttagtgactattgcacggtaatgctttgtattagatcaacattaagttagaattcagtggctgtattaaatagccttttaagacagtatgttaaatacgtggctttcaccaaggggcccctatggtattttggcaattttactattttttaaaattcatacctagtttgaattgagattcatgtaagtgaaacaccattaataaggatttttagttttaaattttagtttgttttattataaaatagtcaatatctatcattcttaaaatcaagtattctttatagtagtcctacttaatatgcagtctgtaggctggaaagcatttgttaccagtctgccttgaaattaaatatagaagttgagtaaatgcttaaaactgtcaccaaagtttgatagagtaataaaaatagggggcttgtattttgtttttgtcaaatttgacttgattatacaaatgtgatgaattagaaatttaaaaaaccaattggtccttcccacagaaggatatttcatttgagaaatatcactttagatgacattagaagagttatccatgtgcttagtgtcagttagcttaaatgccatacattaaaatataaacatatcaaaaacccacttatattagtaacttatgtggataaccatatgatttttataaagtaatctcaaatgaaaatgaaagccaattctttaatgaatctaaagcagtagagcctttactcatatacctagtaataactatcattaagtgcttacaatgtgcaatagggtgtcttttatcttatttaatcctctaaaagcacaacgggataggttccttattaccctcattttagagatgccgagctaagggtcagagaggttaaaaatgtgctgatggtcactgagctaactaaaaaagctaacattaaaacatgcttttgttcataatatgattacacatcttgtgcttcacgttttttcttttaaatgaggaggtaataacatgtactgggtaaagaatgttgttttgtgcttgtaggtaaagctgtgtaccatgctgtatatgaggaattatttagagctagaagaggaaagctcaagcatgcaaatcttcagtgctgagttggtatagggagtaagatgaggcttcatcacgtacgttgctcttgatctcagagtctgttactacaaattgttgatgatcacaaaaatttaaggacatcatgttcaagttacacttgatacagcttataagaaagaatcccttttcaaaaaaagcagatgagatgtgatttttaaatggcatctctctactcttcatatgtacatagatactcaactttcaggaatcatgtatataatattggcatcttacagtaatatttacaatatttgtttccttcaatctcttcagttatctttataaaagaaaggaggatggggggaggaggctgacttttgtcatgtagcttcctcaactaaagccttcagatcttcatcagtatatgaataaactcagtttactagatgcttcccttctgtaaaattctgtaaatcaactagctaatatttaattgaatttttatcttgttttcttcatttggtccatttgattgctgatcattgatgttcatctgcctaataacctttgcacagtgtttaaggattgaggataaaaactatcaagggcaagattagaagaggtgactgactccctgattctctgctgcccttgaaggcactggataatggagaaacaagtgcctgcatgtgttggattttgttagttggatctagctccagcagaatgacaactgaggtcacgtcagcaccttggaaggccgggaagctggaactggagcagctgagccaatccccagacaaacaggttttaaccgggacagagtgagagaatgctttgctttggaaactgtctgcccatcacctaaggcggtgattctattcctatatattttttttcctgctgtctgttcatttttgtatagaaaagattagttatgaggacttctttctctttatatatggcaaaaagtaattgcaaagtagttatgttagactttgagatttttttgttttcttcagattcagaagcaaagaaaatgttacttttgaaattaagatggtgtgatggaaggaccatttatggcattagtaagggtgatcatttgttgattaccatcattttgtgtaaggttgaatgccaattaaatgtcatgggtgcctcaatatgtttaaatatcttcatattatataatgtacattcaaacctcttttgattccaggaggagattttcttgtaagtcctaaaattggaaacttggattctagtctcattttgcgagttgctctactctgtaatttatttcttctcaatatgtagaatggtggttctcaactttggctgcacattagaatcatctggatatgtttaaaaaatactggtatttttaccccatcccataccaatttaataaaaataggggtagggctcaggcattgataatgatttattaatttccccagataattctgatgcacagtgaaggtctagagccatggatatagaatgatatcactgacctaacttctgtgacttttttgagattgattaattcatgttaactgttttaatcactcttgtaaaaaaagctttctctaatttatagggtctttttcaaaaatatagaattagaagtgtcctttgatatacttctgttccttgccagattacattcaaccattaatttaaggtaaggatgtttgacaatagatattgtaaatatgtaattatattttttcaccttttaattacaaataaaatttgatttgtcaacaagtacatattgatagagagcagagggggagagctggaagttaggggaaagggaatggcatttattgaacacttccgtgtgctgtgtgtcaggttctatgatacaatcacccgtaatataggagtcacaatttctgcttatatatgatgaaatcagttcttaaagagttattaattaagtttaattatattttgtaagtcatagaatctcaagggtatgactgatcttaaaagtcatttgaataactcatctactggacataaaggctaaatgtgaaggtttctgaatactttaacatatgaaatcaagagtgaatgttaggattttaaaagtatgtcatcaagaaatatggggaaaaaatagcccagagagagatatttggtaatatgttatacaggttatacagtgtaactgtttgcttcatggtgttgagttttatttttattttatttttcaattatggcagagatagaagtttaattctttagatatgattattaactaataaaacattttgaaatattttatatcaatttatattattataaatctgattatttccaactattgcatattttttagaatcaccatagttaactagaatgattcccagttgtactaattagcataaaaagctaattaattatctcattaaactgcatgccattagccttgattttactgcacatttgatgtattatctcttggtctttaaaaatatagaaaataatagtttagtctttcaataattgattttaaattggcgtttttcaaaaaaacttttgtaatctcaggaattaataattaacgcgacaaatttatttccaatcaatgaaataaatacactttaaaataataaaataccatgttttactcaccatattataaatgatgaaagctactgagattgtttaacatcaataacattgtattggttttattgatagcattacattggtaatgttgttttgacacagtactcatgcaagtttaaattgacacctcattttggacggcagttttatatatgggaactttataatgcgcttgcctctcttttcacctaataatgccttctttgagacttcaattttaagaaacctgttagaaatgcagacagatttatccacaaaagtagacactggagtattattattggaaattactttcggtacagtgcttaggtaaatcatggttattccataagctgaagtatatatagtcttaaaaattatgctgttgaagaatttcttaatgacatggaaatgcttaggttattatagagaggtaaatactttatagtatattatatatacatccatacattatctgtttatcgatagataaaatctcaaatttttttgaaaagaaacatgctaaaatgttaaaaataggggtaggtttaggtactggtttaaatatctctgtaaatacctgtttttatataatgaggcattgttactttcataatcagaaaaagaatagaaaacaaagtttttacctatagttagcagctttattttggtggggccatgcttgatttgctattcttagacattctaagacatctttagagtcaatatttcctattctctggctattaagtttctttcatgtgaaattcgcatcaagtttagagtatgatgctctcttgatcaagtaacatatccttagtgaatgggtgctatttttttgagacttcagctataccctcttctccagtacctcttctaggctcttttggacatatcttctgggttaccttctataagctgtataatatttagtggttactgtaacccagagatgggttacttatgactatcaattattgttaaagatactacaaatgggaatttttttttctccaagtcatactacttaaatttgatttcaaatatctataattacctcgttagtagatagaaacaatatccttagatgagctacaaataccaaattaggaatttcccctgaattttgcttctgcagaaatacttaacaataacttatttttctttagttctagtgaaatagtgaagtgtgcttcttagcatgaactgaaatgttaatttgttcaacttttcattaacacacatttctaccataagaaacttcagtaaaactagatttaaaaaatttgaaagacatatttttacaaaaacatattttaatctttaaaatgtttatggtatacctgtggttaaataacatatgcatggttttaaatttttgattcttctatgtttaaactcttttcagtgtctagataaatctttattttttaaagctcttcattgaagattattcaaatctttttctaagacatttgactttacatatgttcattttacagactattttaattcagtgattcccatctaattggtcaaccaccagtatgatggtaaaagttacccaaagagtctaatatgcaccccagtttggggatcacgttttaaaatgaatcttggatatttaaaaatgatttatttagtatattttataacattctaatttttaatttaatgatatgttttggccaattttaatttactttatcctaaaagaaagctatctatatgcacatttattcaggattcattttaatgttcacttacttaaagagtttatttaccattatcacatttatactgatatttgcatagttttccccaaagtctgtaactttgagttcagatctgaacttaaggtttgtacttttagttgcctttatggatgtttatgaacaactatgaataacattttcaagtcaataatttaaaaactcataattctcatataccacttcactcaaatttattctgtttccaaaataccttatgtctgtcattaatataacactctcccctatatggttcttcagacttgaaatcttggaatcagattacttaatattcttggcttctcctagtctgtcatctggtatcttgttctttttccgttaagatttcttttgattctgtcctctctcctcttgccagtccacatcttatctagcctttctttacctgtggctgatatgcacccagcacttacagatatgattatactggttgctaaaatattaaaatgcttttgtacaggtttgagaaagagacttttcctcaagtccattgagtacctgtactcccttcatcccaagagctttgtgggtaccctgcttaatatgctgtcaactggctaaccagcctgtgtcccagcctgcgcatctcagcctcagtgtgcccatggatgttctctcccaggctaggggggtctcagattcttccaggaaagatggtaccctggtgtcagtgctggtacaccagcaaatagtacttacatgggcccctgaagctatggtagtctcttgccagctctctcttgctattgcttgtgggaactgcagcctctctgctgttgccagtttatagaattgttaaatacgtagctatcaccctatctttactccaagcttcatagctgttatggtagattagtagccccttagttactctttacctctagctacttatccttctgacatactcagcatagtcagatgaatctccctaattttatcactcccctaagagatggaaatttgaacccagttcaaactttttatcaactcactttaccatgtctgcttaacctgttaatccatacctttctcagatgaactcaggtttaccttttcccacaagatgttcttatttacctttttatgtttcccttttcacatgttcacatctctggagttctctcttgtgcagtttcaaatcaaacatttcctctaagttatatctcgattatatttcaagtaattcctccttgaaacttttactaacttcccaaataagaatgaccttctgttcttagaatccacatagcagcttatgtcttataatgtattagttcccgaactagaaagttcaaggtcatgtagttgatctttcttcccaacttaggaaacctttgtgcaaagcatgcgatgtacaatgaagatttcttccaagacaataaagctagtggttctgttattgggtatttctagttattccttcaattatctaataaaatatttcttgaggcttactttgggtcaggcactaagcttgctctacagcagtgaacaaaacagacaaggctctatccttatagagcttacagttgaaagaagacattaaaaatgtaagcacatatataagtatgtaataatttatattagatgctgtaaaataaaaataataggacattataattaaaaagaacatcgggtccttagtttatattgacgatttagagtaagtgaaatttaaactatagaggaggtaaggagtgggggccactcagatagcagtagagtatgagagaccattgtatatgtgcaaaggccccgcagcaggaaaaagttggcttttataatatgttcaccaaagaactgaaagaaggcctgtgagcctgctatggagggagatggagtggctcatgatgtatctggagaggtgagaaggagccatgtaatatactttatttttttgaggatttggggaagaattttggatttttatcctaaaagcaacaaacaactattgaaatagtgttgaacactgacttggtagaatttgatttatgatttttttaggtgttgggtggggagaagagcagatatttaaatctagaagtgcttatcttctgtattacttgaataactttcttagtttagcaaatatagagcaggtacacaatcattgtcatagagcagaaaactttctgacttcattaatagaagtctgccactccctgagaatgcacttgtattctgctctgtaaagctggttcctggggaggagatcagtttacacaagatgtccagcgccttctgggagagaaagaactaatacctagtctctgctgacatagagctgtgtggcactgactcaatcccagtctctggaaagggcaatgagattcctgaaatgaagttacattgaggacagaaaggggtaccccacctggcaaccaacattcaagagaaacccttttgaactcatggggctgatgatagaaaattttgtatatgctgtcatcagtgtagttagtaccttagggaagaacagctggtaatggtagttgcaaacacagaagtagagcacccttgactgagctgggcaccaaaggaaacaggatcatcactgtggctcgaacagggcggtgggatggtaccatcacaggggggactaacattgttgggtgataaaagaccagtagccttgtgaatagcagattaattatggtttaccaactctctgcgtggcatggtatgtagactgaagttctggggcaagtgagaactctagccagctatatttgaagtaaaaatttaaatatgaccataactgtacattgaaatggtagaggaggttattatggttacagtttataaaagatagcttataggccaggcacagtggctcacactgtaatctcagcattttgggaggccaaggcaggagaactgctttagtctcaaagttcaaaactagtctgggcaacatagggagatctcatctctacaaaataattattttaaattagctaggtgtggtggtgtgcatatgtggttccagcgactcaggagactgaggagggaagatcacttaagcccaggaagtcgatgctgcagtgagccttgattgtgctactgcactccagcctgggcaacagagtgagaccctgtctcaaaaatagtatgtgcatgtatacaaatactagtataataaataaaatagataaattaataaaaatataataaacaaaaaataataagtaaataaattaaataaaaatagtatgagtacacacacacatacacacacacacacgcacacacacacacacagcttatactctctttcacagaatggattggaaaatgtaagggaagaaatggggaagctagtggccatggagaggaccagcaataattaagtaaaatatattagcactagagtgacagttacacagagaaatagaatattctttcttttattaaattttcacatcttttctgcataagctactttgatcccacatctttcataaatccagccttgatctcatatctacctttccctgttgtttttctcttggttaacatctctaattcctttcttcttcagacggatgatttctggaatcaaaactcagccattgaggggatcaggtatccttgtcagctgtgtgtcatgcacaggtttgataaacatgccttgcaagtttcttttcaatcttgtgaaaatagccctatggcacacaacctgagatctgttttcaagttaacatcttgtcattaatcaatactgtttgggtgtggttgttcaacctgttatgaatgtacctctcatattatttcctacatatttctccatttcgacctcaagaacattatgagagctttcttcagatatggaattaagttcagaatgaacaaagtccacatttttaaactaccagtttaataattccatcaacaataaaaatattatttttagtgaacccatattggatcatactttttaaaaagttgttacacaaacacttaccttccaattttaattggttcattgtaagcatatttatcttagattgacaaaattcacatcctttgcctttaaaaatcgtaaaattagcccgtctctggtcctctggcacctttcctttcctctgaaggacttcaaaatgttcttgctgtagtgtcccaatcacttctgcatctcctttctggttcctaaaatatatctcatctgagcctgtattaatttccttggtttgctgtaacgaactaccacagactgggtggcttaaacaacagaagtttattgcctcacagttctggaggctggaagtctgagattaaggtgtaagcagtattggtttcatgtgagggctgtgagggagaatctgctatgtccctctcccctggctgctgatggctgctgatattccttggtgttcctggcttatatctgcatcaccctcatctttgccttcatgttcacagcattcaccccatatgcatgtctctgtctaaactttccctttttgtaaggacaccagtcacaatagattaagggctcaccctactctagtacgacttcatcttaccttaattcattacatctgcaatttttaaagaaacttagtttttaaattggcttgttttttggtgaaaagagtatgatataaaaccattttattgtataattgtcttctgtctgtgtatagccaaagcatcaaatagaaatgtgaatgccaaagtaagtgagcctggagttagtgacagaaaagaccatgtttgtttagtctcctttagaaatgttagaggaaaagaaaaaaaattggacttaacctaaaggcagaaccaagatgtttacagaccttaccagccttgacttctgcataaagacacatatactttagtgagcagattgagatagttctgactctctactactggccgcactggcctgattcatataaattcaccttcttcaccatgtacctgtgaccctctaggaatccactgtcacctcacatatgtccagaaaccattctctcagctgtcactaacacacactgcccacatttctcccctagttcccaaatgtttgcatccacagctcaggaggtccattggcaaagaccctttgttgttacttcctatcaggagaaaatcagtgcttgagtcaaggatgcttctgggtgttttggcaggaggtctctccccagctgtgagtgatattgtattgtctgggccttaaagaagctctgctagtgcccagagtatcaggaagtgtctcttgatcaatttctggtcataatcttttttccagaatttcatttcagttccatcattttttggctgtcattcctttactgtaagcatttaatctcaggcaaggagtccaatgaagaacagatgctaggctctacttttttctctatacttaccttttactaacccttctcccccttacagaattctcccaatttcctcctccttctccccgtaacagaataagggttgccatgcatgtacatggttacaaaccagattgggcctggagagtggaggttaaatttttggcccatgatctgtcagccaaaaatgttctattacaaaagcagtggattttctagttagaatcattcttgggaattgtgacactttgcatatttggtgtacttgaggaacagcaagggtaccagtttggtttggttacagtggtctgaaagaggggagagatctgtatggggtgatcatatatttcataaattatctgggtcccatgtggagagtggacaaagggagaggactgcaagcaaggtgccctgtgaggaggcctgcacaaaagtgaagaggcctgagctgaagtgagaaggtctgcgtgaaagtgatttgtgtgaaagatggtggtgtatccaactagagcaggagcagtgggggtggtgagaagtggtcttttgtaagtagagcctgttggacttgctgatggattggaagcggcatgtgagggaaaaagaggtctaaggagtgactgctagtgtttggcttaagtagcctggtgaatagtgatgtcacttactgacttggggaaatgaagagctctgttttgaacaggtgaagcttgagagtcattcctagaatctcaaatcaagatgagtcttaatcaaaatgtcttagccaaaggattcgagagccaggatgctcctgcagagtagtgcaattatggtttcccttacaatgcagcagagaaccaaagctacccaacagttttttaagacttagtgtcctcacttataggtggggatatattacttatttcactgggttctttgaaggtaacattagataatgtttgtgaaactcagtgtaattcttgacaagtaattagcacctaataaatatcttccattattgttgttattaattttggtaaaatgaactttatttatttatttattttgagacagagtctcgctctgttgcccagactggagtgcagtggcgcaatcttggctcactgcaacctccgccttctgggttcaagcgattctcctgcctcagattcccaagtggtgtgattacagccgcccaccactatgactggctaatttttatgtttttactagagaggaggtttcaccatgttggccaggctggtttcaaactcttgacttcaaatgatccccctgcctcagcctctcaaattgctaagattacaggtgtgagccactgtgccaggccaggaaaattatttttaaagctgtttttaaatatcagctttggaatgaaattggatataaattaatacttagtatatccagaatctacaaagaacttaaacaaatttacaagaaaaaaatcaaacaaccccatcaaaaagtgggcaaaggatatgaacagacacttctcgaaagaagacatttatgtagctgacagacacatggaaaaatgctcatcatcactggccatcagagaaatgcaaatcaaaaccacaatgagataccatctcacaccagttagaatggcgatcattaacaagtcaggaaacagcaggtgctggagaggatgtggagaaatgggaacacttttacactgttgctgggactgtaaactagttcaaccattgtggaagacagtgtggcaattcctcagggatctagaactagaaatatcatttgacccagccatcccattactgggtatatacccaaaggattataaatcatgctgctataaagacacatgcacacgtgtgtttattgaggcactattcacaatagcaaagacttggaaccaacccaaatgtccatcagtgatagactggattaagaaaatgtggcacatatacaccatggaataaatactatgcagccacgaaaaaggatgagttcatgtcctttgtagggacatggatgaagctggaaaccatcattatgagcaaactatcgcaaggacagaaaaccaaacaccgcatgttctcactcataggtgggaactgaacaatgagaacacttggacacagggtggggaacatcacacactggggcctgtcatggggtgcagggagtggggagggtagagggatagcattaggagatatacctaatgtaaatgatgagttaacgggtgcagcacaccaacatggcacatgtatacatgtgtaacaaacctgcatgttgtgcacatgtaccctagaacttaaagtataattaaaaaataaattagtaatggatagtcatttcacatgtgtagtatacatatatacacacacactaatgtaaataaaacaactttttctttttaaatgagttgcatttttttcctcagaactacaaagtattaaaatcaggattttgaattttatgcgtacgtgtgaggaactttctgaattataaatgtgattagtgacatgagtaaatgtttgctgcctcttaagatctgccatgcctttatgcaaatcaaagttcccacagaaataatgagtcaaatacagcaacaggcagtgtttaaattagcattttttttcctctgctctgcttgggtcaattttactatacattgcatatcaagacctttgatttgctcgggttttccactgatttgacttcccctaccttttgtttcaaaacctaagattaccactttaagtgtactggtattgtggaaggtgttttattcagaatagagtaaatatatattagagaatattatattgatattctccctattgcattaacagggagatctttatggagcaaaggccagatcacttaataaaaccacagtctgttttttggcaatagtgttgaacctggaggaaagccagtcttagccagtataacgttagattccattccatcagcataatagttaataatagttgattgacagggatgtgaagggtgccctcttttcctcccccctccactcccattccacactaaactctctagggaatgtgccattcctttacccattcctaaaattctttgcttgagcacagccaagggctgctggactagtgcctgactgtttggaggctggagagtagggggtctctctgaaagagttcctagtccctgtagtcgttgctgaggaagcctagcaatctctggacatctttagggatatgcagtaaaggtgagcttccgggctagtgtttatgtgtgaaaaataatcatactcacctgaactaccttaataccatgggatcaggatcacttttgcttcccttcagccacacacattcatcttgtccacagaaatttagagcttattttagtctgttttttgtaaatcatacatcatatcatatattattttgcattgttaaaactatattttgttggtatttagtactggtaaattagtaacatttgcagctttcttccaactcttttttctttttggatgtaggtatttgcaggatatgaactattcaaacagttacagcattccagggattctttccctggattctacatgctcacatggagttatatttgaataccaagaagtattgttgaatattgctgaacataaggttcactagtacagtgttttaggtgaaaattcaaaagtttttttccaagtccaagtaatttatgtttttagtggagtaatgattcctatactaagcagttttgttttctttaatttggtgaagttagcatttggtgttgccctcagtttgggttagcaattattctttaccccacgtctcaccactgcagaaccacttgctaacttttaatcaccaccttctcaagtttaagaacttggccagagtgatcccggaaagtctcaacattttccagtgagtaaagtccatctcttttggagatcttgacacctatcattaacaaggctgcaacctccttaaggataatcgagaaaggagacctgctcttctgtatgtatagcaaaacataaagaggttatacagcaggatgcgatagttcttaaatagagtgcatgcattgtctggcaggctgacccaacatcgtgttgcctccttcattctacagattatgttatgggtttcaggtgtttttctcaggtcagtttccctatcattaatgctgcaacaccttcactgagtagatatccactctctcattcttttattacaccatggtgaaaccagcactgccaactctcatgagttgatgaataaactctaattttactgcatttataaatgaaactttaaaaatagtttctggaataagatcatggtgaataaagtcctaactagattttttaaacaaagccatgtttaaatgaatcatattgaaatacattaatatatttagtgttggtatgttaatatataagccatttataccctaagatgtcaatagtgtcacttgccttgctggcaaatttggctcataataagaacatgtgtccaaaaggctttttgtttaaaaacatacataatttgaaaaatatttaattgctctttagtgccttttaataaaagaaggagctcacctttcgattttaaaaatgcttactaaaaattgcttttaacatcatctgtttctaagtgtttctaagtgttttaaaagagaaagatgacttggggaagaattcaacttgattctgtaaatattttttgaacattattgtgtgccagccactgtgttaagtgcctggaatgcagagatgaataagattgttcttgggtcatagcgtacagcgtaatagaagtgacagttaaattatatttgcggtgtagattcactattctgttaggagccattaatttcaccaaaattggacagaaagggatattcttttaggaaatcacattgaaactggaccttgaatcatgaaagctagaaaatgaggacaaatgaggacatccaatgcaaagggatactacatgagcaaagagagttgtaagtatgtgatgggctctaaaggttacaaagtacaacttcaacaaggcttaatgttattttattctttcagattagagaaggcatgataatattttttcatgtgtaaaaggccttcagataatgctcatcacctgatattgggacttaatagtaacagaaaatacttaagaaagggagagttgaaggaacctgttttttttttcaatttcttttttagagtgacatagatacattttatcatcttatgatttttttcagaaacctttttgtagaccttgttacttctgtaagatttcatatatgttcatttccttaccccaaagccataataaaaatgattactaagcacttttctaacactgaccataagaatgaaattaacattgctgtgagtaaactgtagtttaggagaatcttttgtagtgaggaacagaactattatttattgagtatccctatatgttaggtactgtgtgaggtactttcatacagtctatacaatttactccttaaaacttccctggaaagttattatctcaaatttctaagccagggaagcattccagggaaactgaggctgactaatatgctctgtgatatacagctggtaagtgacaggtttgggatgcaaaatcaaggtggttgaattccacatcatatgctcactttactgtagtttagacaggtgtacttgaataattggcaggtgacaagtgtcagtttgggtatttaaactgaggcctgacttactccaaataatctgtattctatccccaacctttttggcaccaagtactggttttgtggaagacaatttttccacagcctgagggttagtgggggatggtttcgggatgattcaagagcattacatttattgtgcactttgtttctattattacattgttatatataatgaaataattctataactcatcataatgtagaatctgtggagtcctaagcttgttttcctgcaactagatagtcccagatagtctgggggtgatgggagacagtgacagatcatcaggcgttagattctcataaggagcacacaagctagatctctggcatgtgcagttcacaataaagttcgccatctgaggctgggcgcagtgcttcacacttgtaatcctagtactttgggaggccgaggtgggcggatcacctgaggttgagagtttgagaccagcctgaccaacatagagaaactccatctctactaaaaatacaaaaaaaaaaaaaaaaaaaaaagttagcctggtgtggtggcgcatgtctgtaatcccagctaggctgaggcaggagaatcgcttgaaactgggaggcagaggttgtggtgagccaagattgcgccattgcactccagcctgggcaacaagagcgaaactccgtctcaaaaaaaaaaaaaaaaaaaaaaaggttcgccgtcctatgagaatctagtgccatagctgatctgacaggaggtggagctcaagcagtagtatgagagatgggaagtgctgtaaatacagatggagctcacctcctgctatgtgaccagtttcctactggttcctactggtccctggcctggggtttggggatgcctggcactagaacttatagaagttatttgaacttcaaacattgcaaaagcagagtgttttcattacttaaagtagtgtttaattttagcatgtcttactttctggctatagtttttaatagacttttcaactatatggctcattttaactgaaggtttaaaaagtcattttgttgtctcagaggtataaaattaccatcaaataagcagacatatgaaggaaaagaaatataaaatgcttttctggacctctaaatgaattgttaaaaaccctgcaaaccaaagcaacatttctcctctgaaattatattttgcaaacatattttagaaggaaaacttgaactgtcaaaagtagtgaaatgtctccagtgtttatggatgtaataagtgaattagctgaaaccccatccaaaaaaattgtcagtgaaatttatgagtctttttgctatctgtatggagttggcagcaggtgtttcttactcacacactcaccaaactttgaagaagagtatttttaacccttgctttataataggcaagttccgatgatctaaatatttcttcatattctgtatactgagtgccttgagtaatttgaatgatatgggatattgactatatatgtgttaagttctaaatccaataaacttagagttttaaagttgacttagtcagagggtttgcttcattgctaacattagcacagattcttttgtgcaaatgtaccttttcttccttttatctatgtttttatcattataaatcttatttcttgcattaatataacatcacaggataagctagtaaaaagtacattggtttataaaagaagggacctattgacatttgctgtaccttgtatatttgctcatatttctagatagctgttttatttccttaatttggctgtaaacttcttgagagtaaggatcattctttatcgttgtatttattcctcaaacttagcacaatagtttttctattgcaagccttaagtatacatttattgctgcttgatttcatgaaaaactgttttaacatatagcacataacaatgtatagaacaaaactagaaaaatttctgtttgacatgttatcaaaatcctagatatttcatcactattgggtaacttgtagtcagcctcacttgcctttaacttgatcatgagtcccacttgctcttcagagacctttctttaggtctataaagttcttattaaaatataactagtttagttcttatttaaacttatttaaggacctccctgactttgcagccagacctctgacctttacaattgaatatccttaatggactcagattctggaccctacccccagaggatattaggaggggtgtagtagctcactagtttagtacacagtcacatttaatgtcagttctggtctgaccttggtacctcggttcctcagggcatgattttatggtttgctctcagggtcttgagtttcttgccctgaaaacccactttccccatgactggtacctgatgatcactccatttttcatactccattgcattcgcacatgttattcccccttctgggatatcttcccaccaagtgtgactggtgaaacttcatgtttcaaggtggaattcagtcatcttctttgagaaaccttccccagcctacccaaacttctttgatcatcccctcttttatatcttctccttatacctgagtccacacatctattagtgtactcatcatgccatattctcattatttgttgactttctgtctatattggactttgagctcattgtagttcaataaatgtttaaaagagtgaattacttctgtgactttgttttggatcattgccaatcacctgtcctgcatctacttggaggatattatagatactggatttaattgaatctactaatgacatacttacatacctattcttttttttttttcttctgttctggctaatatcctgatttcagtgtacctacctttgggccatgctccagtttgttgattgctgggataaccactggctagctctatatgctgtaagtctttctgaactacctcctatactcttcccaggcctggtatgtttccacttgaccctcatgggccttactcatcttcctagactctaggccttcacagacctaacttaatacatgttggacatataaatagaaactctaattgcttttttattagttgtaatagatttatactaaaaaaactcaacctaacaccaaatttttttttctttatgaaagggatatttaatgattaggactaatttaacaataggtcaagatggtgccaaacatttcatcccttattaaatggattagagaaggagcttagtaaatagctttttttttccttccatatgtgttgcaattgtcaaacctattagaattagttaggattttgagtcagtgatatgtgtggtatcattaaaccatttcttgccatggctagcccatgtcctttatcttaaatttgtcagtgatttaaagattaatgttgttcctttaaaaatattcactagatacatgaaaatttactagttgtaagaaaatatttaaaagtcttttctgaacatttagttctcatttattttgaactagtcttaatcacttgcattttacccatgtctcttacagtcatacattattagagggttttttcacttattttctatttcaaccattttcaaagttttctgaattataatagttgcagtttcataactgttgctcccccattcattgattgatttgttctcctcgttttctctttcctttaatgttgaaaatttggatatatagagaagtagatatttaatgaacctttgtgcctccatatcccaaaatactttccctcccaaattattttaaagtaatttccaactttagaagttcactgacacagtatttgtaaattatgggtgggcaaaagaatctccctttccatattatcacatcataacagaatatgacaagatatccatatctatctatctatctatctatctatctctatatagattttttaaatatatacttcagggtaaatagaagctcaggcttatctctttttctcttaggagagtgtttttatttcttttgttcctttgaactttgtagttgtaagttgtgggaactggaattgggtacatgtgtttttaagtgttagtcaaatttcttgctaaatattgctcagcagtttatcagattaggtaattggtttcctaaaaagcttttggcagggttccaggtgataggagaaatgaaaagtgaattttgattgggggcaggtgataggaaagcaaatacaaagtgtggttcatttagggtatgtcatagaaagctgggcatcaaactggagggtggaaaaacagtgggcactaattagatgccttaaggtgggtagcactttgggaaaaatggaagttgctgctaggaaaacatagagaaagtaaaaatcaggagatgacaagctttgcctattttaatcatatttttacttgggctggcaaatgtatcctgcccaactgtggtgggacaacacaagctttctctcttggtagtatacaagaaatttatggagatggtttatagatggagatcttttcttttctctttctctgtctctctctctcccctttctccttccttccctccctcccttcctgccttccttcctttcttctctctctctctttctttttttctctctccccctccctcctttccttccttccaccctccctccctcctttccttccttccttccttccatgcttccttccttccttctttcttcctttcctttccttcttttttcttttctattgtgtcacctaggcttcagtacagtggcacaatcatggcttactccagccttgaactcctgggctgaagcaatactcctgtcccagtcccccaagtagttgggactacaaacacgcactaccacacctggctacattttaaattttttgtatagatgggatcccactatgttgcccaggctcgtcttgaaccagtgggctcaagtgatctacccaggctcccaaactgttgggactacaggcatgagccatgacacccaaccaagactcttctttatagaccttctcccccttctccctcaacaaaaaagagaaaattagacagcaacaacaatagcagcaacaacaaatgctgtatttaaggtttatttttcaatagatttaacgtcacaatgatgggctagtactactgtcatatcaacctgacatatcagaagttagtactcaccatgccactattgtttctcataggtttggaaacagaaaactcagagagcagtttggttgtgaacacagttggttaacatgggagagaagaatcactttggtttggcaggaaatgacagcagaatttctttctatatggtgggttgtccatctggtctgtccatgctaatgagacagataaatgagaactgtcaaaattaggtagccactcactctatttcactaaaatgtaaaattacccttctggactcacttttcaaagggatgaagaaacacagtatttagagggcaaagcaaaaaacaaaacctaagccctgctcttcaacttacttattagctgtgtgattttgctcaaataattctgttttctgcgattcagttttcttgtacgtttactggggataataatagcacctacttcatattctagtgtgaagatctattgagatggaagatttgaaaacactctgaactaaataaactgtttaagtatgagttacattataatataactcataaagaaaagagtttctcataacctctgtaggatactgattttgtttctggaaagaatatagtaattgttgctgtgaattgagaactaatttaatgttaagaaaagggaagaaaaacctttgttaaaaatagaaagtacatttttaacattttcaacagaagataaaatattactgtctacatttttaagttttcttagcaatttcagccaatttctctacccttctaaaataatatagcaaaacataaatctttgtattcatgaattgttttttttaaaggacacataatctgtttacacttgaagagttgaggatacaggaatatgtgcgtttatcagtgggatccttagtaggatcataaagctcttaagacggaaaggaatatgcaatcttctactctaacccacttgtgttatagattaaacaaagctagattttagagagaaatatagatagaattttatcttccaaactaatgtaaacatgattaaaaaaaataagtacatagaataggccgggtgcggtggctcacacctgtaatcccagtactttgggaggccgaggagggtggatcacgaggtcaggagatcgagaccatcctggctaacacggtgaaaccccgtctctactaaaaatacacaaaaattagccggacatggtggcgggcgcctgtagtcccagctacttgggaggctggggcaggagaatggcgtgaacctgggaggtggagcttgcagtgagccgaaattgcaccactgcactccagcctgggcgagagagcaagactctgtctcaaaaaaaaaaaaaaaaaaaaaaaaagtacatagaatagtatcagaactggaagcccaaaggtcaattaaaaaaatttaaaaaacaaattgcaaagatgttaagattgtttcccatttttatagttcatacttacttactattttcattcattttgttgattgacagcattcactatagataagaggtagttcatggcagtggtttaatataaattattagagaaaatttaaatacagaaagttcaagaaattcatatttctgtgatggaaaaaagactagtattattttacttgagctatggaatactacaatttaatatctataacacttgtgcttttcatggatacattttaagaaccactgatttaaacaagtgacacattaatacacttatttgttcactattatcatattcatttggtgttagaattctgtaaatgtagaattaagtcactagaaaatccttaaaataagccttctctataccagattcagtgaatttcatgtatataagatggataattattaagcagcttgcataaagatattccacagtccttttggtaaacttgtacctgtgtgtaacaatcttttattctgaggaaaccaaaattttccttgaaatgatttatatattagttgtatgtcttcagactcactaagttcatatacttttatttcttttatttatttattttttttggagacggagtctggctttgtcgcccaggctggagtgcagtggcgcgatctccgctcacgcaagctccgcctcctgggttcacgcccttctcctgcctcagcctccggagtagctggactttcatttcttaatagtgaattgtagaatttggatgtagaatttggaagttggacatctctcaagcttcttttaaattaaaatatcataatttatcctaaacaattgaagataaatattccatggtattaagtatagggaaatgacatatctcatttattctcttttctatattaaacatttaattccaaaaatattataataactagcagtgaatatatgaatttacgtttggagttaaaaagcacttcacatacatatttcatttaatttaaataaagtacaactgctgaagatagatcgatagatgttatatcagtataactgtatcgcaaaccaccatgtttaatttttgtgttgggattctatgcctactacacattatttgtgtttaaatagttctctgtcttattcttctcttgtttatattacatgtttaggaattttgtgtttattattttagaaaatgagattatatagtagttttgtttcggtgttttagaataaatggaatttctataaaaagagttttctatttttttttctagtggaaaactaggtgatggtgcatatgactttatccctaggaaaagcattagttttgtaatggggtttcatgaagtttacttccgttcacttttttccctgcctgtttaaaaaaaatcctgtttagtttacatgatgtgtaggaaagcgttgggaaaacattatttgtttttttacaatcagtgatttaaaaacatagtcattagtgtgtctgtgtgttttaaatttgtatatgaatttctgtgatgccttatctgctctgctctttgctatgtgccattaacctaaatttggtagagtgtggtggtggtgtattcttagtatttgcttatgattattttatgagttctttgataatattattcccaggcagagaaaaatcaaaaatcttctttccatattctttagtttgaatgtcagtgcttgtataatatcaagatagaggtcacatgaaaatattttatcttcatttctcacccaaacgaggatggttaaaatacactgggtgtgtttaacataaactacttaatggttattgtgtcagtatatatcaattaatgaaaaattaatcaaatgtttcctttgaggattctagttttgttaaaagaaaacttctgtgtgacctcagaaaattaatacttgtattcagattctcaaatgagagttgataaataatatttatttaggtgacatttggagcaaactgtcactttaaaaaataaacttctactaatgacattatgacaacatccacattgggggaactcaaatataggataagatatatattttttctactgagtataacttgaaagtacttggaatttgtatttatttcttggttcattttaaatattatggcaaaaaccacagttactttggcatcaacctaatatttcagctgataccctaatttgaagtattgcaatggccagtttgctaatgaacataaacacatacagcatgtgccaatagattgtttctctaaagcagcgattagcttttagaaaacttttcccagtgagatttactatttcatatttaatgttaataaatgggaatgggacatgtgttgaaataacgataaggccatattaagccagcagaacagaacaaaactttaactgaatgaaataaggtagtatagacttcagcatttctcttttattgagactgtctcagtatctgtatagtgtatagccaaggaacagtttgaaagccactttagaattgtcttctagaaaagtacacacatagttgaaatctgaaataactggaagttagttttaagaaaacaaaccgccttttcaggatccatgcttgtctgtctagcctgaataatttaggcttacgtttaataaccatttaaatacaattgtaataaattcatcatcatattaataaggtttgatgaatcaaatttttatagaatgtttaagagaaggttttttcctaaatgaaataattgcagaaaatttggcattctattactgtagcaaaacttggcaaagaccaaagtacagaacagaatctagattcttagtgaaatttgaatcacagcttctatgtggtaatgcaatgtcagtcttctaaaaaatatgtatgtaagcaaaatccattgattggatttaagagttttgttaggcttttgttattctaactaatgttccatctggattttaatatttgacataaatatgtacagtcatgtgcatgcctgataacttttcaatgatagaccacatatacaacatatacagcggtgatgccataatattataaatagggtatttttaagatacattttctgtgtatatgtagataccattgggttacaattgcctacgtattcagtacagtaacatgctgtgcaggtttgtagcttaggaacaacaggcaatatataccatacggcctaggtatgaagtaggctatcctatctagatttgtgatgtacacttgatagtgttcacagctaaggacacatttctcagagcacattcctgtagttaggtgatagttgattttatgtaatagttataaacatgtcagaataatatattttccttgacttagttttatatggggttaaaaagtaaagggcaaaatactgctactctgttcatgtttttagttagagatggatgtgtctcttcaaagttttttcttttcaatctttttcatttgtatgatatttaaatcagtgctctggatatttgcaacaaaataatgaatattgtcactttctcagtttgagaggatagtaaaatacattcaagggaccagggaaatatgattataggagctgggtcaaataggtacaagtttctggatcccagggattgtgaaatggaaatttatctttttctacttctggatgttactttagagataattcttcacatcattctgaatttttaaaatttaggttatttagagatgtggaggttgcagtgagccaagatcgtgccactgcactccagacctggcgacagagcaagactccatctcaaagaaaacaaaaattatgttattcataagtttttatggacttctgattcgttggaagttaacatggctttcacttccctgattttcatttaatttgtttaatatatgctttacattttggaaaggtgaagtgtacattgatttttctgttgtttttcttgtagtgttacctctttcaaaggtgtaacatgctattaaacatttgaataactgtcatactctaaaacctttttatatttatgaaacatagatcatgtcaataatggattactatcatttgtttaatattttcctggcagtgttttctgtttaatttttgtatacttcttctgtggtaaatatggagttagtttatttctggttacttattgggaaactgagacactaacatttacctccactcttactcactgaactccatcactaaccagcagtagaacctaggaatcttacttctagttcagtgattggtttattgtatctcagtttctagtaccagattaactatattagctcctcgtattaacttttatttgtgatgtttttcaaggagtggaaaacagtcttcattttccacttcccccaaatcacaaagaatatggacttatgacgtattaagtagaccttaaggtttttaagtaaaatatttacatctattcctgtaaaccttctgctttcttgcatccgagacaatactgtacttatactggataagtattctgtctgaatatttactgaatttatgcattattaaatgaatgaaaaagggaaacattgattgaccatcatttgagagtattgaagaagcttaagcagaataaaatttataaaacagcataaattcagtctctaactgaatgggttccagtgccttagatttcgtgttcatgtgtcattttcgttgaatttttatttcttcttctaggccatacttaatttttattgagttgctgtgtttaacagttttcagcaaaagcttaaaataaaaacactagaaaaatataaagtttaatgtacctaatatgcaaggcttgataaggtgccctataaatggggaaacaaataaatatggtcctttctcttaacagacttaactttcaagagtgctgaaattcactaaggcttataaaagtttgtaactaaagtcagtatgtagaaaggtatctttgccttgtctatctttgaatgagctgcttggtgacattattccactacagggataagttcaatacccagtgagcgataatgataataaaatctgcagattgtagactgtctactgcattattccttactattgtataacactgtgtattttcagcaagttttactcaatatatttgctggcgacaaaatgagtttttaaagaaaagaattagctgactagtaacatttagggagatcatcagtcgaaacagaaaaaaaaaaaaacaaaggaaaggaatctgaaaactaatcttttttttgaccaattagtgacagaaatttccgagttactcctttcttatatttcatcgttagctgatttgattgggaagagaaaagagattttaattttacccctgtcatgctgggcgagaattcactgtttcaaaacagaactgacaagtgaatttcattaagactgataatatggcaacatagtgaaagcttgcaactattatcagcatgaagaaattttaaccaagctattatttttgaccaatgttctattacttgtcatagcgatcttgtatctaatgatggtgaatataatgggttgaagaggtttcattttaggttttcattcaaaaattgtgtgtgtgtatgtgtgtgtttcattttaatatttttaggttttgcttgcattgtagggctaagttcatagtgaaaaaagacaattatataattttgtttatatttgtcgtggaaacaggctaggaagtattagtgagaactacctgttgatactgacatgtatcttcaacagtaaatattctgctttctgataaagggatatatggtagggctacaagtccttagttcaggaatttaagtgaattgctgtgagcctttcttcttcccaattctgtgcctttatacttctccttccacacagacagacacttgtagtgggattgtgatctcttcttgtctccacctaatataaacagaatatgaagttaggactatggcttgattgttactgtatctttagtgcttagcagtgtgtccggtacaaaagtcttaccccataaatatttgttgagtgaaggaatgaacactcacattttatcaccttgaaaccgggcgtaaaagcccagttcttaaactcactcgttacattcaattccttttctctgcagattctttgcatttttctgtttattttgttgcatttcacttgtttgcctattacttagtaaatgagcttgtgtttttaaatgttgtatatttttattccgtaatattcagatatcagttactaataaatatttgtttgtatagtatctagtaaaatataaatggccccattaatggaaaaagactttgaattcccatatttgtagaaacattttcccccaacttggtttcttttatagccagtgatatacataatgagggtatttggctggttcagacacacagtattattcaacattgtgatgtggtcctgatcctcagcttccttttgtataaaatgaaactaatacttattggaaagatttgtttggtaattaaatggctaatgttgaaggacgcccaactaaatgcaccttcactagatgttagtacttttccctctctgttcatttgttccttccattgctaccaatttcacaaaactagcccacagaataataaattcctgtggccctattggccctggattttaacctttaagggccttaaacctttcttttccagcagtattattttgagagacctggacaccgtcgttgactctcctgtcttttccttttagttcctctcaaacactgtattttgcactgagaccgtgctcccatgtggcgttctgtactgtccttgtctgagaactgcttaccttgttaggtttgtctgtatccccaaccagactataagcctgtcttgtctagtcctatagatccaatacttaacagtgtctagtgtgtatactgtactctataaattggtatttgataaattgatttgcttatttgtttttctgttattatgggaaagtgatgatggcaagagggaatcctgaaaatcagtgaattccctgatcctggaatacaatccagtgacttcccaagtccacactctttatatattagatgtaagtagtaacaggagttttaatatgtaaaggaagaagctaagttttctatctgtaaaagctattcttcaggattctattccttaatttaattttgctatgtagttaaagataatggcatttaaagcttaacttgatctcttaataatgggcatctctttcccataactaagcatagttaaaatatgtcctgtgttttccatattatttgaaaaactatcaggattttactgtattcatatgaaccttgtaaactgataattcttgtgtcagctggaagtttaaagcttcctgagtttaaggaagttagttcaaatttctgaaaggaatttttttttcctctgagtgtgaaagtcaaaccagacatttgaatataaacttaatctcaaaatgcatgaaaataatggagtgtgttaggtgtttatggttaaaaatatcgcacaattaaaggcaggctaccttgccactaggtaaattagtcatgcaataggtttaatctacttagaaatagcactgtgaggaagaaaataaaattaattcagctgatagtgtcagatcatggaaagaaaagaaaaaaacagcttactcttgccaaaaacatattgctaactttgttcaggttgcagcttttgtgtttcatttaggtttcagcttgtcccaggaggtgttgctatactgtgaaggtgttaggcagtgtagtaatggaacaggagataaaatggaaggcacataaaatgtctcatcatttggaccttcctggagaaacatagcttgaatttctgagtggtcagcttgacattttaaagcagtgagaagttgtgtcattatttctagaatactacagtagttagaggaaaaagatttaaagttcattgcggtaattgctgtcaggtgcttttagtaagcatgtgctgccttgaacaaaggcaacctgctaggagtgtgaaagaagacagtgggctttggaagatgagagtgacaaaaacgtagattcgggaattcttgtgctcctgaaataattgcattcattttaaaggtggaagaagattaaaataacttttaatggttattcatatgtttatagcctttgagaaaacaaagactttaggcttctaaaactaataccttacataatttcagtaacaaagagtacaaaacctggtggctagcttgtcacagggatcaaggtagctggtatcactaggctaggcacaatccatgtgaagtgctgaggtgatccctactgtaacagcattcccctccctggaaacctgcaatttgcctcttagtcccagatggggaggaaactcccaaacagccgcaagccatacataatggcagggttcacatcctgacggggacaaagtagagaaaaagagaaattcagaagagtcttcataacattatttgagtcaaggactttatgctttttgtgaaggttgtagcacttcttttttctttttaaagttttgagtatgttgtgtgtgtgtgtgtgtgtgtgtgtgtgtgtgtgttttctccctcctggatactagccaacattcaactgaaggaaacacatggggatggcacatttatcacattgcactatcttttgcttaagtagctaacatttattgcttaataaagaacatcctgcacacataccaactaggtttccagtatattactatgcatatgtataaatgtacattcacatttaaacatgcttttacctatgaccctcagaaacacctgaacacatagaattaggcatgcccagacgtttcaaatatgtgatctaaaaggtagatctttaccccagattttgtataaataaatttatactattacttataatagtgaccaataactattaccaattacacaaattgttatttatggaaaacttaaagaatagtctggttgtattctgtaatcatagatagataccatagattgaagggaccttaagatccttaacctcaggcaggtaggcttttaagcattttacctgcacaaatcggtagcattacctttatttcacagaaaaggaagctaagccccttagaaggtaacttgtacaattccaggtgacatagatgtgatcttcagtgctaaatcaccagttaatagaagaaataaaacccagtttgtataaattttgatctacttacctgaccagaaagcatcaagtattctaaaagaagtatttttaaatggataaggaactctcttacatctgattaatgtaatatcatacatacacaagtgttaaaagaaagccatttatatgccctttaactataccttatatgtctgtctactttgctttctatggtgcatagtcatcactttaatatgtttcacatactttagttatgagctttagaaagctgtttaaatggtgatttagaaatggagataactcattgaataaccaagacttcagttcacaaatacaaggatggcatgcttaaagtaactacttacaatgagaagttatctttttgaattagtgatcttatgagaatatttgttgattatttatgctctatattttgcactgaggttttatagtagctgaatattctggcatggtggtgtggggcttatgaatccttgtattgctcattaaatattttacagtgcatagttgcttatatatataaaaagacagacacatatatctatatgcatacctttcactagtgccataaaagcagggaatgtatctgtcttgttcactactgcttcgtggtgcctggtacagagcaggtgctcagtgagtaattttgaatgatttcttaagagttcttcatacttggttgtcctattaataagttaacagcatttgtcttttgaaattactgccattcaagcattaattctcttgtactttgctcaattttaaatgctctgtggttttaggatatgttctattgtatattatccattgagtgtatccattctggaaaaaatggtgtcattcacaaccaacatgtactcatgcctttttcctctcttcatgcttgcccctatcttgagtgccatctagttcttcttttatataagtcatatccaacttttagtactattttacatgccatttccttcataaagccttctgcatcttccctgtacttccatgtagtaaacataaccccagatttatgtttgtttgatgttgatacttatgaaatgctttcacatagatatattaataagaagcaagtagttccaagtgtattgtttatacaattttatttaatactcaggacaaccctaggaggcagctgttgtcattattttgattttatagatgagaaaattgaaacttaggtttcctaaggtcatgtgacttataaatgaacgaacctggccttaaactccagggtgctggacctagagcccttgctctttaaccacagggctatgttgcttgctttaagtgattgcatgcttttccagtcttcacagttttaatgtagaggatattatcttcacttcatacagtaggaagcagggagctagcaagtggcttccaacatttatataagtaagcatttaaacatagatgttataatctggtatttccatgatttaaatacttcataggatgctttgtaaatgtttttgcctttaattatatgcattctgtaatcctacttaaaatgctcattcattcattaggagtagaaaactaggtatgagcattgtaaagaagaagaacaacaacaacaacaacaaactgctgctgctacattgtggaaaataagtatctggcactgatctgtcaaaaattgctttaattccccttaatcaccacaagttttctctttctcactacttgatagatcttactggttattaatttctaactgaccaatcctgcatctgagggccaaatgcagactctgtttatttgactcagaccagtagtacagtgtttatggaagaacagcagggaaagaacttttgtagcctcgatgtcaatgtgtatgacccagggatgtttgaacctggggtatggaaacctgttgggttcccctgttgcttcacttcagagacccggtaaagctgctgaagtgtgttgcactgctttggctcctccctccctctgttgccgttgcccagtgatagaaatcctgctctcttccttatctctttctgttgagccgccagcagccttgccttctgaccttgtttaagcgtccttagtgacctctgggaatccttgtgggtccctttgggaacatacccactcagctggattttacttcttacagaagttaggttaagaattttgtactgctcttattaaaaagataatttcacaatgtaatttgcttcacaagcctcttacagttacgttaagcagatggttgccaaacaccagttcaaagtagattgctcttatatgaaataaaatccacgctcaggaaatttggcagtgtaagctggtcttcttccaaaacaggcttactcagacaactttctctttacctcagactggcaatctgctacacctgtattccactgtggagtaaaaaacatatcagattgtatacactgattcaaaggtatcattctttaacatattgacatctgttttttttctttactattactagctactttgcttttggcatcatagggaaatagcaagaatttggtcatcagttctgagttccagtcacagctctgactctcaatatttgtatgaattgaatgtgttacttcactgattttaagcttcagtttcctcatttgtaaaatgaggataaaattacgtacttcatggagtggctattttgattaattcagttgatatatataacgcacctaaattctttcctgtcattaatggttctttaattaatgttattttgatattgatgatttgtgtttaaaacctaaatctgtattgttatatttattcatcatgtgtatttgatttatgtgaggtcttcgtaatttaaaaacagaaatatcatacgtaaatcagcagcactggttcaacaaatacatattgggtgctttgtatgtgtaaggatagaatgattttacaactatgaataaaggcagtatttcctctcaaggaccttacactataggcagtgtagaggtaaggcaaatgttctgatgaaatcactgtaatgtaaagtgaagtaaatttcataggagaagaactttttgctcagaaaacactcatgaaaaaaggtagcagataagtgatcctttaatgatgtgctaatttttatagaaataggggaagtatcccagaaaataaaagcaggtgcttgagaaaaaacaaaaaaacctagtaagtagagatgtataaatgtgtttctggaaaactgaggacagtggtttcactggaaaaggcatgagtaactaaaagactcataggacatatggctgaaggatatgatgggactacactttggatattcttaaattccataccaatacctttgtatttaatgtgtaggcagtaggcagttcttgaagacatttagaagagtaatatgaactctgcaataggaagattaatctaacaactgaatatagatgaattgggggagaagagtgtggcaaaagtttgaaggaaagagaccagttagaaagtttctgatattgttccaggtgagaggtcatggggatcttaaagtagttgtattgcccaagaagatgcaaaaacagatgtgcaagacatagaagatattgaacctttaggacttggcaaatgaaatcattcatcgaaaggttggataaagggatagactgctgtttggctgaggcatttctatttaaagtaggtttattttctctgcattgtataattctcatgaactagttgtccgccaaaaaatagaaactccgtctgtgggccacaatggtgctactgtgttagctaataaaggtgtgtattttgatgatgtgcacgctatacaaggtttcatggagtgtgcaggatcatcaggaagttagatattttgccctccattcgtttacattttttttccatgaaagtagggaggaaactttttggtctttattttgttgccaagatctgtttaaatgtagataaaattctacattaccaactcacaggtgtgatttatctttatatcaataattgacacaagactgtcaaaacttgaccacaaaggaaatcctgaaatgctatccttcataaactattggtttagagattaaaattgataatattgacttactgctcaaattgctgccttaacctaccaccgataattcatagcaggacaaggagtatagaaattaatgttaaaaaaatagagaggaggaggaaaagattgaatattgacagattcttcagcaactaggtgtactgcatggacatacccttgacaacagcaaagcaaattttaaaattatgaggtagttgttttgagtgagacatgcatcccagttttgatagaaagtcataattcaatagtttggtttatggaaacttaacatttctttatctgaccattcgggacttaagtttctggtatcctcttcaactggtggacatagttttacagctaccgtaagaactgtttgcctatgactgatttttgtcctaaatataatactaatgtaagttacttgtttcattggagagccagtatcatgtcatagaaggatggataggctacgagtcaaaagatcttgaattgatatcaagttcacatttactaacttgtatcctttcaatagccacttaacaaccatgcttcagtgtttatctgtggctacccttcctacaatgcagtcattctgagtatcaaaaaagaatatgtcagtattttataaatcataaaaactgttttatttttctattattattatcattactattatcattaaaaggattctaaatctgtagtctaccagagattattttatctagcctgctgttttcagaaacatcaagtgttatttccccccatattttagatggtactagtggctgagtcaggataaacaaatttcccaaggtcccataacaagttaaatggcagaagaggtggtagagaccctggttttctgccttcaggtgcagggtaactttactacactattgttggaataataacagtatttcagaaatcagtactgtcttgatctggagtaactccagtgggtttaagaaatacataatatagctatattgaacagagaagaaagaggatggagagaaaagtgaggtgatgtgatatacagatttagaatgctacccagaagtggctcagctgcagctcagtgacaccagtgtctggacatgtcccaccgtttataaaacaaattctttaaggatgttgtgaagaaactgtttttcaagactcatccatttatttatccacagtcaaaaggaacaaatacattgcatgaaattttgctgaggatattaaaaaatgattgcatatacctttgttgtggttttactttaattgtttagactgttggtaaattcttagtaaataattgggctttacatcacagttttgaaggcactttgttttcctaacacattatttaaacagctgttgggatattaacatggatttaatcatgtttggtgtttctgtttagaaagtttacgtgagagagagttatattagtgtagaagcaacttcaataaattattagatagccatattggttcaattagtgtatataacaccatttcaagtgtctggcatagactgggaaatgttcatgcttataagttataaaatcccttaagtgcccacttgaattgccagggaacagaaaccaaccatataaaaagtccacttacacatcacacctgaaatacactttttttcatttggtaagtaagaatggtattttactatcttctgttccacactgtgaaaaaaatatttaattcccataatatttacttccttgaaaaattaaaaattaaaattaaaatatacactttggcaactaaacaaaaataagtcaccaatttcatagaaattaaactagaaagcttattttatactctataagatcaataaatacatcactttaaatagagaaaataaatcataatccctctgcattattttcctggttaggaaacttgactaacttgtttgtagattagtagaatcaggtcatattaataataaagttttaacttgtagaaagtttaatatgtatatttaaaaatgtcttaaattgatattttcagttctcattactatgtatggtgtttccagaacttgacatttaaaaattatagcagtttgtgaaaataaaattctaatgatgagggtagaatttattatcttaatttgaaaatttaaaattaatatatttatcatgcatataaagtgatctttttcttaaaaattttaaaaattgagaattcatattattgaagaaataaatttccagactgcaaaatcatatgctgtcttgcacaaatgggtgactttgtgaagttatagcataaaaagtagtaaagtaaaatgtccaggttttaaagaagtgcctttttacatttatatattgttcagttcaataaatttattatgtaaccaaattacatatgtttgtaaaatatttcctgtattctaactcatgaactcttttatggccatttttggagtacatactcttgtgttaatatgggttacttatttaagttaatagtattttttttgctttagctttaaacctttactattattattgacaaagtattaggcaagtagtagatacttaataaatatttactggtttaatgattttttaagagaattcagtgctgtctttgttctttattggctaaggtagatgagttctataaatgaatgtaaggtctacaaatgtatgtaatgactgttgtgcattttattatccttttcattcttatgtgagtaaaatttgagttttgattttttttttttactttttcaagtacaatggtggcaaagaaaatgtccattatgacattgattggattaaattaatttgtttttaatagattcacaaaataacatgattttgctccatttggttttagattctacttttttttcacaatgatctatacctgtttacctgtgtaaaggcctttattcagttatccttttattatcatattagtgaattaaaggcaagtctagtgtaactgtccattcactattatgtatctggaagcaatgactattgtgaagcctttgattgtagagagcctagaatggttactgaaggaacttttataattgaaaattgtttggcttgaaattcactttaaaaaagagtatctgtgcaagtgtctttacttcttgtaatttgtaagtggaattgcttaaactaggtgtttttatggcatcaagattacattctagtgtttataaatagtattttgaaagcataaagaagaagaggggaaaagcttgaattcatgaaaatgtttatatcctgaagcttttttttgtttggaaaaatatgtggcactttttatcatgaaatatcttcaaagctttttctacataaacttgaccataacagtttttttctattaacttattctttccttgtgttcacaaacctgagtctactaaaatatggatgtaacagttataaatataaatgctaaatagaagtgccattacaactgaagtataagaaacatctttatcatcaaaactaaaatcttgtctcatatccccttttcattgctaacagtttgttctggcaaaattctttttaaattaaaccattattgcttattagcatatgataacgtgtgtacatttccacagatatgcccaagttttacattctttatttcatatcagagaaacaagcttgaaaaataactcttggtattgagaattgtaacagtgatttttcattttactcaaatgaacaagttttcatctttatagcccatttatgaggatgtctttgtgttaccaaattgtaaaataaatttcatataaatctaatttcaaatatttgaaaagtaaagcactctgaatattattgtacagtggttaatagttgtaattcaatagttgtggcatcatatagtgtagctacgaatattataacaaaagtcttatatatatataagttataatttagaatacacttaagtttactacttacatttaatcattaataagttaaaatattaggtgttctacattaattgcaaacatcttaaatagtgaaaataaaccatgggagagtttctatgagtggagaaaacttttctatgtaatagttaagattcaaagaataattgcttttattctagaatttcaacctaattgtagtgagatgggtgaaagattagttacactgggattttgtttgtatatatgcatcatgtaaatgcaaaatttaatgtatgacatgcgtatattaaaatgtgttgcactttattgatttactgtataaatacagcaaaattatatacattcatatattctaaacatacttctttatagtattgttagagaatagattactacggctttactatttcttgtctatttcggttttagcaaaatatacattttttctgtatttatttgtagtatattcttctatagataaaaagttctaactatatttgtaatatctcacacttttaaaaatcacatttttatctgtttcatgttatgttttaatttcccaattaaacttagtagtgtaatgtcagtcatattcttcataattctgagctattttgaaatttaaaagactcatagtttcaggttctaaaactacactttcagttatgtatttcttcattcctgaaatggaattctgatacactttatgcacactaagtggtataaagtcatgctgtataaagttacacttttcagtgattttctttcttaattttaattttaaatggcaattatgaatggaatataacactatatgggataaccaatagcatttcgcattgattttaaaagtctcccttaccagaaaaaataccatggaagaattacttagattatggaaaaacctgttgatgcaagatatagaatccaggagccataactgaagactttaaaaattcaacatgtgaaattagcatatatgccaaaaaatgcccttgagtaaagagataaaccaaaatgtttgcaaaagatatcataaacactgggctaagttccttaatatataaagagcatttaaattcagtgagaaaagactgacaacctgatagaaaaatgggcagacaactttgataaataattctgagaaaaaataaagaacgtttcaacatggaaaaagatgtctaacttcactcataattttaaaatgctttttaaaaaccataataaaataccagttttaatctgtcagattgacagaactatgtggtattgtaaaataatgtctgtaatagcaagaaacaagaaataatttcaaatttccaatgattaactggatgaacaaatgatgctagatttataaaatggattcagactatttggaatttgaaaattatgatactggttgcttttgagatagggcctgaaagatagtgagcttggttacaaaagaacttcatatgtaatttttattctttatgttttttgttttttctttgccatgtttatgggttctttaaacatacaaagcaagtttaaagcaataaaactcttaagttagtaaaacatctcagtgaatactacttgaacatctcctatataccacaacattcttgagcatgctggcaatataaagatggataaaatactgtcccaactctttttaaaaactagtaagggagtaacaatttgtaaatagggtaaaactcaatatagttggtactctgttagaaatgggtccaggaaggatgcatatcactttgtagatctcagagtagtttatgtaccataagttgattgatcttcataaaaaccctatgaaatagagcactatatatccaggaaaaaaaagataagcacatttcctgtatttactcatttaatgctcacagcaaccataggaagtcggtaccattatcatccccgttttataaaggacatggtgaaattaaacctaagttcctcactatcctcgttcttaatacctaaactgttctgctcgaactttattcagtaatctgagttcatctgtactttgcaaacatgagactcagtttaaataattagttcaagattataatgccagtacttttctaccacagagccattttgatagtgcaccctcttgtgataagtactaatgacaaatcacagactgtgtggtttctctaagaaaacaacggcaatgaaaagaacttacttcctttttatgaaaatatttcctctaatcaagtagtaccaaataagttgttttgcttagtaactttgatatgacttcttaagaaattttctatattactggataaaaaactgatactcacttaaagcttgtttctgctttatgaaaagactctgtcagcaatccagaatgaaggctgtgctaacaaaattccaacttttctgtgggatgatccaaaaacagcaaacagaaggcattaaatttcttaacagtggcaagtgtgtacatgcatgctatgccattactgtgattagctgctttttggtgtgcatagattagagttggaatactttgattttcaaacaacatgttccataggtaattttttagatagaaaaaataggcagagtaacttaagagcatgtgaggtctgtgtactacatttatatagctcgaaactgaatatatgtgtttagtttgattttagaacattgccttgacagtgtatcttcatatgtcaaagacatttgctggattaagatcctgaattgtttctcttgccctgttcagaagcaggtttatacaagtactctgatgtgaaacagagaatatgaagtatacataatgatcattaaattaagaggaggcatagttcagtgtgttaagcaatatttaatcttgtttcttccataatcctatctcagtcctaggagtcttagtgacagactcaaaagccaataccaggattattaaaaaaacttgtttaaaaagcaaatgtctaagtgaaagaaacaaatatgtgaggaaatagtcattctgtttacctagagttttcctgagctatcttcacttccatgacatttcactaaaatatcatagaacacagtatgtattaacatggcattcgatgatgaactctattctactttatatatcattttcatccatttagtaggatgacaatgagtagaaaagagatgtaggtgagaatacataacatgtatttaaaatttattttcttcttttcaaattacagcaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtaaactgaaatgtgacgtctgtggcatggtttgcattgggcccaatgtgcttatggtacataaaaggagtcacactggtaagcagctgaaagaataacctgatgactgcctgtgacatctgatgttgatattacctgacatctattctctttcctttttattcaggggtggcagtggtgaaaactaccttttcagaattctgctagtattggattgcagaaaaacagagagtagcctgggtcttgacttccagccttcaaatctgaatagcatatcagaaaagaagtttgggattcagtttccacagttcttaatatagcaataatatgataagggttcaaaaggtataggagaatttcattgtgtaacttaatctttttgtatttctatacatattttaccatacccatgggcaggtaatatagtttgctagataagagtttatactacaaaatggcactcatgcatttatgtaagtgaataattttcactttatccagttaactccttgaaatacaatttctgattgatctctgcctatcctaccacaggtctcagtagcaggcaggtgcttttgtagttcagggatgctgccatctttctgtaattgttggttataactcctgcaaggatatatgtaggattgaatatgacaaatagtggctggtgtgtaggttggaatatgtaattctgggatagagtgaggaaaaagcccacagattaattcatactatatttttaaacatttgagaggtgaaaagttttttgtgaaaactaaccaaatgagaaggtcttatttaaatctttttaaaccctttgccaatttttttttcttaaaaagcaacagaaatgagatagtaagtggtaagctaggaggatcgttgactcttaacaaaaaagattcaagttcatatgaaaattggtttaatggtaatgatggctttcactataatgcaaagtagtgtagtgtacagtaaaatattaataactaatattctcagagttttaaaatatacttttttggaagaaatataaatcacaataaatttatttaaaagttttatttctaaatagtttaacaattacgtgcttaaaacattaaagatacaaggtaggtcaaagtcttttttaatacgaacctttcataatacacatgaagaaatagtcaagtgatttgtccattatcccttatgtaggtaaaggcattgctgaagctagaaccagggttttctgatgcccatactggttttgttcagacacactattgcttggtatgtcagtataccaagaatttattataaggtatgactgtaaactaaggctgatatcttaaaaattacagtgagaagttatacattaaaatcagtgttatcataatcaaaatcatcagctttagaacatatatacttattccaatcatacagtcaattacattttgctttttcgttttttatctcctttttaaacagcacgatgttttaaaaatatttatattactagtgtaacttttttctttaatttttaagtgtcttagaaaaggttgaaagatcaagcttggtatctatttgataaaaagtaaagggaaaagactataaaataatctacttttcatctacgctcatgaactaattctaacgacagttctaaaaacaagttccacaaagttataatatattcattatgtaagagtgattgtattaaggaagataacactcattcaaatgtgtaacttctcctatctagtaaaatacaaacaaaacacagtcagtctaatggcttttcagttgaattgcatgcaatgttaaattttaagctaaaaaataaactaaaaacagtatttagaagatttttaattgatcttttatgttctgaattaagaaaattcaaagaaccagagtacagtttttagaaaatttaggttaatcagtgattcactgtagctacaataatattcatgaggattgttaatttgtaaataaaataataattatatacctactctactgtccttttgttatttactagtgtggtagagttgaattattttatttttgaaaataatctaattggcataataaagtttataaacagttatacagatttccacaaatcctaggaatacgagactcactcaactcataacagcaaggagtatgttaaagtacacttgcaacactatctttttttgtcaatcagtgtttattggagcattgtgcaaattattatactttcttcagatctagatttttcttaaagttaatagccatttacggagaagctgtgatacctgtcatgctttgtcaactttgaaattgtggacatgatatcattaaactagacacttcaacatatttctgatgatccagtttatacatctgagtgtgtcctgccattttcaacaccactttaataccattttgagactagaaagtctatttgcatttctttaatttaaactagattgagaaaaatgtagtcagaatctagtcattagccaaatgttttctccttaataccatgaactagtctgcattttagaacttcccactcttatcaattgtttatctctcatattaaagatactgtcttaaaaccttataatgcctttgagaaacatatattttagtgtttttagttcatagaatatctaatataattttttttttgagttggagtcttgctctgtcgcccaggctggagtgtagtggtgcaatcttggatcactgcaccctccacctcctgggttcaagcgatcctcctgcctcagcctccaaagtagctggaattacatgtgcccaccaccaggcccagctaatttctgtatttttagtagagacagggtttcaccatgttggtctcaaactcctgacctcagttgttccacccaccctagcctctcaaagtgctgggattacaggcgtgagccaccgtgcccagcctctaatataaaattgatagtgaaaaaagatgcacaaccatccaattcattacaatatcatggagcatcagatcgaatccactcttcttctttctctgtcatactctactaaagtaccaattgcagtgtcttattaatggtttcattttattgatttggtattgctactttagtaagtaatcaggtaatattgtcatcggatactccaggcactcttcactgggaatatagagagaagagtaaacaaatagggaaagtaaatcaattcatgcatgaacacatatcgattggacattatacatatagaattccctaattttgcctctccttaatctactcccatcttgtaactcagttttcagcaccgtattttgtacattgcagctttgtgcagccatcctcttgctcaaaggcactcagcagatgcttgagattctgcatatttgtaggttgttccacccaacataggagcaccctctgtctttgattcaccaaagcaagttgatgcatatatatttgcattttaattgaatatttaaatgcaggtctagaaataggtgtttatgaaggtacataaaatattgaaatataagatattccataataaaaataatatataatctcacccagtcaataaataaaaatggaccagtagattgttctattgtatttaaaaataaaaacaaaacaacagattgagatatggacatgaaggacacatgaccatatcttaattattttcctcgatccagttttgttagtctagatttttgtgcctaccaggggaaagattagtcattatggttataacaaactacttaatgttttcagcttggattgctccattaatgtagttagtaaatttgtgagattttcttaaaatttgctttgatatacatatgaagaacatgattaatatttttttcagacaaagtctaaactgactcaaaaagggcacattgatgcagtccatttaaagaacaaattatttacaagaaataattgagatatgttgttttaaagtttcatagacagtatggagtttttgcatatgtaccatttgcatagagaaaagatagttaactgaatcttttacttaattatcagataattttacacttattctgtaatgcctaggccaggaaataccccacgacattcaataaaagggttgctaatgttttatagaaataactttatcaaaaattgagtcaattcactttttatttgttattagacagatatggatattacttctatatttggattattcctggaatataactatgtatacttttagaaatctcataaattaccctctgttttaatccttgtttctaacagtattgtctttgagggtttcattatgattccaactaactttagtttggaacacaataaaacaagtggactcttgaaatgataattatcttttcatataatcaagacatcaacaggagttaggcaaataaaatgaggaaagccataaaattagtatccaaatgtaaaaagcatctatcaagacttattcataattcaaatcaatatctcccaaagccttatcttattgaaattaaaatgtattattttcaatgccccttttctcttaccaaaaaaaggaagaaaactatttaccttcatcatgccttgttcttctttacttcccatgtccttgggaacttaccaaagacttccatagtatgacttccacactcagcaacaggacaatatttccagatatacctagactaccttttgtttattctacgccatttggggatgatcatataaattcagaagtaatttcttattaaaaagatttgaagatatctagagtattttgtaaatgtaaaaactaaattaccctttcattaatcgcataaaactcaattaggtgaattttaaaattctttttaaaaaagatattgtttcatttttagactagcattatttttgaagaatgaactactctaaattaaggattttgaaatatttcaactcaaacattgtaggaggtttaaaggaaattggagtcagcatgctataggagtattatctaaaggcagacatgacttcaaattgttcaccacacttagtaatttaactcttgtaaagttattgttaacgaatttgttacttgcttgggaaaccttacgtagacatattttaaaagtaaaatcaaatttttggagtgaaaagtataatatatatttgctattttcctttttctttaaaaatttaatttgtgaaagaagtgcctccatctttccttttattactatggcaggggttgggagtggaaaattcttgatttaaaaagttgcagcctgtgattccattaggaaagagaatcaaataatcaagttgtatattgtgtgtaaaagtaaaaaagatctgccaaaattcaatttattttaaaaccttatgttggaagactttatatgaggagatatagatcttaccaggttaatcaaactaagaagataccacttaagggaactttaacattacagatttttttcaatccaatattatcaatataggacctcttgtacatgtagctcataagagctccagatttcttaagaaaaaaccttaacccccaccacacccacagattacacctttttggtatgtacctcattaactcccaaaacactaaatactacagaatttgagatatacttgatctaccaatttttaaatttatctaggttttataacaaatctatatgatagaattttcaatagctgctgccagaaagtgaccatagtttatcttatttatacctcttagctttctttaataaaagaatcattgagtttgcattatgtcaaagggccaaaaaaccttgagtttttattattcattattatgtgtgtttcagttaaagatatatgaaacatgacatttcaatgtaagctttttgaaaaactattaaaattagaatcatcatctaactgcaggtaaagaaatggtccagttcaaccaaaagggaattgttttgtaatgttctttttattctaaacttagagctttagtatgatacatttttactgtttacttatatttgaatttttattttatttcatgtatatctttagtgataggtgaatttaaaataatagcattttctttatggtttgataaggctatcaatttgatcctacttgccagttgtactgcttcattgcgtacatgtttttatatttgatgacattggtgagtcagttgatggcaacagaccaaacttataattgaggcttaaaaacttcatagcaatatcagtaatagaaaaacggggagaaaagattcctttcaaataataagtttcacaaaaaacatgtacaggacatgtctggatgaataattttatttggaaattttgctaaaggttttactattagtgaccaggagaatcaagaaacctggcccgtatttgaaaatcttgtactaatgagctttgtaagctggttcatttgctgtaccttttagttgcccctttccatatcagctaaatgagggaattagactgaaggtctctttcagctcaaattttctgccaattttaaagagatagcatggtaaaagaaagagaacaagattttgtagccaaactgaccaggttcatttctcaccttgactactttctagctctgtgtaacaaaatagtttgatctatggactttaatttcttcatttgttaattaaaattaaaacaacctaattattgtcattgtaagaaaggattgaaatattctatggaaagcatccaattgtgtgtaaggccctaaagtgacagcacctcattgttcaaccaatgataaatgctattattgttattcttctgagtcaatgataggacttttgtcacatttatacataaaaggggaaagatcatttgatgtaaaacttaagactttggtaactgtattgacaaagcaaattctgggcagataaatgtttaaaagacttatgagggagatgaacatttttagcatagagaactggctttggttaacagtggatggcaaattagtcacttgcacataccctagttgatacttcccatttcttctccttactgcaggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctggtaagtgtcaccaactccttgagaagaaaagtaaaacaggaaaacttagaagagtactattgaatgatagtaacatggctttgttatgtagtcggtaatgaccaagacaatcttgttattggtgtacattaaataattgataaatcttgcaggatgcatgtatctttcatttttagactttacggcttggcagaaaggagcactgtcaactgaagagtatcttacttcaatatattagaataagaaaacagctgttatatattttctataattttaaaattttacaattaagaatgcagtcattatggttaatccaagataagtgaagatccagtataatatcagttatgtttcgagttactgtagaggaaatgaaaactgattttttttgactgatacagtgctcaggctgggatctctgtggaaaataaaattgtttcttttaaacttgaaaatatactcagaaaattggcttttcgagtattttatattctaattttaactctccttgtatctttagctctcaaacaatagtcattgggtactctgaaactttgctgtgtttcataaatcatttaaaaaatgtagaatatgacaaattcttgggaataaaagaaaaaaataaatttaatgtttttaagaaatgccagaatataacacataaaagagaaaagaatgatcagaaatccaaatgaatctcatttgacttttagcataaattttcataaccaagaagttaaaggtggaaaattaatatagaatactctcaagctagttttatttgtttcttctgttcataatgacatactaaattttaattttaatatgaagatatgatggaagtgatattttaatctacattgacaaatcgtattagagagtgctgaggttgtgagataatgcttattgtattatctctctttacatttttcctttttatccttaaggctgtactaagtttttaaacatgaatgtgaatcgtgtaaaactattttaaacattaactcttaaatttggggcagttacaaaaaaattaaattaatgcaaacacgaaaacaaaatattttaagtatatatttttctttcctccctgagttaaaggaattcacttacttccaactacaacattttatgttttattttaaaattgtgctatcaaaattatatcttaaagcagtgttttcttatgaaaagttctttattgaaatatacttctaggacatgataggcatttccattagcttgtcatgtactctagccagagttaaattattattgagttgtccaaacattatataacaaatagatcttcgtgaacatatagttttgcctcaacttcatgtaataaataactttaaacatccatcttatttttgttgaaatcatgttgtttttaatatcatggttttgtgtgtacatattatgttatctgtcttcataaacaaagacatggtccttataactgccatcgtgctcattttgcaatttagtatatcaagttatgatttttgcagatatataaatagatatattcaaattgggtaattcaagagagtttaatggagtccctattggagagggggcatccagctactgcatcaccaagagctgataccaattcaatcatgcttccaattggaatctaaaatactagccatccctaattttcttcataatagatagaacataaaacagagttgctacagtttttgctcctctaagttgccagaaagctcaaaattgatcattatagtgttcttcttccaatcaaatacatcttccccttactcttactctgtttagcacatcggctaaataaggttctttatctgataggtgactaaaattttataggttctgagtcttcggtggtcctgtctttcttatattactataatttttcaataaccaaaattatgaggtaagtatttctaaagagatgccccagtaaatcccctgagttccaagcatagttcttcttgtcctcattgtatagcaacagctccaaatcattctggtagtctggaaaaattataagccagtccctgtttatttagcagcttgacatgcccaaaatagctagtgagtattctcagcttccaatacaacaggaccagatctctgaaacctggagaaaacattcttcttttgggaactgagatctccaaaaccaccaatctgaaggtttcaggacacagagtaaaaagcctttaaatgggttatttgatgttattagagtggccacctctaccttaacctctttgttctcagactcatgccttctggctatggaggagatgatgttggccatatataatatcctgtagaacacacccaaactcctcccagctggcaaccatacatgaaccactcctttggtaggccatttcaccactccatgtagccagctgattctgtgtggtgatacatggggtatgtatgacccatgcattctatagatttgcgctaattgtatttgtcttgccataaaaaggagtttcctggttgaaggcagtatagcaaggaatacaagggtaattggtaaggcattctgttaaatccatatttgagggagcttgcaaaaatattgaagagaaagggaaggcatatcctattaggatatatttctgcctcttccatgttggaaagagtcaaatgtaacctgcctgccactacttgcttggttgatctctctcaggaatggtgccatatcagggtctcagcactggaaaattgtgagctcagcagtggtggtagttaaatcagtcatagagaagaggaagtccatgtaattgaatctgttcatagtttctgtgtccctactcccattgtcactttgtacattggcccactgagaaagtaccaggtaagcctggtaaagagtctgagattcatgagattacccccttcagattactaacagccttcactacagtgaatgccctctggagagaatttacatagaataccatatcattacattttgtaccaattctgaaggatatatcctcataactcttccctagactttctgtcaccagtcgtccaattttgttctttgaagtccttgaccatatggccatgccatatgtcattacctcaactatctcttcttccatgcaaagtgtgcagttaaatgtactagctgaatgaaattctggctgctggtgggatagccactgtatttcagggccctctcccttaagtggacctgtaaacaccatagtcatagctggtattgaattccagacattctatttacaaaccagatccacactgtttcatcctcatcaactagtcactgagaataccccatgaggccatacatacgggttgaagcaaaaagattgagaagcagtagtagcaggtattagggacctctgagctacttgctcatacagtatacttgggcttcctcaactagcataagaaggctgcagggaaggaaaatataccaaggaaatgctgttgtgtacatttttgatatgttgaattagataacacctagttcatgatgcacggtttgggctacattgtcattccgagtcctatggcccagtacacagtcttaaccaaagttcattagcaagtaaggagtggcttttcaaatggagattagttattgttagaaaactgcatgtctttactataaaaccacgagggatctacacactgaggcttccattgacagagatgtcatacagcatctccatttgccacagacatttccaaaaatcattgcatctgctgaatcttagagtcaaaagtagagggagctttcatcatggcagggtatgatgtagatccttttcttgccccaggctgctctcaaaatggacagcctgtggattacctagtatatgcaccacagaagtatacccaaatatggtctatgttgcttctaaaatctagagaggccaccacattttgtgcttcattttcacgaaggaaggtgcagggtgcagcaatttctcacttacaatggaggatctatttcagcatgcacagatcagtggcctgtaaaattttaccaatgctgaaagtccctgaactttcttggagtttatctcctattctatagcatatatctcttactgaggcatctcaggtatttactatatcctgctcccaagttcactcagtgtaatgtcatgaatataatattccagcttgatgttttttggaatgtcaagatgaacaagatttctccagactatgttatgacaaagagagcagagttgacctagccttaaggtaagagagtaaaagtatgttgctcctctagtgtgaagggaaaaaactgcttctgattatctttgttatacaaattaccttaaacagaaatcaagttcattgcttcatacctagtgtcagtggctatgttaatatgctccagtaaagattgtacagctaaaactgcatctacagttagcattatgaaatgattaagtctgcatcaatgccatgccttagcctaataaacagtttaaatgggtgtatagtgtgaattatcacccctaacatctttcagttttctgatggtttcactactgtctgttattcatccatagaattattatttcttttagtttgctgtcttgggagaggtgatagttgtagggctacgctgtccaatatggtagccactagcctcatgtggttactaaacacctaaatatggctagtgctacatgttgaaataatattttttatttttcttgtttttttactacagttttatgtatattaactatacctcaatgaagttgttgagggggagggagaagatactaccatcccatgtttctctgctttctcatcattgccccactaccaaaaactaatggtcttagtattgtccattgtatttagcattgcaattctataacccttcaaatcagattctggagttggtcttcagccttatttgccttgaggcttaagtggataagggacccttttaaagctgacataaactttcttgtattttgcctgtattctgacttgagtattccaggcactcagtttgtcttccacctttgtgtgctccttagggccatcagccatatcatgctgtaatctttatacactaccattgctgctttagcaagtaatgccatggctcccaaagccttaccctgcgtcgtacatccttccatgctaccaccagtgacaatttgaataatcatcttgccagcacatgccttggatcactactgtcccattaactctgtgataaggaggctatcctgctatccttgaattccattttgactggagctttgggccatttctgatatcagtaactctttatcagtcaggatcctgacaagaaaacagtaagacactcaagtggggaaaatttggggacatgcatgaaagacaaatattcacaaaagaatggttaggaataagggtagtctacaaaaagatggctgggtttacagtagtaggaagccatttactagctctagcctgaaagagaaaggggatctagaggtcactggaattaagagaaataggaaagggtcttgagcactgaccttcggtagaagaatgtagcctttgctaacctgtgacttggggaataaacaccctgacctcactctttccacccttcagtctcttactggtgcctcacattgactaaacttaaccagaagccagaggacaacggagccaattgattaaagccaataaaaattagcttttgcgggagcagagcaaagaagtgtggaaaatggctatggcagccaaacagagaatgtgccacagattaactaaaatttaagaactagtgcttaaaattactagtatagagcaggggtcctcaacctcctggctgcagaccagtaccagtcactgggtggcctcttacaaattgagccgcatagcaggaggtgagcagcagtgagcaagcattactgcctgagctcctcttcctgtcagatcagtgacagcattagattctcataggcacgcaccctattgtgaactgcgcatgcaagggacctacttggtgtgctccttatgagaatctaatgcctgatgatctgaggtggaatagtttcattctgaaatcattcccccaccacctgtcatttgtggaaaaactgtcttccatgaaactggtccctggtggtgaaaaggttgggaactgctgatatagagtattaaggggaatataatgattttaaatttaatctctgtttttatgaatattattattggtagaaagtattagcctccctttttcctccaggtaataactgtccattgttttattgtattgttatttatcatgttggatattaataccacatttctgcttcctatattgtagacttaaaaatttctcatcttgattttcatttagtaggttggtaacaacaagcccagcacagatgtgcatgaatacattactgtgatagatgttacagtttaagaaatgaaaaatactttgaaaaatatctggaatacaatatgtattaagtaaatactaggggtgtgtgtgtgtgtgtgtgtgtgtatttcatttaccttgcatgatccctgtacaaggtggtatttttctgcttaggaactcttcttgtcctctagaggacaccaagtagtgtcttggtttttattgcataggcctggtagttaggttatttatctggaatttcttgagctttccctagacagtagacactgtactaagcattttccatggtttctttaataaaaaacctttttatctaacataagcaaaaagagattgtagattaatagcaataagtctagtaacatggaaaatgcagttttgaaaagtttttactttaaaaaatataaatgtaatatatattttgttatttatttttttatttaaggccaagctcttttctttcagtgagaaatttggttgttgaatagcatgtaatttttctggtctaaaccatacccataacacattatctttagttacattttcagtgtctttaagaacttaaagatcctgtgaagcagtcagacttctattcatttttaaagattttttcactaatttttatgattatttttgcccacattcacttctttagaaatatttttatcagcttatctttattgaatcttttcaaagtggtaaaatttggtttcatagaaacagtattctttttgtaattatttcgttggaaataattatagcaggtaaactggcataaataggttttgctacagtcatacctgactcttagtgactacacatacagctcaactgctagaagtatactcggacgtaccaaagaagaacgtatgagctccaagaattcagtgtttttggttatccattagtatgttttacaaagagaatggagaggcaggttcattatgagaacctctcactttggttattcctattgtacactgaggcagaaaagaagtcacacaggcagaccctggatttaaactaaggcagcaaactccacaatttgtgctcatcacaccctgctaagtgataaatgacacagctcgtatgtttattctataaagctttaactctactgaagtaactcctaatgattgaaaaaaaaaatgtgctgtctagaagtcaggagatcaaggcatgtgggttttagggtggccttgctgcaaattagctatgggacgttgtcaagtcacttaatttactctttaaatctcctggtgctgctattatatgatgcgaactacaatgtgttatcttttctccctggattttgctaatcagtcactactaccacaccttaaagagtagtctccactccctccaaattacttctttttgtttgtttgtttgtttgttttagtattttgccctagtttattttaaaataagttttcatgaaaccgaattaacatcttaaggtttatattgagtcttcacagtgccaatatttcttgtctactaatgtctgatatcctacatctgatagccttgaagtgtctaagaaagaaaaaaataataactttttttatgtgaaatgtaaatgacattttaagaaggggaaggagaagagggaaaggggagacaaataaataattgaaatgttttatgaacaaagatcagctaaagattacagtcaagttgtggccgggcacggtggctcacacctgtaatcctaacactttgggaggccgaggtgggcggatcatgaggtcaggagatcgagaccattctggttaacatggtgaaaccgtgtttctactaaaaatacaaaaaattatctgggcgtgatagcacacgcctgtagtcccagctactcgggaggctgaggcaggaggattgcttgatcccgggaggcggacaccagcctgggtgacagagcgaaactccatcctcccaccccccccaaaaaaagattacagtcaagttattagcccactgagaaagtgaactgcacttttaaaaacatgtcccagagatacattacataaagttataggaaaataaaaggtattagtctatacttgaaactcttgctattcagtaatcttgagatttactatacaaatcatctactttgcttcatgtgaacttttaacatagtatttaagtgacttttttttttttctgagacagggtctcactctgtggcccaggctggagtgcagtggcgtgatctcggctcactgcaacctccgcctcctgggtttaagtggttcccccgcctcagcctcccaagtagctggaattacaggcgtgcaccaccacgcccaactaatttttgtatttttagtagagacggggtttcaccgtgttggccaggctggtcttgaactcctgacctcaggtgattcgcctgcctcagcctcccaaagtgctgggattacaggcgtgagccactgcgcccacccttaagtgactatttaatgatgaatcaatgcttctgctacaaaaatgatttttattagttttgtctcagatttttcagttttataggtgattatagttctttcattcttataaatttgattactatattttgtacattatatatattacaccttttactttctttattatacctaatactctgaggggatagtgtttaaattaagccctggacccctattagtcaaaataatatttagcatacttgttattttaaaaattctaggaggaaatagaattacatgttttttctaattagcatttcaggtatattttgtatatactcttatgctgtacccaaattaaaggtttaaaacagaagagtgtataattttaatatatgagaaacatatatgtgtgttaaacatttctttttataaattataaagagaaaaatgatgagtaagaatgtgtctccatagaggggaacaacacatactggggcctattggagagtagagggtgggacgaaggagagaatcaggagaaaataactaatggtgctagccttaatacctggatgacaaaataatttataccaaaaaaaaaaaaaaacccatgacacaagtttacctatataacaaacctgcacatgtacctctgaacttaaaataaaagttaaaaaaacagaatgcatctcatagagtctcaaaatatttttctaccagatgaaaaattcaattttggctaagaattatactggttcagattactatgaaaattatgtattgctcaaaaacggaatcatccaaatacatttgaaaattaaaaatcttaatttcccaaagacatttattcattgtctcttataaatctaaaaaagccaaatacttattaagagaaccatatatttaagatacttgttctaaaactaagttatttttcaaagatttataagtaaaatgtccttaagtaagaaaataagtaaaggaattgttttctaatatttttgttcatcctcattttaagaaaataattatttcatattttcaataatgctattaaattctaagtaaaacctcagctttcaattgaaccagcattgagcatatgatataattatttatttgtgtatatcacttaaaaataactactttgtaaaattagttatctgtatcacataactttggagtaagagagatttgaggaaccttacaatgtcaaagttcttttgacttagggttgatatgcctaaactattctgaacatgtatctatcttacttccaaacactttaggaatgttgatttttacaacactactttgtgacctactcagtgtttgacaattttcagcttatgaagtttgttgtttatgtctaattgtcagtttaaagctattctgttccataaaaaggaactggaccagatgtaccatataaatccccatctgttaaaacaacaacttcctcccaactaaaaaacatttttagtatatatatatatatggactatctttatttttcaagtccctatagttccaaggactatattttatccacaaaaatagctcagaaatattgaatgcttatactagttcctaatattgacttttaaaaatcccaattaaatagcctagtttatattttgttcagttatgccttcacctattatgagaaaattcctgtaaagtattatgactcatcctatgtttaaatatatttccacatttatctttaatagaatttcaaactaacatgcatttttaaggtatatgctacatgccctcagagaaactggtaagacaagatggtcttcttcatatcttcaaatttcttaaaaataaaaaaaaaaaattaccatgtggcttggatcaagatggagctcatctttgaccatattgtttaaagaaacaacataatgttagttgctctagttttaaatctcatagaaatagtctcttaatttgtcatgagagtagtgtaccctcaaacgcattgttgtctcttactaaggaatttattatcacaatcacatcgtttctttaaattgacaactgatcagtgatttcataagctaaagtattttctaatgtactgtagcatttgtcatcacttactctcttttataatacaccagtttctattttgttttatttaaattatcttcccttcttcttttgagtgttgtggtctgctctttcttgtctttcttataattctttttccctcaatccttcatccatttgcttccatattacttcactttttttttttgtaatgacttccagaaaaaaagtacataaataacttcttcagtgtatgtatcatgcaaaatgttttgtataagatgggatagctgcaagttatacacctttggatattaatagtagtgatagttttgtggaatgattgaggacaaaaatttctgaaaattaggtacttggccagaagattatgataatcggttgttttcctatttacattattttaaaaccagaccagctggacacggtggctcacgcctgtagtctcagcactttgggaggccgagacgggcagatcacgaggccaggagatcgagaccatcctggctaacacggtgaaaccccgtctctactcaaaatacaaaaaaattagctgggcgtggtggcaggcccctgtagttccagctacgggaggctgaggcaggagaacggcgtgcatctgggaggcggagcttgtagtgaacagagatcctgccactgcactccagcctgggtgacagagtgagactctgtctcaaaaaagaaaagaaaagaaaagaaaacaaaacaaaacagaccaccatgactttttttttttttttaaggatagaaacattgaattaaattatgctggtttagaaattgaatccagtgcagctcattcataatagtgatggtgcattaaatgtagtataatttgtcttgcaggcttctcatgagaaggaactattttaaactgattgtagacctggcacctggcccactttctgggtgatctcccaagtgtgaactttccctacccttagcccctcctttccttcctggaggtggctgatagaggtggattatactgataattacacaagcagtgtcagttgcacacatgatttgcataactgtgctgtgctgatttcatccatcaaattctgatactgatgcctttggtgcctcactgtatgctttgcaataaacactgaagaaaggagatgggattaatattttcattcttgactcttaaaacacacacttgaggtcctactcattgaaatttacctagaaggtaaaagccactttcagtcaggccggctgtaagacagcccatttgatttccaccaggtagaacgcctctttccccctaccccagaccaataaccattatgtatctactcttcgttattgtttctaataaaagaaagtaaaacttttaatagcatatacaatcacatcttttatgaattgtagtagtcatgcttctttcagaccctatttctagaaggaattaataagcctttcttttataattctatgcagctcttttcactttggtgcaagtcatttagaagatcaatttattgtaggaagctttccttcctcctttattatcaggagttctaaagaaatggacaagattcttagctgctttctcattgttctgaataatctacactgtcctcattccaaagcttgctaccacagttttgtaccctgcatggatttagtgtggacagtcttaactttggccatttcacattttcccttccacaccatgagtctgcaggcctggcatgtttggaactccacaacagaagtggagaggctgcacgcaaactttattactcacctagtatccagaagggcatgacacactgggacttgtgtgtgtgtgtgtgtggtgtgttttgtttggatgtttttttctaacctaacctttaccatttctttcaatttttcctacttgatctgtgtatgtgagtcaaagaaacctgttcatccacagcctatactatcttgccaaatactgtaagaaaactctcaaatggtagaaaatcagacaaataagaaagataaaaaccaagtatcattcatcataagcatgtaaaatatatttagaatataattaaagttttatgtaaggtatcagaaatctatcagagtggagtccttttaaattgtatagaaatatgatgataatgattataagcaactggataagaataaaaggtcctagatccagcatctcaaagaagagattttgcatcttcatcccttatataccatattgattgaatgaattaatggacagattattttatttgaatattcttatattaactagaaaggttttaaagattcatggaggtcaatggtggttgagtatgtttcctagtacatgataagtattagtctattacaaaaattaaaaattatgggtatatttaataagttcttattttgtgacaagcattatgttaagggccttattttgcagataagtagagtcaagtttaaagaaagagatgttacctaatgtcctcacgcagtcattttacatattacatgtatgcctcctcagcttatacagattatactttcaccttatttttcttttattttccatggtttggcaacaatgtattattggattagatttcaaacacacacatacacacccacagcacaataaaacaagaattgtatcatgctcttataaacaaaaatctatcatgaggcttgaagacatatagcagcttagaacaaaataaatctaacaatagtataaattatatttgtgagagtactttaaaaattatgatctgatatagcaaaaaaaatcataccttgataactatatgtttacattttatccaacttccaatatactcatgaaagctgagatataagctctggtctgtctgaatctagggtctctgctctcagccactatactcctgcttcccaatctattctccagatagcaaccagaatgttactttaaaatctgatttaaaccatgtcactcctcttcttaaaatattttaatggttttccattgcttttataataaaatcaaactactaatcatggccattggtggttctgcccacttctactgtctgatcttctactactttacgcctcactctgggttctttgcaacccgaggtttctttattctttcctactgtttttgaactaatggatgatcctagaagtcaacaccttcagcgttttcccaagatcttggcatagttgccttattatctttcagatgttagtttaaatgtcatctctttgaaaatagtggcatccataagcaacctaccagaagcatccccttagttctattttgctatctctatcttaataaaaagcttgaatcagaatctgtggttatgttggtgtttagtttatacacatgttgtctgtgccatcccacctcactcgaatgtaagctctgtgagattgtggactgcaagactgtctttgtggagcttacattttaataaggggagacgggcaagaatcagttaaaaaatcatataatatgttagaagatgttgactgctacagggaaaaaaaaagattttccacttattgatcatttattgtgtgttaagtaccttgagttttccatatggattaaaaaaaatatatactcacagaaatgctgtgagttaggtacaactatttcctaatttaacgtaacctaggcttcacacacaactggcattagggttagaatttgagatccaagtaagaaaccacattctggatatacaaatacatattctttatatgaatattccataagttagagcaagctatttgcttctgaattgattattttgggagctgtttgctatccaaattatttgcttactcataagcctgagttcttgtaatatagaaagtcagatgaaaggaaatacaagcataacttcctttattgtgccttactttattgcacttcacagataactgcgttttttacaaattgaaagtttgtggtaaccctaattccaagcaagtcggttgataccatttttccaatgacacatgctcacctcatgtctctctgtcacatttaaataattcttgcaatatttcacatttgctcattattatgtctgttttgtgatctgtgatctttcttgttattattataactgttttaggataccacgaaccacacctatacaggctggcaaacttgaatttttgtgttgataaatgttgtgtaagctctgactgcttcaccaactggccagtttctctgtccctctgtccctcacctggcctctatattcctgaaatgcaaaaatactgaaattaggccacttaataactgtacaatggcctctaagtgttcaagttaaaggaagactcacatttctcactttaaatcaaaagctagaaatgatgaggcttagtgaggaaggtatgtcaacaggcaaaacatgccaaaagataggcctcctgtgccaaacagccacattgtgaatgcaaaggaaatgttattgaagaaaatttaaaatgctactccagtgaacacacaagtgataagaaagcaaaactgccttacttctgattatggagaaagtttcagtggtctggatagaagatcaaaccagccacaacattctcttaaatcaaagcctaatcctgaggaagaccctaacaatattcaattttctgaaggccacgagatgtgaggaaactgcagaagaaaagttggaagctagcagaggttggttcatgaggtttaaggaaagaagccatctctataacataaaagtgcaaggtgaggcagcaagtgctgatgtagaggctacagtaattcatccagaagaactagctaagctaattaatgaaggtggttgtgataaatggcagagtttcagtgtagacaaaacagccttctattagaagaagaggccatctagagaggagaagtcagtgcctgggttaaaagcttcaaaggacaggctgactctcttggtaggacaatgcagctggtgacttaaagttgtagccaatgctcattgaccattccaaaaattctaaggtccttaagaattatgctatatctactctgcctgtgctctataaatggaacagcaaagtctgggtgacagcacatttgtttacaatatggtttagtgaataagcctattgtttagacactgctcagaaacaaaaattcctttcaaaatattacagttcattgacaatgtaccttgtcacccaaaacttctgatggagatgtacaagcagattaatgttgttttcatggctgttaatacagcatatattcttcagcccacgctcaagggttaatttaaactttcaagtcttattacttaagacatacattttataaggctagaggtgccatagacagtgatttctatgatggatgtgggcaaagtaaactgaaaaccttctggaaaggattcagaattctagatgtcattgagaacatttgtgattcatctgagaaggtcaagatattaacaggagttgggaaaaagttgatttcaacctcatggatgactttcaggaggttcaggacttcagtggataaaggaactgcagatgtgatagaaacagcaacataactagagttagaagtggagcctgaagatgtgactgaattactgcagtctcgttataaaatgtgaatggatgaagagttgcttcttacaatgggcaaagaaagtggtttcttgagatcgaatcttctagtgtagatcctgtgaatattgttgaaatgacaacagaggatttggaatattacataaacttagttgacaaagcagtagcagagtttgagagagttgaatccaattttgaaagaaattctactctgggtaaaatcgtatcaaatagcatcacatgctacagagaagtcctttgtgaaaggaagtgttgactgatgtggcaaacttcattgttgccttatgttaaggaattgccacatccatcccagccttcaacaaccaccagccttatcaatcagtagccatcgacatcaaggcaacaccctcctccaccagtggaaagatgatgagtctgaaggctcagattatctttacagtgtatgtgtatgtatgtgcagtgcagtggtgcaatcacaactgactgtagccttgactgtctgggctcaagtgatcctcccacctcagcctctcagcctcccgagtaactgggaccactggtgcatgctaccacaccctggctaattttgctagttttgtattgtttatagagatgagatttcatcatgttgcccaggctggtctagaactcctgggctttagtgatcctcctgcctccacttcccaaagtgcttgtattacaggtgtgagccactgcacctggcctctttagcattttttagcagcaaagtatttttaaaattaaggtatatacattgtttttatacacaattctgttgcacacttaatagattatggtatgatataaacataacttctatctgagtgacccactttatcaggatatattctttattgtgatggggtggaactcaacctgcaatatctccaagatatgccagtggtttgattaattgaaatctcattaaaaggaagagtctatataatttggattattcatttaatctgtatgcctagtaaattcttactagcctttctgtttactttttattccccataatgaatattttgcacatttgtaataatgaaacagtatcaaatatatctgctgtttcccttgtgtcttagattttactgtaccatctcctggccacattaaaactttttgcacctttatactttctctagctctttttcctaaattcatcaaccaatctgttaaatgacttatgttacacttacagtgaaagaagtgagcatccctaggggttaaatggtttttgaatataatgtttattattatgattatatatgccacattttaggcagctggtaaatacatgttgtatgaataaacaattgatatgtatatatttttaacagaagtgtaaaaatttagagagattaagcatctacttccatttattgtttaattattttattagcaatgttcaaaataagcaataaaatattttatgtggtaaggaaatgctggggaagcataactgttacactgccaaaatatgttacaatgtatattttgcttcgtcccattttgcttccttattttaggtgttaagttcaagaaatctgaggaaatgtgttaacttaaagtgatagaaaagtagatgccaacaacttagactcttataaaaataatcttacataagacattttcttgtttgaattgtgactatttttcttccagagtgcccaaaacaataaaaagcagttttcttttgctttcaaatgtctgtgattttactttaaaggtcccatacatacttttttatatacaatttttcctggtgtttgtgtcctgtataagattttcagtttactttgaacattgttattacattataaaacaaatggcaatatttggatggttaataatgttagctctgtggatgcaggaatgacatttgtctcgttcagtgctgtatctacagcacctagaacgtggacatggaatactggaggtgctcaagaaatttatgttaaaggaactagatgtctttgctcaacacctggcacatcctgttaaagaagagtaaacagtattaacacagcctggcgcagtggcacacgcctgtaatcccagcactttgggaggctgaggcgggcggatcacttgaggtcaggagttcaagaccagcctggccaacgtggtgaaaccccgtctctactaaaaatacaaaaattagccagacgtggtggcacgtgcctgtagtcccagctactcaggaggctgaggctggagaatcatttgaacccaggaggtggaggttgcagtgatccgaggtggcaccagtgcactcctgccctccagtctgggcgacagagcgagactccatctcaccaaacaaaaaaaaaaaaaaaagaaaaatagtactaatgctggctttttttttgcctcttttattgtcataatgcagtgttcttcacagtatgttcacaaaaaaatatttacttgcgaaattaatagttgctaaaataaaaaattgtactatatttagtttttttggaaaacgttgggttaaataagttttgtctgtttgcttttttgttattgaagctaaacatccaaaagaacctctgataatgctcatatacattgtaaatctctaagaaatctgcattaaatatactgtctcacagacttgtttaaacatttaacattcatccccccaccctcaactactttagtaaactgtccttagagagataaaagttattttactgtaatgatttttgacagacacctaattatcctgaaatgatttcatataaagagattctctttgaaagatatctcacttttcaggtagagatctgtttcctttctaaaatattcttttcccacttaatcatttcattaaaaaaataaacctagagtcattaagacttctaaagatttagaaaaaaaaagtaggtgtggatatttctaaaaaattccatatcaggcagaattttaaaacttaaaacagtaaagaaatcacaacgtgggaaactaccaacaattttgaccacaagaaaatgtaaaacctctaaacctcaaaaaaagcataagtcaaataggtaagtcaagaaataagtcaaaggcgttaatatatacagagatcaatttctgatcaatttattcagaaagacaaaagcagtaagaccataatttaaaaataggtgaaggcatgaaatagatcatttgaaaggacagcaaacaaatgtctaatgatttttttgtttaatatttcgtttcactaatgcaagtgtaaaagaaatacaaattacattttattacctagctcagtgtttcccaaactttcttggttcattgtgactgtagtgtcttaataattttgtcaaagttcatttagggctaaaagtaatactgaaaagtttcattttattaagtagtaaggtccaaacaacttaataatgatttgtgccctaacaactttttatccatttgggaaaattataatatataaattgaaacccaaaacagggttttgttttgttttttcatcttagatggccacagttacttactaatgggatggtatgccttgttgggcataccattagtaagtgaaatcgaagactgattatatgagaagatcaataaaatagataaacctctagcaagatcaatcaatattaaaagaacacataatttggcatcatctagaataaaaaccggaaatcactaaaaatcctgtagccattggaggcataatatggggatattttgaacaactttatgtcaaattcagctagatgaaataaaaaatagaaattattgactctcattcaggaagaaatatacactctgaatagcccacatttattccaggaattgaaattgaagttaaaaaccttcccacaaagaaaactccagatgacttcactggtaaattctgtcaaatatttaaggaaaatgtaatttaagaaaaacactaaaggaacaaattcctccagacaatagaaaagggaggagggaggcctccaaacaaattattatattcagcattactctgacaccaaaatcaaagacattacaagaaaaaaatactgcataacggtattcttcatgatgcagatgcaaaagtccttaaaattttaacacattgaatctggaatatataaaaaggataatatatcatgaccaagtgggatttatcttaggaatttaaagttggtttaacattgggtgaaaaaacatcacaatgtaattttccatattaacagactaaaaaagaaaagattttagtctgtctttaaagtttcagaaaaagtatttgacataattcagctttcacatattataaaaattctctgcaactgagtagaaaagaacttcctcatcatgatggttatcatttgatgagtgtaataagaaaatatggacacttaggttgatgccatatcttggctattgtgaaatgatgtaataaacatggtaatgcaaatatttctttgacatactgagattaaatcaaatgtatttctatatgctaggaatgaacagttggacattggaatttagaaagcaatgccattgattataacatcccaaaatgtatgaaatacttagagttaaattaagtaatatgtgtaagaactgtgtacgtaaaatcataacacattgctgagataaattaaagacttcaataaatgcagagatgagacattatcatgatttaaaagattcagtattgttaagatgttggtttctcctgaaatcgagtctgtcagttcagggcaatttcagtcaaaatcccaacaagctttttaacagaaattggcaaactgattacaaactttgtttggaaatgtagacctaaaatagccaaaataattttgaaaagccaaatttggatgacttgcactgcctgattccaaatctattcctaagacttgctctgttggtttaagtatagacatagaggttaatggaatggaatagagtccagaaatagaacccaaattggtcagttgattttcaacaaaggtactgagataatttaaggggggaaaggataatattttctataaatgggtctagaacaactgaatacccatatttttgaaaaaaatggtctaagactcttatgccatatataaaaattaagttgaaatagccatagacttttgacaactatagtaattatgtgaatcgatgttaaacacatatttatttgttttatctgtatatatgtaaatattctgcaatatgtgaactagtatatacttattatatatagtacagacacatatttcttgagaggtaagggacaaacatccaaattagagactttagaccagagacatacgctcatatttcagaatttcagaacatcaaggacaaactgaagttactaagagctttcagacaaacaggccacatataaagctatatttatataatacttttaaaaagaaactttggaggttagtagcccagtgaaaaaaaggcttcaaaatgttaagagaaaatgagtttcaacctggaaattctaaacccaacgaagaggaaaacatgggattcaggcagcagaagactaatcatgagggggacaaaggaaagttctggataacaactctgtaataaacccagggtaccatcagttccaactggagaaaggggtgccaatggttgtgagaaggcaagaggaaagtgagactggcaaggacctatgacattgttctggactctgagttatcagtaaggtgtgagaacctcagaggaaggggtaagtggcactggatacagaaacacaagcaaaataaaaagtgagacgattattaactctaggaaaaattaaaagctgcacaaaaaaggaaatgtaatcactgttgagccaaatagtatgttatgattaatatttctatgactactggaaatttccatttctatactattaatcagaaatgattatgtaactattgaaagaccacagaagtgagaattgagagggtgtttgtaagagagctgaatccttaactaccaggacagagaagcaatagattatctctaaacctgatgaataaaaataacccacatattatttagaaatatgaatataatcaccaaaagaaggagctgaaaatggttgctttttgggattttgattagattgcttgggggcaggtagggcagcatggggaagagactatgtatttcattaaatgccacttgggactatttgattttttttatgggcatatctaactatggtaaaaagaaaaataatcattaacgtaataaaagaaaaggactcagaatgctgagcaaactgaataattcagcattaccataaatctagaatctgggagaaagtgttacctaacagcagcaacaaaaatatcatttcataataatggggaaatgttcaaaatatattgttcagtaaaataagcaggttataaaataacagtgtataaaataactcattttcataccaaaatttatctttgcatgcattaaaaaaatgcaagaggataatacagaaataccaaagacagttatttctaaatgatataattatggattttactggtgattaaaaaaattttatatgaatattttacatttgaaattagaaagagatttaagaaaaactaatgagaaaaagctatttaaagagtttggaaattatatcattcataaaaactccacttattagttctttctatgagcaaagcaccctgctaaactcttcggaagttacaaagacacataagatattgttataatttctcaaaaaggtcaagatctaacatggagatgaaaattagtgtgtaaataccagtataaaagtcacagataatataagatagaatgatagagtgttggaagtgttctaagcatgagggtcaatacaggaatagttctttagtgagaaagatcacgttaaaatggaacagagttaaggaggttggaagtggagggatgtggaggatttcaagaggtagaaatcatgtgaggcaattacagcaacaaacaaaactcagaagtgtaggtgtaaagtaattttgactggggcctggggtaggagtgggatgtgtgctttgggaccagatggtatagggtgctgaatgccaggtaaagggatttcaacttagttcaataaaacttaacagttattgaaagttgttggagagaggaataaaataattacacacgggaagagaaagcctatttgaagtctattatgccaggcccaggtgagcaataatgaaggcctgaactaggacgttgattgtgagaatgaaaagaagggatcaacttgagaatcattttacgagtccagtatatagaatttagcagctatttggaagtaggaaaagaggtagaaggtaacagaagtgggtgcattgttgtgaatcttgagtattagaacagtggtaacactagcaaagaagggcagtcagaacaaagagggagttttgtggaggaagatagttttgggtatcttgaatgtttgatttggttttgggggaatatccatgaggcaagactcaaaaggcagttgcaagtatggatttgaagtagtcattcagcaaatatatattgtctcctgtgtttaataactcgatatgtattatcaatttaaaattgagtaaaacaagatctctctcctcatgaagctcatcgtttggactagggaaataaataggtatccgatggttaattacaacacagttgttataagtaatgcatcaacagagtacgaacagagttctgtgagagcagaggagacagctatgtcctgatgctcttcgttttggttgtagggaagggattcactcagacattgaaagatgtacatggccagttggagcttacaaacatgggaaattattcttgacctgatgtggatgtttcataaaaatgagcacttcatttagagagcaaagagggagaacattgtactgagaacaatatctgaagggatgcatataacatatttgcaagttaggaaaaaactgagaagcagtgagggcgaatgtaggatctgatctcagacgctaaaacaggagacccaagacacaagaaagcttgatagtggccaagagattaggaagacagaggattaataaaagcctatggatttagtgattcagagaccactgacttttcaaaaatgtttcaataatagcagaaaaagtccgcttcagttggtaacaatatgaagtaataataataataactattataaaaataaaggtagtgaaatattggtccttttatatgaatatatatgttatatgtattaattatgtattactttaaagataaactcatgtacagataaacacatgtatagggagtaagtataaacaatatttttatgaactttatttttaagaaatagtatgaaatggatagaaaatggtcttgtggagatagcagagccattgaatttgcagggattgttggcaatgtctgaaaacaattttcaaaggagtagtaagggcagaatgtagactttgaaggagctaagtatatagaggaaaataaatgactacaggtctcagaaatcctgaaatcctgcctttccaccctcactccccaattatataatgactactttttcttcagttgtcacctgaaatgttcctacttcaaacagaccttcttgttcaaccttttcaaactagtttccccatgttatactacaccaccattcttttgctccatgcaactgcccacaatttgtaatacattgcttgtgtattttttaaatgtctgtctgcctcaccagactgtcagttgcatacggataggcaccatgggaatttgttttccactttacattttagcctcatgagcattggtacatagtagtctgttattgaatgaatgagagaggtaaaaggatagattgtctccagagcaaatttattggaaatagaatagttttaagaaatgtccaaatccaacatgtcacctaataattgggttgctggaatagaatggagatgaagagttgggaaaattagggttatacatttagaacaaagcccaactcagatccctgttggactcaatgatgggatgaagaggaaggagtgatgctttaaaatagaggacaaaatgggagaagctgaaataaagcagatgggatagtaatgagagtagtaaaaagtgagtagaaaacataaaaactacatgtctcttccttcttatcaggtgagttgggacttttagagggttgtgagccaaggggaatagaatgaaaaaccatgtttcaattaaattgagaaggtataatgattgcttacagaaataaggggaagaaagtataggagaatatgctcatagtacaacagaggcatcataatgtgatgggactaataccagggaagacccggaaatgactgaagtagtttacagcgggctattagcttcaatgtaagtgcttaataagtattaattggttaataaacttgttcagtataaattcattactatttctagagaaatattacagaagaacccaaaactgagtaatgctaatagtaaacagatactttttgttatgtagtcccatatcttggaacataattacatgtaaatttatttatagtatttacactagatgatttcagcaaaacttattttttgtatttttaatgctgtcacatggtgatacaagttctgcacagtattctcagctacttcccagaaatccatggttccaaaaaatagaagctggtttgccaagcatttttaaaaaaatgaataatcactccataaataacttttatgatagtttacaaacaattatgattatgaaataactgatcttaatttatgtgtacctcaattacaatctaaaggtttggtgtagctttctttttcatcagtacagtgaaactttcaggcagccatgaataccttgccttgttatagtatttcagttcctcttgtgaccagtgtcatagcatgggtttataagttaactggagttagggatgctatctttggtcatgaaaaagagttgtctgttattctctgagcaaaactaaataattcctctgggagtcaaatagagacacgggcctggatcacacggtgctttactattgagggctttggcacagaccacctggggttttatgttgttctgtattcattcaaaaccaagagtcaaagtgctctgtgatgtccttatgaagagtcatgcattaaaacatgcttctttccatagagattatatttttcttctttgttttttaaggttttttttaactgaacatcttagtattttaacagatgtattgagataatgacactgagataatggctccctagctattagggaataattcatttccttaatgtgagatgttttggaaaatattactgtcattttgtaactacatattgtgtaataatcctgactcaaagcacctagtaggttaattcttggtattataaaagctgatgagggagaatatacttacctgccacaaaattttgcatgtgtgaatagttgttaccaaggtgaaaggaaaggaaactctactttcatctgtactttttctcaccacttgagttctttttaatcttcttggtacccaagaataatctctttattaaactgtgacatttcaagttgttgcattttaagttttcttctgtaaaattctctgcccttacccgaaaagaaaacaaacaaaaaaaaaccacaactcttgatgtatctaggatactcctgatttgtcttgaatgagtcaaagctgagagtcagtgtattaagagtgagataatttgaagactgatcaccaggccctcaaggtctagaatacagtcagagcaagaatttgattcagtatttcccctgttgccagaggtacgatccacctgtcttcttgtctttgtcttggtagaaatgtattatcttttcttaccaaaattggaatgtaaaggaggaagagatttaagtctgaaaattatatgagtgcttcctccagactatcaaaagttagctattgattttgctttgaatgttgatgagactaacagcacactgaccttgagttctcttcacagtgggtaaacctcacaagtgcaactactgtggacgaagctacaagcagcgcagttcactggaggagcacaaggaacgctgccacaactatctccagaatgtcagcatggaggctgctgggcaggtcatgagtcaccatggtgggtagaaactggattcatcatttttatttccccctactgcttttgtagttcaacatgaagataaaatcttatgctgtttaaaactattttagatcactttaacttaaatgagttgagctttgggatacattgcaaataatctgtattgaaattttgcaatactcccaaattatttctgataatgtttgaattagcataataatgacaaccaacctttcatataccaaaggaaggtactatttactcttctttttcatttttctaaccagatagttagagctgattccacattagtctgtaggctgcctaaaagaatgaattggttatttgaatattttcatctcaggtaagtgatcagtcatataatttgaaatttctccaatcggccctttaatttttatgagaaattactgattttgctttctacaatgtgcaaaacaatctagaccaacattagacaccatctctctccacaaaaaacattctccaagcaaaacatcctgatctctaaatacactacggcaggtaggaaggcaatccctaaggtttatcaatgaataattctctgttcctgaaaagttaattttaataatcagccttatataggaaaataatgaatttcagttcttaagatttgtaatgtctatcttgtcttactaattagatttgtctactgataagttttatttatacaaagacaaaaatctcaacttattagtacatgtatatcttttatttatttttttaaattattttttattttattttattttttttgagatggactctcactttgccacccaggctggagtgcagtggcacgatctcagctcaccacaacctctgcctccccggttcaagcgattctcctgcttcagcctgccaagtaactgggattacaggcatgtaccaccaggcctggctagtttttgtatttttagtagagacggggtttcgccatgttggccaggctggtctcaaactcctgacctcaggtgagctgcccgccttgccttcccaaagttctgggattacaggtgtgaaccattgcacctggccacttgtatatcttttatttaaaaaaggtttgcccagtattaaatggtatagttggggattgcgacagtcacttttaaaatataaatatctatgtattggcttatacatagccaggtcaaggattatttctggggttttctgtttgttttatagagtatatactatggaactgtgtttgttggacagctagtgattatgtgttgttgagttgggaatttttttaatttaaaattttcttgttatcctgaacaactgaagcatttccttctagtttctataatctaaagaatctaattctattgatttgtgatcaagtccatttatttttatattatcatcattaacattttttacaaacctatctgtgtatcaagtcttggtaaatttgatatttcaagcttgcattacctgggctgatgatgatagcataggatctggaacatagagattacaaaatggtgcgtctcggagtctgccaaacctgctctggctacttcgtgtctgggtgtgtcactcataggctgggtttgaggattaaatgtgactggatatgtaataaatgcccaataaatggtttgctttactgttaacatttgagaacattaaataactgaataacagtaaatgtgctattccctataaatttgttatttaccagcagtaacttaagcattagaagtttcatttgctgaccaaatacactgttactatttaaattgatggatttaagggtaaattgaggctggaagctgatggaagcttgggggaataccaaatcttccccaaaacaccttatggcattgctactagtttttctatattatattctcccattgcagttctgacatttgatggcgttatgcatttccaactttatttattatttttgcattcctagttatttatggaattctgtattgcatacatactcctaaaagactatatggtaatagggaggtaaaatgtaaataaattattataatcaacattgaattatctgttggcagattgaacttttagagaatcaaatgctaacgaaaaatttaaacaagtatattttgagagaagcaaagatatgtcccctaaacaactggggcctcagtcttaccccctattccccagattgcttagccctccgtaggcacaggccctttacaggtggtgctgaagctggaaccaagtgggggaaagtgggaaaggggtaggggcactgggtaggtagagcagagggtaaaaccaaagaggagaagggattgagggtagcttctgataccatttatagaaacaaatattcttaaatactggatatcattttaaccacataagtttacattcatgccatttcataccatactttttctgggtttgttataacaataatttgagattatccttaatctcctctatctgttgttaacatgcctaatgggaaattggccatacgaggtaggagttgataaatactgtaagagaaaaacatggttctgtaaaatgctgctagagttcatcattaacatacgtctcatataaatagcttggcatatgtctctttttaaacagaaccatgatgaaacttatatctattcccttctcttgctgtatgttatttatttgttttccttttttgtggtagatatgagagttgctgtcattttggttgccattagctccatgaaatgatttaactattacttgtttttaaaaaatatctgacatagttatatttcctagtctttcttttctaaaacatgtttttccctgtgttgttcttctagtatggaattttttaaaggaaaagaggcattttcactttactcagaaaacatttatcaagagcttactatgtgccaagttatatagaacattctggggtataaaaataaatggacccagtcttttaatacacttactttatagtctggtaggaaatacatactttaaaaaaataaaataaaatgaaacaaaagctctaataaggatgcatacacagttctctgggaacatagatgtagaattaacctaactctggaaaagtcaagagaggcttcttacatgcaggagtgttcaatccaaacatgagggatgagtaaaaattgcatatatcaggcaggatacagagaatgtatattcaaggagaaaaagaggcttggagatataaaagataattaacagcttccctgatgttgagtgtaactgcagtttaaggagtggtaggaaatgaagcttctgaattagtaggttgtgatgcaaagacagagtcttctctgctaagctagtgtttgaatttcattgtgtaactgatggggggccactagggtaaccaaatttgttattaatgaggtaatcaaatcaagaaaaatgtctccaatgaggatacatgataaatgcctacctggggcagggggtatggtgtgaggagagactggaaatgacaatgccaaaaagaaaaagatgaagaggcaactgcagaagagaacaaggtgaaatattgaagacatatttaggaggtagaataaaatggtcttagaaaagttgattgagtttgaggaaaaaggaggaatatcatgactcatagttttggaaataataattgagttttatgacagattatttatataattaaattatttaattgggtttgatgagcttatttattttattgggttttagggcagattatttatcttatttaattaggttttatggcagattatcttgttcaatattatctttatgttgctttttcaagtctgcttagcttgaaggaaaagctttaaaatcaaactatgttattacctgttttacctaaacctaaagacaaaaatgaaagtgcaaagcagaaggatagccagcttaaaatctatatatattgttaggtttatgctttgaattgtttaggaaagggaggttctttttcacagtatagaggcattattgggcacccccaaactttggaataagcttttttgacttatttctattgggtgacatcattcagtgaacatccattgaatattatatctctcttgtatcataaaaggtattagagacacaaatacaacagaatgcaacccctgaggttatttaccagctattggaagatagaccagtaaaacaacaattatagtactatgtgataagcattatggagacagagaggacatacctaaatcagcagaacatccaagaggaatttcccgggattgagcttagttttaaaaagcaaaaatgtgaactaataaatggagaagggtgtttcctgcagaaagaacaacatatgtgaacaactaccaatgtagcatctaaaacacttggggtacctccatgtgtgtttgtaaagagatgtggtagggaggaggagaggcagaagactggatgaaaggaagaaatctaggattttccacaggtgaccagcatggctgatggagtaaatagtagtgtcattcagtgatatggtaattagtagtgtcattcagtgatatggtaattaggaaaatagaagaaagaaaggtaaatggagattttacttcttttactatattcttttatgtctccatgaaataggcaaataaaactgtctaggaagatgttaaatatatagatctcaagctcttgagaggggtctgagtaggaaacaaagatgcaggatccatcataatttatgtagtggcccaacgtagtagtttttaataaggcaatacggataagtacagagtgagacatcaagaaggccaagaacacactagcaggaaaaaaatttcatgaagatgagtaagagcagagtaggaacatggaaagagtgatttcacagaagccaaagcaagtgtcagcactgccaaatgctgtcaagaaaataactaagataactgtaaagttttttttttagatcgggtaacacggagaaaatttgcaacaatatagaagacatctgttgtttttgcctgcctgacatcccttcctccttctcttgataacaagagtgcccatttacctttagagaactttcctgttgcatgtagctgtggtgggaccctcagtccaggtactctgccctctcctgctcaggagctagacatgtgatctaacctaggtccctcagacttggaatttgaatttgggtgaactgtttggtttttcaaaagaatacattgggaatacacttatggttttgccgttatggttatcaacgtgatgaaacagtgatctaatggaattatgtttttatcttggttataacctcaaatataaggtcaaagaggatgtttggttataaagtttgttcaaacaggggaagttaccttattggtatatctttagcccagattaatgtctatgtgattaatgcattattgagatatctagagaatttgttttatgtttctagcaatgtatacacagaaatattttgatgagttgcacccagatccatgaagcaaaacaaaatcagattggttacataattaaggaaaaatgtatcaggtctttacccaatggcagaccttacaaatatgtctgactggtttacaaaaagtcaatgctcaaaatacctctacaaagagtagtatagcgtaatggttgagccaggctctggagccagactcctagtagtaataacattctctactaactagatgtaaaccctgaggaaattacttaacatctctgggtctttctgttctcatctgtacagtgaagatggtattacacctatttcatggagtttctggatgaattaaattaattattaaatgcaaagccttaaaacagcacacagcacatagtatacgtcagttattattattgccgatgtggtagaaacaagatcaatttttgacatgaaaagggctattgtaaagtagaaattagatcagaactctgccaaaatcatcattttcagttcacatgagcttcattgaactcgggacaaatcatctgaattgaggtggatgtggtgtgggacagttcccacacccaggtttcgttttgagtgttaaatgcgttcaaatatgaaaattcagggtgaaacttagagcaaaactgccaggttttggactactcatttcagaacaaattagcatatttaattttgggggcaaacccgttaaccagttttgctagttttttagcaaaatttacaacaacattttttttttcttgccaaattctttcattttggatcttggtctgacatattttcatctttggtccatacttggactgctttttagttttattgttatttctaataatgtagtaagtgcttgtaaatccatcatccaaacaaaaagttaagattttgaccacaatttgtatctcattacctggttctccaccacaccaccaccactgtgccattcttctgactccccttacgtgaattggtcatccttgaatgtttttgaaattctttgcttttttgttgactcttttacttttgtgtcacttttttagttgttactaaaagagaatataatgttttatacataatatcttgggacatactttttcaatgtgataacatcattaatgatatagtgcagacaatgatctgtttccttgttcctcagtctcaccttgaaacttttttccaaattaacatctaataattgcacaatcccttgctattcatagccagaggacttcttattaaatgtagattacaattcaatttttttgacccatctataggttaatcaaaacttggatggaatgttgatttctttaaacatgtccaaatcatgaaaacattttttcacactatgtttcattttcttcaaaattggtaacctattggttattaaatacatcaactgaattgtgcactgaattttcctacctttcatctcttgctttctcacttatttttgtgtctgccaagaggatccccttataaatgagactacatttttttgtacacagtgggactatcttatacaaatgaaaggatcagagtaaatcatttttacagggcatactgaacaagtaaagttattctaagaaatgaaagctttaggtttccaaacagtactggcacaagtgcaactggagaaagcaacaataaacaacaagtaataagttatgttctttcctgagaacactgtttttaatcaactgattcgccttgtctgagttgttaccctatccttaattaaacatccttgggcagtatttctactcaaaaaacaactgttttaagttctttgtctgcactatgaatgtcagtgtctaagggctaacagattagtccatagcaacttagaaaaaagggatgaatagggaacagcattggagagtatatgtcaaatgaattagggcagtagtttagtgagccaatgtgccaatttagaaatggattcttcttcctgctgaagagctctttttaaaataaattacaatcataaagctgccacagtttactgtgcatagacatgttcagaattggctcttcttcatgagggctgcttggtgcaggtatttaagctactgccaataaggctaacagctcatcaaatagaaaatacgctgcaggaaaatacatgggttaagaaaaacagttaaataccgtgctgttattttgtatgtctgtttaagcttttctctcaaacaagttgacttacttaaattgttttttaaaaataaactctaaaagccttgaaaatagtgcagtcttaaaagtagtattgattttagtcaatgctaaaaacactttcaggggaataagatgagaaaccacatgaacaagtttgccaccaatagtggcaacagtgtatgtatgtcatggatttaagagagactcaaataaaaacaaatcctctggtgtatatgtgtgtgtgcaaatttaaagagtatactttcacactagagggtattctttctacttgcttctactgaagcatactagcttttataacacatatcgagatggcatatccagctttgtatcatccaaaatgttgaagaaattatcagattgcatttttctatggttttgtatagtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggtaagcattacaaatgcttttgtttctttgcaaggtgatgttaataggaaaaggcaagttcccatgtattagaaatatatatggtgtgtttcagttcatgaaataaatacccatctcttttcttcattattcttttttactctctgacattctgtgtacaccacacttacaccaacacacacaaatacatatgagagataaagactgatagtggtcaccatctcaaaatatgatgatatataaagtgaaataacatgtaaaatagaaaatattaatttctttaaaaaatagtcatacagaagagaaacggtccagcctattggccagttttgtaatctgttttaaaattttcattagtgtcaggtttttagctgttttttttttaacctattaacatttatgcctagtgtcccattattggaacgctaagcatgtgggagttatttatatcctactgctcaaggtcatcgccaaggtctgattgcaaaaattaaaacaaaatttcaacctcaggcataaatgggttaattattttcgtattttatctggaatgaagataatttttctttttttcaaaattctgcattggaaaaataatgcagcaacctacattcaagatgtttcatatttggatctttatttcatattttaatctttgttttttcatccagcctatgattttgaactgaaaagattaaatatcatatatttagactttcccttttcatttccgaacccttagatattctttgatacgcccaatgttggcatcattctggatgcatgctgtaagtcatagattgtctctgaggatacaaaaagcatggactttttaaaagaaatatgaataatagagattcaataaaaagaattgttaaagaattgttattcattgtatatatatctattaggataaaaatattcaaacagattctgacattctagtgtcagcattgtgggacatcctgattaaatggtgattgagacaaaaatctttgctttggttatttcagggaggagaggataacacaattccattacttcaataaacacttaaagaaggtttcctttcaatctgaaattgaagaactaacacatcaccaaaatagcactctaagactagctggagggatttattgagaactcattgaaagaaggcctaagggtgactataagggccatagtttatattatgtcagttgaagcccctgagttatacacaaaggactgcctcaggcctttggtaactttttttctgttcttgtgggtaactatgaaaaaaaagaaataaagagccagtttacttatttttaatattgatttttttgttggatgtcattaaatgcttaaatctaactagactctaatgtagttggaaaaaataaaatatattattattctttctgtcttctgaatttatatagtctactctttgtaacgttttttgtcatgtttcctgacttagagatgttactctcaattttatttcactgcattccttagaaaagagggacagctttttagaaaattacattatgattctaaaatatttatattatatttttaattaagaatttgagagaatatttttctttatttggattctatcctagtaatatataacaaacagtagccatagtaactacttctgttattgttcttgaaacaaaaaatttcatcattccacacttattataaattgccttctccatgtgcctcagttaaatactattctttgaccttatcaaattctccagaccaatgatccctgaacttcctgcatatctacttgtctctccctctcttttcttattcagcttagattccatggctcatcacttctacatctctcttgccaatattcttgcctttcttgccttgttggctgatctatcatccccttctcctgatgaaaccctactgtcttgtaaagctgactgagcatcctttccatacttatatctcactagatgtgcaccccagcctgcacatctggtacttttggagaaaattccacaggctgatgagttggcatcaatatatatgaatgaataactgatcccaaatatcagctggcctatccatcctatggagcagcatccctgaagaatcatctctcctgctctccaccatgacagtgccaaccttctgtactctctgcaaacatgctgtctcactccttggtgatcttatatctatattcacaggggataaaatcgctggaactgatttcaacataatattaccaattctataggattgtatgcatccatgtaaatgcttttatcttttttccttatttcaatgaaacaactatccctctttctaattaaagttcattccccctcttgtgcacaggaattctcctatcctggccccccagatacctcacgctattattttttcctctttctcacttacatttttaacctctcccctctattgggcatttcctatcagcatttaatgcaataaattctcataattaaaaaaaatcttcctttaagccccatctccttccaggtacacctgtagtctccttttcttcaaagatgaaattgttgaaagaatctctgtgcttgttttcaatgattttttttttctattcatttccaagtccatgcaacctgtttttctttcccacactccattaaaattattctcactgaggtcacaactagcttctgtagtgccaaatcgagtagactcttttaagacagtatcttacacagctcctagagagtatctgccactattgaatagtcaattcctttggaaacacactcttttcttggtttttatgacattgttctctctctctgactgtgttcagtattctttcagacttatccttaacagctatgcaagttggccaaagtacaaatggaggcccccattcatatgcctaaatatttttatattataaatctaactgtaaaacttattttctacccttccaacttgactaagttcaaataaatatgcaattctcagatttcctgtagctctacactacaccttggcagcacgagcagaaccacccactggcttacagctaggctcctctcttagacccactcctagttctctccattaccaaagggggtctcacacacacatgtatggacaccccagcctgcatatccaggttctttttacattgcacataaagaccagtgccttggccatcaggcctaggtgtgtgcttaccagtagcacagttggtatgaagtattatggaccctatgaagaggactgtgtagtccctagaagcaggctcagaaccatttgggcaaggaattctggggtctgagtgagcttcagatcagcacattcccttggccctagaagctcctcatttcctgagaagaaaggcccagggctccctaaacatggggcccagtgcaaaccccactttccctgatctatgagtaggcagacaaatcttagtgttattccaggttctatttttgacccatccccttcacactctacctgggtgatatcagttatttccttggcttcagaagttgctttctatcaactgatgactcatttctacatctcaagcccaggtctctcttctgaatttgagactaaacttactggacattattattactcatagtcctcccaaactgaattcatcatcttttctatctcattgtcatctactaaatttcctaggccagaaacctagagcagcaatttcctaaacccctttgctaaatgaataccttcagggcacttccttgtttatccccttcaatctgtattcctttagggaaatattcaaatttttagcatatttagccttatattatctaacttgtctttcgcacttgtttctactctcagccttattgaagtataagtaattcctcctttataagtgatgccctcttgcctctgatcctttctaccagctactccacctcatgtttcttcacttggctatgtttacaatcacattattgtccttgattccatttccttcaagaagtgttcctttagtcccctggtatgagctaaatattcctcttatttgaccccatcacacataccacactatgttgtaaatgatccttaaattgctattgttccttccaaactgtaagttctttaagaatatagatcaggtttgtctagcttaatttggaaatgtttgacattgcatatatcttaagttgagtgccatgaaataatttctcctcgttttgtgtttccatttttgttactctaatcattacactgatctcacgtaggggccatttcttccaatctgagagtatctgccaaaattacaagttaagcatccatccacaacaacatattaaccatttatgggaaacccacatatgtttttaagaagcatttatctgtaagatttacctatgaaagtaactggtaaatagagaccacattgtcttagggacagaatttgtaaaagactaaatattctgttcaaaaccagcaatatctggctaaaaatcaaaactacttttcccgttgcaagcaccaaaactcttttatagttaacagtcagacattatgttgatgtaaaatattggagtaaaattatatttgttgaggtgtatatttaaggaaaaactgcaaacatgcttcaatcaagaaaaagtatgggaagctagcaatgggtgtacgtaccagcactatcacatacacatgtctaaataagaaaggatgatataaaaatgaatattaatttcaagaagcaaaacaatgagacaagattttccgagtttatgagcaaatactttttgaaataactcttaattttatttttaaataaccctacatttcattcatttttattaggtggtactacttttaaaagtattttaataatgacttttaaaaaatctccagaatgacatatatgggttatatatattttttctgaaatagtggccgaatcaaccaacttaaccctacacaacagtgaggtgccatatgttataaccagatcactctcacttttatgatctttgtagcttggtcactaatgtttaaaatatgcaaaatcaagcatagtctctgacttgtttttactgggtattcttcctcctattctccttgagctctattcaagtgagaattttaaaagaaaatgaaaatatgataaactcaatcatatctttcagactagatgcattctcatgatggcatcagaacaaatgcaaatggcttacaaaatttttattgcttttatatttttttcatgtcaagaatatttgtggccagacccagtgtctcatgcctgtaatcgtaacactttgggaggccaaggtgggtggatcacttgaggccaggagttctagaccagcctggctaacagggcaaaaccctatctctactaaaaatacaaaaattagccaggtatgggggcgcatgcctgtaattccagctactcaggtggctgaggcatgagaatcccttgaactggagaggtggaggttgcagtgagcagagatcacaccctgagactctgtctcaaaaaaaaaaaaaatgtgtgtgagcatcagcagaattgcccaaatacttgacttatagagtaatatataccaaattatgaaaaatggaggcaatcagtcaacttcgtaccctttcatatttttaatctgatactaatgttgttgtattttttttttttttttacttctttcacctcccactcccttccaggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccactaggccttttcattccaaaggggacccctatgaagtaaagaactgcacatgaagaaatactgcacttacaatcccacctttcctcaaatgttgacataccttttattttttttaatattattactgttgataattcttattttgtggaggcagtgtcatttgctctgcctaattacgataaggaagaaacagaagagagaaggggcgggaatattgtttctttatcacctggcttgtttattttgtgggaatttaagagcagtccatttctaccaaggcatatcatgctttgaaaaatcacttgattcataaagattcacctaagagattctgatttgccactgatattcagaattatgatggaagacaggaaagttcagagttttctgggtaggactttggtggtttaaaaatggtataagtaactttattcttgaaagaagaatgtgtttcaaactgtaaaccaattttttgttcttcagagatcatggaacacaaacacattgttattttcagtgataactcctaagaggagctgagttgttgtgggttctatgtttacttcccctatggaatttataattcagtatgttttacactgtaccatatagcaaaacttttaaactacaggtagttaagggccacctacaatacatctgaggtcctgtgatcttatttttctaaacgtaagcactgtttttccatagttttgatgactggcattttatagacaccctggcagccttacttttaacacctttaaggaatagtatttttatgtagttttcagaataacatatggtctaagagtggataaaaggcagtcaataatttctgggagggacttctactttcataaatttgtttgagaggttttcttttaaagttgtaatgtgatggcagcatagtatatgtatttgtttctaaaagtatgcttacgattgtcactttatcagcatttaatcagtgttaaccagtcagcagaaaaatataattatgctaacagtagggggagaaaacccacttagaaatcccttttctggtatttctcttttcactagtttttttcaagatgtgacctcccggtgttctgtccatagttcattcatcctttactcttcgagtagaaggtcttaaaagtcttcctgtcggctgtttctttcaaaatctcctcagagcaattgctaatttggcctgaatctggtaacttgaaccctgtaaggttacagaactagggctatttattttagcatttcttcagtagtatttactactcttgttgcaaagaaaagggaatgggacttctttgtaacctgtaccttggacaacagataaaagaaacaaaaaaataagaaagtttacttttacccttcttggagtctagaatgtgacagaacccccaaaggaaagtcctgcacatttttctgtttccaaaacatttaattgtgtaagtccttgtcagaaatgaatctcaatcccttagtatagaattccccttacatggtataggttgccatatttcatgtgcagattttaatttcatttatgtgggcgctctgttttttctttgcagtccagccacattagaggggaggaaccgagtgatattgattcaagtcattttagggggacatacttggaaggcagaacttgctgcttctgtttggggaggacagacctgactgtgactggattatctgataaccatttgtgaatactgaaattctgttaggcagtaactgataactgctctaaaggatcattaaataggatgctgaaattatgtatcttaatacagtgtggtatgagaattaccaagtcaagagaattgtggacataagcaagtttggccccaatactgctcttaactcattttccagcttactatttgctatttaaatggtaggcaccagctaagcacttctaagcactaacacagctagaactaggcaaaaatggttagaactcagctctcttctactagtccctgtcataattatttttgggaaaatgtccaaactgccccctttaaatctaagggaatgcaccaaaacagagatatatagaatgtcaaccatttcatttttttttttctgcatgccttggtacatagtgaacatacaacctatttaaagataaagcatgtttttgagactcgctcacccccccccacccaaccactcccaaataataattgggatgccattttttttccttttggatgaggtaaataattttaaggttcacaattttgtcttttactgcaatttaaggaaacatttggatgtcagtcaatatgttcataattttggctgtgtgcgaatttctgctggcattatctatgaattttcttcctacttatttttttttcagtatatgaacaatcatgtatctacctgccccaggatgaaactaaatttaggtggaccctaaaccttatgaagacagtgctgaggcactttccttttctgatttcatctttttgggaatctgttttattgaaggtagttagtagttgagagtgcatttgctacaagcatatacttgtatcttcctagcttcatgaggaacagaaagaggtggatatggctcagggtgtggcagggacaattgaggacaaagtcaattcaaatttgtgggtcagaaagaatttttgtggacgtagtgtttttggagaaactctggatggttatatgtgcatgccttttcttcaaaaggaaatacgcaaggttgtagcatctaaaaataaacataagagtcagacaccaaataaatcaagttttacataacagttgtatgcccagtttgtttaggtgagatttcacattacagaaagtatttgaggagcatgaaaatgggttatcttctgtattttccagtttggcaaaagttcagaatttcatcacattgctttgccctaattttgcccagaattttatcttagcctctctctgacagtgatgaatcatgctcaaaagccattctaattggacctttttaagacagggaaagggatcagtaggcggattggaagaaatttcaagtcattgaaatattccattgagatttcctaaagggacaaaattgggaaaataagaaactacgacttagatttggctacgtagtagaaagtatctcccctacatacatacaggcaattgtatgtatgaatcatagggtatatgtgtgtgtatactacacacacattcttttaaagagaattcatggaaaaaaaagcagttggagtgatcagatgtattgcaaaaacatacagagaatttaaatgacagttaataccaagaaattagttgggtttactttatcaggtcgtaataggaatcactaaagaagttactagtgtgtctttaggaccagtggcaactcttaaactaaaactttgggtccttattatctacttacagaacaaagtgaaacaaacaatgattaagctgattggatatacattcaaagatatttaatgtaaagttttttggaatacgaagaaaattcagaaaataaatattatcaacagttacttattggcaaatagagaaagacaagaatagtttagtgagcccggtattttgtttttatagtttttatctcagttgtacaactcacaaaaccatgaagtctttggtattttataaatgtttaacaaaatttacatcagattaaggcatttagatgaaaattattatgttctcactatcttccaaattttatttcatcctatctccaaaatgatttcttagggtacaaaaagagcagacggggctgtaaaaatacaagcaaaaaactgtgtgcccctagtttcaggcagaacttaaactgtcagaggtactagctacatgatttgttttttaactttggattgttcacgtccaaaaatggataaattacatttgtgtttatcatcagttgcattttatgtattattttaataaatactatctgaatgaagactattctaaaccagaaaattccccaaatccaaaagaaaaaaaaagtgggaagaggtgaaattgaagtttgtgtatatgaaagttatcttagacatatttttaattctccagtttctgcaaaataattaaaatatacagtaactggtctcctaaatcctgaatttaatgtattaaatacttatgttctttatattggtgcctttttaaaatgcattgagagtgttggttagctgttgcagctgtacaacacttttaatatgcatttttaaaaatcacttaaaattgagtactatataattcatctctgcatttttagtgcaaatctttagagcaatttctaatagagaaattttcagctcagctgttaaaaggaaaaggaaactttgaaactagactttactacctttttagtttcatagtatttctgaatatgattacaagattatgcaggtaaaatatagagtgaaactttacctgtgaattgaattataatttgtgtttttgttttgtttttaaggaagaataagttctgtatcaaacaagaatttattagataattttttggtcaataaaatacagtattcatttggattttcatctccagactagtattgttctagtcttggaatctgtattttctaatctgttagaaaatagagattgaaaattgatggaataatgtgaaaaagcaggtaattaattctccttgaacaaagcaaaactgaacagtcatatcacattgctattctccaaagcataatctcaaatggtttcatatcatggttgtgtattacttgcaatgggtgtgttaggatatgacagctttttaaaaaaatgagctgctggttatacaaagcaaatggcatatgaccaagaagctgtgatatgctagtgtttctttttatcatagtgtattactaggccaaataatgacaccttgaatatttttacatttattgcagaaaccttaaactttggaatttccataaggtttttatgtaatattctatttctagctttttagttttatcttgctgtactgtaagtttgaggatatttttcacctgcactcttaggaataagttcataattctgtttatggggctttcctcccataacactgcatttgtatattttctgtataaaatatgtgttgtgtattaacctttatcccatacagagagtggtacatgaatgactagttttctaagatgtcctttttattgtgaataaaatataaaagttaaaggccctctgctaagtcacataaagtacagcatataagttcatataggtacaaataaatgagtttgcagtgaattgggccttcaaattacctcaagtgacagatagtaagaaaagcttcttgagcaggtggaggtcactgaatcccctactatgcacttaccaagattttacttactttaatttactggaaattgattttttaaaaaatgactacactgtaacaagggaagggatctgggtttttttgttgttttattcttgttttttttaagtagttcaaattctgaaactgtgatttaaaaattttttacagtcaagcattctgattttgaacataactcccttccctttctgtgtaacaaaggtctctctgttatctcttaaattttgttacatctccctcagcctctttctttgtccgtctcccttctgtcattgtctatggatgtttacctctctgttctcctaaaagtttgaagattaggtcaactcttatttctagttcattggtaatttaatcttaatttttttttcgtgatttttgttggttgtataatctgctgacgtatttttatactcaagtgtagttttctattaaaaagaaaagtggttggattaaaaatagtaagctatgtaaccctcatgttactttcactttcaaatattgggtacctaaaacattacttcagagattatgtaatcctattatagtatgtttgctttcctttattgttggattttacattctgatttggctttcctccaaaaaatgtatatcatgaaagactagacagttatttgcaagtgtttagaaaggtgttaaaaatgtaaagcaaagagtcttaactttctcctaattgggagaaaaatgctttaacattactataataatattccaggtttggagggggtctccaggccccatatttgctgttaatagttggaccttttagaccatgtgttatttgcaatcccagaatgattgcttctgctattagttaaaaagatactattcttttctttctgtacaagtgcaatactccccttgaagtcttaaaaactatggtgatttttttttcttttctgacctattcttcctttagctaatgacaaaaagaaactcataaaagtcatagtatgttaaaggacacaacaagcaaagagaaaaacactccacaatcaaaagattacagaatgtggaaaccactagtctgatctcatggtatctttatttaagctaaatttccatggaaattagtaatcttttgcttgaaaaatgtgtcctaaagttgaactttttacagattgaatcttcttagaccctcgcccaatgctctaaattaagaacctaatacttaatatttttattttacttctccccttttagaaataaacttttaaataaaagcaaagcacttagctgagttttaaacacttacatatcacctattggagaaattttttttaaaaatatttggagcagtcctgttttcatacaaatttaagtaagaggtatttttcttatacatatttatatgtagtgtgctaattttctttttttatacctgtgtccctgtagtaaaactgctgtaatataaatacatgttttgttaaaagataacatttctttggcatttcttttaaaggcagttactgcatttctgcatttgtacagtatgtgtcttggccattttagatattctttctttaacaataccaaaggtaattagactattttaaagactaattgcttgacagtttctagggtattttgtgttttagaagcaaaaaaagaaaaaaaaataggtcaaaccagtaaacctcattttttttcaaactaataatttggggaaataaaaactattgtttaaaaaagaaatatatatatatatatataaatatatatgtaaagttaaaattccataccttgtatgtcaggtttgctaagtgtaatgtagtttttttaaggctcaaataccatacctcagaaaatgaggtttactatggaaatactgaaacagtctttgcagctgtgtgacaagtcactctactacatactgatttggagacctccgctaaatagttttatcactgcagactaaaatgtgggacttgtatcttctttgtttttaatgcacacacatacatgttctgtgcatgtatgtggttactgtgtatatgtgtatgagtgttgtatatgcatgtgtgagtgtgtgtctgtatgtgtgtacaactaaagaagctgcagaaactttgtaatactttgtgaaaaggattatattataaaggtttgtactgtctgagtgcacagctactggaataaatttagggaatctcaggaacaagcatataatttgtccaagatttatttcttctcagaagtgtaagtgcagtttttaattctgtatattatttaatattttaccaataaaataaacttctgacataaaaagtttgctataaaaactttctggtgaaaattctgtacacaaacatgtcttccaaatacagaagactaaattgttgactataccaggcttaaattatcattgccattgtgtatttgtcacacttccagttccagaggtttggatacatgtgatatctgcaatctacatctcaatcgttagtcatctccatcattactatatagtccaccagagcagaaaacaaagtattacatgatttgttaagaatgacatctctgctctttagacagctttcatacatatcttatttcacttttaaaaaagaggagtgtacatcagagcctatcccctttctatataagaaaatagaccttaggaaagaaaattgtgacttactccaaagagcatagtagtgcacttaaattacacaagaggtctgagtgctctgatctggtcaataacactactagaatacactactgctatagaaaccagtatttcttagaagtcaaagataaaattaagaagtcatagcgattgttcaatcacactggcaaaaactggttgccagttctgcttcatgttgtacaatcactacgtgagtcatacacatctctttgtttcttagat

SEQ ID NO: 3 - DNA, 107795 bp, Homo sapiens
  source 1..107795: mol_type = genomic DNA
tgtaaggtgtgtatttagaagaaggcaggctgggagaaaataaacacactctataaaacactccctctatttatatagagtgggtagtattgcgtgagtggtggcaaataaatcaagcagttcccagagggaaaacaaaagtgagataaatgcttccaaaaattaaaaagttgaattgatagaagtgccaaaatcctttccaactgtctgaaaacttggaccaaaaacttttaccatgtgaaatgaaaaagtgaggaaggaaaaaaaaatactttgaaagaagggggttctcattgatactttccacggctgttatcactgaatctagacaaaaagttccagatcttcctcaatttaagataaacgtctttgaacttcagcttgccaagaaagcaaggctgacccccgaccctccgtgtgatgtcacttttctcatccattatccttctaaaatagacattttgagacaatttagtgatgcgacatttccatcctgtcggggttccttgggtttgacattcgaaagggggctggaggcgcggaacccgcggcactccgcggccagcgtcctttaaagccgccgccagccagcgccgctgtaaccccgcgcaccggcaggtcgccggctcctgcccttttcagtctgcgccgacgcggctgccggatcccggggactccccgcgccgggaatctcccgccagctgcgcgctgagtccaggcgacggcaggagcacgtggagaggccgagtagccacagcggcagctccagcccggcccggcagcgacatggaaggtgagcctccagcccgcttcttttctccggggtctttgagaaacttaacctgtgcaagcagtagaaagagtttcttctgaatctcagttttgcttctatttacttcttttgtcaaaagtaaatatgaggtatttcagaggacaaagcgcgtcggttctcttaacgtctctcacttgtccttcccccgccctttggaaagtgataacttcttcgcaattttatttctaaagagttgtcagacatggggggtctaccctacatctgcagaggaagagcacttaaggggtgtccctgaaatggctgcaaatctggggtggaataatcaacccttttggatattttttccttcatggcctcagtgtttattggaattagactaatctgagtaacttgttttcttccaagggtaaatggtgtcatatgggaaaggtacattttaaagcggttggcatttgcagttcccttggaactcgatcttctaatgagtcttctttacccagcgcaaaaaatcaggttcacattaggagtttcatatgaattaattttgaaagtcgtaacactatccttgcttctcaaacatttttcattctgtacttccttgggatacctaaaatttcacactgctactgtaaactcttcaatggtcttttcaacaagtgtttctaaaaccttgaaaatggcaaatgcatctcaactcggggggggatggtgggattaagaaattcagaatttcattctattaaaaatgagatattgacacctaactaaaatgttactttgataaatatgaagatcgtgggtattttcttcataataatggaagaatctctaattaacaatgaactgatctttggcagctttatttctagagtatataaagttgttaaaatatcaaaaactttatgcttttaatttttttaattagggcttgaaaactttaaaaattttgcatttttaacatttaaataatggtttattgaagtgtaatttttaacaaaggttttattggccaataacaaggaaaaaaagttgggctgttgtatactatgggaaaaataatttttgaaaagctcatatgctttaattattacaaatgattaatttgtttttatgaaattataaatcagtactccaacactatttttcctagccgtaaccataccacgtatcaaggataatgtacagaatcatgtaaatccatagaaactggagtatttctctataaatgttaatacttccaaattcatttatttaatgtgcaataaagaaactaaatagcatatcttaggttattggtgtactaacacagatgtttaataagcctgaagactcccttaagaagaaaagaatgaaaacttgtgtagaatttaatttagagtgtgtagactataaatggaaagagaatgtagatcaataactttttaaagttgtgtttacatgattagattaaatctataggtccatggagatagatagtatagattatgcttaatgaaaaaactttaacatttaaattttaaaaagaggttaattatatgaatttggctattttaatgtcaggtccttgcgtatagagtatttgatgtggtaaaatatctggttcattatagctacccaaaatagactaatctggcccatgtttttgatatttaaaaaatttttatatcatcttgtggttttgatctcagtctagattagagatttggtctatagtgtatacctctcttggcaatgtaagaaagtagaatacttcatggaatacctttattttctgcccaatttgggggtcacattgattcataacagtttattgtacaatgcaatactgtatatcattccttcagcatataaagtcatttaacaaattcatttatcttttaattaatttattgagactttctatgtatgaagatgtcagatgaataagcttttgaaatagaaatcttgtagaaatgaagatttaatgaatatactaaaatcagaagctataactttttaattttctcacaatctgtgaccttaaatattttctacccaaagactgtttatgaatcactaaattttattatttgtactataacatctcttctgtctgttatggtcgggaaatgaggtggttcacaaacatgaatttcacaggtaatggcagacttcttatttaatgtaaaacatttttatttttaacaatattgagtaaagatttttctaagtatatttattacacagctccctgctttataagcaaggtacataagagataagtttatctcggcaactctgagttactgttctcaccatcagatcatatattattccataatatattctcacaggggttttgagctgaatgcccctacaccaaacatacctaggatattgtacaatttttgtctccttcttgatgtcagcttatatttcttaccacatagattttgtggtagtagggttttgtttcaggtccctttccatgttctggatggctgagattaacagagataaagcctgtagatgatggaatctgtttgccctccattgtgagcgtatggaggatgaaatgcgaaggttttaagaaacaaaagccacagaaaggccgtggcgtcctggtagaacataatttttcaaaattcctgtttgtacttcctttaatccagtatgctatttcatgcttccatgtctttgctattatgttcacccattctctacttgcttatgcctactcatctttcaagactctgctcaggcatcatcgccatgaaggctctctgatgaccttcttctgatgacctcctcctagtgccttctggtgatcacccctacccagtctctcatgttccttcacctcagctaagggcctctcctttctgtgtatacttctaccacattgtgttgaaattatttattgatgtaggtgtttattccccctctggtctttaagcttctggagaacagagactgtgtcttgtttttgtacactgaatgcgaaggacagtttctggttcatagaaggccctcagaatttgttgcattcaattaaaatagtgctgaaaaagtactttttattatgtttattgtgatgcattttatttaaataaatttttaaagtaggagattggggctagtaattctttgataaagctcagtaattttttaagcacttaaactgtataattgaaaaagaaagcttggaaaacaaaaatgttattggatattttttgctagactgtcacttaaagccaattttaacaaaattagatttagggggtacatgtgcaggtttcttacataagtatattatgtgatgctgaggattgggcttctattgatcctgtcacccaaataatgagcatagtgtcctataggtagtttttcaacccttgccctcctcccacccttccccattttggggtcatcagtgtctaatttgggggtcacattaattcataacagtccatgtgtacccagtgtttagctccccgtcataagtgagaacatgcggtatttggttttctgtttctgtgttaattcactaagaataatggcctccagctgcatccatgttgttgcaaaggatatgatttcattcttttttgtgtaaagccaaattttaactgttaaatttacataagcagatttcaggatcgcaattagtccattaaaatatccattgaatatgagcagaaatttgttttctcagtcagtaagatatagatgatgccataaaaatagcatacgctttcaagtgatactttaaaaatacattgttataaacttttcattcatatataaatgtatatacttacacttatttaggactaaaagattattgattattggtagatcttttatcagacatccagttttttttttttattattatttttaaagagatagtccttgctctgttgcccaggctaccacaaactcctaggctcagacgatcctcctgcctcaaccttctgagtagctaggactacaggcatccaccaccataacctggtaatttattgtacaaaccgccatgacctgctgatttattatctttcattttttgtagaaatgggggtctcactatgttggtcaggttggtcttgaattcttggccacaagtgatccttccgcctcaaccttcccaagtgctgggattacaggagtgagccactgcacctggcctagatagatgtccaatgataattggctcatatgtcaagctatagttccagaccatgtatgagaagaactgagtgattaacacagaaacttgcctatagtaggtgctcaataaatgtcttttgtcctaaggattcattaattattttgaaaagagaccctacacatcacaaggccatagaatgtactctgctatttagcctttattaaatttagctccatattaactggtatatttatgataattagaaattataaggatgaggccaggagtggtggctcacgcctgtaatcctagcactttgggaggctgaggcgggcagattgcttgagcctgggacttcaagaccagcctgggcaacatggtaaaaccctgactctacaaaaactacaaaaattagccaggtgtggtagcacatgcctgtagtaacagctacttggaaggctgaggtaggaagatggcttgagcctgggaggtcgaggctaccgtgagccatgagcatgccactgcactgacatgagcatgccacagcctgggcaacagagcaagaccctgtctcaaaaaaaaaaaaaatcataaggatgacaccaaggccttttggatcactaaaaaaaagatgcaataatgaaacaataattataatttatttataatatatatagattgtttgaaaagtaatctgtgtataactttattaattaatattactatataactcatcaaaatacttttatccctagtgattctgaatgaggttattttttcagtctaattgaaatctggtttttgcatccaaaattaaaagttttttttttaattaaaaagttacttaaaaaatttaagggctgggcacagtggctcacacctgtaatcccagcactttgggaggccaaggtgggaggatttcttgaggccaggaatttgagaccagcctgggcaacatggtgaggccctgtttcataaaaaataataataaaataaaagtaaattggcctggtgtggtggtgtgcacttgtcccagctacttgggaggctgaggtgggaggatggcttgacctgggagtttgaggcttcagtgggctgtaatgacgccactgcactccagcttgggcaacagagtgagaacccatctcaacaacaacaacaaaaaatgtaagtccaaagtggtatacagtaaatgaaaataatatggaattaccgtattatattaaataccaataccatatgaaaatattatgttagatatgtgtccaatccaggagatatttaaaaactgggtacttgatggttacaggcgcctgtagtcccagctactcgggaggctgagacaggagaactggttgaacctgggaggtggaggttgcagtgagccaagatcgcgccactgcactccagcctgggtgacagtgtgagaccccatctcaaaacaaacaaacaaacaaaaaactaggtacttggtaatggatatgaaacaaagtgcattactctttacttattgtcagtacttaaaatcagcttactgagtgattttttataacagtggaattggcaatatgtcacaaattgtacttagtagtgttcttagagatgaaatttttggcagattaggttattgtgggtttacaaaggaaacttttctttctttccttttttttaatagagacatagtcttgctatgttacccaggctggtcttgaactcgtgagcacaaaagattctcctgccttggcctcccaaagtgctaggattacagatgtgagccaccaatgcccagccaatgaaggaaacttttaaaataagaaatggaaaacatttcaatactgtcactcatgaaacagaacaaaactattatttttctactctgaattatctaaggattatgcaactaagtttcttgtttattttgttgattaatacacctatagccccttctagactgttattttccaaagtttagggatctctctctctctctttttttttttttttaatttttgagatgaggtctctctacattgcaggctggtctcaaactcctgggctcaagcgatcctcccacctcatcctcttgagtagctgggactagaggcatgtgccaccatgcgtagctggggatctttttataatcggtggtaactagtacccaacccagtccccagcacatagtaggcctgtagtcatatttgtttaataaattaatgaatgagttgcctgtgtgtttgatcatttgactcttttcttgcccagcttttatagtaaaggggggaaggaaaatagagacattgaaattggtaaattttgctggctaatttgttaaaggtactgttaaaggtttggtagaagagattgaagtattggctaaatccaatgcgtgaatacggccatttttctaatccatgccccccacaaaatacaatgccttctaacttttggctttatttaataattttggtacattttttatttcagtgaagcactgcagtttccactataaaccttttcttcacaatagatgttttgattcttaaaattgttgaacatcacttaatttgaatactctatcacagcaacatcagttcatttaagaagtaatgtatattagagagactattaatttaccagaatatttggcacatcagaacaatttcctctaggagtttgtggatatgagaaaaagatcactctcacaaacctataacatttttatggatatggaattacatcataactgatggaaaatattgtccagtatgaaaactggaagtaactaatttttataatatataacaactagtcatttttgctgtcatctacctctatctcttagttaagaaatcccttgacctagactgctaaaaatcactaatagttgatttatatgcttacatacagtgtaggcagttccacaaaggtgcttcacgggctaacttgatttaaatttcatttgaatttcacataaaattccagactttaaagtattgaagatgatcagtgtataaatttatcctctgacctcaaaatgaggcttttttttccaaatattaaaattttataaacatgatgagtaaaatcatggtgctgctggtcttttttctcctgtgcatattttctcatgtaaaattattattattattattattattttgagatggagtctcgctcttgttgcccaggctggagtgcagtggtgcaatcttggctcattgcaacctctgcctcccaggttcaagcaattctcctgcctcagcctcccgagcagctgggattacaggctcccgctaccacgcccggctaatttttgtatttttagtagagacagtatttcaccatgttggccaggctgatctcgaactcctgatctcagatgatccgcccgccttggcatcccaaagtgttgggattacaggcatgagccaccacgctgtaaaagttatttaagtaagaatattattaacagtttgtgaccagctggccaacatggtgaaaccttatctctactaaaaatatgaaaattagctgggtgtggtggcacgtgccaccaccagcaaggcaggagaattgcttgaacctgcgaggccgaggttgcagtgagtggagattgcaccactgcactccaacctgggtgacagagccagactccgtctcaaaaaaataaataaataaaaaagaatattactaactgtcttattccctgtactttattaagtgcctgtaaaggctaaggctcagctgcttgggacatgcttctgtcacagtatagtaatcaactactcactaacaaagacatgtaacatgtaaccttacaagcatttaaaagagttattatatacatgttatattgtacatatacaatatacatgttatattgtacatatacaatatacatgttatattgtacatatacaatatacatgttatattgtacatatacaatatacatgttatattgtacatatacaatatacatgttatattgtacatatacaatatacattgtatattataatacatgttacatcatcatcgtatattataaatcttaaatacttataataaagttggggcatttcttgtttgatttttatgttcagaatgaaataaaatgaatgttaaatatttttgtgattcaactttgaagcaaagattacttatcatcctttctcaagtttctgggagtagtttaattaaaaactgttctgaaataataaggtgaagcactcctgcctcagcctcctgagtagctgggattacaggcatgtgccaatactgcccgactaatttttcagatttgaattagtagggctaggagaccagaatgcacaaacaagtaacagcttctaagtgtgccaagccaatagcatagtatgcacatctcaataaaaggaatttggagattctcaaaggactctatagatatccattgcaaacatcaatgacctagtatggacttagttgccccagacctaaacaactagaacatttttacacgtaatccaatttggggagggagatccaatctatataataggacaaataatgactgaacaagttgatattaggaattcatgtagtgcttatagattttataactactactctacacaatgagacttgatgattacacgatgaccttgaggtatttcactgttagtctcacatttcaacaagagttaggattagacacaaaaacaagacaaaaacttgttttttttgcaacaggacaccgtttataccaaaatcttatgtgggatcagatgcaagtgtaatgttccaaatgagagattggagaaggagcaagagaccactgctaagcctatcttcctcttgccatagatcagccctgtggcagccctctgcaaccctggattccataaaacacattttgaaaatgactgatttagaccgttacatgtttttcctactagtaaccctgtatctagacttgcctcttttaatctgttttgaggattgctgccagaatgatcttcctaaaacataaagttattctcctactcagacaccattttaaatattttttggttggacatttaagatactccacaacacagcttcagtacctttccagtggtatcttctgtcctgtaatgtgactctggactgctgcttagtgttccctgttcaggcattgctttccctcactccccatttttctaatgctgttcttccaactcaacaagtccaaaaatgaactaattttctctccaatccaaaccaacttctcctgacttcccaacactgttaatagcaccacaatcatgccaactgaaatcctcactcatccattatggtctatcctaaatgccatctcctttaggaagccatccctcattctaagaggaataccttccttttctgaaacgtagtgacattttttgctgtctaatgacttctagactgtaactccattttatgttcaaacatgaagcatttttgcatggttttagtacactcaaatttatctaggattggaattaccacctacattgagagaggatgattttttttgtttagtttagagcttagccacttttaccattattattactgtttttcatttgatccatggctttatttccttgttttttaaattttatttccttgcttgtttttttttttaaattgatgcagcatgcatccttctgcatttagctgttgcattcatggtgattctacaaatacgtctatctaccttattctgtcttctggaataattgtgctggtgcatttacattttgaaatatagaatattatttactgtcatttatttcacctttatattacaattgggactttgtttttttgtttgagacggagtcttgctctgtcacccaggccggaatgcagtggcgtgatctcggctcactgcaacctccgcctcccaggttcaagtgattctcctgcctcagcctcctgagtagctgggattacaggcatgtgccaccacgcccagctaattttgtatttttagtagagacaggtttcaccatattggtcaggctggtctccaactccagacctcgtgatccaccttcctcggcctcccaaagtgctgggattacaggcgtgagccaccgtgttcttccttttttttttttttagacggagtttcgctcttgttgccaggctggagtggaatggtgcgatcttggctcactacaaccttcacctctcaggtccaagcgattctcctacctcagcctcccgagtagctggggttacaggcatgtgccaccacgcctggctaattttgtatttttagtagagacagagtttctccatgatggtcaggctagtcttgaactcctgacctcaggtgatccacctgccttggcctcccaaagtgctggaattacaggtgtgaaccaccgctcctggcctgttatttttttaaaataaaaaaaattttgacaagcattgcctacagtttaaacagtatcagcaaaacctatcatcactctgatagcatacattgcatcagtggtgggtcatcttcacatatgtacttaacgtatgttttttattgtttaaaagattttaaaagatagttgaagaacgacagttttataaagtttaattttgatgaatcttgtaatacatttattatacaatataattcaaaagtcagaaatcttcattcttcgttctaaaactatttattgaataccttttttgtgtcacatatgcaaaatactggcaatacagtggtaaaaaaggcagacaaagctttggcccttatggaatttggtaaggtaaggaagacagaccaaagaacaagtaataacaattgaaataattaaatttatgctaagtgctatgaaggaaataaaaagaatgctaatttggagagtaatggaaggaggcagggtcagggacctctcctctaaagaagaggtctggctagtgataggaatgtggctttggctgttttcttcagcacatggatgatattttgttttttttgagatgtagtctcactctgtcacccaggctggagtgcagtggtgtgatcttggctcactgcaacctctgcctctggggttcaagcgattctcctccctcagcctcctgagtagctgagattataggcacgcaccaccatgcctggctagtttttgtatttttcgtagagaacagggtttcaccttgttggtcaggctggtctcgaactcctgacctcttgatctgtccgccccggcctcccaaagtgctggagactatgggtgtgagccactgcgcccagccagtgatatttaaagccaagggaatgaatgggatcacctaaacagagaatatattgagagaagggctctcaggatagtgcctggagacatacctcatttggtcaggtagaggagaagccagcaaaagagaaagagcagtcagtgagacaggaagaaaaccagcagagtgtggtgtaacggaattcaagaaaggggagtatttcatgaaggagtgggtggtcagctatgttgaatgtggctgagaaatcaagcaagataaggacaaagagtgtggttggttttggcaacataacacttgctagtgaccttgacaaaagtggtttctgtggagtggtggggatggaaaccaaatcgaagtgggtagagtgggaatgggagtagaagaagtagaaacgttgattacagaccagtttttccagaagtttagctatgagggtgaagaagagaaatcagtgggatgttgctggagggaaatgtggattaagagaggttttgtgtttgttttgtttttcaaagatgagtgacatttacactatctgtagtgtaagtttattgagagaaaaatgccaatattgaaacattttgaaatcccaatttgcagggaagtttgggaaattagtgatcactgaaaatatcttcatatttagtttatgcatgacatgtaaataaatttctgtaccacgtgagttttaagctatatatggagcccattcagtgagtggttggtgtagatgcaacttataaaaattctgtagcaaaatcatgagaaactcttgagattcaaagtttttttcaaaagcaagaagatactgttataattgcatttcttaggtttaggatttagatatttggagatgtgtgagtattcaatggattttctttaaattaaggaataacatataattaaagtgcacatatcttttgtatatagcttgattatttttcacctttatacaaccttccagatcaatatatggaacacttgcatcaccttagaaggtttccttctgtccatttattgttaatttccccctttgccccaaattaatcactattcagacttgtatcctcatagatagtttccctgttgttgaatttcatataaattaattttacaatatatgcttttctggggtctagcttcttttcctcaatagatttttgagatccatccaagttgttatatttattagtagttcactctgttttatgatcgaatagtatggatataccacaatttataccttctgatgttgatggacaattgattgtttccagtgtttggctgtattacagacattcttttgcatgtcttttggtgggcctatatgtattcatgtctcttgaatatatacctaggtggccaggcacgctggctcacgcctgtaattccagcactttgggagaccgaggcgggcagatcacttgagcccaggagttcaagaccaccctgagcaacatggtgaaaccccatctctacaagaaaaagtacaaaaatcagccaggcctagtcatgtgctcctgtagtcccaatactgggaggtcaagtctgcaatgagccatgatcacactactgcactccagcctgagttgcagagcaagaccctccctctctctctatatatatatatacacatagtatatataatatatacgtgtatataacatacgtgtatataatatatacatgtatataatatacacatatataatatatacacgtatataatatacgtgtatataatatatacacgtatatataatatatgtatatataatatacgtatataatatatactatagagtatattatatatgtatatatactatagagcatattatatattatatatactatatagcatattatatatgtatatatactatatagcatattagatatgtatatatactatatattatatataatatatagtatatatactatatagcctattatatatgtatatatataatgtattatatataatgtatatataatatgtgtatatatacatatacatatatagtatatatactatatgttatttatgctatatattatatatacatatataataggctatatattatatgtacatgtataatatgctatatactatatgtacatgtataatatgctataatatgtacatgtataatatgtataatatgctatatattatatgtacatgtataatatgctatatattatatgtacatgtataatatgctatatatttatactatatattatatatacatacaatatgctatatattatatatacatacaatatgctatatattatatatacatatataatatgctatatattatatgtacatgtataatatgctatatattatatgtacatgtataatatgctatatattgtatatactatatattatatatacatacataatatgctatatattgtatatactatatattatatatacatacataatatgttatatagtatatatacatacataatatgctatatagtatatatacatatagtatatatacatatataatatgctatatagtatatatacatatagtatatatacatatataatatgctatatagtatatatgcatatagtatatatacatatataatatactatatattatatatatacacacacacacacacacccccaggaataccatttctgggttctaatataggtgtatgtttaggtttgatacatattgccaaagagtttccaaagtggttggatcagtttaccctcccactagcaatgtctgagagttatacttgttccacatctttttgttaacacttgctattatcagtctttttaatttttaccattcttttgggtgtgtagtgagtggtatttcattgtggttttaatttaatttgcattttcctgatgagtaattacattgagtgtcttttcttatgtttatcggccattttggatcctctttcctgaagcgtctaaggttttctggtcattcacgtttttttgtttgtttgttttgttttttttcctgagacggagtctcgctctgttgcccaggttgaagttcagtggtgcaatctcggcttactgcaacctccacctcccagcgattctcctgcctcagcctcctgagtagctgggattacaggtgactaccgccccacctggctaatttttgtgtttttagtagaggtggagtttcaccatgttggccaggctggtctcaaactcctgacctcaggtgatccgcctgccttggcctcccaaagtgctgggattacaggcatgagccaccacgcctggctggattttttttttttaattgggttgtcttttttcatactgattttgtgggctttttttttttttttccaggtgctatcacacctggctaatttttgtattgttttagtagagacagggttttagcatgttggccaggctggtctcaaactcctgacctcaggtggtttgtctgccttggcctcccaaagtgctgggattacaggcttgagccactgcacccagccagtaatgtgtactttgtcagatgtgtaatacaaatactttatcccagtctgtaacttgtttatttttcttaatctatttgatgaatagaattcttaattttaatgaagattaatttatcagcttcttttttgtgactaagtgctgcttatatcttgctaaagaattctttgccaggcacgttggctcccacctgtaatcctaccactttgggagggtaaagcgggaggatcattcaaggccaagagtttaaaaccagcctgggcaacataatgagaccccatctctccaaacatttttttaaaaattagctgagtgtagtgatgcacacctgtagtctcagctactcaggaagctgaggtgggcagattgcttaagcccaggagttccgggctgcagtgagctacgtcatgtcactgcattccagcctgtgcaacagagtgagaccctgtctctaagaaaaaaagaaaaataaatctttacctactccagggtcatgagaatattctgttttcctctagaagtttgattgtttcaactttcatatttagctctgttacccacttcaaattaatgattctatttttggctccagctattttacagttataaaactaggcattttacagttataaaacaagtcttcatttcaatatgttagattcaatcattttaccagaaacatctttttaaagtagcaatgacatgaaatgcccatttttccttttctctctgcatgttcatttggtagcatgtgatatacaaggtaaaattggaatgtatgtttttgtctacaacttaagggtaactgacctttgaaggaaaataacactagacactgactttgttgttttaacaacagttgtcagcagttcacctgtgccccacggtctacagtttcctcattaaacacttaatctaggagaaaaaatggaatgctttatttttttttaaagggggagggtaggaagaactggaacagtaacttggttgtatttgatctggtgtttttaagcaagcagtcttgatgtttcttaacatttaccttagcccaaggaaatagggaaggaagtatttcatgattggagggattactctcactttaatgcatgtctttgtttttgttatcttcctttcccctcaatcagcaataatgcacagtcagttagggtgtttgaaattaattggatctaaaatttcttctagttctggccttctaaaatctgtaatctgtataggaaggcatttaaaacacgatttaaattctctgaagcaacagtaaaactcaagagacaaatcctgcttctcttcttaatgtgtcactgactttgtgcccttgggcgattactggcctttatgttttttgtcattatttgtatcttggattttgtctgtaaaatggggaaaataataaatgccattgcccctatttatcttccagaattgttatgagaaaaaaagaaatctcattgcactattaggctgagtgcttgatgccacgtttttggagagccacaggcaaacaaaaacgggttcagaagacagctctcaccatggtcaggaaactgggtggtaacatctgagctataacttatcctgtatgttcccagaaagctgaactaggacctgaaatagacatttcagtaaatttgagttcagctacatttaagaaagaatgcttttaactactagagtttttctgaaatggaaaggaataacttctgtattattcgaagtgttcaagcagaggctgtataacccaagtagggggatattagaggacattcaaacatcagatggcagaggaaggagaggtaagctagttatgataataatttcgattctaatatgctaatttcatcggttatagggatcatttttcatcctacagaactgttcttgtactgatggaatttgaggaatgattatatatgatggtgatacggagaactggtaaaggattttgcagaaatgacagcgaacgcttctctgccacatttacagtcccaggtgttaatgttgcaattctgaaaacattcttataatcagtgcctcaatttctaaaacttttaattataaagtaaaagtgaacattttagataagataaactttttctttttaaaaaagaggtatgggtataaaggactccatttactattataaagaaggaatggaatatcaacattcagagatagctttctggcagctgccaggcacctctggtgcctgacagctggcaaactcaatacaggattctttctgagatttgacattttgctttcctttagtgcttatacttcctactgggaaaacagaccatggagtagaaatatgatctctgaaattaagaggaaaattttgttagctaatattcttaattcaatggtaattatttaatagtacagtattagaaataaaggcaagtttataaccattagaattaaaaacatgattgtacatttttgttgctaatgtaaatatttgattttcttcatagctaatttatacttgaactaataggtaaagcattcatatgtgaacttccatgaaatctcaattaactagactcctgccatttccaagtttaaattttatttataatctgccccctttcgttttacagcacatgaaatgttagaaactctgaaaaaaaaaaaaaaaaaaaccttggtatcttaaaaaaactaaccggtgagaaaatttatatagtgtcatcgtcaatgaaaagagaatctaaaaagccaaaaacgaatttgaaattatcatctcttagtaagcatgtgatgaagtcttgttttctatactggtagaaaacaatttgagattgctagaatggctatgagtagctatgaatgggagagtgctgtggaacactgcaaaagaaataagctttaacttttacagtctaatatatatgtcagtatctcagtagtagactagttgtgtcctttagaagatgtgttatttaagagataaggtgttaatggaaaacacttatgttttaattaaaccttaaaacatatcaggagattggttatttagttgtggaaagagcaaaaagacaaatgaaataggtattaactagagttttctgctagattacaactctggcttttaaatgaaataagaaaatggttttatctaatgatacccaaaaatcctgatgaaaacatgatgctaagtatatatttgtatacatacatgtatgttcatttaaaaatattttatctggatatttggatatcctgtggtcgatatttttaccctctgaaaagtcgcattaatttttcaatcagtgacatttgtaaagtgctttgtatgaaaggtgctgtctaaatgttttatttctttgattactgcgtcagtgaaaaccgcaactttctcttgaaattccaattgcagggttttgaaagtaaacatagaagtattagtaatagacctagaacagggctccataagctttttctgtaaaggaccagatagtgaatgttttaggctttgcaggtcatatggtctctgtcacaactacttagttggctgatggagtacaaaagcagccatagacaatatacaaatgaatgggtttagctgtgttccaataaaacctttttttttttttttcttttttggagacagagccttggcctgttgcccaggctggagtgcaacggggcaatctcagctcattgaaacctctgcctcccaggttcaagtgattctcctgcctccccctcctgagtagctggattataggcacctgctaccaagcctggctaatttttttttgtatttttttataaagacggggttttgccatgttgaccaggctggtctcgacctcctgactaagtgatccacctgcctcagccttccgaagtgctgggattacaggcatgagccaccgtgcctggcctccaataaaactttattgatgaacactgcaatttgaattttgtaaaatgttcaagtgtattcttctgatttttaaaaaccatttaaagatgtaaaaaccattcttagttcacaggcagtacaataacaggcagtgagccatagtttgcaaaaccatctagtgcgttcatagggatcaacaatagagatataaaacttcacttgttcattcaacaagtatttattcagtgtccatcatgagcagacactagtgtaggggctgaagatacagcaatacaaaacagacaacatcactgttctcatggagcttacattcttgtgggaaaagacagatttaaaaaaatgagtgatggtgatctgggagcctgaggcaggcggattacctgaggtcaggagttcgagaccagcctgaccaacatggagaaaccccatctctactaaaaatacaaaattagttgagcatggtgtcgcatgcctgtaatcccagctacttgggaggctaaggcaggagaatcgcttgaactgggcaggcggaggttgccgtgaaccgagatcgcaccattgcactccagcctgggcaacaagagcaaaaaaataaaaaataaaaaaacaagactctgtcccccccaaaaaaatgaatgatggtgataatgaaaaaattttataaagaaaaataaagcaggtaagaggaaagaaaatgacaatggaagatgacattttatgtagggtaattaggtaaaccttgtttgaataggtgacttttaagcagatacttgaatgaaaagagaaattgagccaagtggcttctaagaggaaagcattatagtcagggggaagggtaaatgcaaaggccttgaggcaggagcatgctttccctgttcaaataacaggagaaatgtagacaattgatgagatttatatttcaacaaaattaacccactccaagattcttccatcatttgccccagatatgtataaacatcagtacaggaaactatatttctatcaaaagcaatatagggactcttcctacttaactttgtagagcattcttttggaaaatatttcaaagctatgttgactgcctacacattaaaaagaccacagtagaccccatcaaatggaaactttttgtggatttgttaggggggactgaagtgtaaagaagttacaaaatgattaattaaaaactaagatgtttttgctgcatttagaaatatttaacaagtttctggtcttcatttgggagttccttcaccatctgtccactcctgtctttgaatgggcttcatgttcttataaataccttgaagtttaggggggaaatacacttcaatctatcttgttgttgtgttttaatttagtagctaataagaacattcatgtgacctgatcacagatggttttcctatctctttttttttttttttttaaaagacggagtctcactctgtcgcccaggctggagtgcagtggtgccatctcggctcactgtaacctctgccccccgtgttcaagcgattttcctgttatgaatgctaaggtagtcagaatctcatttttctcttcgaaactacactcagttacttctggtgctatttacagtacacaaaggacactttaaaaatattgagagctatttaaatgtcagagggtttaaaatattcaggggaccaagaccttttgcctttagttcaccaaatcaaaagccatctttctgtgtagtgactaaaagatgttcccacatgaaggttgctcatgtgaaagctttgagctagttttcgttctttctttttttttttccctccgttaagaatcttttcagcatgcagttgaaagtttcatagttagcgtttttaaaaaacactatcgaacttacagaaaagttgcaatacagcacaaaaaaactttttttctgcaagtttttaaagtaagttgctgacacggtgctctatccctgtcctatgctttagagtatgtgccctgccttcacaataaaatcatcaaaaccaggaaatcaacattactaccattcaatcctcagaccccattcaagtttctccagttgcttctgtgatgttctttaccacaagatatttcggttcaggatcacacattgcttttcgttgtcatgtctctttcctctccttcagtctggaacagttcttcagtctttcatgaccttgaaacttttgaggattataagccagctgctttgtaaactatccctgcaaattcgagtttggttgtttctttatggtttgatttgtgttttgcatatttggcaggaatatcacagaagtgatgctgtgttctactcactatcagaaggcacataatttttgtttgtcccattactgatgatgaccacatgtaactttgctcagttacagtgatatcgaacaggcttttccattatataggtgctcttttctttcaaccttgtaattaataagtatttttggagagaagcactttgaaactatataaatatcccatctgtcaccacacttttaatttattcatttattaacatcagaatgttaataatatactcatgaatttgtacatgaataatgtactcatgaataatgtactcagaatgtactcatgaatttctgtttttttcccaatgagttatagtctaatactattattattttatgctcccttgtcccagatttggccaatggaaaccccttcaggatgactgctctatacttttaagatatccacttcattctgtgagcacttccttgctttctggcacagcaaaatgttcccgacttatactttcaattctcatgccttagccaccccagtagctagtactacaggcatgcgccaccatgcctggctaatttttgtatttctggtagagatggggtttcgccatgttggccagtcttgtctcaaacacttgaggctgaggcgggcctcaaatgatccacctgcctcagcctcccaaagtgttgggattataggagtgagccactatgcccatcttggagtaaaccatttcttcagggagcctcattcctttaaatggagaataatatttagaagttaatatctagacgctattgtagtttcactgttcctaggtgctctcagtgaacggggaaagggactgtatttatgtatacccatatacacacatttacatctatattttatctggttttttttttttttttttttttttgctttgttgtcatccaggctggagtgcagtggcaccttcatagctcactgtagccttgaacttgtgagttcaagtgattctcctgcttcagcctcctgagtagctaggactacaggcacacttcaccacacccttctaattttaatatttttttgtagagacagggtctcactatgttgcccaggctagtcttgaactcctggtatcaagcaatcctcctgccttggcctcccaaagtgctgggattacaggcatgagtcacctcgcctggcctatctttttatatatagtagaagccatgagttcatgccagtacatccacttccaatacaggaccacaggcttccatatttgtaactcccttctctcacagaaaaacctggctctcattatccttattatatttacttatttgaccattcctccactatgtaatacaaccagtctcccattggcacttggcatctcctactcagcacagttgctctgcttgactcatttggttttgataatcccgtactgtggcctccactctgtgtggacattcttctcaactgcttgagctctgacagccccatgcaggactgcccctaaaatgtagatgccctcttcaccccactcgagttttggtactttgcactggactactcctggcattgaagttctcctcattccatttagcctctagtgggggctgccagccacttcattccctcttcattcccctcttcattccctctcaggctccagcagtccaggccaggcagtcctcctccctgcatgtcctcttatactgtttgagctctgacttttcatgctgtctgtccctccatgtggaaacttgcctttttttttttgttccagacagaatctctctgttgcccaggctggactgcagtggcactgtctcatctcactgcagcctcgacttccagggctcaagtgatcctcccacctcagcctcccgagtgactgggactacaggctcatgccaacatgcctggctaatttttgtatttttcgtagagatggggtttcgccatgttgcccaggctggtcttgaactcctgggctcaagcaacctgcccaccttggcctcccaaaatgttgggactacaggcatgagccaccacacctggcctgaaaccctccttacctcattcacactgcctaccctgcatggacacactcttcatgctggctggtctccagcagcccaccctgggctacccctattcattgcatggacatttttctcatccagcttggattttgacatcccatgccaagctgctcctttgcgtatttttaaactatgtgtatttcccctgcctttatgctttgttgctaacctctaatggctaacatattaaaagttttaaattagaatacaatatgactatagtgactgtattttttaaactaaaaattggaatatgacctgattatagaccattttaaatcagtacttttctaaatagttttgagtaaatgatcacagtcaaaataaagaacaaaggagagttttaatttctgtaatggaatagtacctcctattgaaccaacctttccacggataagaatgatagattctggacaaaatattaaaaaaattatcacctgaaagcactggaaaaatgaccaaaagcagcctgttttatggccttttatacaagggcaggccccatttcagcctcagagtggatagaaaatccacagttttgacaaaccataacaaccacagcaactgggaagtgaagggagacattctgcagtggagaaagccagagagggcttttttggatataaattctctggctgatccactaatgtgtagagttacactctaaatagcccaactgaggctaaaataactaaacagatttatacttctgcaaaggtgactaactcatcccagtttctttgggactctgaaagttctgtgttctaggaaacccctcaatccaggcatattgggacatttggtcaccccagctgctgcccacaacaggtgagacggagtttgagtttaagccaaggcaagttagttgcaaggtttaaaaagtaaaaagtctggtcaggcattgtggctcaagtctgtaaccctagcactttgggaggccaaggcgggaagattaccttaaggccaggagtttgagaccagcctgggcaacatggcgagacccccactctacagaaaatagaaaaattagctggacatggtgacacctacctgtagtcccagctactcgagaggctaaggtgggaggatcacctgagcccagggaggtcaaggttgcagtgagctgtgattgcacctatagtcccggctactcaggaggcagaagtgggaggattgcttgagcctgggatgaagagggctcaaatcataccactgcactccagcctgggcaacagagtcagaccctgtctaaaaaaaataaaaataaagaaacagtcctctcttcctttctccgccatcgtggtgtgtgcttgcctccacttctcaccatgtcttctcacaagactttcaggattaagtgattcctggccaagaaacaaaaggaaaatcttcccattccgcagtggatttagatgaaaactggtaataaaatcaggtacaactccaaaaggagacattggaaaagaaccaagctgagtctgtaaggaattgcacatgagatggcacacatatttatgctgtctcaaggtcacaatcatgttaccatatcaagctgaaaatgtcaccactgtctggagagttgaacatgttttattgggaatatattttttctctctgaatctcttatgaacgtgttggttgactgcattcagtaataaatatatgagacctttcatttcaaaaaatacgaaaaaaaaaaagaagaaaaattaaaacaaaaaggactgggcatggtggctcatgcctataatcccagctctttgggaagctaaggcaagaggattccttgaggccaggagttcaagacaagcctgggcaacatagtgagactctgtcactacaaaaaactttaagagccagatgtggtggcacacacctgtagtcctaggtactcagtagactcaggcaggaggactgcttgagcccaggagtttaaggttacagtgagctatgatcatgccactgcactcaagcctgggccacagagcaaaatcctatctcttaaaaaaaaagtaaaacaaagattcctacatgcttcagaggaacatatagaattcacaatttatgtaatatatcattcacaaggtccagcatacaatccaaaattatgtggtcatatgaagaagcaggaaaatatgacccatactcaagagaaaaggaaatcaatggtaactgacctcaagatggcatacataatggaactggcagacaagaacttgtataaactatagtaattacacttctgtaattgaagttatagaaggaaaagcgtgggagaatggaacagaaaaaatatttgaaagcatttagagaaaaatgaaaaggatataaaggaaaatatgcttttaataaacaaatagaaaacctcagcagagaaattaaaatgaactaaatggaaattctagaactaaaaattacaatatctgaaaaagaaaactcattagataggcttaacagcaaattaagatgacagaagaaagagtaaatgaacttgaagatatgaaaatagaaattattccttctgaatatcagagaaaaaagcattgtggggagataaaacaacaatgtctcagggaactgtgggaaaatatgaaaagatctaacacacatataattgaacttatagaaggaaaaggggaggagaatggaacagaaaaaatatttgaagaaataatggctgaaaatttcttaagtttggtgaaaaacgtaactgtacagatttaagaagttccatgaacccaagtgggatatatgcaaataaaattacagctaggaatattatggtcaaactgctgaaagccaaaagtaaagagaaaaatcttgaaagcacttagagaaaaggaacacattacctataggaaaacaacaacacaagatatcactgacttctcatcagaaacaatagaagacaggccaggcgtggtggctcacgcctgtaatcccagcactttgggaggctgaggcaggcagtttatgaggtcagaagaccgagaccatcctggctaacaaggtgaaaccccgtctctactaaaaaaaaaatacaaaaattagccaggcatggtggtgggcgcctgtagtcccagctactcgggaggctgaggcaggagaatggcatgaacctgggaggcagagcttgcagtgagccaagattgcaccactgcacttcagcctgggcaacacagtgagactccgtctcaaaaaaaaaaaagaaaaagaaacaatagaagacagaagacagtggaatgccatcttaaaagagctggaaggaaaaaaaaactgggccaggtgtggtggtttatcctctaatcccattttgggaggctgaggtgagaggatggcttgaggccaggagtttgagatcagccttggcaacaaagcaagaccctgtctctaaaaaaaaaaaaaaagttttaagaaaaaaaccctgtaaactcagaattctatatctagtaaaaatatcctataagattgaagacaaagtaaagacatttgcaaataattgaaaacaagaaaagtccctcagataattgcactattaaaaatgctaaagttctttagactgtagggacgtgataccagatcagaactcaaatctttatgaaaaaatgaaaagcaccaggccaggcacagtggctcacacctgtaatcctaacactttgggaggctgaggtgggaggatcatttgagcccaggagtttgagaccaacctgggcaacacagtgagacccccgtccctacaaaagaattttttttaaattagccaggcatggtggtgcttctgtagtcctagttacttgggaggctgaggtgggaggattgcttgagcccaggaggtcgaggctgcagtgaaccatgattgtgccactgcactccagcctggcaacacagtgagaccctatctcaaaaaaagaaaaaaaaggcaccagaaatgttaaatatgtgggtaattagaaagtactattttttcattttaatctttaaaatacaatatgactattcaaagcaagaattagcattgtatggtaaggtttaaagtatatgcagatgtgatacatatgacaactatagtataaaggttggagggtaggaaatgggcctaaatggttgcaagctttttacattttatgtgaagtgtataattttaactctaagaagcctggaaaattaaagatatattttatagtccctagaacagagagaaatagacaaatcaacagtcatatttggagattttaatactcctttttcagcaactaaacaactagtcaaaaattagtatatccatagatctgaatgacattgtcaactaccttgatttaattaacatttattaaacagtacacttgagaactgcagaatacctagaagcagtttctccactacttattgaatctccccctgtttctgcccacctgcctccactcccgcctccaccatgtccatcagggtgacccagaagttctacagggtgtccacctctggccccggaccttcagcagccactccaacatgagtaggcctggtgcctgcgtcagttcctcgagcttctcccaagtgggcagcagcagcagtgtccagggtgtcctgggtgccagcatgggtctgggtggaggctatggccgagctggtggttataggaggcatcacagccatcatggtcaaccagagcctgctgagcccccttaagctggaggtggaccccaacatccaggccgtgtgcactcaggagaaggagcagattaagaccctcaacaagtttgcccccttcatcgcggttcctgaagcagcagaacaagatgctggagaccaagtagagcctcctgcggcagcagaagacagcttggagcagcatggacaacatgtccaagagctacatcaacaaacctccgtggcagctggacactctaggccaggagaagctgaaactggaggcagatcttggcaacatgcaggggctggtggaggacttcaagaacaagtacgaggatgagatcagtaagcatacagagatggagaataaaattgtcctcatcaagaaggatgtggatgaggcatacatgaatagggtggagctggatggagtcttacctggatggcaagcttacaagatcagcttcctcaggcagctgtatgaagaggagatctgggagctccagtcccagatcttggacacatctgtggggctgtccacgcataacagctgctccctggacacggacggcatcattgctgaggtcgaggcccagtatgagaagatcgccaaccgcagccaggccaaggctgacagcatgcaccagatcaagtatgaggagctgcagaccctgcctgggaagcacaggatgacctgcgttgtacaaagacggagatctccaagataaactggaacatcagccggctccagtctgagacagagggcctcaaaggccagagggcttccctggaggccgccattgtgtatgtgtggggagcggtccattaaggatgccaatgccagggggtccaagctggaggccgccctacagtgggccaagcaggacgtggcgcgtcagctgctcaagtaccaggagctgatgaccatcaagctggccctggacattgagatcgccacctacaggaagttgctggagggcaaggagagccagctgaagtctgggatgcagagcatgagtatccatatgaagaccaccaggggctactcaggtgggctgagctcagcctacaggggccttacaagccctggcctcagctatggcttgggatccagctttggctctggcgggggctccagctcctttggctgtgcctgcttctctagggctgtggttgtgaagaagatcaagaccacgatgggaagctggtgtcccagtcctctgatgtcctgcccaggtgaaaggccatagcagcccctcccagcctaccccctcccatggcttctccagagcctgtggaggaggccactgtgcaggggagtatagggaacaggagacccacctgaggctcagccctaaccctcagcccacccatggggagtttactgcctgggataccccccttgcccatgcctccagctacaaaacaattacatcatttttttttcctccaaaataaagcctcagccagatctgcaaagcaaaacaaaacaaacaaagaaaaccaacaaaaaccagaacatacatatgtctcaactacacatggcacattcaccaagaacaccagtcttaatacttttaaaaggactacagtcacacagactgtgttctctgactgtagtgcaatggaatttgacttcaataacaagttagctaaagaattcccgaattcccccaaatatctggaaattaaacacttataaataaccagtggatcacagaagactcgagggaaattagaactgaattataatcaaaacatcagcatttatgagatgcagctaaagcaatgcttacaggaaaacttatagctttaaatgtttacaattagaaaagaagaaaggaataaagtcagtcactaaaatttcaccttaagagggcagaagcagagtaagttaagcccaaagtagagggaagaagcacttttgaacaagttctagtttcactgtgttgaaactagatatatgcctcctaccctataacccagtaattccacacctaggtattgactcaagagacatgaaagcatatgaccacagaaatacttgtgcaaggatgatcatgctccagtctaatcatgagaaaaacaaacacatcccagttgagggacattctacaaaatacctgaccagtactcaaaactgtcaagatcatcaaaacaaggaaagtctgagaaactgtcccaaccaagaggagcctaaggagacaagatgactaaatgtaatgtgggatcctggaagggatcctgcaacagaaaaaggacattagatgaaaactaaggaaatctgaatacttagatagtaataatgtatgaatatatattcattaattataactgatgtaccatactaatataagatgttaataataggggaaattgggtgtggggtatatgggaactctctgtgctatctttgcaatttttccataaatttaaaactactctaaaatttaaaagtttattattttaaaaaggaatatcatagtagtttgacttataaatagcccaaactggaaacagtctaggtgtccatcaataggagaatggataaacaaactgggatattcatatggaatactacttagcaataaaaaagaatgaactactgataaacaccacagaatggatgtatctcaaaacattatgttgagtgaaagaagcctcgggcaaaataatttataccatatgtctgtgtgattctgtttttatgaagttctaaaataggctaaactaatctatggtggaaaaagtcaaaacagtagttgtctctggagtatgtgggtgggaattgactaggaagagatgtgagggagcttttctgggatgatggtaatgttctgtatcttgataggggtttgcattacatgaggatatgcatttttaaaaactcattgaatggtacacttaagatccatgcatatcactgtatgtaaaatttactttttaaaatcttgtaaacagatattgaattttagttaataataatcatgctgaaatgttaggactgaggtatactgatatctgcaacttattttgcaatgcgttgaaaaagctggatcgatgaatggattgagagatgaatgggtggaaacatatgtgagaaagcaaaaatagcaaaatgttaattgtagaatccagagggtgagtaatggatgttcaactgtgcagttctttttttaaaattttttattgatacataatattttacatatttatggggcacatgtgatattttgttacatgcatagaatatataataatcaagtcagggaatttggaatgtttatcaccttgagtatttatcatttctatgtgttggaaacaattcaagtcctctcttctagctattttgaaatatataatacgtcgttgttaactatagtcaccctactctgctgtcaaacattagagcttatgccttctatgtaactatatgttttacccattaactaatctctcttcatcccccattcccacccacacaccctgtctatcattctactttccacctccatgagatcaacgtgtttactgtcatatatgaatgagaacatgtgatatttgtctttctgtgcctgacttatttcacttaacatagtgacttccagctccatccatattgctgcaaatgacatgattttattctttttaattgctgaatagtatttcattatatatatgtacatatatatgtacatatatatatatataccactttgaactctttttttctatgtgttcataataaaatattggggaagaataaagaatatatgaaattatttctaatggaacccactttttgcatattgttcttggtgtaagtagatctctggcagaaagttcttagagtaacattgctgcatcaatatttttttttcatgctccttgaaaggcttttagggagaatatagagaaagagtaatcttgctctgaaagaggaattctgctgttaagttttccgaaatggtatcccttattaaatttggaatgctcaaacctgtagaaatgtaccctaagtagctaatatttataacctctttcttttcagatatacaaacaaatgcggaactgaaaagcactcaggagcagtctgtgcccgcaggtaacatcaggtctttcctctctggaaatatacctttactaaaaaaaataccaaagagagaggaatgtgcttattctggttgcagtaaaaatgctatggaaatgtgtgtgtgtttttttttaaatatggttgtctgttgctgatgaaaaaataactcatccatttagatcatgtaagttctttagcatcttagggccaaacatacactacctaatgttgatcactgtttgtttgtttgtttgtttgtttttgatttttcagctcattgctctgaagacagtttttgttgaataatatagtagcttgtatttgaatgacttgcgatttttactgtagaacagtggtctgcaaacttttttgtaaagggccagatagtaaatatttagactttgcaggccacatatggtctctgtcacgtattattttttgtatttcttacaacccttaaaaaatgtaaaaaccattcctacctcttgggcccacacagaagcaggctatggtcagacttgtacctcaggccatagtttactgatccctgctctagaataaattataaacatttacaataatttatttcctgaaaataacttctgtgtcctccattgattaactcactgtgtgaatcaaagcttacattatattacaaaatttaatttatcactgaaatgcctttttgtgttactgtaacttgtaatcacatctttctttctttcttttttttttgagacggaatttcgctcttgttgcccaggctgaagtacgatggtgtgatctcggctcaccacaaccttagcctcccgggttcaagccattctcctgcctcagcctcctgagtagctgagattacaggcatgcaccaccatgcctggtgtatttttagtagaggcggggtttcaccacgttggtcaggctggtctcgaactcccaacctcaggtgatccgcccgcctcggcctcccaaagtgctgggattacaggcatgagccaccgcacccggccaatcacatcttttaactgaacctaaatctcatattaattgtttaggtgtccactatattttagtcatttcaggcttgagaaaagtgaaccattgtttagaatggtatcttagagttcacagagtatacatcataatatgcagaaattagaaacttcagatattcataaatcaaacactgagtccatggtgaccacattttcaggggctcttagtgatgggttttgtgaaacgtgtttctctttcgctcccgcgtgtgtttggtcatggctcagctgaaaggagtgccactgcagagcttctagttacctccctacttcagcccagcctcagtgccacaggattcagtgtctttgactattttttatattaatgggaactcagcaaatattttattgaagattgggaaggagttagataaaaagagggtaacttttcagttcatttcttctgctatcatctttttgttaagctactatgcctatatgtgaaaataattccagttgcattgttcctttttcttctagtattatcaatatttattatacatttctcttaaacttattttgtaacaagtttgctgtttgtttaagcatgtctttattatgtctgctccctttcacttatgaactttaataacactttgtatttcctttatttaatcctttgtctgagcctaccatgtgttagagttgtgggcttctcttatctatatctcaaagtatgagctccttgaagacataatgacttattcatatctgtagccttgtcaacattcagtaaatgttcaaacaaataaagaaacttggggtccaatatgttttccacagaatcacatttataatgtgaacagaagggaaaaacagaatttggcatacagaaagcttacagatcttcctcctaggaaagcatctttcccaaattaggttatgccagtgtttgcacgcttagtatttctggtcactactttgtcataaatagcaatggcatcctactgaatatcgtgagatataccaatggagagatagtttttctccagaaggaacatgagtgccctttaactcagatgaaagaaatattcaagaaagttgtgtgtattcagagggtacatgaaccctgggcatatttcttttctcttttttttttgtttgagacgaagtctcactctgtcacccaggctggagtgcagtggtgctatcttggctcactgcaacctccacctcccgagttcaagtgattctcctgcctcagcctcccgagtagctgggattacaggcatgcaccgccatgcccaactaatttttgtatttttagtagagatggggtttcaccatgttggccaggctggtctcgaactcctgacctcaggtgatctgcccatctcggcctcccaaagtgctgggattacaggcgtgagccactgcgaaccctgggcttatttctaaacctcaagagttttcatgtatacatcccttttgggtatgtgtgaaggggtactatctgtcactaaaggattgtgtgaagctgaaagttaaatgggggtctacttaatatatatttataaaaatcctttaacgttgaaccacatacttaaaattccttttatcttttagaaagtgcagcggttttgaatgactacagtttaaccaaatctcatgaaatggaaaatgtggacagtggagaaggcccagccaatgaagatgaagacataggaggtgagccgtagtgtgcagactaaacactgatgcctgtagatattgcttaggcttagtgtagagaagaaatcagctctccaaggaaaacttgcaattgtaggtttagttaggagtgattctgttttgttaatttcacaaatatttgcatttcatagttgtaggttatcagcaaattttgagaatttaaggtgcttatccagtgttccaattatattaacttcacttaaaacatgatgtttgactattttccattataaaagtaatacatgtttattgtagaaagtttgtctggggtgttttctgacaccaacaaaccaattttctggttttccgacaatgacttggtgtccaacaattcaattcaattattattattatttttttttgagacggagtttcactttgtcgcccaggccagagtgcagtggcgcaatctcggctcactgcaacctccgcctcccatgttttagtgattctcctgcctcagtctcccaaatagccgagattacaggcgcccaccaccatgcccggctaatttttgtattttttttaatagagacggagttttaccatgttggccaggctggccaggaactcctgacctcaagtgattgccaaactcagccttccaaagtcctgggattacaggcatgaaccaccatacccagcccacttcaattcaattctgattgtaattcctgatgtttgcgtagaccgcacaggttacaggctcagtctcacagtactgcacagtactctccctactttagacaacagtctcaagtaccaggtccccaggttacctgcacttttgtctgacttagctacaaaatcggggactcccctccacatgtttaataatttgctaggatgactcacaggacttgttacataactcatttattcattcctttactctcagctactcttcctccttcttactgtttatgcccaaactttcccacctttgggaggaatattaggtttgaccactgcactggtctagatcgtaggcagcaatactagtctagcaagcacctctccctcagtcaactcccatacatacaggatagggttacacaggtacggaactagtgggctatttctaccactaggcaatacatctgcattcaatgttaaccccaattttgccagatggggtaaaggcacagcctgcctcatcaggcccttaggagttctgacataaaggtttaaagctacggttacactttcttgctttagaaatcatctctgtttctggcacttgtaattacagtgctggtattgcaaccactgcatctggtaggaaagagaaagctgatgtgacaatgtagggaaggaaaaaaagaatagtcatcacaccaatatcctcctctttggcaagaattgtatagtcatatcagcatcctcccccacctcctcttccctaatccaccagaaaaacagaggaatctacctagtgggaacactcccttagctcatttcaatgtgacctcacacttgtgcaaatgcataaaccaacccttcacacctataccttaccctattttagatatctatcaaactactactctgaggaggatagctctttgcccattgtttgacattatgggctgtaagtagagtgctccttagtctaaagaaatgtggctcagccattcaaattggtgcaatatcttctgttctggttctttcgtggtactgaccatttgtgtatacaaccaggtaagcaaagccccttccagagtctgtgtttatccccgtcaaaaacaatttataggttgggtgcagtggctcacacctgtaatcccagcactttgagaggctgaagcaggaggatctcttgagcccaggaatttgggcccagcctgggcaacatggtgaggcctcatctctataaaaaaaaattataataatacaaataaattagccaggtgtggtggtgtgtgcctgtagtcccagctactcaggaggctaaggctggaggatcacttgagcctgggaggttgaggctgcagtgagctgtgatcctgctactgcactccagcctgggcaacacagtgagatcctgtctcaaaagaacaacaacaggccggccgtggtgactcacacctgtaatcccagcactttgagaggctgaggcaggtagatcatttgaggttgggagttcgagaccagcctgaccaactggttaaaccccatctctactaaaaatacaaaaattagccaggtgtggcggtactcacgtgtaatcccagctactggggaggctgaggcaggagaattgcttggactcgggaggcagaagttgcagtgagccaagattgcaccactgcactccagcctgggtgacacagtaagactccatctcaaaaaaagaacaaaaacaaaaatgatttgtagcccttgggactaccagcattggctcttcaatgcaatccacttgccaactatgtgtggggtcttccccctggggaatcttccccatagtcacctatagtaactgtctctcttattggcagacataatagtttgttgctattttgtgcctcaggaggtccaagggaaatatgtccaaattcagcccatcttctgcagtggctgtagcatcccccatcctgtctactcattttatggacccacgtgctcacctcaaacacgcacactgtgatacctactaattggttctgatcagcttccaaatctggaaggggggtcttttaccgagcattgacatgtcctactttaatgcacccctcaaattcccatagtgatttccacagggctttgtcccatatgagagtccctttaatatgccaagtttccattgcactcttgccttaccacatggccaggctgttggccattgcccatgaatcagaaactgaaacatatgggcatgtatcactgttcagttcttccatcacttctaggaaaacagcagattattcagctcaccaagctgatttgtttttaccttctttaattacagttgtggtgttccaaacaggatgttgtccatttaccttaaaactaccatccacagccaggcatggtggcttatgcctgtaatcctaacactttgggaggccaaggcaggaggattgcttgagctcaggagttcaagaccaacctgggcaatatagtgagacctcgtctctacaaaacatttaaaaattagctaggcatggtggcatatgcctgtggtcccagctacttgggggactgaggtggtaggatcgtttgagcctgggaggttgaggctgtagtgaaccctgatcgtgtcactgctctctaggctggatgatagaatgagaccctgtctcaacaaacaaaaaaactaccgtccacaaatcaagcatctctttgttggtcaaagactactttatagggcactgtctaagtgatgatagaatccagcagctcctcacatagttccaaagtcaattgtaggggaaaagaggatccctgcccctgagtatatcatcctttcattcctaaggtaccatgattctctataaactatttctatgttattatagaactcttctgggcaatgctctccccattagaacacttctctgacatcacccaagacttcgttgatattttaggttttaaaattatttttttgtccttcaatcataagagcaatttcaattaatattcaatagcaggctaataatttcccctcaaatggaaattctctggtccaaaatccaattgttgtcattgctggaaagtactcacaggtttttgccataagccattacccaattctcacacaccaagcctatatttcacctaccctaagtcatcccagggccagataccagtcaaccagagaccatctctatgctttagagcccaccagaattattaaaactagccaatcctaaactgtttacccagccctgccttgccttttccacagaaaccccaattaaggctctgacctaaggtttttcctcattcctcttttctgcctcctgaccaacactggtgctttctcctgtgatcttgcatagcacataataaccacctctctgggacctgtgaatgtgataaacttctttctgggccttgttctaattttctttgtgtccacactgactttaccatatcatatctgacatgtacattcttaagacaccacttaaccgtggcatttaccatgacctggtgtattagtctgttctcatgctgctatgaagaaatacccaagattgggcaatttataaagaaaagaggtttaattgactcacacttccaaatggatggggaagcctcagaaaacttacaatcatggcagaaggcagcttttcacagggtggcaggagagaacgagtgccagcagggaaatgccagacacttatgaaaccatcagatcttggccaggtgcggtggctcacacccgtaatcccagctctttgggaggccgaggcgggaggatcacgaggtcaggagatcgagatcatcctggctaacatggtgaaaacctgtctctactgaaaatacaaaaaattagccaggtgtggtggtgggcacctgtagtcccagctacttgggaggctgaggcaggagaattgcgtgaacctgggaggcagagcttgcagtgagccaagatcacgccactgcactccagcctgggtgacaaagcgagactccgtctcaaaaggaaaaaaaaaaaaaagccatcagatcttatgagaactcactcactatcacaagaccaacatgggggaaaccacctccataattcaattaccccattattcaatcccaccgggtccttctcacaacacgtggggataatgggggttacaattcaagatgagatttgggtagggacgcagctaaaccatatcattctgccctggcccccattcaaatcgcatgtcctcatatttcaaaacacaatcatgcctccctaacagtcccccaaagtcttaactcattccaacatcaacccaaaagtccaagtccaaagtctcatctgagacaaggcaagccccttctgcctataagtctgtaaaatcaaaagcaagttagttacttcccagatacaatggaggtacaggcactgggtaaatatacccattccaaatgggagatattggccaaaacaaagggactacaagccccatgcaagtccggaatccaatagggcagtcattaaaccttaaagttccaaaatgatctcctttgactccatgtctcacatccacgtcacgctgatgcaagaggtgggctcccatggctttgggcagctccgcccctgtggctttgcagggtacagtccccttcccagctgctttcacgggctggcgttgagtgtcttgcagcttttccaggtgcacgacacaagctgtcaatggatctacccttctggggtctggaggatggtggccctcttctaacagctccactacacggtgttccagtggggactctgcatggggggttttaccctacatatcccttctgcactgccctagcagaggttctccatgagggctccacccctgtagcgaacttctgtctggacatccgatgttttcatacatcctctgaaatctaagcagaggttcccaaacctcaattcttgacttctatgcacccataggctcaacatcacatggaagctgccaaggcttggggcttgtactctctgaagcaacagcctgagctgtacctttgccccttttagatgcagctggagcagctgggacacagagcaccatgtcctgagcctgcacagaacagggtgaccctggacccagcccaggaaaccatttttgcctcctagacctttgggcctgtgatgggaggggccgctgtgaaggtctctgatatgccttggagccattttccccattgtctttgtaattaacattaggcacctcattacttatgcaaatttatgcagctggtttgaatttctcctcaaaatgggtttttcttttctatcacattgtcaggctgcaaattttccaaacttttatgccctgcttcctcttgaacgctttgctgcttagaaatttcttccaccagataccttaaatcatctctctcaagttcaaagttccatagatctctagggcaggggcaaaatactgccagtctctttgctgaagcatagcaaaagttacctttgctccagttcccaagcagttcctcatctccatctgagaccacctcagcctggacttcattgtccatatcactgtcagcattttggtcgaaaccattcaacaagtctctaggaagttccaagctttcccacatcttcctgtcttctgaaccctccaagtctctaggaagttccaaactttcccacatttttctgtcttcttctgagccctccaaactgttccaacctctgtcttttacccagttccaaagttgcttccacatttttgggtatctttatagcagcaccccactctctgtggtgccaatatactgaattagtccactctcacactgctatgaagaaatacccagctgggcacggtggctcatgcctataatcccagcactttgggaggctgaggcaggcagatcatgaggtcaggagatcgagaccatcttggctaacatggtgaaaccctgtctctactaaaaatacaaaaaattagctgggcgtggtggtgggcacctgtagttccagctactcaggaggctgaggcaggagaatggtgtgaatccaggaggcggagcttgcagtgagccaagatcgcgccactgcactccaggctgggcaacagagcaagactccatctcaaaaaaaaaaaaaaaaaagaaagaaatacccaagactgggtaatttataaagaagaaaagaagtttaattgactcacagttctgcatggctggggaggcctcaggaaacttacaatcatggcagaaagtacctcttcacagggtggcagaagagagagtgagtgccagaggggaaatatcggatactcattaaaccatcagatcttgtgagaactcactcactgtcacgagaacagcatgggggaaaccacctccatgattcatttacctcccactgggtccctcctatgacatgggattatggggcttacaattcaagatgagatttgggtggggacacagccaaaccatatcacctgggtaatgggcatattcaatgtgtgaatcttgattatcacaaagccagttccacatggcttgcatatgaagcatatcagctgcttcatctggggtattccagttggcattgaaaggggaagtcagatagttccctttctcagggtaaatagaccttataatggctcttatctggtccaccaggtcagctgttcccttaggaatgacctgctctgtgtctggatcatgtgtagccatctgtgattgttccatagtgagctgtggatcctatataaacccaaacatgctcttccactctgaagcatttaaaagccaagatactgcccctaaattatttattcttacaattcattttagtcaaaattctttaggaagctgatggtactgatctacaaaataaagtaactctttcacactataccccctggtttcagtagtttcttggttttgccctcccctacctggactctctttttggtgaccagagatcttagaggtattttctgtttttcccacatattttttccattagaggcagctttgggtctagcatccgaagttcaggcccaccagaatctgagcgtggttcagcatcaaggtccaacccaacactctttttaactttcattttagctgttgcagataacaataaccattggattgtgtatttcacttttttcttattagtttgcatttccttaagcattcagtgaaccagctccccaggagttgggttcatctttaaatttcactggtaacttttacctttagtaactgatcacagcccctttgctgctccttaggtgactatatggccactcaggaatcaaaatttcctcatcccctacccttttattttatccttccaaaccatatgtttctgtgagcctgggctgatctagcaaatcccactccaattctggggtgttttccatccacaaccaaatctctaggccattccgttgttgggtgtccaacaattcaattcaattctgatactaactctgagaattagtacagaccacacaggttaagggctcagtcccacagaccgcccctacttcagacaccagtctcaagtaccaggccctcaggtgacccacacttcttccaacttgggcaaatttgagtgttcccacatccttcctcaggtttagtaattttctggaatgacccacagagctcaaaaagtttctttacttatcattaccagtttattataaaagatacaactcaggaacagccaaatggaagggaggcatagagcaaggaaggggtgacgtggagcttccatactttctctgggcatgccacacccccagctcctgaagctctctgaatcttgttgttcaagaacgtttataggactcaatctccagctccctttcctctctcttcttccctgaggtcactgggtggggctgaaaggtccaacgctctaattaccaggtccttctggtgaacagctccatcctgaggctgtctagtggccccagcctcagctacctcgttagcataatctaaggtgctattgaaaggggctcgttatgaatacaaaagacactcctgcccctgctacttagaagctgaggcaggaggatctagtgagaaaaacaaaacaaaacaaaacaaagcaaaacaatactcctatcacttaggaaattccaagggcttagaagttctgtgcttgatcctgggagcaggaacaaagaccaaatatgtttttgtattataccacagtttggaaagttcattgaaatagaaagaaaaaagaaaaccccacctctatcctaccacccaaagatagtctctaatagcattacagagtacttacttccaggcttttttgtgagttttgagcattatattcacattaatattgctcacattttatagtgatatttatgttgataaatctaatcacttttgctttatgaatgttactctgttactcagatctcatcttaaatgtcatttcttcagaaaagcttttcctgacaattcagtcactattttaacacagccctgttttaattcatcgcatgacactgaacacagataactttggagttttgtttagtgtctactctccttccagccttcttgggctatttttttccttgctagatatttacagtacctagaacagtacctggccctactgtggtaggtgtgcaataaatatttgttgagtaagtgactaaatgaaagccctaggtcagttatatgtgtgtatacatgagtttcttaaaggaagaaaagaaaataggctgggtgcagtggcttggctcacgcctgtaatcccaatactttgagaggccaaggtgggcggatttttctggaggtcaggagttcgagaccagcctgggcaacatggtaaaaccccatctctacaaaaaaaaaatttttttttaattagctttgtgtgtgcctgtagtcccagctacttgggaggctgaggtgggaggatcacttgagcccaggaggcagaggttgcagtgagccaagattgcaccactgcactccagcctggacaatagagtgagactccttttcaagaaaagaaaatcatttctcagagtctttttcttttgtcttaaatttcttaagagaccaagaaattgagttatttgtctaaaacttctgagattcaaatcacagcttcaactaacattccagctgtactgagctatacattaatttgtaccagtttttaaccacagagacagaaaaatgctgcctggcacaggatgtggtcatttgtgctgaggaaaggcatatgatgttgtatcagcttcgtgcaacttcttgaaggtgacatccatggagtctcactcagaatgaggcaaatgatagctgtgcctgtggcttgttttttttctgagttacagtttctgagttctggtaggtaataccattaaatttgtagttgaaatagtgatccctatgccactgttggtttgggaatttataatttatgttataaatttactttgggttcaggggtagcctatgagttttatattttcagatagtttgtggtaaaaaaaacagattaaatactcaaacataaacaagaaaccaataatttccattcaacaataggaatttaaaataaatattgtaaaaacccaagtggtgaagcaccttgttaggaacccatacaatagtatcgccttacctccaactacatgtcaatgggttaataggatgtttgtgtaaataaattatttttctcagttgctaggggtctaggagtgtcatgcagttcttgatagagaagatatgatcaatcactctaaataatttgggaaagagaaaagaaacagttatttactttaagcttcattcagaaagaaatgaatacgctaattttgttgttgttttgtttttgttgttgctctgttgcccaggctggagtgcggtggtgcgagctcagctcactgcaaactccgcctcctaatttcaagtgattctcgtgcctcagcctcctgagtagctgggactacaggcatgtgccatcatgcctggctaatttttatatttttaggagagacggggttttaccatctctactagatggccagactggtctcaaactcctgacctcaagtgatctgcccacctcagcctcccgaagtgctaggattacaccactcctggcctgagtaggctaattttaacgttgaaagatcattccattattttgtgattaatctgcaaacagattatagtagtcccccttctctgcggtttcactttctgcagtttcagttccatggtcaaccatgatcccaaaataggtgagttcagtacaataaaatattttgagagagagtcaacaattatgtaccttttattatagtatagtgttgtaattgttctattttattgttattgttactaatttcttactatgccagatttataaattaaaactttatcataggtatgtatgtataggaaaaaacgtagtattcagtcatataccacataatgtttgggtcaatgatgaactgcaaaaacagtggtggtctcataagttaagacctatttttactgtatcttttctaagtttagatatacaaatacttaccattgtgttacagttgcctacagtattcagtacagtaacatgctgtacccataacatgctgtacagtctaagagcaataggctgtaccatatacccgaggtttgtagtaggccatactgtctaggtttgtataaatacagtgtgctctataatgtttgcataatgatgaaatcacctgatgatccactgcttagaatgcatcccatcattaaggagcatgtgactgtatatagggtttggtaccatctgcaatttcagacatccgctggaggtcttggaaagtatcctctgtgaataaggggggactactctactgtactattaacactggcacttaatctgggtcatatatgtttggaaaaggtggttaccaggaccctgcttttataaaatatataatatgatcagtatgtataaattatacatgctaaattataaaagtaatagaatgtaaaaacagcattgcccgatagaagttatacctactgcataataaggtaccactgccatatgcagcacaggaaaacatttaagactagagttaggcccggtactgtgacacaagactgtagtcccagctactcaagaggctgaggcaggaggatcacttgagcccaggcgtttgaagctgttgtgcatcatgattacacctatgaatagccagtgcactccagcctgggcaatatagcaagactccatctataaaaaaagaagactagaattagaattatacttttaaaggtaattaggggaatttagctacaatgtaataattattttaaaatgttagagaattctattctgtttaagtttaatggcagcatttattttttaatgtcatttgaaagattttaggaaccaaagacttgtctggagtgtttaaaattcagaataattgcaatcatgtttaatgtatatgttaaaaaatgaaatatcgtgtgcatctttataagcttccagttcttagaaaattgttttgtagaaaatttcacaccctgatggaaacactcactttattttttctcttgcggaaagacagactaatttctaggggaaaaggctttacccaactagagtaagtttggaaaacaaaacaaaaatacttggaaaaaactttaaaaaactattaatattggccaggcatggtggctcaggcctgtaatcccagcactttgggaggcccaggcaggcagatcgcctgaggttaggagttcaaggccagcctggccaacatggtgaaaccccgtctctactaaaaatacaaaaattagctgggcgtggtggcgcatgcctgtaatcccagctactcgggaggctgaggtgggagaatcttctgagcctgggaggcagaggttccagcaatgagctgagatcgtgctcaagcctgggcgacggagtgagactctgtctcaaaaaaacaaaaacaaaacaaacaaaagtattaactttgaacataaatacgcttgcttgtgatcccacagacattgtggtaattttttacagtatattcccaggagcctagtgcaggtattggcaaactttttctgtaaaggaccagatagtaaatatcttaggctttgtgggctatacaatctctgttgcatcttctcaactctgccattgcaaggtaagacaactgtagaagatacataaatgaatgtgagtggctttgtaccaataaaactttatttacaaaaataggcagtgggttggatttgacctgcaggccataattcccttactcctaacctagtgtatacatttattgtatgatgagttaagaaaagatttggcacttgaaaggaacagtgtttgtgtaaccatttcaaaagggcatcagtgaggattctaatactattattttaaaaatcagaaaaatatatatcactaacagctttttatcacatgcatgtatagttgatatagctctatttacataaattaattagacataaataaaaccatatgtagactaaatgttgtgttaccatttagttggttttctcctttattcctgaaaatttaaatctaacaagcactggcaaatatttacatcaactcaagtttatttcctccaggaggcctttttatactatcccaccttccacagtgtggtctcagagctgaatttttaagagtatgtttagttagtacttatttaaacacgtaattatattctgttcgaaattgtttgtgcatgacaaggtttcaccccattatatcataaactttttgaaaaaagaagccctaacctatatttacatctttctaagaatacttacctatgaagtgatataatccaaccagtttggaaagcagaagaaatgaagaaattcagagttctttttctagcctgctcattcactcttatgaacaattattatttctcttcctcttagtgaccaactgtaactatcttcagactgatcttgagccataggaaataacccagagatccccaagtctttatttttgtttagcatccaatgagtgctgggaatatcacagccactttactgaatacaaaagatttgggaccttctacttggatctaaaatgtcagcaagatactgtgatgtcaaaagcaagagttaaaacaattttataaaatatcatgagaaagaatattattgttgcttatatcttggcctatgatcacagagcaactaaaaaataaaaacctctgagtatcaaggctagagggaagatgtgtttcatgctagtgtactgagggatattaactaggtagagataagactcgagatggtggaatgtcatcccagcaaggcaattgtcatgcacaaacaatttcagagagaatgtaattaggtgcttaaattagtggaaaagacatggccttaaggcctgaacgactgaaatagaactcttgcgactttttgtttcagtccttcttcctctgaaattggattcccgtctttgccatttcccacttagaaaatagaaggaaaggttctgtcatgagcatgtatatgttatgagggtatgatgcatttgccgatagtattttttcctctttgaatagctgaggaataaaagttgggctactgaccccctgtatttaacactctactggccccctatttttcagcagcccccatcatccactatctctatttacacaagccatttgttctggagctgctgtaggggtaaaaagtacacacgatacctcgatacccatcaacactagctgtttaccctgcctgagttgcacatttaccctctgtcttagttttccagggcttccataataaattaccacaaactgggtgacaaaacaccagtttgtcacaaagcaacagaaattttttactattctggagggtagaagttcaaaattaagatgtcagcaggaccatgggaagggcctagggaagagttcttttttgcctctttctggcttctggtgacttccagcaatgcctggtgttccttggtcatggcagcataactctaatctctgcctccattgtcacgtgggcttcatctctgtgggcatctcctgtttctgtcctcctctagggagactagtcattggatttagggcctaccctaatccagtgtgacctcatcttaactaattacatctactatgacgatatttccaaataaagtcacattctgaggttccaggaggacatgaatttaggagggacactattcaacacagtacacccttcatctcctggctctgtaactaaatatcaaatagacccaatacccatcatgaaatagatttcttttttaattaattgtgcagtggtgatcaatcggtgtatgtatttaattacttccattctgtacaagctatgggtcttgcctgattccaagagaaaagttgaatttgatcacttttgaaaggccttatattttctccattgtgacatcttgtgcagtgtagatgcttagatcaaagataagaactttaattagaaagcttgctgaaaaaaataattcttcttccccttaggagaaataactcccatcttcagtagcttcccatggcatagtctcggtcccttttgaggtgtacaactctttggtgcttagaagaagtacaaactactttgatgcttctgaaaggggaaaaaaaattaagccacatgtgtggtttatggatgctaattaccgccacatttagatgtgtgaagaagggtggctggagcgagctgggagagaccgcgtcagcctcgcttaggcactcgctgaggctcccgcggtactgcttagtacacgcaaatatatgcttcaaaaggaagtgccgttttttatcaaaatattacatctctcaagcccagatcaagacctgtcactcatgggatgcaggggttatcttacaaaatcagcagtgctgtgctgcagaagttcactcacaggggatttctgtaagtgcttaccatgggcagagctttacagcttgtactgtgtcaaacgggtaagttctgttgatgataaaaccaagacatctctccctgaatctttttttttttttaatcacgctgtcacccaggctggagtgcagtggcacgatctcggctcactgcagccccaacctcccaggctcaagtgatcctcccacccgagactccagagtagctgtgactacagttgcatgccaccatgcatggctaatttttttttttctgtagatacggggtctcgctatgttgcccaggctagtctcaaacttttggactcaatcaatcctcctgccttggcttcccaaagtgctgggattacaggtgtgagccactgtgcctggcccttgaaactttttaacagaaaacatctctggattttcttttcttcttttttttttttttcataactaagctgcgtagcacagcaaactaaaaggaatgtgtaggaagatatttgacatctgccaaagagccacagacagttgcaatggttaacagaaaaatggggctgcccactgtccttagtggatcctctggagagttttctcccttctttttgcattctttttatttatttatttatttatttatttatttattttttgagatggagtcttgccctgtcgcccaggctggagtgcagtggtgcgaactgggctccctgcaagctccacctcccgggttcacgccattcttccacctcagcctctaaagtagctgggactacaggcgcccgccaccacacccggctaattttatttttgtatttttagtagacacggggtttcaccgtgttagccaagatggtcttgatctcctgacctcgtgatttgcccacctcggcctcccaaagttctgggattacaggcgtgagccacttcgccctgcctctttttgcattctttgtctgttaagcagtagtgttggtgcctccttccagctggattaagagaacgcatgaaacacccttatgtgcctgctaactttcatagcagcttttcttgccaagtgagttctgcttctcaaataaactttggcaccaggcacggtgacccgagccattgtgtaccttggtgccatttgatagctagaggaaggtaccctgctgagggataaatgaggagtcccagtggtgtgccagaatcagctcatagctgcttgttaaattttcaacaattttgtatgctggctgactccatgatggtagcttgaaattggccatggtggagtatttataccactgaaatcagcaaatgccacaaataagcccttccccgatcctgagctggttgttaagcaattaccagaacaccactggactgaagactgaacaaaatcctgtcctggctcatcagaactatcaaattttttgtaatttgtcagcccaaagggattttgggttttgggtgttctgttttgtttttgtttttgtgttgtagtttgcatgaaagagaatagtgtcggagacaagaagagtacatgagctctctccacaacatagtagatgggatcccgaaattttatcttgtaaaatatgggggtcatattattgcaagtaaatgaaatatagtaggtggttttgtgttttgttttgtttttaatatactgttacaaaaagcacaaagcaatgacagtgaagtttaaactggacacaattgtaaaccttaaaagacccaaatgatcttgaagcaattacttcctatccattcccataagaactttagcttgctcttgtctgagaagtgggcaagggtagacaactggaggcaatcagcaatccttttacatccaaatttgtacacattgtattgtggcagggtgttattgtacttgattgtgtcccaaagacatggcaagaacagtcttttatagttgtgttcttttgtgccaaagttctgggaatatagtaaatgctcagtgtttatccatttttctttctgcctttccatccaaccatccattcatccatttatcccctgcagtgttcacaagtgctcactaaataattattggttcattaactaaagtgaagaacagcatgtgagtacattttggcattggggtcttttattttagtagtgttcttgaaatgctgggaaaactttccaaggattgcatgggataattttaagagaatcaatttccaggtccgcaacttcagtatattctctgcccttcttcctaaaactgatctgcctagaagactgctgccattgagcttgcttgctttttcttttctttcttcctttctttctttcttccttccttcctttccttccttccttttcttccttcctttcttccttccgtcctttcttccttccttccttccttctttctttccctttctgtctttctttccctttctttctttctttctttctttccttctctctttctctttctctttttttatttcctccctttctccctctttttctttctctccctctctttctttctttctttcagacagggtcttgctctgctgtccaggctggagtgcagtggtgtaatcacagttcactgtagccttgacctcgtgggctcaagcaatacttccacctcaacctcctgagtaactaggtctacaggcacccaccaccatgccctgctaattttttaactttttgtagaggggtctcactgtgttgcccaggctggtcttgaactcctagactcaagtgatcttctcgccttggtctcccaaattgctgagattacaggcataagccaccatgcccagccttccttctccactttataaaagaaaagaacatttctcacccacctctaaggtgtaagtacttccaagatgccaaacaaaggatcaattagataatttcttttatcaaattttcataacttatttcctctctatccttctcattaggagatacagtcccaataaattttactttttgtatttggtaattattgatcaaattataatatacctatattgttttactcaattgtatgttaataattgtaatgctaattcaatccagaagaaaacacattttgacacatagcacttcatgttaacaaaagtaactatattttattgggtttttttttttttttttgctgcagatacatataatgggatgaacaatagatgactcaagcatacaagagtactacattaggagaaaattccatgggggtagtgaaatggaaatagaagttcaaagagaaatttcctgactttcttgaaaagcttacttatgtatttttaaattcatgatggagtaaattaaatcacaaatatgtatatattcctaagtagatacatttgaaagggtgtggtaatggtttaaactatcattatttacaatacacaaactgtattcttttgtttaaaaatagttaaactcatgatgaaaaattatagatgtcaacttagtatgtacttaatgtccactatgtataagagagatatataatttttcaaaattcttttgaggatgcttgagcaaaaaatttgaaaaatgccaaccactattatatgaagtttacaaaatataacacagaggtgtctttaagcctgagcctatttaaaggcctgaggcagagattctcaacctgagatctcagggtttgagaaatcaatcaatagtatgtgaaatttcgtgtttatgggcatgtgtacctttttctgggcaaagagtacagttctcaaagatgttcacaacctaaaaaagattaaaaagatctgaccaaagcatgtaaagtcttaagaaacttgactttgctgttttcataatagtttcatatgcactggcatgtggaattgcttccaacataaacaattttggttcagttgtttgtatataaaataatactaattatgaaaaataatgaaagattcaatctccctttcttcatcatcatcctgaaatctatgtcaatcttttaattttattccttgcttattcagaggccttttcttttcttaattttgatggagaaaattacaggtgcttcatccatagaaaaatgctgaatggaaatgtggaaaacagtttttaaaagaatagacatcatctgcccctgacagttgtgtcttgcggtttcttccaggatctaatcagcagtctagcaataggttgacatagtattcaggcttagaattaggaaaattacatttggaagtgactaataacacagtactttcaaagctttggattttaaagcgtcacacacacagcaggtgaatgaagatgtcagagccaggcctaaaggagcaccctggtaatccagcccttggtgtataaccttcaaacaccaataaacaagattatctgggtttaacatgtgacctttctcattttgactggtaggtccagatgttttctcgtttagctgcctggttgaaatctgctttaactgactgcatttggatctagatttgaggttgcaggaatgacagtcttgaaaactacattctgcactgggtgatactgtctaaacagtaacatatggagacctatggtttaataactaaaaataatttgtgaccttacccttaatagatttgctaattatgtcaactgaataagtaaaaatgggatcacgatagatatttcactatattcatcagtctggtggcattaaagacattgacaagatgtgttagaaccagccttttctgcacctgcgtctacagaagcaaaaatctcagccagggcctttgttatcctttcttactccttaagtaatttcatagcagcccttgagaaaataggaaaggctctttgttaacttctgttaagtcagcctaggctgtggtcatacctcccatccaccgacatttgaacaaacactacttaatggtccattattactcttttcattacttggagcccagaactctctcttgcatttttatatgacctattccttacttaccatcagcttgattttccggttctagtccttcaggttgtactagttctctctctctctcagactgtaaagaaaagataattctttattttcttattttgtcttacgagagaactcacattcctgaatgaaccctcagtgaacttccccaggtgcatttatatcactgctgctttgcagtccagagatagtgctcattaactaattaataaccacagcctagtaaaacacactaaattattaacagaacagaaagtgagacagtatggtccataaaagcacaaaatataacaaagaagtaatatatccttatgtatctggatccttatgaattttagcccaatttagaaagagcaaaaatcagtagttaggtatttgtctgaaatcgataataaaataactaagtatgggaaatgtccaatcaggccaactcttttatacttgattttttaaccttcctttcctactgtcaagtagcgggtgcccttgtgtttttcaacaagctaattcttgttaattttgttgaaagggaatgaaatcagttgtacaggaacaaaaggagttaaaaatagtcttctgagcatttgtcatatggaagtaatcatagctcagaaactatacttaccatgatgcttcttgtaatcattggtgaatgggtagctaaacattcccattttgagacagagtcagtatcagagtcagtatcagagctatcaattttattttgttttgtttatctgtatagatttctttttaaaacagctttaatgtggttataatttacatactttaaatgttcaattcaatgacttttctagtaaatttacagagctgtacaactaccaccatgttttagaacatttccatcaccctaaaaaaccctttgtacttgtttgtagttgatccccattcttgcctgcagcctcaggcaaccactaattatttctgtctctaaagatttactgtttctgggtatttcatataaatggaataataccatatgtaatattttgtttctggcttcttttacttagcataatgtttttgaagttcatctatgttgctgcatgtatcagtagtttgtttcttttatttcttttttcttaaaatttttgagatgggatctcactgtgttgcctaggctggtctcaaactcctgggctcaagtgatcctcccacctcagccgcccgagtagctggaaacatatagaggccccgtctctacaaaaaaaaaaaaaaaaaaaaaaaaatttaaatagtcccagctactcaggagactgaggtgggaggattgcttgagcctaggaggtcgaggctgcctagtgagccatgatcatgccgctgcactccagcctgggtgacagtgagactctgtcaaaaaaaaaaaaaaaaagaatatcattgtatttatatagtacattttgattatcaattcactgtttggtggacattcaattgtttgcattttttagcttctatgaacatttatgtacaagtctttatgtggacataggttatcactgcttttgagcagatacctagaattgttggcttgtatggtaaatttatgtttaattttataagaaactacccctatattagttattgattgctgtgtgacaaatgaccccaaaccctagcaacttaaaacatattatctcaggaatacaggcacagcatatctgggtcctctgcttcagtctctcccaaggctgctaacaagatactgacctgaactgtggtctcatctaaagacttgactagggaaggatcagtttccaagctcactcatgtggttgttgtcaggattcagttcctgaaggctgttagactgaaggcctcgtttcttcactggaggttggccagacactgccttcagctccttgctctccagcttacttcatctgagaagcttgcaagaaacaccacagagagaaagagagagagagatggaagtcacagtcttttataacctaaacacagaagagacaccttccatcacttttccatgtttttttcattagaagcaatcacctaggtccagcccacactggaggggaatattacacaagggtgtgaatgctaggagccgttttagaaggctgcccaccatactgccaaactgttttccaaaatgactgtatcattttacactcccaccagcaacggatgagggttccattttcttcacatccttgccagcgctctttattgtgtcttttttatagtagccattctagcaggtgtgaagtggaatctcattatggttttaattcacacttctctaatgactgaggatgatgagcatcttttcattggtctgttgtccattcatataatttcctggtgaaatgttaattcacatcttttgtgcgttttaaaaattttatttattaccttagaaaagctaaagactactttcgtgcattttaaaattggattatttgtcttactgagttgcaggatattttcatttatgaatcatgttttcagtgctgtatctaagaaatctttgcctgtctcaaggtcatgaacatattatgtgccagctactatgctacgcctatatatacattattttgcttaatttcattactgtaacagccctattaggtatagatattatcattactgcaacacaaatgagaaaattcacggaagtcaaattacttgctgaggtcacacagctagttagtacaacttgaacctgggttctgtcttactccaaagcccatgctcttttatcagaaacttaaaaaaaaagaagtcattcaactctattttcagtggaaaaaaataagttaaataccatattgttccttccctgtaattaccaatacccatccaatattacttgaatattgttagtagaaacctctctgtgatcattagaagctgctgtttgaaatagaaaggacatagattgcagtttgttggccagattctattctagttttattctgccagacaatattccctctgcagtttccagtagagctaggatgcatctttgcttgcagccttgaagtatgttactgggcagctgttaagcagttgttacactttatccccagggatgaacatgttccagtgacaagatagctcattcccttgtgtgcttttaatgttgtacattcacccaggagaagtcagtgctgagtttcttctattatttctcagtgctctaattccattatttgagaaccagggaagaaggaagggaagaaccagagatgggtaaaagtctaaataatatgttacagatggtaaatgttaatttttattttgtattttaatgtaatgctaaaaagaacaagagtcttctaattcttaatccttatactcatttccttctcatccaagaatcctttatacgctttgggtctgattgttataccttgagttagaaataacaatttattttttccctctgtcttcggctaatatgctagctttaggcaaaattgcaatctgtacaatttttaaaaacactgtcaaccctaattttgtacttgaagcaaataatagtatagttgttcacgctttcctttcctccttcttatcttctttccttccttacttccttgtttgtttggtaagtagtcactgagctctgtatcagtgctaaatgttgatagaagcagcataatatacagacgttgacatcaaacagccactatgtagagaggacaacggggaatattataggaaaagcggagaagaaatgggagagataaggagcttgtttgcaacttgttgagtgtgagataagtgttggaaattcaggaagtgattgtacatgtcaggaagtatatgtctatggctttccaggtctaccggaaataaaaatttgagagtcaacagcaataaaaacttggatcaggagacagagtatgtaaagtgaaaaggcaaaagtttcaagaaataggccctaggaaataattcacatctaggagatagagaaagaggaataaacagaggatataagtaaataagtgaggtaggagtaggaccagcaaagaataagccaaggaaagagatttaaaaagcagggactggccagtggcatcctattgcaaaatggtggagcaggaagggcagaagccagaccacaatgggcaaagaagtcaactagatgactggctatcactgtgggccatggaccttattctgatgcattaagagaaactacatactgcatgacagcatcagaatcttgtagacaagttaggggtctattgtgatctactacttcttctctgcctggcctagttatcatcactaaattggttcaggctgcccaagaagttcattcattcattcattcaatttcttttttttttttttttgagacagagtctcgctctcacccaggctggagtgcagtggagctatcttggctcactgcaacctctgcctcctgggttcaagcgattctcttgcctcagcctcctgagtagctgggattacaggcgcgtgccactatgcccagctaatgttttgtatttttagtagagacagggattcaccatgtcagccaggctggtctcgaactgctggcctcaaataatctgcccgccttggcctcccaaagtgctggagtgttgggattacaggcatgagccaccacacccggcccattcattcaatttctaaagtaacgaggcccttgttaggttctggggacacaaatattatttaaacctggtccccaccttgaaaatactcctagtctatatgggagataaatacactaattgtgatataacacaaaacatgctatatacaccaagaactacttatgctctggatatagaagactttttcattccttcctttacctgtgaccctgagtttggtattgtgacagtcagaatgttttttagccaattgtatttggatgacagtttttgaattttactaagaattgtattttaacatgctttaaactttttcattcctgtttttaaaaaagatagagtggctgggcgcggtggctcacgcctgtaatcctagcactttgggaggccgaggtgggcggatcatctgaggtccagagttcgagaccagcctgaccaacatggagaaaccctgcctctactaaaaatgcaaaattagcagggcatggtggtacatgcctgtaatcccagctactccggaggctgaggcaggagaatggcttgaacccgggaggtggaggttgctgtgagccgagatcgcgccattgcactccagcctgggcaacaagagcgaaactctgtctcaaaaaaaaaaaaaaaagatagagtgaatattctatgcaagggaattttgaatgaaaaaggaagttttttttctttcggatttacaaatgaagtttctaccaggtaactactttagtctatgcttgtttaggcaacatctaaaacttcagcctctttaaatgtatttcatccattgttttttgatgggtagcagttatttctcaaaaatataaatacaaaagagtttaatacatagatgaaaataatcaatagaaaaagtaattgttataatgggcagaaatgaaacaatatacttgtagactctttatagtttagaaagacctttcatacatcatggccttcagtcctcacaattctagataaagaattatacctggccccatgacatttgaagagcttagcccagctgagcagctggaaccttacctactgctgatgtatagtgtatttctgttgtaaaagtagtaaatctcctcttcttctaatggggaagcaaagaattgcactgaaattattaagcaaaattatttttattattatgcccatgaatgagctatttgatagtcagataaggcaaaaattaatgttattagtaaaattgagggtagttcctttgcaaatgaaactttttacatgctaatgctcttgtgtttgaagtgatgtgatttactgatggccagtttagattcttttttttttttttttttgagacaggtctcattctgttgcccaggctggctggagtacagtggcatgatcacagctcactgcagcctggacctctccaggctcaggtgatcctcccttctcagcctcttgagtagttgggactacaggcatgctgtattcccagctattcccaggtattctaccatacctggctattttctcatgactttaattttttttcttttttttttttttgagacagggtctcgctctgtctcccaggctagagtgcaatggcatgaacacagctcactgcagccttgacctcctgagttcaagggaatctcctgccttagcctcctatgtagctgggacaacaggcacacactaccacatccggctaattttttgattttttgtagagacagggtcttactttgttgcctaggctggtctcaaacttctgggcccaagcgatcctcccacctctgcctcctgaagtgctggagttacagatgtaagctattgcacctggccaaaaatgtatttcaatgagaacttaaatctatagaattttatccaaacctgtattgagatatcagtatttcatctctctttttttttttttttttttttttttgagatggagtctcactctgtcacccaggctggagtgcagtggtgcgatcttggctcactgcaagctccgcctcccgggttcacagcattctcctgcctcagcctcccgagtagctgggactacaggtgcccgtcaccatgcccggctaattttttatatttttagtagagacagggtttcaccatgtcttgatggtcttgatctcctgatctcgtgatccgcctgcctcagcctcccaaagtgctgggattacaggcgtgaaccactgcgccctgccagtatttcatctcttgttagcctgatttaccttttccagttttttcctaatttaaaaataggcattttcaaaattaacagacgtccattattttaaattttaaaagagaaaaaaagaaaacataatagattttataaatcaatgaccaaaagtttagtttgcattgaagctttataattttactggttagcaaatattaaaactttccttactctttagttctaaaaatatcatcggtttctctctctcattactttacatgcaaaaatgtaaattagaaaatgaaaatgacccatcgtttcagtgttcttcttcctaatacaaagaggtcattgtgggagagaagtatagacattaattttataaggaaaactgttctagttcatctgtttgtgctagattgggcgacagtaacaaactttgtagactgaatacctacaatacatatatttcccattttgctctttagtgcttcaagcttctaaatcctattaacagctgtcaaaacctggtaaactatttagatgctttcacaatttgtttaattggatagttagaaaatggcgtgctagaaaagcccccccccaccccctttcttttagcctggctgaacatctgtagcattagggcctcattatctctgaattggcatagcaaatcagtggcatcattaaaatgccacattaagatgattaagcttctttgttatccagttgacttttgcatttgctgaagatttcatggttatgacccccgatccttgtaaaacacttaaataacatcacccttttgtgccttccctaacagcctctatcttgttttgtcccttctcacaatatctccggcagacctgaggatattacttccaactttgtccttcgtagagttggattctgagatctcatagtttaaggtggaaattcagaatctcttttttccactgggaaattcctagtatcaacatgctctccaagatacccaagttgtctattctgtttgccatctttgataggttcttctctccaactgagcattttatgtgcctgtaaaaacaaactgaggcacagttggctgagactgctattgcccctgaaggatttagatgttggatcctttccaggacccattgtttttgcagtgtctcaggtgaaaccaaaaatgtctgctctggagtacaagcctttcatgtgataaccatcaccccaggaaatgaaaccatgtgcatcagccccagtgagccaaacctttgttctcttcagcctacaaaattctctctggggtgtgtgtgtgtgttcgtgtgtgtcatcttcctaagaggcctgaaaacatagacaaagatgggagcaattgtaccctagagtagctcctttagaaagtttagtttaatctaatttcctatgggttagattcaatttagacatcttttccctgcctctgggaatgtatagcaattacctttatcagcaatttgtggtttggtatattcttttctccacaagttcagttataaaaaaggtattcaggaagcctttaactttttttattataaaatagatcatatgtacaaaaaggcaagtataattacatgtatagattaaagaataataagaaaacatccatacacccaccacctgacttaagaaataaactttcaccagtgtctatgaggcttcttgatagtcttctaagtgtcaacttaagatgtatgtctaagaaacctgatatatattataagaagattagaatttacaaaaccaaaacatgagattattcatactataaaaggtttattcactttacaaaagacttcaggatactatcaggcagttccccaaatatattcaagtatttgttgccaggtctttaagattttctgcctttattaaatgaacgggtgttgccagtcactgacaactgaaatcagggcaaaataaacatttctgaatgtagaatgttaatcagcgggacagtgttattggttttgcatgaaacattttatgaacacatcaactacataaaagcggatctgtagcaatgtaattatatcatactaaatataaagcagaataagacggatgtatgagaacaaagaaacataattttttttctagttaagggtatcaaggaagacttcatggaagaagtggcatttaaactgcatgtggaagtttgaatacactattgaaatggtagaaggatattttaggtgtgggaaaggctggagaagaggcttgaagaactgtgattagtcatgttgctgctgtgcttctcagactttaatgtgcataagaatccttgaggatcttgttaaaatgcaaattctggtccagtaggtctaggttgaggcctgagcacctgtatttctaacaagctcccaggcggttgcagaactgcttatccaaggacctcactttgcatagtaaagttctagtatagagagaaaggtgattcaagaagactgagaaatgagatgattaagtagtcctggggcaagttctggacaggtgcttgaatacctcattaaggagtttggactttattctataagcaaatgaaatcattgaaagttttaaataagagtttctttgctacagcatgattattctggcaataggatgcagtataacagaggtcacatgtaccgtgggccagttaggaggctaatgcaatgatatattcatacaccaggaatttatgggatgtctactttatgtagggcactgcatctaactcgaggcagtggctctcaaactccattgtgcataaggtttatctggagagcatggttttaaatgaacatttccagtctccacccccagattagtagatctatggtaggagccccaaattttctattttaaaaccacataacctctggtcttacagatacaggttaagcagtgtcatagggaatactgagttaaacacacacacacacacacacacacgcacacacacacacaatctgtatagcttctgccctcagctagtttacatatttattggggagactaggggaggtgagttcataagtaacccaggaagaaggagcaggctattttccagctgggttgatcagagaaggcatagtagccaggagacagcatattagtttgacctttaaaaatgtgtaggacagtggccaggcgcagtggctcatgcctgtaatcccagcactttgggaggccgaggccggcggatcacgaggtcaggagattgagaccatcctggctaacacagtgaaaccctgtctctactaaaaatacaaaaaattagctgggtatggtggtgggcgcctgtagtcccagctactcaggaggctgagccaagagaatggcatgaacccgggaggcagaagttgcagtgagcagagatcgcaccactgcactccagcctgagagacagagaaagactccgtctcaaaaaaaaaaaaaaaatacgtaggatgtgtaggacttggacctgtggccatattggggaaagatcttttaggcagtagaaataagactacttagaagctctttagttaaagatctaactagtcaaaattaatatgccaaagataggttggggccagatcccgaaaggccttttaaattatttgtttctgagtttggactttattttgttggcagtggaaaatattgaagaattttgacagggctatgacatgaccagaggtagaatttaggaagagcaatcttgtagcaacaaacaagacgggctggaaaggagaaagaagggagccaaagagaacagtgcgggggctacagcaataagtagatcgtgggaaagtcatagggtctgagaattaggtaatgggcatgcaaaatacaggagtggagaaacagattagagattggatacacagatagaaccactgaatgattgaatgtggggcccaatcaggagaaaggaaagatgactctaaggattctagcctgagtaactgggaagatacctaggaagccttcagaaatagaggactaaagaaagggagagaggttacacctggagttatatatatggaaattaatcatataagatgttagtcgaagccttaagaatgaatgaaattgccatacaagaggggtgtagagggaagaaaaaagaaggttaaggacagtatttaggatgcagagggaagaaaaggggcaagaaggaagcaatcagaaaggcaaggaaagaaggtacattttcattgaagaaataggagtgcaggggccgggcacggtggctcatgcctgtaatcccaacactttgggaggccaagacaggtggatcacctgaagtcaggagttcgagaccagcctggccaacatggtaaaaccctgtctctactaaaaatacagaaattagccaggtgtggtggcacgtgcctatagtcccagctactcgggaggctgaggcaggagaatcgcttggacctgggaagcagaggttgcagtgagccgagatcgcaccactgtactctagcctgggcaacagagtgagactctgtctcaaaaaaaaaaaaaaagaaagaaagaaaagacataggagtgcagaagaacaaggaatggtgagtggtcagtagtgttgaggattacaaagaagttggagagagtggcatacaaaaaggctctgaatctaggaatcaggagattatggttggtgatattcaagggagcagattaagtgaattgtggaggcggaagccatattacagagaattctgagtcctttttgaggctgtagaggcagtaagcatagcttacctatgtctttttttttttttcttgagacggagtctcactttgtcacccaagctggagtgcagtggtgggatctcagctcactgcaacctccacttcccaggttcaagcgattctcctgcctcagcctcttgagtagctgggactataggcgcacaccaccacgcctggctaatttttttgtatttttagtagagatggggtttcaccatgttagtcaggatggtctcgatctcctgacctcatgatccacccgcctcggcctcccaaagtgctggaattacaggtgtgagccaccgctcctggaccacctgtgtcttaaatctgtctagaaaggaatgttgaggccgagcgcagtggctcactcctgtaatcccagcagtttgggaggccgaggtgggtggatcacgaggtcaggagttcaagaccagcctggccaagatggtgaaaccccgtctctactaaaaaaaaaaaattaaaaaaatagccaggtgtggtggtgggcacctgtaatcccagctactcaggaggctgaggcaggagaatcgcttgaaccagggaggcagaggttgcagtgagccgagatcacaccactgctctccagcctgggcaacagagtgagactctgactcaaaaaaaaaaaaaaaagaaaggaatgttgagagaggatgaatttaggtatgatgtgatgaaaggtgaaaccaaattgcaaaaggaaaggaaaagaggagttggcacctagagagcctgaagacatagagttgatagatcttggagactgacttgatgtagaaattgaaagagaaaaggttccacagtttgatggcttggagactggaagaagggtgatgccatttgtcaacataaataagtcataaattagaagcaagtttgggttcaaagatgacaagtttgattttagacatgttcattagtattttctaatagaatttttctaagtagaacaccccttttctctgcttcaataagtcttccagggaatgggatgaatctcttcattagagcaggctaatgcctttcctggttttggtggactgtgtttggaggctgagtactaagcctaatatctctcaggaatctcattcataccttaaattctaatttattttattttattttatcttttttgagatggcgtctcgctctgtcgcccaggctggagtgcagtggtacgatctcgggtcactgcaacctccacctcccaggttgaagcgattatcctgcctcagcctccccagtagctgggagccaccatgcccagctaattttctatatttttagtggagacggggttttgctatgttgaccaggctggtctcaaactcctgacctcaagtgatccacctgccttggcctcccaaagtgctgagattacaggcatgagccactatgcccagccagcaatttattatggcttaccttgagaaggtaacagagttgaatattagaataaattagaatctggttggacacggtggctcaggcctgtaatcccagcactttgggaggccaaggagggtggatcacttgaggtcaggagttcgagaccagcctggccaacatggcgaaaccccgtctttactaaaaacacaaaaatcagccaggcattgtggtgcatgcctgtaatcccagctactcgggaggctgaggcaggagaatcacttgaacctgggaagcagaggtggcagtgagctgatcgtgccactacactccaggctgggcaacagagcaagactatgtctcaaaaaataaaatagaataaaataaaaataagttgctaatccccagtaaccatgcagagaggtcatccaggtcattcatctaaaactggtaagactccaaaggcaaaccagcaactatgtaagcccaaaggacttctctaatgccctgtccctgtgtccttatgtccctgggtctcaaatactgtcttccttgctctatagctgactcctaaaggactaaagctaagatctgctcttgagccaggatggtgtcgtattgaattccaggtcaggccaactaaagccaaaatcctgagggaaaggagcagctctcttatgtcctctgttttcagaaagactcaacacattgatcttactacattttaaagttcagtgaccatggagtggcctgagtctggtcatgttcacattttctaaccaaaatggaaaggatccttttctgctggccttttctatctgctgtttttggttttaagtagctgaaattttccgttgaggttcttttaaacgcctacatgcaaactgaagagcactcaagctttggtgggatattacccacagctggagtgatggaggaagggaggcaagtacaagcctcaggtgcatccttctcttaaaaaaataaaaaataaacacttaagcaaacaaattccacttgaaagcagggaagacctatccagagaaagaagtaaacctctttccaagtaggaaacagatatcatttcttatataatccagatccatagggtgggcaaatttgaaacatgttggacttatcctacagccctacatctttgctagtcaaagagtgaaaagttgtgaaccatgaactgtggtaggaagcagtggaactgtgtggcagccctggctcctctgatggtggttaggccctgtcttgctctattaagcaaacacagttaatatagatgaaaggtaaccttgatagaagtgaaaattgaaacttgggttgaaggtagcaagccaggcacagctgcagttgtacttcctcctatgcagcaagaggagcaacacttactgatgttttttggaagggatgtagttttttaaaatatcttttttcagactgttttgggttttaacatttacaccattgatgggagggtgtgtaatacagttggtaagaacccacttaggccttatatgtctagtgcatgaggtcaaatccaagtaattgatagttcaagcttaagttttgcattctaatcacacctctgccagtaactggatgtgtaaccttcgacaaatgaccaaaaacatctctgctcccagatttcctcatctgagaaacaagagggttggattagatgacctttaaattcccttccagccctacactccattgatttgtgattcctcatggggaggtacaacttttgatttctaaggaccaatggttgaaaaagtaagcatttatatagcatattttatatatgcaaaatcccttacaaatgtttattattcttacatcaggcaaatttttaggttttactagtgtcagtgcaagtacataaatttgccttctttgtttcttatgttcttcatatttttcccacgttcttcatatgttcttatgctcttcatgaagagtattacagctcttcttttaaatttcattcatttacattgcagttcttaagatagtacttggtcagccaactcacttgagccagtccctcctctcctatccagtcctattacacaatccttgagtccttgagtttagttctccctaacttgggacctaatggtccttccccttatttagattgagccccacttacctgtgtcgatgaaagtgactccttaggttatatgaaagaattacttttccttttgtctttctcaacactttttcattagtactgacaacttcctctacagtcaatgttgtgcctttatcagctccaagagagacagagataagatgtgttttagcaattcatatacgtggaaggggaagaggcaatgcatggcttaaaattagaaaaactatgttaactagtcaactcttaatcttaatggcttacactggatgcatctgaaggattcaaacaatattttctccttgaaacctaagagtacatcaccttgcctttttaactgcagccaccaaaaacagtactagatctgagaacccttcctctcccctagtactggagaagagttgctcaatgtttgtttcatggccactttgagcaaacttagctaaaatctattatctgtaattttctggatagcctaaaacactatcgtctatttttatgtgtgtaagtatgtgtgttttcctctgactgtggttctgaatttaaataacgtgcggtcacgctaatggtgctttcttgttttgtagatgtacagtgttttgttagcttcgattggtatttaattgtcagttgtgggtctctgctgtttgagctgctttgatagcttcaagtatagtcgttgttcatttcttgcattttcttccctgcagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtaaatagtctgcttttttcattcttctgaaaactctcctaatgcagccacgtgagaaatgaagtggtggaatttggaacttttggttactttcattgatttttgctataatgctgatgtatctctctctctgcattgctgttcactcagtgctttttattcctgcctcaccatgcatttggcaaacatattgcaaatgctatgaataaaaaaagactgtagagagcacagtgttctaggtcagccaaatactttttcttcagtttctaagtaggagttgaattgtgtgatttgcttcttagccatcttttgtatatttttgcataacatttttaataagagcagatcttcttattgtattgggttcaaatttggtaaatgaaaggacatagtttttatttccagcttaaaataatgtatcttcagacaataaacaggtagtctatagaaatgcatacaaacacaaccatcgcacttgagccagtcgattccatcccatatctcatatgaagactttaaaaaggagaatttctagccctcaacaccttgtaaggactttagtttctggctcttttttcaaaggagattatattcaaaaggaatactaaaaaattagccaggcataatggcaaacactggtagtcccagctattctgaaggctgaggtgacaggctcacttgaacccaggaggtcaaggtttcagtgagccatgatgggaccattgcactccagcctgagcaacagagcaagaagaccctgtctaaaaaaaaaaaaaaaaaaaaaagggagtaccaagtaatatagtaatataaaaacttagatctagaaagcacttctagagctgctcttgtttctttgctctgattcaatatgatacctaaaatagccttgctagtgggtgctttttctcttctacagaggagatttcataagtaagtcttaggcttgccttgaaatgcagctcccaactgatcccaatgtgaacagtaattgtctaaaagatgagcttttcccctaggcatctaatctgtgcatatgtttctccacttgcctgtgaccagaaatctccttttttgtcctttttaaaggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctggtaagtgagagaaatgagcattccttgtctaggaagtgcattcctcagagcccagtgtggaaactagaaagagttaagtaggaaggtttcaatctgtcattcttgttctgaagggttacaacagcagagacctctagggctaattacacgaccattaaggcactaattgctctccgtctgcctcagtggagatgatcatctcctcaccatccttgcacccatggagaacaacacagtcagataggaagagcccaccctataaaaaatgtccttttaatttttgtgagaaaattatagccatcagtattttcttctggtaaactttattagaagattattttcccaacacacacactctttccattcaaaagacgagacaagcaaatgatgtacagtttgtaaattatgaaaaactgtgatctggatataaatcattgtttgggggctactttttggccatttgggataaatttattaaatccaactttcctcttgattggcccacttttttttttttttttaaataaacatgaggtctcactatgttgctcaggctggtctcaaacttctgagctcaggcattcctcccactttgacctcccaaagtgctgggattacaggcaggagccaccatgcccagccagcctacttattttaaatctctaaaagattgaacaacggtaaagatttgtaaaagttatgtgctgtcgtgatgtttttactccttattttgaccaagataatacttttttttcaagatttagaatttccgtcctagaaaaataagactgagtttattttcaaatttgggacctcatgttcttgatgttatctggcaaaattttaattggaaaaaaaggcttgtttttatgtcagttcacatctactgctttgattaaaacctagccccaaactcaaaacttgttataattcagccactaggattaaacaagtcagccccaacaaaacccaccatgagagatcataattggcaattgaagggtttctctcttttacagctcatttttcaaagtagtgaattaaggatgagtttggttcgaaaaaaattagaatagttctctactgttgcctttttcaagtaagaaacaatagtaggctttttaaagacagattccagagaacccaatttatgcagaaacgtaacatttttagccctagcaaacctgcatttgttttaatttaggattccataaggatcttttagtgctttaaatcccaaagcctctgttttaaacagagcaagagttagaaaataaaaattttagcccctaaaactgacaaattcattgagtatattctatcaacaatcagaaagaccagccttgcacagatagttgaagttattacttacatatttcaagcaataaagcatttggggagttggcagaataaagtttgcatgaattgtatgggtgaaacctttccttcatcttagacatttagacgaagtcatttttacaattcatttttatttctctctttttttttttttttttgagacggagtcttgctctgttgcctaggctgttgcagtggcgtgatcttggctcactgcaacctccgcctcccaggttcaagcgattctcctgcctcagcctccctagtagctgggattatagatgcctaccacaacgcccagctaattttgtttgtttgtttgtttgtttgagatggtgtctcactctgtcacccaggctggagtacagcggcgccatctcagctcactgcaacctctacctcccaggttcaagcgattctcctgcctcagcctccctagtagctgggattatagatgcctaccacgacgcccggctaattttgtttgtttgtttgagatggagtctcactctgccgcccaggctgggatgcagtggtgcaatctcagcccactgcaacctccacctcccaggttcaagcgattcttctgcctcagcctcctgagtagctgggactaaagggtcacgccatcacgcctggctaatttttgtatttttagtagagatggggtttcaccacattggccaggctggtctcgaactcctcacctcgtgatccgcctgcctcggcctccgaaagtgctgagattacaggcgtgagccactgtgcctggctaatttttgtatttttagtagagatggggttttgccatgttaggcaggctggtctcgaactcctgacctcaggtgatcctcccacttcggcctcccaaagtgctgggattacaggtgtcagccactgcgcccggcctatttttatttctattattcaaaaatatcctggccgggtgcaatggctcacacctgtaagcctctcactttgggaggccaaggtggcctttgggaggcccagacttcacctgagcctatgaaattggggctgtagcgagctgtgatttcaccactgcacccaagcctgggtgatagagtgagaccctgtctcaaaaacaaaaaaacaaaaaaatccgtagttaattagctcagtttttattgttttagcctagactttgctatagccacgtggcaaggatgagctaaatttacccaaatatgtcaatgttctgaaaaggccatcagtttctagccgaatggctaactcccacaaactacagcaatctttacttacagccagatggatgtccaaaataaagaatcctttcctatttcattgcttataccacattaacctccttgaagaataaaagggccctattttctttttcccaaagaaaacaaaatttgacctggagccaaaacagtatgcgaaatggtaatcactaaatccaaattcttccatttgtgataggaggcgatccttttgaagaatactctgacgtaataattaatcatagaccataaccttatttaagcttaccaaatttaaaatatagattcaaaagtatccaaatactctggcttcactcagcagtctcatttctagtgaacagttgtcacagagccccaatacctgacctgtgtggaacccctgagggtcactgagtgccccttatgttctccttctagtggagaaaccctacaaatgtgagttttgtggaaggagttacaagcagagaagttcccttgaggagcacaaggagcgctgccgtacatttcttcagagcactgacccaggggacactggtgagttcacgcaacacacgtttagtgagcatctgctgtcctaggtggtgagatacaatagtaagtaaaaagactcgtggagtttatattttactgggggcagacagattataaagaaagtaaatgagcaaactgtaagttggaaggttagcgggtggtaagtgctattgagaaaaatgaagcagttgctgaggagtgagggtgagaatcagggaaggcttcaacgaggtaacattttaacagacttcttaacagggtgagtaagccctgcagataactagggaacagcaagagcaaaggccctggggcacaaatttgaagaagtgtacgtagttctgtgtgtctggaacacagtgagtaatgggacctgaagccagagaggaaatgggaaaccagatcttctcaggccttgcaggccattgtaaaggctttggcttttactctgagtgaaattaaaaatctttagagggttttgtacaaagaggtgacatcattggtcttctcatttgaaagaatcactctacttgcttttgagaagagattgtatttggggcaaggttggaaattgggggtccagttaggaggccattgcaataattcaagcaagagaaaattatgtcttgttagtggtagaggtggtgagaagtattctaattctctgtatatttaaaggtagagtcaacaagatttcctaacagattgatgaagagtggagttgtcgtttacaaaaaatggcaaaaattgagggtagaggaggttggggagtggctagggaagaggcaattaaaactacaaggattttctttgcacagggcccaatttatatccttgtctactcatatgatttattgtgatgcttctatttggctttggggattccttgttttattcagtgatttgaattagatgcctactggcaattggttgaaacactagatgttcttctatatctcaattgtggttttatttccttttaattctttgttggaacagcctgaaacttttttgtgtcacatctcttcagctgtcctttgtcccattgagttggctgtcagcctggttctgcagcttgggttgtctgacatttttatttggtaacttgaaccaattgcttatttagaataggtacaaaccctcatgggatttagtgtttgacagcaattgttgcagttgagtagtttgaataggctaaaacagaaaagttacaaaaagatgacagaaataatgtgaaactcaggaaaaatggagtagaaatgactacctaaactgccctcattttgagtgagaggaaacttttattttagataaaacctattgcatgatgttaggagctggccaactgaattctgatggcttgcagtgcttctgctttttatcctaatggcatctttggttttatcaaggagttggatttctaatttgataaaggaaatccaacttatttggataaagcttatccaaataaaaagaaatgtttacccccttataaaaaacaaacaggtcatacacagtggtacacacctgtagtcctagctgctgaggaggctgaggcaagaggattgcttgagcccaggagctcaagcccaacctgggcaacaaagcaagacaccatctctaaaacaataataataaaataaaaagaaaaacctaatatttgggttttgacatcttctctttgttttgcttttatctgagtctgcttgctaggaaagattttgacatcttaagtggcatgcttcatggcagtcaggcagtactcgctcaaggacggaatgtaggcatctctacttgcagttctgtatttcagacaataggcaaagctctcactgtaagagattcccaagctatgaaatctatcatattccaagtagaatcactaatatttaaagctagaatgaaagagagcatctgaccctacttcttcattttagaggtgacaagactgagactcgcccaaaattcagtgacctacttaaaattcaccagaactttagttgtttttgtgattagtcatattttttaattttcaaaaacttgagatataactcacatgctataaaatttacctttttaaagtgcacatttcaataattttagtatattcacaaagttgtacaaccatcaccactatcttatttcagaacattatcaccatccgcaaaagcagtgctgtgctcattagcagtcactccctattcccccttcctctagctcctggcagccaccaatctactttctgtctctatggatttgcctgttctggacatttcacataaatggaatcatacaatatgtggccttttgtgtcggctccttttacttagcataatgtttctgaggcatttatgtatgctgaagcatttatcaggacttcattcctttttgtggctgaataatattccattgtttggatatggcactttccatttatccattcatcagctaatggacatttgagtcttttacaccttttccactttttggcttgattagctatatttcttaaaggttatatattatctggtttgcaaaagaaatatagtaacacacatactaatagacacacatgtatatacatatatgtgtgtcctgattctcctggaaggagggggaaaaatcagaaaactacactctataaacagtaagttcaaaattattgattttgaagtaaggtcaccaccttacttcccattctcacaaccttttttaggaatttcaaataaggaaaagtatcaaaagtaatattttagctattctcagtgatattaggtgattcaggaaatgctgaaatacagaggttgagttaatttgcatttcatagggtggtttggtggatgcgtgcaccatactttaagtctcagcaaaaaaagataaagggttaactaagtcagcaagttttgatttttttttcaaacaaatatgttgtaatggtggtttcaggttataaatgtgtaaattcacagcttaatcaagttactgcatctcagttcacacattactcttttatatttcaaatagttcctataatttttcctgaggccaaaatttacagaatatttaacccaacataagccaatcctttaactattttgcacccttccatgtgtggtttccatttctgtttccttcttagaattgggagtccattgtggacatggggttctattctgagcctgagcagtatagtacatcttatgcctcatccgtcacttgtaaagagatgatgctccattgctgttgtgcctgcagcactcattcttcaagtgcttattgtaagttgaagtgcagcgtagtgtgagagccaagcctgtggacaaacctgcctgcaaggggaagcaggactttctagaagcaagcagatggataaaaaggccttggagtgaagaccagcctgcaaaaaaatgcaggtattgggcaacaggacttctggccttgctaatcatttcttttatccagatagcatttgttgagttctctatgtgccaggctctgtatcaaagctgtttgtaaagatgagttaaggttaagcccctacactttaaggatttacaatcctggccgggcgcggtgggtcgcgcctgtaatcctgacactttgggaggccgaggcgggcggatcacaaggtcaagagatggagaccatcttggccaacatgttgaaaccccgtctctactaaaaatacaaaaattagctaggtgtggtggtgctcacctgtagtcccagctactcaggaggctgaggcaggagaattgcttgaatccaggaggtggaggttgcagtgagctgagatcatgccgctgtactctagcctggcaacagagcaagactccatctcaaaaaaaaaaaaagacactagtgatattgctactttagtccaagtttctacatcaggcatcacaagtgacaccatcagaatattccaattctgtcttaatggagtatatatgaaaagaaggaaaaatacagaagaacatggactgcattttcccattagcttgattcttatttcagaaagagactaacagccggtatagagattcatttagcctcattcacacagattatgaatacttggagcaggaaatattttatgtaacaatgagttctagcaataataatatttttaaataattgaataatcacatctatttatgaatctaagactccaaacttttcatcttcaatagttcttcaagctaatacaagcatgagggcttcagaataaacttacatttttaccaacttagcagaccaaagaaaatgtttaaaaatttttaagtttgttttcccaacctatttttttttgttttgttttttgttttttgttttgagacagagtctcactctgtcacccaggctggagtgcagtggcacgatctcagctcaccgcaacctccgccttctgggttcaagcaattctcctgcctcagcctactgagtagctgggattacaggtgcctgccaccatgcccagctaatttttgtgtttttagtagagacggggtttcaccatgttggccaggcatctcaaactcctgatctcaggtaatctgcccaccttggcctcccaaattattgtgattatgggcgtaagccaccacacttggccccaacctattctttttaaaaatcaatactggccgggcgcggtggctcacgcctgtaatcccagcactttaggatgctgaggtgggcggatcgcgaggtcaggagttccagaacagcctggccaacatagtgaaaccccatctctactaaaaatacaaaaattagccaggcacgatagcatgcactgatagtcccagctactcaggaggctgaggcaggagaattgcttgaacccgggaggcagaggttgtggtgagccaagatcatgccactgcactccagcctaggcaacagagcgagactccatctcaaaaaataaaaaatgctatctcacaggtagtattcattttacttgttcaaaaaggcaacagttttatgaaacttttttgttagaacctgcctctgtctttgataccaatctcttgttagacaatttctattgcataataacaccactttgttccactctccattagttactgactgactacaccatagaagagacaggaagcaatacctcttatttaagctccacttaacacttcaagatctaatgaaggattttgaacaaaaacaaaagcacaacacaattacatagttaaacctccaatgactttgaggtagaaattgtatcatggaaatagttaactaaaaccaaatattaagacttatacagggaataaaggtagtagtaacctaatctgtaaatgtgactttgtactacattttattattttttcagcacgtctttaaaataacttttctaatgaaaattagtatgtctactatttagtaggcaagcaatgtgaagggaaattgatccacaatgacagttctactcattctgttctcactcatgagctgaatgtgcaaattgttccttgccaccatcccatattccccacacccaccacttgcagcattacaccgccaaacaccacccaggaacggatttcctatttcgtcatttaaacccattatcttgttcccttactatcttgccaaactggtttccctccacttacatttctgagatatgaagtggcaagataacaagtctagactgttcgtataaatagacagttgggtcctaaagttggagaaaaggaccaaaagccaatagtcctcttttttttcttttcagtgcactgcctaaaccactcagtaatcccaaagagcttgtccagttacaaagcatttctttgtgtatgtgtgatatgtgtgtgtgtgtgtgtgttgttgttgttgttgttgtttagagacagggctcactgtgttcctcacactggagtacagtggcaagatcatagctccctgggctcaagcaatcctcccacctcagcctcccaaagtgctggattacaggcgcaaaccatggaacctggctctttgtgtttcaaagggcactcatttcatggtattgtgcatcagctatccaacaagtgtgaggcaaaagactagcagtttaggaaagaaaatgagtgccatcttcaattgaaaactagcctttgcctaacttatcaactagaaaacaaggttaattcagtgaagatcaactgaatcagtaggagtgtgttcacatgtacaatgagagtggagctcttgtagttaatattttgagtgaatgacccagaaagagggtgtgggtgaacaggagtcctctagattcgtttcagaggatttctttcaatctaatcggagttgtggaaatgggaaagtctccacccatgcctcaaaatatgtccatccatgaacactgcagaattttatgtttttaaatgtcaggcatttggtcactgatttacagaataatgtttatctggaaatgactctaaagaccagtttggtgggtaacgaggaataaaaggtgatgttatctcaaaagtttgccatttagtgacctgccctaattacagttatttaggtcaatgtgtccacacttcagcatcctcccaaatgtagaggatgttgattacgtgtcctcagaaagctgcttgttttaattgacattgacttcagtaaaatgtagacagcgactgctgagaaacaaatgggaaaactagatgggtagcaccgtgcttggggtaattcctgggtaatgatttggcagtgagaggagggaccacaatagactaataagaacaatggtaaaatgggcataaaaggtaaacattcccagaatactgtttttcattctttttttccctataaatctttttgttgataactacagggttgaggggaggcttacttggatttaatcttctcaaaattgctggaaccaggatgaatgttcctagtcaaagtgcaaacttagctacctctcccctgattctaagaaaaaagtctcctaacagccccaggaacttactgcaaattccaagctttgtctttcatctctctgctcttggggctttgggtagtgccacatctggtggtggttttgtgtgaaaggaaataatatatataaataatttcctgtttggtcatttaaacctatccctgtgctggacagaatttgaccctcttcactcaatagcagtgtcttgtggctgaagtgcccgttatttacaactgctggtcagccagagtcagtcgagtacttttgcactagcccaaaagccactcaggccccctattctgacccttactgttttcatttacacaaatagttgttaatagcttcactgaaggctatgatttattcctttagagcctagaattatttttgacaagaaactcactgtctagtaaatattgtcattattatcacattattatcatcctcttctttttcatttttggagctggatttctttttctttttaagagatgggatctcactgtgttgcccaagctgaacttgaactcctaggctcaagccatcctctcacctcagcctcctgagtagctgggactatgggctagtgccaccacacctggcagagccagattttatgttattccaggaaaaaaaaaaaagctgagctttggctgagtgcagtggctcatgcctgtaatcccaacactttgggaggctgaagtgggaggactgcctgaggccaagagttggagaccagactgagcaatatattgagactaataatttttttttttaattagccaggcatggtggctttagactgtagtcccagcaactttggaacctgaggtagaaggattacttgagcccaggaggttgagcctgcagtgagctgtgatcatgccactgcactccaccctgggtaacagagttgagaccctgtttcaaaaataagtaaataaattaactaaataatgaaaactgagctttataatgtattgtgtaatgactaacatacagttttttaaggccactaataaaattagaaatatttaatactttggggcttttttttttttttttagacagagtctcactctgtcacgcaggctggagtgcactggcactaataggttggtgcaaaagtaattgtggtctttgcagttaccttaatagttcaccgtaacctcaaacccctgggctcaaggaaccctcccacctcagtctcccaagtagctaggactacaggcgcatgccaacatgcccagctaattttttaaaattactttttgtaggaacaggatcttgctatattgcccaggttggtctcaactcctggcctcaagcaatcttcctgcctcagtctcccaaagcactgggattacaggggtgagccactgtgccagccactacttgctttttactctaatcccttaaactaatttcaatttattcttccttagacataatagaaatgtatggatagttcattttagtttggatctcccgaagtgtttatgagaaaacagagaaaaatgtttacagcatcacaatatgtttgcccagggcaggtgagggcaaactgtggcgctctaggcaagtgcagccagtggcctgtttgtgtacagggatttttacgtttttaaaaggccgtggaacagaaaaaaaaaaaacaaaaaacaaagaatataacatagagactatatgtgtctggcaaatttttaaaatttactgtctggccctttacagacaaaagtttgctgatccctggttcaggctagcatcatgtgttaaaatgtcaagccttgtatttagagaagctggtatctctttgaaatttatttaattaatttatttattttttggagatggagttttgctcttattgcccaggctggagtgcaatggctcaatctcggctcaccacaacctctgcctcctgggttcaagcgattctccggcctcagcctcccgagagctaagattacaggcattcgccaccacacccagctaattatttatatttttagtagagatggggtttctccatgttggtcaggctggtcttgaactcccgacctcaggtgatctgcccgcctcggccttgcaaagtgctgggattatagacgtgagccaccgtgcctggccttctttgaactttagacataaatttgggcactgcctgctgaattgaggtaaattatattcattacagtttactattttagttgattagatattgccactatttatatactcttctagtagttctcaattctggctgcattttaaaatcatctggggagctttgaaaatattcttgcctaggcccctataccagaaatcctgatttaattgatctgaaggggggcctgggcataggtatttcttacctgctattcaggtgagtctaatgtgcagccaggtctgagaaccacaggcctacaccactcacaggtctatttgtggatgggcccacattcataggcaggtttttttgtgttttgtttgtttctttgttttttgagacggaattttgcccttgttgcccaggcaggagtgcaatggcgccatctcggctcactgcaacctctgcctcccaggttcaagtgattctcctgcctcagcctcccgagtagctgggattacaggcatgcaccatcacaccaggctaattttgtatttttagtagagacagggtttctccaagttggtcaggctggtctcgaactccgacctcaggtgatctgcccacctccacctcccaaagtgctgggattacaggcatgagccaccgcacccagcccataggcagtttttattcacatttttattagaaattcaatacagatgcattatacaaattgttctttaatttcaaggcagaagtgttgcttagggaaattttaattgagactattttgaaatgtgaagagcatatgaaacagataatacagagcacagagaaacagaatatgaaagataggaactgagaggagcgtgctgaaaaaaaaaaagaaaaagaaaaagaaagaaaccactcagaatttcaatacacaaatcactgagtagatgtcttgctatctgtgtgagtgtacctattttcaaaagtgctgttaacataaacagagcagtaaatctggggcaccatgcttttttttcaaagttgttaagaattatatcagtctgcaggtgtcacacgcagttactcagaatcagaaagaaggctgatcgggggttagatctccatctatctatctttttgcaaccaaccacgtccaggctgtttatttaatacttcctcttgctaatgaaggtactggttggggatgggtcgagctttcccatctcggcataagactgcaagatttgaatgtgccctggtgtcggcttcactgaccacagcaggtaccccaggcaagaatctgagcagttataacaggtaacaacaatagagtgtgcatccaacttcggacacttctccttccagtttatttccttttctccccatgtaccaatgtgttcttttctctcttctttttacatttttccaaactagcaagtgcggaggcaagacacatcaaagcagagatgggaagtgaaagagctctcgtactggacagattagcaagcaatgtggcaaaacgaaaaagctcaatgcctcagaaattcattggtaagaatacaaacctttttcctgctgttgttagcagatagtttgaatgaaataacataggatgcttgctacacaaggcctttcctagcttaacctctgttaaaataattagtttttcattgtgtacttttattcttcatggttcattctgatagctctggaaaatagaataaaagatattaactaaagcatcatcatgacaggagatgggagtagttgatatccaatttcctaaatggatgtgatgtttaaaatcctgttccttcctctttactctgaatgacatatgttgcatgagagagtttatgcaatttataccagtaagatgttcaagatagcatttttgtaatcttttgtaattatattttacaagttggttcgttagcatgctcaaacttttttcatgctctaaaacatattgccatattttcttgaatttgagaattggctttaagggaaagtggttgggcaaacatagatttaaataacaaggtttgatttaatcatttgtcttactggagttcatttttatgccattggttttcacattgtcaaaaaatacagcagaatgagatgggtatgaaataattatgttcattgaagtgttaggacttccaaatggcagagacattttgctgaactctcccagatgtttacataaatgtgaccatgatatttgaaatagatttgtatttgtgaagtagacagctgaaattgaatgtatcagttttgctataaattgaaagttggtgatattgaacaaaaatagttatcacatgaaaatagacctccctcaacatcagtaatattttagaaacctgtctcagcctctttttccaaagctgcttgccgctgttgattttttatttcagtccttttgtatttattcttctcaatagctcttacagataataagttgcctgcctggtttttaaatctgactaaatttgattctggtatagataatgttttgacaagcaaacagtctatgtgaattatagaggtatattgaaagaacccatatatttcaaaattacctaatttggtatttctgcctacttattttattttgactagctgaaaggtttttgtgcacttttagtcactttaagaacaaattgtgtcagagaaaagttggtatatttcagtttagggagatcccctcctccttgctgtcctccctccccttggtgagacatgtcaggggagcatgtagtgacttccatttagagtttaggcacagtaaacaaatggtgagtttaagacattgtctacaaatattcccatatatcttttgggctttacttatgtgaagaggttctgactggagtacaaaagaaagcttgccttattattctgagtgtattttagtttggagaaagttatgggttttgtttgttggtttgttttgagacggggtctcactgtactgcctaggctggcgtgctgtggtgcaatcacagcctcaaacacctgggctcaagaaatcctcctaccccagcctcccaagtagctggaactatgggcacgcaccaccaggctcagctaatttttaaactttttgtagagatagagtctcctcatgttgcccaggctggtctggagaaagctttgactctagaaatggctgaagccgacaagaggagggacatcatagggagagctgggcaagaaaaaaatgtaaagcgacttcatttctttctttctttcttttttttttttttttgagacagagtttcactcttgtcgcccaggctggggtgcaatggcgcgatctcggctcaccgcaacctctgcctcctgggttcaagcgattttcctgcctcagcctcctgagtagctgggattacaggcatgcaccaccatgcctggctaattttgtatttttagtagagacagggtttctccatgttggtcaggctggtcttgaactcccgacctcaggtgatccacccgccttggcctcccaaagtgctgggattacaggcgtgagccactgcgcccagctgtgacttaatttcaatccagttgtggtcaatgcattttggtgtaagacaactaaaatttgagagacagtccctagtattttcggttgccctaaacctatagttgaactaaagccctacttaatataataaaagttgctttggtatttcatgggttcaaaaatatagcatgtttcatatgtgcaaattcatgccattttgcctgttgcctcggggatctatttcgtttgtttgctacaattgcaaccagaggattttcacttcactacttccagacttgtgagagatgctgatcttgtaggtgttgatttctctcatcttctgaaccatcacattctctctgtgctggaatcagaaacctagtagagttagctcttgattgaggtagcaacttcagggcttcagggcccagaataactgggggaattttttttttttttcgtaagttgtgttgtagtgcaataagaagagtactggattagaagctaaaagacccaggttctgatcctagctctgtcatttcttaactgtgtgtttagaccaatcacagaacctttctgggcctctctttactcatctgtgaaatgaggagactgaataggtgatgaagtgaccaggaagtctcctcccatgtacaagtctgtgagattaattagaaaagaaataaaaggcagcaattattgataccactgtgtgtttagaaagcccatgtgcgtaacttcagatagcctttttctcaaggctatacagatgtggtgtttatgctataaaccatatcctaggtttttagagtcttttttgttttaggaagatggtataaatctgagaatctaaaacacagtcgcattagcccgacattcatgtgttttacttagggcagtactacttgttcactcacttttttgtccattcattcatcatacagaaggttaagagttcctgtcatatatatggtattcttgttttgggcactgttctaatcttcttgcattaacttatatcctaaacttttgtttgaaaggtttttaaaaattaaaaataaaagaaaccccttaaattatttaattgaatcaacctttgtgatagaaaatagtaatgtcaacggagggagaaaggaacattatttgtgacactgtgcagtgtatgagatgaagtgatgtaaaacatggggctactgttttctctccatctcccctcgccttgcctcgcctcacctcacctctcctctcttgtcctcttgttccctcccctcctcttccctcctctcctcccttctcttctcctcttctctcctttctcctatatttcctctccaaggcactgttttttgaagattcagctgccagggtaatggcaatgggtaaggtgtgaaggaagagtgagtcattccctttaatccttgctgacagttgcacagcactcagaccagccttgacttccctgacctcttgaactgtgagggtaaaggtgaaaatagaccatcaggtcagcccgctgctatctcaggcgggctggcgataattaccagtagggagcattttgcatccattagagatgaactggggaagagggacagattggatcaagtctaaatgtattttgagacacaaacatgctctcaagttggagaaaaatatttttttttctaattctccaagaaattttgtatgtctttgtttcaaatgggaaaaaaaaaaatggacacacgtccatcaggaatcaccatctttacattgtcagaataattctgtttaagttgttcttttcaaaggacaagaagaccttcctttgcacatactgtcctttaggggatttcctttctctgaatcttgttctcctgttataaacttgtttgactacacttcaaactaggccatcctcaaatacttggctcaaaatggatttctctcttggatgcagaagaaaaatgactacatttctgcatatttctcctgatttttattctttcccaaaaaaacagatgttattcagaccaaagtggttaccatagcatctccttattttaaaagttttatgtattactttattaaaggaagttttcataacgaaaagaacatgaaaaaaagatctcactcacctgtggtaaacatgttaatgaggttttgacacttcaacaaaccatcggatctccctgtgaagtaactaatgagaaattgcctgctttggttaacagcaacaggaagcagttttaatacctgtttctaagagaaaaaaaagtatttttccaaccccttcccaaaggcctgatgtccataatcctgtaagcattacaatgtcatagtatttttgggctattaggaaccaccagtgaggctcttttaccatggaaggaaggaggatcagagaggtggagtgacacagctggcaggtgacagagcagcgccagacctcggcctataaggcactgcagtccaggcccttccttccccaggaggtgaccaagagcagtgcatgtcatgcgcggggctttggacagcaaggagtctggtcattgaaagttggtgggagaggccaggtgcagtggctcacacctgtaattccccactttgggagtcctaagcaggtggatcacttgaggtcaggagttcaagaccagccgggccaatatggtgaaaccctatctctactaaaaatacaaaaattagccaggtgtggtggtgggtgcctgtaatcccaactatttgggaggctgaggcacaagaatcacttgaacccaggaagcagaagttgcagtaagccgagatcgcaccagcttgggcaacagagcaagactctgtttcaaaaacaaaaaagaaaattgataggaggaaaaaaacccttgaagaagggcaattgttctccctcaagggtgattctctttagaaaagaacagtcttccctggtgactccatgagaacagatgacatccacacagcacttcccagtgtctcacagaggacaaagacttctgcagcaaccaaatgtggtctccagctcagctttccccagtgccacccccacagctctctctgttctttcctccttcctgtttcttcttgttctctggctcccctctgttttccacctcctgcaagaagtgttagtttcacttaggttgagatcagagccatgatgatggttggatattctattttcctgccttttttgttcatttgcttttggatgtgtgctgatagtaagagtgagggataacacttgtgggaaattatatgacgcagcagcaaacacatttaagtgacttgaagaactttaagacttaagacctgaatatttcaccttaccccatcatggtgactctaaatattggatttctgttatttttctaaaggttcgatcattttgcccccctcttcaaaaaaacctttcaatggctcccagtggaatcaaatctaaaccattcagtcaggccttctgagccctccacaatctgtcttcaacttacctcttctaattttatcatttcagccaaccagtcctttacttcagccacaatgaactcctcctccctcttctgtgtaatatctcactcttgtctgaatggcctccctcacatctgcccttaaaatacacattgtaggccaggcacagtgcctcatgcctgtaatcccagcactttaggagaccaaggtgggaggatcacttgagctcaggaatttgagaccggcccaagcaacatagtgaaactctgtctctgaaaaaaaaaaaaaaatcaaaaacctagccaggtgtggtggcatgggcctgtggtcccagctacttagggggctgaggtgggagtatcacttgagcccaggaggttgaggctgcagtgagctgtgatcacgccactgcaatcccacctgagcaacagagtgagaccttgtctcaaaaataaaaaataaaaagaagcacacattgtccacattcccacacccctgtgttctctctctgctccatcctccctctgcactccaggcgagtttactagtctgcttcttattatagatatgactgtctccaccaccgtcactaccaccacctctgatttcaaggagactttaaagaatagatgaatctttgccccatattaagtgtggcatatttgagaagtaattcagttttctctttccactctcttggctactctatgttttgttctaccatgtagcattaaggatactgatttgtcatgaaatgatgtacatgctgcatctcctctagtggattgcaattttgttgtattcctcaaatcacctaaccatggtgctaagcaaataatgggaatagagtgcttacacatgcctggcactgctaagcactttatagatatcataacaactctgcacagcatatacttcttgagtgcatgtgctggtccatgtggaaaaagcagttgctgccactaagccccctttccctctgctatgggaaccatcacagcagaaaacctgaagtcactgctggtggtggggctgtgcagccatgggcaaggtagcagccaggaaacatcagttaacactcaaacatatactgggatatgtgggctcttggattacatcctggttctctgttcaccaactgggcaatcttggaaagtgaatcaaactgtttgtgctttaggttcttcatctaagagatgataattcataccttaaaaggtatcatacaaaaattagccaggcatagtggcacgtgcctgtagtcccagacacttgggaggctgaggcaagagaatcacttgaacccaggagacggaggttgcagtgagtcgagatcgtaccattgcactccatcctgggtgacagagcaagactctgtctcaaaaaaaaaaaaaaagtatcaacccctaatgggctggtaaaatgagacagtatgcaaagtatctaggatggcacggtggatacagcaggggctcaataaatagtacttatcattattgggattattgtatataaggcacagtgcaaggtactgtgaaatataaagaccagaagtaaacatattagggtggtgggtcttccccctcaaccctgcagacagagttctgaatccacttgctcagttaagggcccttttagctctagcctgtgattctgtacttccaagttggcacttagaagccaacaaccatatgtcctcaccacattgcatcagttcaggtcaggtggtttcttagtttagagttaacaaagccaagatataagacattgacacctttaaaaagaaccacaggtagggaatggcccaaccaggcagccttagtttctaaacttaggacttgctgttgccgtgtgtatttttgttggaagaagacctctaattcagcctgaagctggtatcatgaatcattcaaaggcgtggtgtgattttgactagaagctggaccagatgctccgtgcaggtttggaagcagttacacttttgtggtttgactaaccttttttatgagtgtgttactaaagaaacgtgagctgttggcttttcaattactgtagtttaccctttctgggtttcatacacattgagtcatcgtgtctacaggtgactgataatcatctgcactgctcaagttaccgtaactcagagacgaggggatctgatgattgcaagttgcatgcacgtgaacatctgaggctgccccatctccttaacaatgaacaatggtctgtgttaactacagaattcagtcacatatttccactcatccaggggtttatcttcctcttaatgttttgccttttacaattttagcactcagctgttttctgacatatgctgcaggtggtatagattgtgaaattgtaggccttgttaagtaaagcaaactcttaggagaaaggataaaatcagagtttgtgatgtgcctaccccacccagaaaccttctggagcctctctgttgggtgaaaagagaaaaagtttctcaaggacccctggtatttagggagatgaatagaggtggccacagtggaaaataaccaataaccttaaaaaggccaagaaaccgaagttcatcatttctgtgtcacattctagggaatgaatgtctgtgaccttagctggctctcatgttctcagtcttccagatggccttgaccaaagatatatccaatctggaaactgaaaataaaaaacattgattactttggccaaggtctactctgctgttgcttctcattgagtttccaagacagggtattttcgtttgcatattttttttctctctcaggtatttctctggaaactatcaggttggccttaaggtcagctgcttgtggggatgcaaagacaaattcatctcatgggaatagatcttttagctggttcttctggctccaatttctcactgttctggtctgccttacacattgctgttacgttaaccacaaacctgtctttctcaagggcaggtagccttggctcttggttcggattcacaccctacagtgggccattcggaatgcccaccattcggtcctaccatgcattatcttttttcagtgtttccccaaatgtaggatgcaaattgccagcaagcacatggagcatttaaaaacctgggtccacacagcaggataattattttctttccagttctctttcagtctcctggatcatactaaggagaaaaatttagacttaatttgatgccagttgtctttaacactaatctcacacttggtaatctctgtttttaacaaagggggaagggatcttagtctccaagtactcacagtagtatccgatcaaaatttaatgaaatgctctgtttcattgtctttatttttcatagttaccttctacttgtggaaagtgattctggttttccgtttatgttagtgatataaagtttccttaaaaaccacatttattttagaaaaagaaaaaaagaaagaaaatgtgaaccaaatcaaacagaggaaaaaaacccaagcatttaccaagtgttttccatgtggcaggccctgttctaagtgctttccagtgaccctgtgaggtacatacaattatcttcactttacatgaggacacggaacataggttaagtgacttgcccaaggtcacaaaactggtgaccaatggagccagcatcaaacccagcccatctgacaccagagtttattctctcaaccaccacactgccctggtgtgtggatattgcagaatgtgaaggaagagtgaagcctgagaaatactgtatttcatacttcttgatcgcgtttcagccagactcgctttctgcacacgtcccgtgtagtcccacctctctgccttcattcctgcactccccctcaactaggatgtcctctcccgcttctctgaaaatctaaattcttccagttcaagacccttctcaaaatttagattttccaagaatgctttcccttccccaagtacttctgaagtttctgcattcccttaatgcctaaatatacaatttataccaaattgtctcaccgatgcctgtatgttaccttattattctttttaaagaacacgttacttccctgcctaaatgcctcaaatgtgggcatttagaataaaagcaaagacctccatagagcaaagcagtccttttacaacttttttttttgagacaaggtcttactctgccacccaggctggagtgcagtggtgccatctcagctcactacaatctctgcctcccaggctcaggtgatcctcccacctcagcctcctgagtagctgggactacagatgcctggctaatttttgtatttttttgtagagacaaggttcctccatgttgcccgggctggtctcgaactcctgggctcaagtgatcctccctcctcagcctcccaaagtgctaggattacaggcctgagccaccgcgcccggccaacttttaacttatactttttctcgagttctatatatatataggtcttatttctcatccactaggttggagacacctcaagaacaggaactggttcttccaatccttttgttttctcatggcattttttcttcttcttctttattttcattgctgaaccctttactgtagggcagtgacaaattttggaatctcaacatgactaggaaacattatagaaggatatggtttagcaaatccctccaggatccaagctgacagaatggagaggctcaacagtgacctcagcctcacctccagcactcggctccacttcctcgctttctctcccctctccccaccactcaccactgagcacatctgccctcactgcagtccccatccctgccttccctgtgaccagagagcctctgttttctaacacacaacacccacacactctgaaggtggctaaactctgttttcaaaacttctcagcagtttgacatgagaagtaggagagggtgaccacaagtatattttttcagttaagtgtgagataggccctaatgatcacatgtgtatcagagatacacttgccaagcaaaggaagagaaacacacgttaggaaagggacaaagaccatgatctcttctcaagaaaacttttctcaaactttctacatttgaagctctttcacttcagtctcagtcagccaggacgggtgagtttggggagtagccagttggagtctggggggctattttctctatctctgtagctatttaaaaagctgccgaggtgaggcaccgagtgtccccactctttgcagcacttctgtccccctggctacctaatattgcaagcagaaaataacaagcaaaagcaaaataagagttgggagccgactgtgtcttaagagatcatgaactatagcagaagtcccaggctcctgtgggctggtggctcccttgtccctctagccccatcctagtgtcctgcttctttccctcctttcttgagggctctcctctaacatgcacagtgggcataccttaccatgtggcctcaactgaaccacatctctgcccctctcttctgcctgctggctttattttaaccatccaaacacgttgctgccgaccagtgtcccctgagacagcaccatgggaaaccattctgccaggcttggtcttcttgagcagcaggcaacttaactctcctccctttttgaggtctctagaaaatatttacaaactctctgccttggcagtgttccctttgtaaccctttctgtcacttgttccaggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtgaatatctggtctcagggattgctcctatgtattcagcatcgtttctaaaaaccaatgacctcgcctaacagattgctctcaaaacatactcagttccaaacttcttttcataccatttttagctgtgttcacaggggtagccagggaaacactgtcttccttcagaaattattcgcaggtctagcatattattacttttgtgaaacctttgttttcccatcagggacttgaattttatggaatttaaaagccaaaaaggtatttggtcattatcttctacagcagtggaatgagtggtcccggagatgtgctatatgaaacattctttctgagatatatcaaccacacgtggaaaagcctttcagtcatacatgcaaatccacaaagaggaagagctgaccagctgaccttgctgggaagcctcacccttctgcccttcacaggctgaagggttaagatctaatctccctaatctaaatgacagtctaagagtaagtaaaagaacagccataaaataagtatctgttacgagtaactgaagaccccattctccaagcatcagatccatttcctatcacaacatttttaaaaaatgtcatctgatggcacttctgcttctgtcctttaccttcccatctccagtgaaaagctgagctgctttgggctaaaccagttgtctatagaagaaaatctatgccagaagaactcatggttttaaatatagaccatcatcgaaactccagaaatttatccactgtggatgatgacatcgctttcctttggtcaaggttggcagagcaagggtataaagggggaaattgtttggcagcaccaacagaaaacaaacaaacaaaaaacagctacctaaaacttcttgaaagagttcatggagaattggtgatacagacccaaagcaaatttgccaatgatattttccacaaaaaaagtccaaaaagtatggctcagcctccccctccccacaggagaggaattggagatagatggcatgtgtgtttagatcggagttgagctccggaatggggtgaggagggacacctctattgagaggttctccttgatcaggcaggcttcggccctttttttcccatttaaatggaactgctgtattccatgaaaattcctgaaagtctgatcacggttctgcagatgtataagtcatccttgtcactcataatatgtacatactatcaggaggagtgctgttatcatggtaaaattagcactggaataggaggtcacaaaatgctggctaattagctatgtgactttgagaaatcgtttaactttttttttttttttttttttgagacaggatctcactctgttgcccaggctggagtgcagtggtgcaatcatggctcagtgcagcctcgacctccccaggctcaggtgatcctcccacctcagcctcttgagtactgggacaacaagtgcacaccaccatgtctggctacattttgttctttttgtagagataggggtctcactatgttgcccatgctggtcttgaactcctgggctcaagcaatcagcccgcctcagcctcctaaagtgctgggattacaggtgtgagccaccacacccagccttatttaactcttaaaactcagtttccggccaggctcggtggctcacacctgtaatcccaacactttgggaagccgaggcaggcgcatcatttgaggtcaggagttcgagaccagcctgacccacatggtgaaaccctgtctctactaaaaatacaaaaattagctgggcagtagtggcacatgcctgtaatcccagctactccggaggctgaggcagaaaaatcgcttaagcctgggaggttgaggttgcggtgagtggagatcacactactgcactccagtctgggcgacagagtgagaccctgtctcaaacaaaacaaaacaaaaacaaacaaacaaaaacaaaaaaaactcagtttcctcatccataaaataggaattagatttcaatgttctcttaggtcccttctagctttaattcatatgtgattatgcagtaaccacaaggtattttttaaacctcctaatgtatggatattaagcagaagagtatttatatgaatacatgtttcacattcctttggtatgaaaatggtgtgttaagtttttcctttaaccactgagttgtgaatgtgaagaaggtggtggagaggaacaaaaaacagaaaggtattttgatcttgccacaaagcatacacacaaattggcacatgcagctgtttgccaaagccttctttttttttttactttttaagaaattatgttagggaaaataaattctgcttccagggacaacttcatggagcctatttacaaattaagagtcagcttaatttgtaacatttctaccagagccaagaatcccaaattcctggtagattagtgttttatttctaaggggcttatgcattcggctccaactcaactcgtctatgtgctgccagtaattaaaatgttccacctcagactgcacaaatggcttatccttctttgtggcatggcgtctgtctcaggaaaaaaggttttatgaaattccatggcaacagtcccaacatgtttgagacttcagctaaaggaatggatgtattttggtgtgtagtcttcagtatatcactgtatttccgtaatactagactccaagctatgccagattgcttattccctttgtgaaagaggagttgctcattacgttcttgaaatatcgcacatcctgttggttcttcaagggacaagagaaagagaatttggaagcagggattagtagaagagaaaacgagggaaaggaagcctttccaccagattagtgttcaagtctttgcagaggagaccaactttttttgttttcttttgttttgagacagtctctcgctctgttgcccaggctggagtgcagtggcgcgatctcggctcacggcaacctccgcctcccgggttcaagcaattctcctgcctcagcctcccaagtagctgggattacaggtgctcaccaccaagcccggctaatttttgtatttttagtagagacaaggtttcaccatgttggccaggccagtctcaaactcctgacctcaggtgatctgcccgccttggcctcccacagtgctgggattacaggcatgagctaccgcacccagcctgagaccaccttttgcatctcaagattgtgaaaccaaggcccattccaccagcctggggactctttttatagatatgatcctcctttttcctgtgactaatgaatttgctgcatgatttctattcttctgaggttagttttctgagtaaggtgaccactcacaaaggcactttctttgtggcattctgagcctagattggggcccatcaattccagaaaaaatttatgtgtggaaactctgcatccttaagtcttgaagttgaaccagatatgcagtggttaccatcacacagataaacgctgccttctgtacataccccttatgctgtactaattaacaaaccccttgccagggctggggaggtgagggtgaaggagaatcttagcagaagggcagagtcaggacttgcatctgccactgctgggcactgaagccctggagcagcttcagatagtacctgtactttctcatgcagactccctctgaacaagagccttgtaggcccctctccttcatttcccaccagcctcttatcaggcgggctttccaccatacacccaggaggccacggtctgaggaacaaccaaacccatgcaaagggccgggcgcgatagctcacgcctgtaatgccagcactttgggaggctggggcaggcagatcacctgaggttgggagttcgagacctgcctgaccaacatggagaaacccccatctctactaaaaatacaaaattagccgggcgtgatggcacatgcctgtaatcccagctactcaggaggctgaggcaggagaatcgcttgaacccgggaggcggaggttgcggtgagccgagatggcaccactgcactccagcctcggcaacaagagcgaaactctgtctaaaacaaaaacaaacaaacaaacaaaaaaacccaggcaaagtttccttgcagccaaggtgacagaactgggctgagggtggaaaagaaacagaaccagtgctccaggtgttttttaattttttaatttatttttattttttttgtatatgtatatatatgtatgtatattttagaggaccagggtctcactatgttgcctaggccagactcaaactcctgtgctcaagcaatcctgcctcagcctcccaagtagctgggattacaggcatgcacaaacaatgcccagctctccaaatgttttctgtcactacctgaagtgttgcatcggtacttcctacggaaagaaaactaaatagaagtgtctctcccgtgagcccccaccactaccaccagaaaaaaaaaagagagaaaatgaactcatcagtctttagtttcctcaagttattctcccaaaaagacattcgccttggcacagataagccagctaatcttatgctttatgacccactgtgagctgttcctgacacagcttctgactttgtcagtgacaaaatttctcaccttttaaatgcagtgcttaacattttgttaggcccatactcaaaatcggccagatataaaatgacctcagattttgatctcctaggctcaaacaatcctcctacctcagcctcccaagtagctgggactataggcacaccaccatgcacagctaattttttttgtatttttctgcagagatggcgtttcgccatactgcccaggctagtctcaaaatcctgggctcaagcaatctgcccacctcagcctcccaaagtgctggaactacaggcaagagccactgcgcccagccacaacctcagatttctttggcaaacagaaatgtttaaaaacacaaaattttgctcaggtgaaacactgtgttactatcaaatctcacatccacataaagtttttcttttcggctttgtttcgtgaggaacagacagaacaaagtttttccaggtagcatctgtatcactattattctcctatttcctgtaccacccccacctccccaagccctactgaatgtgaggtttagaatgttttaaggagggtcaggtgcggtggctcacgcctgtaatcccagcactttgggaggccaaggcgggcggatcacctgagtttgggagttcgagaccagcctgaccaacatggagaaaccctgtctctactaaaaatacaaaattagccaggcgtggtggcacatgcctgtaatcccagctacttaggaggctgaggcaggagaatcgcttgaacccaggaggaggaggttgtggtgagccgagatcgtgccattgcactccagcctgggtgacagagtgagactccatctcgaaaaaaaaaatacaaaaattagctgggtgtggtggtgcacacctgtaatcccagctactcgggaggctgacgcaggagaattgcttgaacctgggaggtggaggttgcagtgagccgagatcgcgccattgcaatccagcctggacaacagagtgagactccatctcaaaaaaaaaaaaaaaaagaatgttttaaggaaaaaaatagtactgttacatataatcccaggtgataagaccacaatggaaatgtttaagtcctcactttaaagagtaccccactgagaagaggtatgttggactctagcagagatttggaaactctgggacactcaagatgtgaaagagcctggctatctgaggactcaaagagtcagcatcgggacttgtgagctcaagaagagaaaagggagtggtgaaactttgtcctaaaagttagcaccaggaacagaagaaaaaaacccgatatatagtgatacctcatcttttagagaatgggaagctatttttgtgttcacacagaaagtatagttcaaaaaacctctatatccagagttcagacaaggagaatgatttgagatataagtgccgatgaaggaggtcaattttgatctgaaaccagcagctggacctgggccacctcaggaaaaggactctgttctccaaggcagcacgactgaatggttctgagaataagccagggttcaggactcctgaccctttaggaccatggactcagaagagcctgaaggacaattgtgggctttaaacttctgagagcttgtaaagtaacacaagactgtgcctctcccttgccccagctgtagatagtctttgccccaccattgttatgaagatacacagggttttgcagtttgaataaattggatacaagtttcctctttttttttttctttttgagacaaagtctcgctctgtttccccaggctgagtgcagtggcacaatcaaggcttacttgccgcctcaacctcctgggctcaagcaacgagccatcctcccgtcttagcctcccaactagctgagactacaggcgtgggtcaccacacccagctaatttttgtactttttgtagagacagggtctcaccatgttgcccaggctggtcctgaactcctgggctcaagtaatctgcccacctcagcctcccaaagtgttggggttacaggcgtgaggcaccgcggctggcctgagtttcttcttaatactgtatcacaattgtgggctgtcttatgtgttgatatcgattgagctatttgaaataggaatgttaatgggtgtattaaatttttgtaaggatataacaatatctaccttccaaggatgttgtgaggttttccatgattttgtatatgagctaatgttacctttgaggggtggtgtgcattatgttggatgattgtaaattttcagtggaaaatgtaccgtgtcctaaatttaaagacatgaaaaatatcccaagatcatactagatcataatagcaattcctttacaaatgaattatggaggtaactgatctctaacagtttccttcatgttgttttaatgcacaagggcagaggatctgctgacccttggaaccagcgtgagctaaccacgtgctatagacacttcatggtgtcgcacccagggaagtcaaagcgctttgctccctcactgtctgtgagtcctcagccattagtaccccaccccccgctgctccaaaacttgagttatttcaaatgtttctcactgttcatctctccactgaccccactccagaaagcctggagagagtcccaagatgccacccaccttccccaatccctcgccacagatctgtgtctatctcacactctgtaagtgccgctttgcttcttcctctcttgaaaagactgagaacacacattttaacatgttaggaaaatggggcagcctaaaaaatgactgatcccaccgccagtgactcatgtatactccaggctagcagacaaggccctttttggtgggcctgcttctgtgggttcacagaaaccaaattactgtgggttgcaaagaattagcaggtcatttacaaagcagacatcccttcacccagactgtggttttgcatgctcaggttctcagtctatgagctttggtgcaggatcattttggctactggaaaaaccatagcttattttaaatttctggttgccaaagccaccacacgtgtggtctgtggatgaccattgtctgcagaatgacgaggaaggaacagaatgtggtttggggctcagggtggccttcccactgggagggaaggcgggagggagcccttgccctgggttttgacacagcctgtgctcacagcctctcctctcatctgcatttctcagaaatgccctccctgcccagtggtgactttccctcgtcactcctatggagttctacctggagcccagccatgtgtggaactgtgaagtttactcctctgtaaagatggtttaaagaaagtcagcttctgaaatgtaacaatgctaacccttgctggaaccctgtaagaaatagccctgctgatagttttctaggtttatcatgtttgatttttacactgaaaaataaaaaaatcctggtatgtttgaaattagtccttgtgcatccttatcacttatgatttgtgtctgctggtgtcaggatgtagaagggtttcatcccgccccaaggaatactttcttaattttgcccctctatttcttaaagtccagttatcaagaaaaatgaattatcactgaaataaaggttctcacctgacgccttgctgagcctaccactgtgatcctgctgctggcccctgtaacccacaagatagggttcctggttccactggtccttagttcactaaggaaactggaggaggttgaggacctggacttagcatgatcagcacattcagcatggtgcacccctattccaagtcactgctctgccttcctccctcaacagagattcttcttaaaatacagtcaagttctgactttttcatgtgtgggggctcacagacatccacattgggaaacttgagttgcagtccttaaactcgaccatatttaaatacagtttttagatacctggagacccaaaccgaaggtgttagggggaaaagtcctaggagaatggcatagggatgaggctgctttctttccatcatcattgtgttctctcttagaaaacgttagagaaagta

SEQ ID NO: 4 - DNA, 31977 bp, Homo sapiens
  source 1..31977: mol_type = genomic DNA
ctagggcacatgtcgtcaggacctcctgaggctgcgttacgggcgtgcgtccctaactttggcaaaattaagttttgccaaaaacttcctaaactgagacctgtctcagatttttgggattcacaaggtacagagggaaaacttggctctagggcctgacgtgccaggccacatcgggcgtccagcgtgtgcttagattgcaaactgggtgcagattaaaggctagtttttgggttaccttcggcagacgatggtgcctttcggatttgaatctttcggcgctcggctactctcccctagctcctctcccattttcccccagcagtctcccgggcttggggacgctcggtgttcggccacgccgcctgtcgcctggtagtgggaccggaagttgttagggggagggggacggctgtgatggggggggatatttgggggggcaagtcccttctcaggtgaagctgctgatggagatggagccgccgccaccgccgcctctgagcgcccgggtcctggctccggcccggcgactgccgccgcctcagtgaccccactccccccgcactgggccgcccgggccagagtgggggacccccgccccctcgcctccctctcccccaacactgtcccctctccccaacccctcacagcctgcgcgcgcgcggagacacctcaggtgagtgaggggcggggcttggtgggggtaaagcgggggcggggcctcggtagccgggggcggggcctgaggggctctggcgtgaaggaggggggagacgtgggcgggggaaggggccaggctgagagagggtcgcgggttgggctggggccggagggttggaagatggccgggggagtttggcccgaggccccagtattaagagcgaggtagcagggactcgggggtcctaaaaaggcgggagacagatcaaggcaaggggccgaggacaggcaggagctgccaggcgcggggctcctccgtggggatgtggcttgggtatacagcagaaggacagcgaggagaaaggagcagcaaagagggtatctgaacaaaaaaagcgagggacccgggtgttgggtcgaggaatgctgaagacaggaatctggggataaagacgtaggcctaggtgagatctgggaggcaatgagcatattgggagaaacatgtagagaagaaacttaggatggagtgagaagtgcacagtgtagggtgagaggtgtagggaacttgggaagcaaggctgggaaagaagagattcactgcttgagagccctaagaaagaaatgcaacagaggaatccccagggcagacatccagacgggagctgcaaacttgggaagcaggctgcctgctcctgtgtaccaaaacagccctccctgtacctcctcacacaaaccccatcccgttattttcttacctcatggctgctagagttgtaggttctcactttttaaattttctatattatttctgtgcctttaaaaaaaattatgactagtcctttctgggaacagtggagtgtttaatggaacgtggacctttcgatatgcttatgaaatattgtaccccaatccccatccaaggtcaagaagttacgaactctgcttccaggaaattttttttttttttttttttgagacggagtttcactcttgttgcccaggctggggtgcaatggcgcgacctcagctcactgcaacctccatctcccaggttcaagagattctcctgcctcagcctcccaagtagctgggattacaggcacccgccaccacccctggctaattttttgtatttttagtagagatgggggtctcaccatgttggccaggctggtcttgaactcctgacctcaggtgatccacccgcctcagcctcccaaagtgctgggattacaggcatgagccaccgtgcctggccagaaaattttttgcataccttatttcgccccattcccttctctcatgaacaaaactgtctattgttgttactcttgtcaaccctcgcttatatgtctaatagttcctactgtgcctgtctccccagcctgtgccctcgaagcatcaatggtgggcatggtatgttcctgacatgctcacatgtcagctttagatgttccaacgtcatggtcaaccagatgaccattgaagcctgtctccccttactgatacttgctcactttgcctgggtgaagatacatgttcatatccttaaggaagcatgaaaatgttatctgactacctagaatatttccacttaacgataactcagattctgaggacaaggccagataaaaggccaaatcagacaatctttgaaagcatagtgtttcgtttggttggggtgtatggtaatccctcacctccccatggacctgccagtaggtccgtgaatcttgctttatggtttgcaggaggacttcagctttccctactaacagcctaaagccagagaatccagacagacccttgggtcatgagaacagtcagatgagtagctcctgagataaaaataaaatcttaaaagaagaaaatagctttaccattgggaagcaagagaagtttcactggagcttctggggtgggcttcagaatggtgagagctgccctcattttggcagcttcagaattataccttgccctagtcaatagaagttgatgtctcaaacacaaaatataatgtctttgttgcccactgagtgcagttagttagttgaattgtagcttctgggcaatccccttcctctgagtaggttaagggtaaggcatatttcaggacttggctcagaaataagtcctgacttttttccagtgaaaatgcaactgaaagtctctttatgtcctcttagtttgaaatctgcagcttgaacttcccagcatgggagaaacttatcccacctttcaccgtaccccctgcccccccacacacagtagtatccacaaaggaattattttttttccccaactggccatctctctctctgtctctctctctctctcgccacacacacacacaccaaagaaaaagaaagataggtatttctagcctcgttttactctgtaggaacaaataagagtgtgggaccctgtgagtgtggacaatgccacaggcagagggactccctttctttattggtctgttgactctgctgaagaaagctttgaattccaggctttgatttgcttcctcttcttagaagagttatggactctgttctggaaacagagagataggttcaaagtttatcagacaaaggcatctaagaaagggtagtgctgatatgaatgacctggtccagctgcggggaggatccacattgtaggctctgatgcaatacctatgctggggtagtcctcaagctaagaggccgtttctactttagagacttaatgaccatctagtttggtgttttcctcaggtccattatctctgaaaagagacataggaggaagaggattatttatcctcccatccataagcctgtgtttccagttcaaggatttaggtcagcaagacgcagacccaaaaaccattcatttaataacccaagaaagaaggcaagcagggtggcccaaatcttccaactggggcaagatttaatatagtgttccagtggaaagaatcttctaatggtaacttggactctacatgtcctaattctgttgttaatgagattgatgactctgggcttagtttcttctgttaaaaactaggaaattgtagataatctctcaactccatttcagctccaggatttctcagttcctggagtacccctgccctagaccctccacatgaagaagagagtcttaccaaattggattatcagagcaagactattagcagagaagcctgaaaacatctttctggcccaaacaacagtgaacttttttgatcagcatgtattagggacttttgggcacaattgcaatcaaaggcaattgttcctgtctcttctacattttttttgctactgtaccctagtcagaatagttcactgagacttccctgtaatttcaaacttcacttgagagtgctaaccctgttttgggcatctttggctttaaaactgattattataaaactcaaattgaatttccctgctctactttctccacccctgccaaacataccactgaacagcagtgattttgtgaggcctttctataaacagagagctatacttgggggtacacccaaaaaccccaaaaagtatcatctcctatgtatcatatctagtgatttaaagtgtttagtttggcccttttccttagttccttatgtcctgaaatgagacatgggagggataggatcgtttcttctcccactcataaaccttagccttctctctcttctgcagtctacatggggaggacagagaagcgcaaagaacaagagaaaagatgcatccatctgagatctaaaaggagacaatgaggtgatattttccccctgcccaggtgttgtgttttcagctcttacccctttttattttagagactggccattcctaggatgacctgggaatttatcccctctgcactgctccccaagcacttggataatgaagctgagctgtcttcaactgtattatatgtagacagaaggtcaccatctacagcctggtgaaaaatgagtcagacctttccactgtaaagacaaacacatgagctgagctaaggttggtgggtttccttcagagagcactaggttgaccctaaagataatttcagagctgcaaattgcttgatgtctaggtctttttcctggaaaggggcttgcagacagtggctaattccatacctttccctggcccctttgcgtcttctccctcctcaattatgcaaagctttgatagagagcagagccagctatgtgatttcaacatatttttgctcagatttcactcagttcaggaaggcatagacagtagaggatgatgctgtgggtttgctctacttgtagcacctgaatggctcatacccaaggcacatgggaagttctgtcttgttaataactactgctctccaccgcagaacccaggtactaagggaaagtggagaaaatttttctacttctgcctgttatagtttgggaataataacaaaaacatggtatggatccttgccattctctctaccttcaccatctttcccaacaatattcttctcatgaccttttaccccctaggccacattaagcatgtgttaaatgactttttttttttttttttttgagacggagttttgttcttgttgcccagtctggagtgcaatggcccgatctcggctcactgcaacctccgcctcctgggttcaagcgattctcctgcctcagcctcctgagtagctgggattacaggcatgaaccaccacccctggctaattttgtattcttcgtagagatggggtttctccatttggtcaggctggtctcgaactcctgacctcaggtgatccacccaccttggcctcccaaagtgctgggattacaggcgtgagccaccacacctggcctaaattacttttttttttttttagtgtctcagtgtgttgcccaggctggcctagaactcctggcctcaagcagtcctcttgccttggcctcccaaactgctgggattggccctcactgctgcctgtcagggagcctggtgggaaatcttctgtctagaaaggatgcccattcctgttcaaatgtgttgtaatgttgataggagaaagccagggccatggaggactcttgtttatgccaagtggattcaaacaaatggggaagagctaggcacagtggcacatgcacctgtagtcccaactactcattaggctgaggaaggaggattgcttgaggccaagagtttgaggctgtagtgtgctgtgattgtgcccatgaacagccactgcactccagcccagacaacatagtgagatctcatctctagtaaatataataaataaataagaacagggaagaaaatttgtgtctccagagattacaatgaccagcgtttcctccctcctcacagagatattccttttgaattggaacactgattgtttctagttgctttgtattcagctcacagctgaatttacagtacatatgtgcctacccgccatcactgagtgacggttatgttgctttatttatttatttagtgacagagtctcactctgtcacccaggctggagtgcaatggcgtgatctcggctcactccaacctctgcctccggggttcaaacgattcttatgcctcagcctcccaagcagctaggactacaggcatgtaccaccaagcccagctaatttttgtatttttagtagagatggggtttcgccacgttggccaagctgatcttgaactcctgacttcaagtgatctgcttgcctcagcctcccagagtgctgggattacaggtgtgagccaccgcccccagccgacagttatgttgctttaaagctaatctccttccaagatctttgatacaaaactgtctcatgtaactaacttccctctccttatacctcctcctcctgcaatgtgtctccaattggcagtgcctaatttgacccagttcctctaaggagacgctaagatctgcctggctggttaggcaggggaggaggtgctttaaggggtagagagtgggaaggctgggcgggatgtgctgcagggagacttgagcaaagagtaaagtacatctgctgatttgctcctggtttgcacagaaaaccaggactgagaacagcagtttcaatccaatgccctctcacttccagggttaccaacttggtattaaaaccagctggagagccgggagcagtggctcacgcctgtaatcccaacactttgggaggccgaggcgggcagattacctgaggtcaggagttcgagaacagcctggccaacatggcaaaaccctgtctctactaaaaatacaaaaattagctgggtgtggtggcgcacgcctgtaatcctagctattcaggaggcttaggcaggagaatcacttgaacccgggatgcagaggttgcagtgagctgagatgagatcgtgccactgcactccagcctgagtgacagatcgagactccgtctcaaaaaaaaaaaaaaaagaaaagaaaagaaacaaacaaacaaaaacccccagctggaggcagctaagtacagctaagtactgattttgcccaagatgtgtcacagagcacgttaagagcaattaagcaggaaagctcagtttactgttagaaagaaagtccagctcagaaaacaactccttcagacctttctggaaattgttttggggccatctgggcaaaagtccaagaatgagatacacttcctgagccagtgctagctgaatggtatgcttcacctgttctacccacctagtgcttgtctagcccttcattcacacaggcattaggcatgctgcatgcacacaaacacacacacacacacaaaataatatgagcatatttcttcctgtagagccagctcgacttgttcttcctttttaagtaacagaccccactcccacagggatcacaccaaaggaaaagatgacttacctgatgttagaaccagggaaaaagccgagtgtggtggtccatgccagtagtcctggctactcagaaggctgagccaggagaactgcttgaacccgggagccaaactcatgccactgcactccagcctgggcaacacagcgagactccgtctcaaaaaaaaaaacaaaaacaaaaacaaaaaaaacagggaaaagcaagaaaacccatgaaggccttgagtagcctggcttttgaaaggtttaatagaaagaggacacctcagagaggcaggcaacttgtgttcttgttcttgttctattctcaccatgaaccttgggacaagtaacttcactgctctcagcctcagtttctctcagtttctccaagcagaaaacaggaaaatcctgctcactcttgcctctcctgcatggggaggtgtgtgagggaagtttcaagtggcactttgtgggcttgttttgttttgtttgagacagagtcttgctcttgtcgcccaggctggagtgcaatggcacgatctcggctcactgcaacctctgtctcccgggttcaagcaattatcctgcctcagcctcctgagtagctgggattacgggcgcaccccaccacgcccggctaatttttgtatttttagtagagatggggttttgccatgttggccaggctggtctcaaactcctgacctcgtgatccgcccgcctccgcctcccaaagtgctggaattacaggcgtgagctaccacgcccggcttcaagtggcactttgaactttgcattctttttttttctttgaaatggagtttccctttgttgacctggctggagtgcagtggcgtgatctccgctcactgcaacctacacctccccggttcaagcaattctgcctcagcctcccgagtagatgggattataggtgtgtaccaccacacccagctaatttttttgtatttttagtagagatggggtttcaccacgttggctaggctggtctcgaactcctgacctcaggtgatccacccacctcgccctctcaaagagctgagattacaggcctgagccaccaaacccagcctgagctttccattcattttgaaaaacaccaaagggcaattgtgaatctaatgatttggaaatgaaagggcccctcaataccagactgccctcctttatcttttgatctctatctcaagttttgtggctagttccagagagtctgagttctctaacaactgtggacttgggctcttctaatgattgttcctaagggacccttcaaggaaccacttattgtgtggcttgttctagtggtccagtcccaaagttcagtccatcccaggaaggatgtcaagaaaagagggttaataaacttagagaatttttttctccatgccctggcttcctagggaaatagggttgatactaggaatctctctgacaaaatgtaagagagaattgctttgtgaagtgaaggtaagcgcatgtttcaggttttctcatgagccttcaaaaggagtctcaactccagacaagaattgggtgggggtgggggggcaggaaaacagatactttactctgtgtgccttcctgcaccccctcacccccacccctacctctctcaatataccattacaagcaaaccaaatctttatccatctgcctcagtgagatccttcaaaaagaccaagaatccccacagcagctcagactgttcccttgccccattcttgcttctgcctctgcctcagggcgtttagttttcatcagatgagtggggaatgcaattttctttttttttcttttctttttttttttttttgtgaccgggtctcactctgtcgcccaggctggagtgcagtggcaggatctcggtgcaccgcaacctccacctcccaggctcaagcgattctcctgcctcagcctcccaagtacctgggattacaggctcatgccactaccgcccagctaattttgttttgttttgtttttttaagagacggagtttcgctcttgttgcccaggctggagtgcaacggcgtgatcttgggtcaccgcaacctccgcctcctgggttcaggtgattctcctgcctcagcctccctagtagctgggattacaggcatgcgccaccatgcctggctaattttttttgtatttttagtagagatggggtttctccatgttggtcaggctgaactcctgacctgaggtgatccgcccgcctcggcctcccagagtactgggattacaggtgtgaaccagtgtgcccagtcataattttgtatttttagtggagacggggtttcaccatgttggccaggctggtcttgaactcctgatttcaaatgatccacccacctcagcctcccaaagtgctggaattacaggtgtgagccaccatgccagccgcaattttggaggtatgtaaatttccaccagatagattttttttttctctcctcccacaggctcctaaagagataattcattgcaacaaattatcacttttaagtgttctcagttcccgattgtttaatactctactgccaattactgcttgccttccctcaaatagacattaaccttcattttaaattcagactccccccccgccccccccgtccttttttcttttttaaaataagcgtggtagagtaattgatgagagggtggggataaccatttacaggtttaacttgtttattacaccttcagtagaggacttatcactgaaccctgagtgggctgctctcctccatcttctccttttcctgtcaccctccctcacaccacatactccaagacagaatcccttgaatgactgaacccagcttcttaatactacccccatctttaactgaggtatagagcctctaaggggctgtttctttgcctcctcagggctgaggtgtaatctccacctacatacacggtctaatctcaaaatcaaggaccactctggattaagatagtgtttccacctcagaggctggggaggcctgcagaagttgtaaattggaggaaatattggggtttgtaaatcagggtatgttgtctttcaccaggacaaagtgtaaggtttttcttggagacagggtctcgctcggttacccaggctggagtgcagtggtaagatcacagctcactgcagcctcgacctccaggctcaagtgatcctcccacctcagcctcctgaatagctgggactacaggcacatgccacgatgcctaatttatttttatattttgtagagatgggatctccctatattgcccaggctggtctcaaactcctgggctcgagtgatcctcccaccttctttccaaagcactaggattacaggtgttgagccacagtgcctggctaaggtttttcttctttaggatagtatcagaccatgcttaagtgctcctttaggtttcttgctaactcaagaatattaaagaaaagcctcctcccttcccagagctatcttctaatagcagctattttctttttcagcagaatctctttaaaatggacatagaagactgcaatggccgctcctatgtgtctggtatgccttctgtgtgttctatttgttaggggctggaagcagacactctcctgtagagcagagcagagagccttcagcctaggcacacagcctctaaatgtcctagtcacacagctaacactcatgcttggtggcccttggtagtgtgcaattattttggtgatcttacattccctaaatagtaattttcttttccagacttcttggggtatagaggaagggaataggaggggaatatgagcagacatcattagaagaataatttgaagcaaatagatggaaacaagatgtggttccttctcctttgctctgaagtcaccagccaaacatgaaatcctgttctatccagatagagcggcacaaggtttcaaagctcaaaggcctgcaagggccaggtagtcatgtgaatgagtgaagtaaggttaggataataagcagaaaagaatggtgggggctgtcatgaagtggagaacatatgtcccacttaaagggacagcagctttcagtgtccactagaacttgtcatgcaggaatgtgtatctagtgtggccaaaacttctaaatttttaagagataccagaaatccaaatttttatatgaaatgtctccattttaaaataatgactcaaggctgggcatggtggctcacgcctgtaatcccagcactttgggagacaggcagatcacctgaggtcaggagttcgagaccagcctgggcaacatggtgaaaccccctctctactaaaaatacaaaaattaggccaggcacgtggctcacgcctgtaatcccagcactttgggaggccgaggtgggcagatcacgaagtcaagagatcaggaccatcctggccaacatggtgaagcctcatatctactaaaaatacaaaaattatctgggcgtggtggctcacgcctgtagtcccaacttctcaggaggctgaggcaggagaacccaggaggcggaggttgcagtgagctgagatcacgccactgcactccagcctggcgacagagtgagactccgtctcaaaaaaaaagaaataatagaaaccaaaactgggttttgatgtgatgagtatccctatcagaaacccaagcaccattttggaaatgacctatagcttattttttactctaaagtaggaccaatgaaggaattattggcatgcactaaaggagatagcaagatgggtcagacacacatatgagagtcattggcaacacccgggtaatgtaaggtaagatgttgactctgacccctttcttgtttaaggagggaaggagttggaatgggtaaaggcattcaatatctgccacatgcttgacagaatgacagggatcaggtagactcatttaattggaagagactttattagaacaaaagtcatggccttgtgaggagcgagccagtcctcctacctcctgctcagttaagcctattcagaaaaccactactgagcaacaagtacacttgggaccctgctttagtgctggggaacactcagatggataaaatccagtccctaccataaagcaggcaaatgagaaactacaaatcctgtggagctcttttcaaacctgtttatggagaatattggatactcatggaggtgatttgcttcaccgtgctatgaaaccaagttttccctgctttcagcagattggcatgtcctagataagaagtggcctctgcacaggatggtttttgaatggaagtggaggccaggaactcctcctctgtactttgagaattttagatgtcagcagctaccttagaaagggaaaagagaaagtcaaagtaaggctaccattagccctgaaaagtacccctcttaggactgggcatagctgcgctcatcactgacccatctctcctggaagagtcatatggaagcagagggaaaggctgcagcatgcagccctcccgaagcacactgcgggcctccacaggccatcatagttcttctgctccacgccaggtgaccttccctcatcaggccacttgatgaaaagggtatctctcctgttcttccaagagatttgaattcctcacagaatgtgattcagaaaatccatcttttctccccttttctctttttaaaggtacagatcattcccttctggggcagggaaaagggggagataagcaggagtctattaatttatggcacagataaccaccccctccattttcctatctttcaagggaaacagagttgctgaaacccagctcctgccattaattgactgagcagggcttgccccaagttttctgagactgtgaaatgttttagagaactttagtagaaagggattcaggggtaggcagcccgaagtttcacagcccctcccacccccaaattcttggaatcagaaaatgtagtcttccaaagtaggtcagtttgtcagtgagcagctcctctgaaagggaagggagcagaggccttgtcctgatagagggcggattcatccagggctttcggctgctggggccccaggggcacaggaagggggggaaatggggagaccgtaccagcctttttgcccgggatctgaaaggtctcagggccacggagcaacactagcctgacaccagactctgctgggccctctcactgctggaaaggaggaggctgtcagtccccacatctagagagcaaccttcctcctggaggaggagggaaagagttcggtgggggaggggctttctgcctctctgagagccttgaagctgtccgtgtcctgggccccatgacctctggggccttggcttccccagctggcagaggattgggccttccctagggccccccctttctccctcccacccgcaggcccatccatctctctctctctctcttgcacacactcttgcctctctcaggcatttgttgtgcagttcctctttgtctgctgggcacgaggggcaacagcatctgcctttccctccctgtgcacacacccaccacccacccccttcactgtcttggaaaagggatgctgtagcctagcatctcccccactatatacacatatacattctctccagccccctccccaagcacatccaagcgtgctctcccctctccttctctccctctctctctctctctctctcacacacacacacacacacacactcaacacacatacaccctgggctgagctgctcttgctggctgcagccgtgggcctctgctcaccgtgccgctgctgctgcctgcgaaatgacggcggttcccctcacttccaggaatccacgcttcctggaaggtgagtggctgggctcacccctgcctgccactgagacgcagacatgcatacaccacccgcactccctcgccgtttccaaggcggcggccgcgttcgcaccccagggtctcaccggcaagggaaggataatgtaagttcaggcagaaggcggctagtggagggaggaaggggggtgctggggctagggagccaattcagtaactactcccaagcctgaggagatggagaggatgggggctgtgtgtgtgtgtgtgtgtgtgtgtgtgtgtgtgtgtgtgtgtagcgggggagggggcgttcctccttatacttgcggaggatgggaaagggtgtttggtgttgctaagcaaagacggcgacttggacagtgggagcctatggtgacatcttgattaggccttggggatagaggtagagctgcctggggtgaaatgccgagacaggcctgggtgggaaggggtagtggagaaaatcaaggtaaggaagaggagccttggcaagcagatgcctcagcctggaacagagatctgaggggcctcagcttcctccccacaaaagagctgaggatgggcgtctttctttgctcttgtacccactattccctgctagcaggctaggatcctgccaccctggagcccgtggtcaagaaagggaaagcagttatgatgaggtgccttgcagagaagtgtggaggttctccatgttctccaagctcatggctattccctagatgtagccactatttaaaggtctcaaggaaaggcagcaggagtggaggctagcctcctttaacatatgaaacacctttagcttgtggcctgtttcccagttcctttctctccctcctgcttttcagcctttacctacctcccagttcctctaatgtgaacctcatgatttggctttgccctctgggggttcagccaatctatgatgactttgagcaagaaaatttctcagagacctaagagcagccttaattcttggctttgacagtctgggatttcttgggagtcccagacttttgagattccctcactggggtttctggggctgagagaggaactgttaaacttcctaaatcttactctagtcccctcacccctgagctagaattcaaaatccttggtgcccaagaaagcctttctgactcctgaaacagattcattatacatatattttagccccatgaaagtagacttgatcctgtttttccccttaccttaatcttagggagggcaacagcatcaaagagcctcttgaattactggtgtgactcctgcttctcttcctgtttctgggccttaattgggaaagccacaaaggatgcttttccctcagtgtttcttttttttttttttttttttgagacggagtctcgctctgtcgcccaggctggagtgcagtggcgcgatctcggctcactgcaagctccgcctcccaggttcacgccattctcctgcctcagcctcccgagtagctgggactacaggcgcccgctaccatgcccggataattttttgtatttttagtagagacggggtttcaccgtgttagccaggatggtctcgatctcctgacctcgtgatccgcccgcctcggcctcccaaagtgctgggattacaggcgtgagccaccgcgcccggccgccctcagtgtttctaagtgtacttttaaatatattttcctccacatagcaagtcgctggcctcaagaggcagcctcagccgaataagctgggaccagtgatggatttccaacaagttcactcatccttttagggtcaaggcctcaaagccaagtgccctgttcttccctgatagtaacatcaagtgatttagggcaattcagcactaataaagggaataagaagataacattttcagcccttttaaccctagtaaaccctacacttagttgaaaggatatatttaaaactggctctgagtctacaagtctcaacccacaatcctgagatgctcacttcatgatatgtgtgtggttctgatttcgggcaacatttttaggtaaccatggctcacctaaaaagccccagctgggaaaactcagattcaagccataaggctttttctatagatagtgaacatttttcctcaggctttgacgggatagaatggggaaaatgggactttaggagaggatatgaatgggaaatggtcccaaagtcctgagtggttgttttcttcccactgaccaaagctggagagggatccctcaggagggtgtgttccggatttcttgcctcaggcccaagactccaaccattttataatggaatctttattttgtgaaagtaagtatgtgcatagctgggcaattgggtaaagtactaataggtggacttcagggatgaattttaggcttcctctctctttcttgcactctcccttgctttctctttctctcttccatatttaggcctgtgtgcacacatccatgcatgttcatgttagcagataatgtagaaggctaccagtatgtttatcccaataggtatgttctctttatatgtatctacatatgtgttgtgtctctgtgtgtgtttatctacatgtatttttatttcacctatatatttgtgtagaagattgtggggaggagggatttttttaaaaaagacattcatatcttcctttaggtatgttatctgattttgaaaaacattatgaaagcctcaaaaagcatttttacaaagcaatttatcagaaaattaaaacaaatatactgagaataagttgatcacttaactgtcaatgaatatcaaatagtaaacacgtgattggaatcagatcagtaggagactttctagagtaatcaaatttttgctgaatttagaaagtctaagaggcccaattcttccctactaagattgtgaccaccaattggctgtgtaggtgctggttaaggacatggacgcttactgtctatgatcccagttccacactagcagctatgtaaccttagacaactaacataacctcttttgtacctcagttgcctcatctgaaaaaatcatgatattagcacctacttcatatagttgttgtgaggattaaatgaaattattcatttcaagtgcacagaataaagcctggcacatagtaagtactcaattaatattattagctgctatgatgattactaatattatcattagattcaaggacaattccagttacctttcctgcagatcctgaaccggctgggggctccagagaggaacagtagggaagaatgtctgtcctgaacatctgtcccttctgcagcccccattttcctctccgccctggcgcacccagcctcagccctgcttcctgtggacgtcaggcagtgatgctcttgccctcaggcctagataaaggtagggtaggcaaggcccagccactgagctcacagaaggcagcaccaggcatccagactggcgggggcacaaggtatggtgcccagcatacaacactgctttgtacatggctatgaaggaaatgtttgtgaaatttaactgaatttttttaaaagattaggcaatggacagtagatatctccagctccagctttacctgccctcttgttctctcctccaaggtagcggggactcatctctggagaaggagttcctcggggccccagtggggccctcggtgagcacccccaacagccagcactcttctcctagccgctcactcagtggtaagtgtaacctcctaccacctcctggcagtaggcaccccctgttgcatttggccttcaattcaccttccactggcaaccatttccccatcgtcacctcctgctggtctcctgcttagtctctaagagcaatgaggagcccagtgtctagggtttttctggcttctgaaaggagagtgaatagtcagtcactggcagactagggtggaattaaaagaggtatgggtgggattaaggtaaaagtttcaaagagaaattatggaattagagaagcaggtagatagaaatggtcatcagatggagttagataggaaatagagagtagaaggacctccccatctccccacagctcattctcctacccagacagggtccccatgactgcctctccccagctggccctagatctgtgacattctctaaatggttggagtagccaggaatgtaggtcggagctagagccaaagccttctcctcaatggttcctccttcctttacccctcctgccctgcctcagagttttatggaggtggagggggtagagggattcaatctaatgaggtgggagggaactgtaagcatgtctgtcttccaggtttgctctctttggcctgttgtcccccttcagtcttcctagatctgttttcccgccactgttaacttgctggcttcctgcccagggccaagccagacatctttcttcatgacaagagagtagagaaaaaagctccccttagtcgcctcctctcctcagcgacagaagacatcctatgtacccccaacttttttctttttgagatggagtcttgctctgtcgcccaggctggactgcagtggcgtgatctcagctcactgcaacctccgcctcccaagttcaagcgattctcctgcctcagcctccgaagtagctgggattacaggcgtacaccaccatgcccagctaatttttgtatttttagtaaagacagggtttcaccatgttggccaggctggtctcaaactcctgacctcaaaagaatccacccgccttggcctcccaaattgctgggattacaggcgtgagccaccacacccggcccaacttttttctcttgaagcctatttgcctggtgggtgggagaaaaggttgtctcaggttccagtaggattctgatatcagcagcagggatactagggtgttagttccatttaaggcttgaatgtctcagggcctaaagacaccctcttaaactttaagagtgccattctgggccaggcgcagtggctcacacctgtaatcccagcactttgggaggccaaggcaggcagatcacctgaggttggaagttcgagaccatcctgaccaacatggagaaaccctgtctgttctaaaaataccaaattagctgggtgtgatggtacatgcctgtaatcccagctactcaggaggctgaggcaggagaatcacttgaatctgggaggtggaggttgcgtcgagccaaattcgtgccactgcactccagcctgggcaacaagagcgaaattccatctcaaaaaaaaaaaaaaaaaaagagtgccattctggcctgggagcagcttccccctttgcctctctctattctaaacaccatcccacccctcagccaactccatcaaggtggagatgtacagcgatgaggagtcaagcagactgctggggccagatgagcggctcctggaaaaggacgacagcgtgattgtggaagattcattgtctgagcccctgggctactgtgatgggagtgggccagagcctcactcccctgggggcatccggctgcccaatggcaagctcaagtgtgacgtctgcggcatggtctgtattggacccaacgtgctcatggtgcacaagcgcagtcacactggtaagtaagccagaggggctctgggaggagctcccagggtggggttgagaggcaggggcagtgctgggacaacagcaggtatggggctggaggaggtcatgcagggagtgggtagcctcagaaagggaagggtctcacccagcacattttccagctgacaaaacgaggcagggtgggggtgggtacagaatttgaattcccctcaatacctttagtttcctggcactctcctcaaccgctgttataaagaaaaggtgatatttttctcagtatatgagggaggggaaagtgtagactcaagaatgagccaatggtccaataggggtaagactcctgggtattctttgggccctcccatctaataattaaaaaacaacaacaacaacaacaacaacaaaacagaaaacaaaagccttctcttctcagcttccttgggccttgggctcctagggcagagtgggccctgtggggctcagtttttggtaacagtcttttggcctcgtgttccaggcccctctccctgccacacacctctaatccacaccttccccagggtaagaccccagggatccccaagggatcttcataaatcagggtttgtgtgcattgcacttctgcccgtcagcaatgtgttcagagcaccttcccttgggcaaggtagggcagagggtgggtgataggttcaaccttcaaagttcctcacatgcagtcctctgggatttgttctttcacttgcaggtgaaaggcccttccattgcaaccagtgtggtgcctccttcacccagaaggggaacctgctgcgccacatcaagctgcactctggggagaagccctttaaatgtcccttctgcaactatgcctgccgccggcgtgatgcactcactggtcacctccgcacacactcaggtcagtgtctgtccagtgggaggagggaatgaggctccaacacacccagtatgtagagctcagtgacttctcttgtatttggggagttccagtgtctgtcattgaggaggggggagcatttacagagcagatagggccccctgcctctctcacccatcctctgggcagcaaggcttcatgggaaatttgggagacaccttgatggggtattttacatccaccatatggatgtaaaatccatctacttggagcaaaaggaataggagcaatgttcttggctgttaacggtcacccttggcctggcgcggtggctcacgcctgtaatcccagcactttgggaggccaagatgggcggatcacgaggtcaggagatcgagaccatcctggccaaaatggtgaaaccctgtctctactaaaaatacaaaaaaaaaaaaaattagccgggcatggtggcaggcacctgtagtcccagctacttgggaggctgaggctggagaatggcgtgaacccgggaggcggagcttgcagtgagctgagatcgcgccattgcactctagcctgggcgactgagcgacactcagtctcaaaaaaaaaaaaaaaaaaaaaaaaaaaaagtcacccttccctgacttttgtttgttttttgttttttcaagacggtgtctcacactgttgcccaggctggagtgtagtggtgcaattttggctccctgcagtcttgatctcctgagctcaagccatcctcccacctcagcttcctgagtagctggaaggatactgccacacccagctaatttttctatttttttgtagagacaggatctccctatgctgcccaggcttgtcttgaactcttgatctcaagcaatcggcccacctcagcctcctgaagtactgggattacaggcgtggtgtcagccactgtgcctgggctttcctgacttttatattttagattgttgatgccacaatccatgccctattacccccaacatctaagcccctgggaaatccctcctctcactcccatctgcctcaggccccacacctgtcatcatcttcaccctccttcctcccacaccaccagaggtcttgagcaaccagtctgtcctctcctttacttccttctggctcctggaaatatccatagccctctcaaattgcaaaaaagggagcctaggacttccaaataggaaaagagtatcctatttctgttccctatcccaagaggttgtcaccttcccagaggcaagagaaagtccagagggtacctagagatcaatgtttggaacagtgctgggaaactggattaacaaggttttctagcccagtcaagactgtttgaatcagtatctgtctgaatcagctggacagatagcactttttaggcattttactaagctttaggcttttggcttggacttgggaagaggccaggagctgagctggcaggaaggagggagctcaagattgggaaaaggcaggaagaaaggaagggggaaagaattagtagggtctgtccttggaaactttcctggctatttttctcttcttaaactctcctgagccaggatttaaagggacagagcaatcatttcatcaccctctactttctccatccacttaccttgttccaggttctcccaccttgtaattgggacctctttatctctatgtatgtggagaaacagggagaaagatcatcgttgatacatgctttatatctacagagcatttcacttgtgtttctccatgtgatcctcataacaattccaaggtagatgtgattatccccattttgttgacaaggaaaatgaagctgttggggggaaaaatgagttgcgggtcacatagctattcttcagcctagctggaaccccatcaaggtccagtgccagaaatacatactccttttcctcagaaaaggggctgctccccctgctggtttggaacaaggatggcttggcccctagaattcttaccaactacacagtgaggtgaactgggtagattttggatgttgatctgtagattattaacaacagaagcagacacagatatactgtcaggtcccatctctgtgaagtgctttctgttgcaacccctattataagaaaaaagagactggatgcatgttctcactcatgtgggagctaaaaaagttgatctcatagaagcacggagtaggttgggtgcagtggctcacgtctatcatcccagcagtttgggaagccaaggcgggtggatcgcttgagcccaggagtttgagaccagccagagcaacatagtgagacttcgtctctactacaaaaacaaaaacaaaaacaaaaacaaaaactaggtgtggtggcacacacctgtagtcccagctactcaggaggctgagacagggaggatcacttgaacctgggaggtcaagacttcagtgtcctgtgatcatgtcattgcactccagtctgggtgacagagtgagaccccatcttggggaaaaaaaaaaaaaagccaggcacggtggctcacgcctgtaatcccagcactttgggaggccaaggcgggcagatcacctgaggtcaggagttcaagaccagcctgaccaacatggagaaaccctgtttctactaaaaatacaaaattagccaggcgtggtggcgcatgcctgtaatccctgctactcgggaggctgagacaggagaattccttgagcccaggaggcagaggttgcagtaagccaagatcgcgccattgcactccagcctgggcaacaagggtgaaactccgtctcaaaaaaaaaaaaaaagtagagagtagaacagtggttaccagaggctgactgagaagggttgggagtaggtggatagcgagaggctggttaatgggtacaagattacagttagatgggaggcataagttgtagtgttccacagcatagtagaatgactgtagttaacaataatttattgtatatttcaaaatcgctagaagagattattttgaatgttcccaatacaaagaaatgataaatgtttgctgggcatggtggctcacgcctgtcatcctagcctttgggaggctgaggcagacagatcatgaggtcaggagattgagaccatcctggctaacatggtgaaacccgtctctactaaaaatacaaaaaattagctgggcatggtggtgggcgcctgtagtcccagctactgggaggctgaggcaggagaatcgcttgaacccaggaggtagaggttgcagtgagccgagatcgtgccactgcactccagcctaggcaacagagtaagactctgtctccaaaaaaaagaagaaaaaaagataaatgtttgaggtgacagatatgccagttaccccaacttgatcattacatgtcgtatacatgtatgaaaatatcatatgtatctcataaacatgtataattattatgtatcaattatcaattaaaatttgaaaaaaaagaaagcggttggaggatcattcctctcccagatgttaaactgtttccccaagggtctttgggtggatagggaccatggtgttcatttcatccctaaaatacaagagtttacagagcctgagtaaaatccagtagattgatttagtcaaggcccagctttggcatggcaaggttaggggtgaatagtaattaccagcttcctagagataccttctcctgtctccctgctcaatagcagcaagatacatcaaatatacaatacgttgtttctctccacttttttctctcctctcacaaatatctcttaagagttatctttaaatgaagtaccactctgttttccaattttctttcttcggtagaaacaagacctttataacctgttccttattacaagcacataaacaaacaaacccaaatttggtgttctcttttttttactcttctttattttcttctctgttcttttattctttttctcttttcagaattactctatcagtgatgggctttctagacctctcttggtatctcagcgtagagagacagagagacaaaataatcagtaaaccaggctagggctggaggtcaaaatacaagtaaaggtcaatgaaaactgccccctccccacattggcagatctaagggctctcatttctgtcatgccagcccaccagtttcccccaaaccaaagcagatagttcataaaaagcaaaaaatccccttgtctgataaacagcacttacatccgcctgccaaatggaaaggttaaaccacagtctgggccccagactctgagatcctgcagtggggcctgcctgtctgctagggatgatcctctacaaagtccctgggtcctcacttcctttaagcaacaatttggccatcttgaacaaacccaccaccagtctgaagccacatgccctaggattcccttgcccccataactgcacattatccttcatcatcaaatattgttcatctgtccgcggttgcagttgaagttgggaaatgaaagacaagtagaagaactcaatttagcaacatcctttccttcccatgccagtgtatacttgctcttggctgaatctgcagagccagggcacagcgagaaatagctctgatgccatctttccactctcctccacagtctcctctcccacagtgggcaagccctacaagtgtaactactgtggccggagctacaaacagcagagtaccctggaggagcacaaggagcggtgccataactacctacagagtctcagcactgaagcccaagctttggctggccaaccaggtagggctattgatgtactactggggagttaaaaggggatggtgagagggagtgctagacaagggtgactccagtatctcccacactgagggatgaagtctgtggaacaggggatgattaccccagtaaatctgccaaacaccccattctaggtaacccagttggtggagagccttgtctgtggtgggtgctttagggagatcgacaggtaggggaagtttgggtctatatccttggcctaatgggagagagagaacacaagaagtataaaaccaagggccagatgtgtatgattgtccccaaatgatctgaaattttattggagtgagctttaggtagggtaaacagagcccagtttcccaggcaactggtatgtccttccttggctactgacctgctggttttctccaggcaaaggctgtggggccaatgctacccctactcaactgctactactgtctccaccttcttcccactaggtgacgaaatacgtgacctggagatggtgccagactccatgctgcactcatcctctgagcggccaactttcatcgatcgtctggccaatagcctcaccaaacgcaagcgttccacaccccagaagtttgtaggtaagaatccagttggaaagacgtatcagctttaagccacagctcaaataaatctgagctgtgtggtctggagattcggtttggggaaagaaagcagtctggtgggctgggtgcagtggctcacgtctgtaatcccagcactttgggaggccaaggtgggcagatcaagaggtcagaagatcaagaccatcccggttaacacagtgaaaccccctctctactaaaaatacaaaaaattagccgggcgtggtggcgggcacctgtagtcccagctactcgggaggctgaagcaggagaatggtgtgaacctgggaggcggagcttgcagtgagcggagatcgcgccactgcactccatccatcctgggcaacagagcaagactccgtctcaaaagaaaaaagaaaaaaggaaagaaagaaagcaggctggtggcctctgatactgaaaatctatcctcaggacaggaggaaagtaaagagggaggatgtactcaaagagagtggagaagggttggcactgaagactctcgtggtggttgtaagggacagggtcgaggcatcgggctgtctcagttttttgtttgtttgtttgtttttgagacggagtcttgctctgtcgcccaggctagaatgcagtggcacgatctcggctcactgcaacctctgtctcccaggttcacgccattctcctgcctcagcctcccgagtagctgggactacagacgcctgccaccatgcccggctaattttttttgtatttttagtagagacggggtttcaccgtgttagccaggatggtcttaatctcctgacctcatgatctgcccgcctcagcctcccaaagtgctgggattacaggcgtgagccaccgcgcccggcccgggctatctcagtttttaagaataacaagaacttcagtctgtgtgggcccttagagagtcagagagaggaaataagtatgtggttccccctatccccagcactagtcatgagctctctaaggacctggagctttatggagagtaataaaatgaagtattaaaaatcaatgggctgggattgagtccttctctaagattatagcctagagtcagggagcagtcccacaaaggacctgaccctgcgaaagtaaatgccacgtagtaaataatgttacacccagccccacaggtaaaaaaacaaagaagtaatcctgaggagagtgtaggaattctccaaacccagccctgctgagttcctgttctccacaggcgaaaagcagatgcgcttcagcctctcagacctcccctatgatgtgaactcgggtggctatgaaaaggatgtggagttggtggcacaccacagcctagagcctggctttggaagttccctggcctttgtgggtgcagagcatctgcgtcccctccgccttccacccaccaattgcatctcagaactcacgcctgtcatcagctctgtctacacccagatgcagcccctccctggtcgactggagcttccaggatcccgagaagcaggtgagggacctgaggacctggctgatggaggtcccctcctctaccggccccgaggccccctgactgaccctggggcatcccccagcaatggctgccaggactccacagacacagaaagcaaccacgaagatcgggttgcgggggtggtatccctccctcagggtcccccaccccagccacctcccaccattgtggtgggccggcacagtcctgcctacgccaaagaggaccccaagccacaggaggggttattgcggggcaccccaggcccctccaaggaagtgcttcgggtggtgggcgagagtggtgagcctgtgaaggccttcaagtgtgagcactgccgtatcctcttcctggaccacgtcatgttcactatccacatgggctgccatggcttcagagacccttttgagtgcaacatctgtggttatcacagccaggaccggtacgaattctcttcccacattgtccggggggagcataaggtgggctagcaacctctccctctctcctcagtccaccactccactgccctgactacaggcattgatccctgtccccaccatttcccaaggagttttgctttgtagccctcactactggccacctgacctcacacctgaccctgacccctcctcacctattctcttcctctatcctgaccgatgtaagcattgtgatgaaacagatcttttgcttatgtttttcctttttatcttctctcatcccagcatactgagttatttattaattagttgatttatttttgcctttttaaattttaacttatatcagtcacttgccactcccccaccctcctgtccacaactcctttccactttaggccaatttttctctcttagatcttccagcagccccaggggtaggaagctcctcttagtactaagagacttcaagcttcttgctttaagtcctcaccctttacattatctaattcttcagttttgatgctgatacctgcccccggccctaccttagctctgtggcattatatctcctctctgggactcttcaacctggtactccatacctcttgtgccctctcactttaggcagcttgcactattcttgaatgaatgaagaattatttcctcatttggaagtaggagggactgaagaaattctccccaggcactgtgggactgagagtcctattcccctagtaataggtcatattcccctagtaatatgagttctcaaagcctacattcaggatctccctctaggatgtgatagatctggtccctctccttgaactacccctccacacgctctagtcccttcaacctaccggtctattaagtggtggcttttctctccttggagtgccccaattttatattctcaggggccaaggctaggtctgcaaccctctgtctctgacagattgggagccacaggtgcctaattgggaaccagggcatgggaaaggagtgggtcaaaattcttctctttctcctccacctctcaaacttcttcactatagtgaccttcctaggctctcaggggctccttcagtccccatcctatgagaaactagtgggttgctgcctgatgacaaggggttgtttcagcccctcagtcatgctgccttctgctgctccctcccagcaggattcaccctctcattcccgggctcctgggccctgttcttaggatcagtggcagggagaaacgggtatctcttttctctcttctaattttcagtataaccaaaaattatcccagcatgagcacgggcacgtgcccttcaccccattccacccttgttccagcaagactgggatgggtacaactgaactggggtcttcctttactacccccttctacactcagctcccagacacagggtaggaggggggactgctggctactgcagagacccttggctatttgagtaacctaggattagtgagaaggggcagaaggagatacaactccactgcaagtggaggtttctttctacaagagttttctgcccaaggccacagccatcccactctctgcttccttgagattcaaaccaaaggctgtttttctatgtttaaagaaaaaaaaaagtaaaaaccaaacacaacacctcacaagttgtaactcttggtccttctctctctccttttctcttcccttccttccccttccatctttctttccacatgtcctttccttattggctcttttacctcctacttttctcactccctatcagggatattttggggggggatggtaaagggtgggctaaggaacagaccctgggattagggccttaagggctctgagaggagtctaccttgccttcttatgggaagggagaccctaaaaaactttctcctctttgtcctcctttttctcccccactctgaggtttccccaagagaaccagattggcagggagaagcattgtggggcaattgttcctccttgacaatgtagcaataaatagatgctgccaagggcagaaaatggggaggttagctcagagcagagtagtctctagagaaaggaagaatcctcaacggcaccctggggtgctagctcctttttagaatgtcagcagagctgagattaatatctgggcttttcctgaactattctggttattgagcccttcctgttagacctaccgcctcccacctcttctgtgtctgctgtgtatttggtgacacttcataaggactagtcccttctggggtatcagagccttagggtgcccccatccccttccccagtcaactgtggcacctgtaacctcccggaacatgaaggactatgctctgaggctatactctgtgcccatgagagcagagactggaagggcaagaccaggtgctaaggaggggagagggggcatcctgtctctctccagaccatcactgcactttaaccagggtcttaggtacaaaatcctacttttcagagccttccagctctggaacctcaaacatcctcatgctctctcccagctccttttgcataaaaaaaaaagtaaagaaaaagaaaaaaaaatacacacacactgaaacccacatggagaaaagaggtgtttccttttatattgctattcaaaatcaataccaccaacaaaatatttctaagtagacacttttccagacctttgtttttttgtgtcagtgtccaagctgcagataggattttgtaatacttctggcagcttctttccttgtgtacataatatatatatatacatatatatatatatttttaatcagaagttatgaagaacaaaaagaaaaaataaacacagaagcaagtgcaataccacctctcttctccctctctcctagggtttcctttgtagcctatgtttggtgtctcttttgacctttaccccttcacctcctcctctcttcttctgattcccctcccccccttttttaaagagtttttctcctttctcaaggggagttaaactagcttttgagacttattgcaaagcattttgtatatgtaatatattgtaagtaaatatttgtgtaacggagatatactactgtaagttttgtactgtactggctgaaagtctgttataaataaacatgagtaatttaacacctctggttgtttttgcagacttcctttgccttgcttttctactaagagggaggggtcctccttgcagtatgttataccaactactgaacagtttaggaggtaaacaaaaacgaagaattttcagcctagactgggaaatggaggagattttcctaccaggaaggagtgccttcaggataggagagaaagtctgacttgcctgtctacccaaagtagaaattaatcaaacatttgttgagtacctactgtgcacaaaacacattctatgcccagggtgaaataatgcgaatgatgacaatcatagggtctaccactgcttgagcatctcataaatgctaagtactcattgaattgtagaaacacctacaaaaggatgatcacaattattggtaaaaaaataaaagcccaagagagaataaataccttatccaaggctcatcagccagtaagtgacagaaccagaatttgaacccaggggtgtgtgatccaaagcatgtgctcatcgctttatgccagggaatgcagccagtgaaacccctaagctagaaaaatccaaagtaggaacataaatttgtttctgtcttaaatagtaactattttc

SEQ ID NO: 5 - RNA, 20 nt, synthetic construct
  source 1..20: mol_type = other RNA
tgtaagcgatactccagatg

SEQ ID NO: 6 - RNA, 20 nt, synthetic construct
  source 1..20: mol_type = other RNA
caacgccatcaactacctgg

SEQ ID NO: 7 - RNA, 20 nt, synthetic construct
  source 1..20: mol_type = other RNA
gatactaccaaggctcctaa

SEQ ID NO: 8 - RNA, 20 nt, synthetic construct
  source 1..20: mol_type = other RNA
aaactgaaatgtgacgtctg

SEQ ID NO: 9 - RNA, 20 nt, synthetic construct
  source 1..20: mol_type = other RNA
acatcacatagtccaggaag

SEQ ID NO: 10 - RNA, 20 nt, synthetic construct
  source 1..20: mol_type = other RNA
gttttaaagtcagaacccat

SEQ ID NO: 11 - RNA, 20 nt, synthetic construct
  source 1..20: mol_type = other RNA
ggtggagatgtacagcgatg

SEQ ID NO: 12 - RNA, 20 nt, synthetic construct
  source 1..20: mol_type = other RNA
cccttccattgcaaccagtg